(19) United States
(12) Patent Application Publication
(10) Pub. No.: US 2026/0315873 A1
(43) Pub. Date: Oct. 15, 2026

(54) NEURO-MODULATION THERAPY METHOD, SYSTEM, AND APPARATUS

(71) Applicant: NEUMO TECH PTY LTD, Payneham (AU)

(72) Inventors: Lyndon Matthew HUF, Highgate (AU); Peter Graham FOSTER, Hyde Park (AU); Alexander William WALKER, Henley Beach (AU)

(21) Appl. No.: 19/485,273

(22) PCT Filed: May 17, 2024
(86) PCT No.: PCT/AU2024/050503

§ 371 Date: Nov. 17, 2025

(30) Foreign Application Priority Data
May 17, 2023 (AU) 2023901520

(51) Int. Cl. A63B 24/00

(52) U.S. Cl. CPC A63B 24/0075; A63B 2024/0068; A63B 2024/0078; A63B 2220/40; A63B 2220/51; A63B 2220/58; A63B 2225/50

(57) ABSTRACT

Methods, apparatus and systems for providing Neuro-Modulation Therapy are described in which a user is instructed to perform multiple exercises involving a target musculoskeletal joint. A target force level is displayed to the user who is instructed to match the target whilst performing the exercise. The exercise may be repeated actions involving the target joint such that the target is a sinusoid like curve. In some exercises part of the curve is suppressed or hidden for at least a portion of the exercise requiring the user to estimate the amount of force required to achieve the (hidden) target level. The actual force is measured and compared with the target, and feedback on the deviation between target and actual force is provided to the user. Over a series of sessions the amount of suppression and complexity of suppression may also be increased over time.

The present application claims priority from Australian Provisional Patent Application No. 2023901520 titled “NEURO-MODULATION THERAPY METHOD, SYSTEM, AND APPARATUS” and filed on 17 May 2023, the content of which is hereby incorporated by reference in its entirety.

### TECHNICAL FIELD

The present disclosure relates to rehabilitation systems after a musculoskeletal injury and/or surgical intervention. In a particular form the present disclosure relates to methods, system and apparatuses for assessing neuromuscular skeletal control of a joint or joints and subsequent application to rehabilitation programmes for improving neuromuscular control after musculoskeletal injury and/or surgical intervention.

### BACKGROUND

Due to a range of causes such as ageing and traumatic injuries the functioning of musculoskeletal joints often degrades, and thus surgical interventions and/or rehabilitation programs are often prescribed in an attempt to restore function to the impacted musculoskeletal joints.

Control of a musculoskeletal joint typically involves a multi-system combination of the neurological and the musculoskeletal systems where the nervous system provides both control and sensing/feedback (often using specialised cells) of musculoskeletal components such as muscles, tendons, ligaments and joints. Muscle, joint, ligament and tendon injuries, surgeries, lesions, insults, or neuromuscular diseases typically lead to an impairment or reduction in function, including specific timing and coordination of motor skills, leading to a loss of control of a musculoskeletal joint or joints, and subsequent impact on activities of daily living. Joint control, or in fact any muscular control, is a complicated process which is simplistically thought of as a command message from the brain to the muscles via the central and peripheral nervous systems. This is only part of the story. Consider the act of picking up an egg. A complicated feedback process occurs in order to ensure that you grasp the egg with just enough force to create the friction required to pick it up; without crushing it. A command signal is sent from the brain to the muscles in the hand that causes the fingers to press on the egg. Sensory receptors in your skin respond to mechanical pressure that has been exerted by the muscles and send feedback via action potential signals back to the brain where, through learnt experiences, a sub-conscious, closed-loop process controls muscle contraction and applies just the right pressure to the egg.

This is a finely tuned closed loop process whereby the command system is constantly modulated by the feedback system. Within the command system of the central nervous system, an excitatory and inhibitory balance exists that has an overlying effect on cortical drive and hence controlling the desired level of motor output. This ensures a particular action is performed optimally every time. If there is an error in the feedback modulation system the egg is either held too lightly, in which case it would slip from your fingers, or it is crushed. This example demonstrates that for the execution of voluntary, smooth, coordinated movement both the command and feedback modulation systems need to be functioning properly.

It can therefore be said that the smoothness of human movement is an indication of a healthy, functioning nervous system and conversely poorly controlled jerky movement can be viewed otherwise. Injury (including without limitation ligament, cartilage, muscle, tendon, bone injury, degenerative conditions such as osteoarthritis, Parkinson's disease, stroke patients and even mild cognitive impairment) and surgical intervention have been shown to exhibit an increase in the jerkiness of movement and longer times to complete standard movement tasks—and throughout this disclosure injury and surgical intervention should be considered to be mutually interchangeable. Smoothness may be measured under the guise of neuromuscular control. Neuromuscular Control can be defined in one instance as applying the right force, to the right action at the right time.

Loss of control impacts both the gross and fine motor skills. A loss of gross motor control, which would include a limp, has severe consequences for daily life whereas loss of fine motor control may be imperceptible in daily life yet amount to a barrier preventing a high-performance athlete reaching the pinnacle of the elite level, relegating them to the ranks of mere mediocre athletes.

At the highest level of human performance, minor or small losses of control significantly impacts the execution of highly skilled functions where goal orientated accuracy is important. A small loss of control creates an undesired outcome where unwanted variation or error in motor skill execution renders a poor, undesired outcome. This is of particular importance where a small loss of control in one joint directly impacts a subsequent joint or joints causing a compounding adverse effect. Under these circumstances the performer would try to correct or right the adverse adaption to execute a goal orientated task such as accuracy. This is all happening under a constantly changing, dynamic environment where timing and coordination are crucial variables. Where the body is an interconnected system of joints with multiplanar degrees of freedom, the adverse effects can be dramatic. A minor loss of control can be easily understood when a novice person playing golf mis-hits a golf ball due to a loss of control in a joint or joints within the lower part of the body, only to try to correct the swing with the arms, striking the ground instead and feels the immediate painful effect on a body part from the sudden impact the ground has on the body.

There is currently no reliable way of quantifying neuromuscular control, and in which joints an impediment may exist, thus clinicians typically perform subjective examination of such impediments to determine the severity of the loss of control of musculoskeletal joint(s) or components. This is often a visual observation of a patient performing a task while focusing attention to a joint or joints, limb or body part and overall perspective. For example, often the clinician visually observes the amount of lateral deviation a knee joint may exhibit while performing a balance exercise, graded as minor, moderate or severe loss of control. Clinicians then use that subjective information as the basis to develop or assess a rehabilitation program. The subjective and gross information lacks granularity and measurement accuracy to effectively characterise the loss of control or determine if the rehabilitation intervention is indeed working. As a consequence, most rehabilitation programs, including post-surgical rehabilitation programs, focus on building strength in the musculoskeletal components around the injury site. This problem is widespread and causes patient frustration due to lack of progress. Also, the economic cost of utilising traditional obsolete assessments and subsequent conventional rehabilitation management demands advances in efficacious outcomes.

Measuring a neuromuscular impediment has traditionally involved a measure of maximum strength as an indication of the ability or capacity to perform a function. Insufficient strength has traditionally been used as a primary marker to indicate a lack of recovery when comparing to an opposite unaffected limb or normalised data. Accordingly, many rehabilitation programs focus on building strength in the musculoskeletal components around the injury site with a typical treatment regime revolving around strength exercises and quantification of the strength of an injured (or replaced) joint with respect to the respective healthy collateral joint. For example, a typical Total Knee Arthroplasty (TKA, also known as total knee replacement) rehabilitation programme looks like the following:

Days 1-14: Cryotherapy after exercises; circumferential compression dressing from ankle to thigh; elevation of the affected limb to minimise swelling. Range of Motion/Strengthening Exercises: Quadriceps and gluteal sets; Straight leg raises, supine; Knee extensions supine over a roll; Knee extensions from seated position; Passive knee straightening with a heel roll, supine; Heel slides, seated and supine.

Week 3-6: Range of Motion/Strengthening Exercises: Isometric quads, hamstrings, gluteals, adductors; Core stabilising exercises; Active and assisted range of motion exercises; Supported standing heel raises, calf stretches, mini squats, hamstring curls; Hydrotherapy.

Weeks 7-12: Range of Motion/Strengthening Exercises: Core stabilisation exercises; Squats and single leg stance mini-squats; Resistance exercises for quadriceps, hamstrings, gluteals and adductors; Active and assisted range of movement exercises.

As can be seen, the typical programme revolves around strengthening quadriceps, hamstrings and gluteals with lip service paid to core strengthening exercises and no quantification of progress. Strength alone does not improve control and typically there is no requirement or attempt to measure or quantify joint control. A patient recovering from a knee injury or total knee replacement (even someone at the end of TKA rehabilitation) who tries to make small, smooth, controlled movements against a resistance force will, more often than not, produce jerky motion rather than the desired smooth motion. Improving maximum strength alone will not help them navigate stairs or impart the small, controlled forces required on the accelerator pedal of a car. Thus, despite the best efforts of surgeons, physiotherapists and other clinicians, many patients are dissatisfied with their new knee replacement. From a functional perspective, it is common for patients to report their knee feeling unstable during common activities such as traversing stairs. A 2022 study by Marsh et al (Marsh et al. Health care costs after total knee arthroplasty for satisfied and dissatisfied patients Can J Surg 2022 65(5): E562-E566) noted that over 36% of recipients were dissatisfied with function. On average, these dissatisfied patients also accounted for over $5,483 of extra incurred cost following knee replacement over and above the mean cost of $13,523 with satisfied patients.

Instability in flexion is often underestimated and has not received attention on the rehabilitation side due to the continued focus on strength. It is common for patients to report that instability is greatest at the mid-flexion point. A common functional task for which patients report instability is traversing stairs where their ability to control load at mid flexion is tested. In some instances, the unstable knee may dislocate and be associated with subsequent fractures, requiring more complex revision surgery. Mid-flexion knee instability has received much attention from surgeons about how best to manage this through surgical technique and implant selection. Notably there have been significant advances in the design of knee implants which now replicate as close as possible the natural knee. However, despite all of the advances, patient reported knee instability is still common and instability after total knee replacement is still a cause of revision surgery. Additionally, patients have an average of 60% decline in quadriceps strength following surgery and 50% of patients experience quadriceps muscle wasting 1 year after surgery. Further complicating recovery and rehabilitation is that recipients of total knee replacements are often in the final stages of severe osteoarthritis and have been poorly functioning with a diseased knee for years. The diseased knee joint space is narrowed and mal-aligned, leading the adjacent supporting ligaments to be lax and offering little structural support. The diseased knee is painful and often swollen, compounding the already altered biomechanics and patients generally walk with an antalgic gait. This poor initial state thus creates further rehabilitation challenges.

The current focus on restoring maximum strength is thus not generating the desired recovery, particularly for such patients. Even when a patient regains full strength in the injured area, they frequently suffer further injuries at or adjacent to the original site indicating that a full recovery has not occurred. Such subsequent injuries to adjacent or associated body parts may indicate that the insufficient recovery of the initial injury site has transposed an effect to other connected areas of the body. This phenomenon is often observed in professional/high performing athletes, who return to competition following an initial injury, only to suffer a re-occurrence of the injury or a further injury at a related site due to incomplete recovery of the original injury. Knee injury is especially susceptible to recurrence.

Similar issues are observed in the recovery from a rupture of the anterior cruciate ligament (ACL). The ACL is one of four major ligaments in the knee and connects the femur and the tibia, acting as a stabilising structure to restrain excessive anterior translation of the tibia in relation to the femur. This ligament is often injured in sport when the foot is planted on the ground and knee is in a position of mild flexion while experiencing a knee valgus moment. Under extreme loading the hamstring muscles are unable to restrain the anterior translation of the tibia and sudden rupture of the ACL can occur. Rupture of the ACL thus destabilises the knee creating joint laxity and functional instability. Further the ACL is rarely injured in isolation with damage occurring to other joint structures such as the meniscus, other ligaments, joint cartilage, bone and capsule, among others, resulting in pain and swelling.

Musculoskeletal injuries such as those to the ACL are often treated by clinicians as injuries with solely musculoskeletal consequences. One surgical treatment is ACL reconstruction in which a tendon autograft is surgically implanted in an effort to restore mechanical stability and therapeutic rehabilitation is then performed to increase strength of the musculature surrounding the knee to try and restore the previous natural ACL function. A conventional rehabilitation programme typically includes exercises to strengthen the musculature, commonly comprising resistance strength training. Despite these invasive and timely interventions, deficits in clinically meaningful measures such as quadriceps strength and patient-reported function, as well as altered mechanical loading at the knee joint, persist for years, if not indefinitely, following surgery. It is also noted that biomechanical adaptations are prevalent even after surgical interventions leading to altered and abnormal joint loading patterns of the knee as well as adjacent joints that even extend to the contralateral side. For example, surgery alone creates a new series of issues for the already damaged knee, not limited to lesions to the skin, capsule, graft sites to harvest suitable tendon for reconstruction, blood vessels, nerves, drilling into the bone and debriding or the removal of associated damaged tissue from the initial or subsequent mal-adaptive injury. Exact surgical placement of the tendon autograft to fulfil the previous natural ACL function is also highly unlikely making the morphology of the joint permanently different from the pre-injury state and thus creating further rehabilitation challenges. Over time this abnormal joint loading can lead to early onset osteoarthritis within 10-15 years after injury or surgery. Quality of life is compromised due to limitations in daily activities. Further, despite the lengthy list of challenges facing ACL rehabilitation, there is no standardised rehabilitation protocol, nor is there a standard objective test to indicate rehabilitation recovery or progression. Conventional rehabilitation programmes typically include exercises to strengthen the musculature, commonly comprising resistance strength training. However, strength is a poor proxy for the progress of rehabilitation. This is symptomatic of all joint injuries and despite extensive efforts, such surgical interventions and rehabilitation programs often fail to fully restore musculoskeletal joint function. Not only is the ACL reconstructive surgery an invasive option, it also a costly intervention that can take 12 months of lengthy rehabilitation to recover to the point of an athlete returning to competitive sport. However, even then recovery is not complete or guaranteed. In a recent unpublished study conducted by the inventors, it was identified that professional athletes who had ACL injuries, had then completed the entirety of their post-surgical rehabilitation and were back playing professionally, could only voluntarily activate their quadriceps strength to 87% of their non-injured side. This was despite a rigorous rehabilitation programme and extensive strength training by a group of highly motivated patients. This demonstrates that despite the world-class rehabilitation being available to professional athletes not even they can attain acceptable levels of recovery (taken to be 95% or higher). Something is seriously absent in rehabilitation despite decades of research and effort by all involved.

In addition to the physical damage that occurs to the various structures in a joint during an injury and/or surgery, other problems may arise in essentially undamaged tissue such as muscle such as Arthrogenic Muscle Inhibition (AMI). AMI is a persistent neural inhibition of uninjured musculature after distension or damage to a joint or other forms of injury or surgical intervention. For example, following an ACL injury and after the immediate inflammation has subsided, it is often the case that quadriceps muscle strength decreases from baseline before the injury, and this reduced strength can persist for years, if not indefinitely, following surgery. Likewise, subsequent hamstring weakness is also common with this type of injury despite these muscles suffering no direct physical trauma. Less common, quadriceps and hamstring strength reductions can also present in the contralateral limb following these injuries, indicating reduced cortical drive via central mechanisms may play a role in the overall reduction in muscle strength from baseline. It is thought that this inhibition is a natural mechanism designed to protect an injured joint via normal modulation within the Central Nervous System (CNS) that exhibits controls via an excitatory and inhibitory balance, managing cortical drive and motor output. However, AMI can become a mal-adaptive response persisting long after the physical injury has healed. Volitionary muscle activity can be severely reduced with co-contraction of opposing muscles around a joint causing dynamic joint stiffness and reducing fine motor control; significantly limiting rehabilitation progress and hindering recovery. Increases in the GABAergic inhibitory interneuron activity release neurotransmitter gamma-aminobutyric acid (GABA) that overregulates the excitatory system and prevents action potential firing. The result is decreased motor output to the muscles, presenting as muscle weakness and atrophy typical of that seen in AMI.

Knee joint effusion models have been shown to cause quadriceps inhibition in the absence of pain. Quadriceps inhibition has been reported after an injection of as little as 10 mL of saline. Furthermore, a linear relationship between inhibition and joint pressure caused by saline injection has been demonstrated. Something is clearly happening to inhibit movement and protect the joint regardless of the presence of pain.

Whilst injury and subsequent surgery can cause a range of issues including potential structural degradation and surrounding tissue lesions, not all of the issues are purely mechanical in nature. Deafferentation of the nerve cells involves an interruption and destruction of the ascending and descending pathways to and from the spinal cord and brain. Interestingly, animal studies have shown once the ACL equivalent has been severed, an unexpected hyperexcitability exists within the nerves of the joint and surrounding muscles. Despite ACL reconstruction in these animals the electrical activity of the nerves fails to return to baseline measures, indicating that the ACL itself plays some unknown role in the nervous system baseline activity of the joint. Disrupting the healthy sensory input cascades into a protective state, the mechanisms of which are unresolved in the prior art. Further, this highlights that mechanical restoration of the joint through surgery isn't a complete and final solution. The anterior cruciate ligament impact on proper function is supported with Functional Magnetic Resonance Imaging (fMRI) studies which have shown differences between ACL reconstruction patients and normal controls. Most importantly, ACL reconstruction patients exhibit greater activity in the frontal lobe, vision, somatosensory and cerebellum despite decreased motor output from the cortex to the target site. The mechanism is derived from deafferentation, pain and swelling of the joint, altering the afferent information. This in-turn changes the somatosensory cortex due to the impaired sensory processing. A smaller representation of the knee of the somatosensory cortex has been observed after knee surgery, indicating a reduction in sensory input from the knee that has been adaptively mapped. An anterior shift of the knee representation on the homunculus has also been reported, indicating a new adaptive motor skill maybe in development. The central nervous system is in a state of flux in its plastic response to injury and/or surgery. An increased reliance on the cortical areas devotes increased resources to neurocognition, motor planning, motor inhibition and vision than otherwise would be the case in a healthy knee joint. This increases cognitive load and results in decreased descending motor output. Decreased corticospinal excitability and motor cortex activation results as well as increased intracortical inhibition.

All of these mal-adaptions can be summed up as arthrogenic muscle inhibition and lead to the reduced fine, or even gross, motor control reported by patients suffering joint trauma. At the neurological level, excitability was found to be reduced. Specifically, altered afferent signalling coming from an injured joint can result in a neuronal inhibition and a reduction in the motor neuron pool serving a muscle surrounding the joint, thus confirming that neurological mechanisms contribute to AMI. This reduction in motor output was present concurrently with clinically observed decreases in electromyographic activity, central activation ratio and force production. Together, these alter joint biomechanics during functional tasks including fine motor control, demonstrating the wide-ranging effect of reduced motor output arising from joint injury.

Although afferent input can influence reflexive motor activity at the spinal cord level, afferent sensory neurons arising from joint mechanoreceptors, proprioceptors and nociceptors also ascend the spinal cord. These neurons ultimately synapse in the thalamus or somatosensory cortex of the brain where neurological signalling is processed by the central nervous system and has the ability to influence behaviour and efferent motor output. Thus, the exploration of brain activity in response to joint injury has continued to illuminate our understanding of how altered afferent signalling from an injured joint influences somatosensory function. Studies using electroencephalography have confirmed that there is an absence of somatosensory evoked potentials following joint injury, like ACL rupture, indicating that joint injury results in a lack of neurological communication between the ligament and the somatosensory cortex, likely due to deafferentation. Although it is plausible for some reinnervation and new mechanoreceptor connections to the somatosensory cortex following injury, it has been shown that these new connections are significantly impaired and likely do not result in the same neural signalling that was present prior to injury.

Interestingly, disuse of muscle groups has been shown to negatively influence the nervous system in many of the same ways as the present discussion on AMI, leading to reduced muscle activation. In both cases there is a diminished area of motor cortex size, greater levels of intracortical inhibition and spinal-reflexive excitability are increased. These factors combine to produce reductions in muscle strength, size, timing and co-activation of the agonist/antagonist pairs. For example, gait changes are observed with quadriceps/hamstrings co-activation, causing dynamic joint stiffness impeding optimal function. It is also possible that co-activation may increase joint contact forces and precipitate degeneration. A healthy individual, in the presence of muscle weakness and atrophy due to disuse, can typically engage in resistance training to improve muscle strength and muscle mass and improve neuromuscular outcomes. Unfortunately, muscle atrophy in the presence of AMI does not respond to traditional therapeutic exercise in the same way as traditional disuse muscle atrophy and thus it is not surprising that post-surgical rehabilitation programs fail.

AMI thus accounts for a significant number of the issues associated with injury rehabilitation. Therapeutic exercises are therefore not fully effective in conditions where the motor neuron pool is inhibited, and AMI is largely unresponsive to traditional strength based rehabilitation. Neural inhibition creates many rehabilitation problems for the patient, leading to muscle weakness, atrophy and decreased neuromuscular control, and accordingly poor patient outcomes. To date, an appropriate treatment intervention for AMI is evading researchers.

Many interventions have been attempted to counter the effects of AMI. These local knee modalities include ice for joint cooling, Transcutaneous Electrical Nerve Stimulation (TENS), Neuromuscular Electrical Stimulation (NMES), blood flow restriction and eccentric exercises of the uninvolved limb. As previously stated, traditional rehabilitation exercises are ineffective in combatting AMI. A 2019 literature review of AMI following ACL reconstruction was performed by Sonnery-Cottet et al (Sonnery-Cottet et al. Arthrogenic muscle inhibition after ACL reconstruction: a scoping review of the efficacy of interventions Br J Sports Med 2019 53: 289-298). In the only randomised clinical study of TENS in patients with ACL rupture cited by Sonnery-Cottet, there was no difference in isometric strength and quadriceps central activation ratio among three groups (exercise only; exercise and TENS; 20 min of cryotherapy immediately prior to each exercise session). While all groups demonstrated a significant improvement in quadriceps strength and effect sizes suggested potential clinical benefit to patients with AMI, the disinhibitory modalities were no better than exercise alone. In laboratory-based randomised trials, TENS disinhibited the quadriceps motor neuron pool during the treatment, but its beneficial effects were lost 30 min after the cessation of treatment.

Traditional therapeutic approaches such as strength-based rehabilitation programs are largely ineffective for patients with these impairments, perpetuating poor clinical outcomes, and thus the conventional long term prognosis for AMI patients is unfortunately poor. As discussed in a podcast recorded in April 2022, Lindsey Lepley, a leading researcher in the field of AMI, due to the various challenges in treating patients with AMI, the AMI research community is currently focused on using very high levels of electrical stimulation on the local area, at and beyond the pain threshold of the patient. This is a return to the failed techniques used in the 1980s because all other conventional avenues for treating AMI have been exhausted.

Thus, to summarise, conventional rehabilitation still fails a large proportion of patients and fails to fully restore neuromuscular control leading to ongoing complications and poor outcomes including revisionary surgery and reoccurrence. Further, many patients acquire AMI after injuries for which there are currently no effective treatments. There is thus a need to provide methods, systems and apparatuses for assessing and quantifying neuromuscular control to allow the development of more effective treatment and rehabilitation programmes, or to at least provide a useful alternative to existing methods, apparatus and systems.

### SUMMARY

Embodiments of methods, systems and apparatus for performing Neuro-Modulation Therapy, and associated assessment of neuromuscular control of a musculoskeletal joint, will now be described with reference to the figures. Neuro-Modulation Therapy, which for convenience may be abbreviated to NMT, covers a range of applications. NMT may be used as a treatment, or it may be used to assess the progress of a treatment. This includes rehabilitation therapies or applications where there has been damage and NMT is used to restore the injured joint or neuromuscular system to a former condition and level of function, or to at least improve from the injured state even if full functionality cannot be restored. NMT also broadly covers therapy to achieve a higher state of function that was previously held prior to the therapy. This is broader than rehabilitation and may be performed by a patient to manage a condition as well as by a person seeking to develop or enhance their capabilities. For example, a child with Cerebral Palsy may use NMT to help them manage their condition but would not strictly be classed as rehabilitation. A child or amateur athlete may also use embodiments of NMT described herein to accelerate the development of motor skill function, providing a more targeted, faster and better way of reaching a desired capability. Development applications may thus include performance optimisation or performance enhancement, for example by elite athletes, or other persons, who may use embodiments of NMT to perform highly relevant targeted motor skill training to fine tune their neuromuscular control of specific joints and neuromuscular systems to achieve a higher-probability of accuracy in an outcome-based task (i.e. to achieve optimum performance). It will thus be understood that methods of providing NMT, and associated systems and apparatus, as described herein may be used for a wide range of applications including rehabilitation, development, and performance optimisation applications. In the following discussion it is to be understood that the term NMT will be used broadly to refer to any of these applications.

Embodiments of the method maybe used to assess or determine the particular range of parameters such as exertion force, timing, coordination or particular movements/joint angles over which a person (e.g., a patient or athlete) has poor neuromuscular control and neuro modulation therapies comprising repeated exercises may then be used in an attempt to improve the neuromuscular control. NMT may be used for both medical and non-medical applications, and we will use terms such as the user, patient, person and athlete interchangeably to identify the person receiving the NMT or using an NMT apparatus. Repeated exercises, as described herein, may form the basis of rehabilitation programs, or equivalently rehabilitation methods or rehabilitation therapies, that is designed to retrain the brain to adapt to, or learn, the new signalling environment in order to restore control and functionality. That is, rather than focusing on building strength, these rehabilitation programs are configured to focus on the areas where the user has poor neuromuscular control and may provide improved assessment and/or enable the development of more targeted rehabilitation programs that are more likely to lead to an improved or complete recovery. Embodiments of these rehabilitation programs can be used for treating arthrogenic muscle inhibition, increasing recruitment of muscle motor units, reducing muscle fatigue (and the cognitive fatigue typically present with someone suffering the aforementioned issues), as well as optimising the performance of people requiring high levels of controls such as athletes, sports people, and dancers. Further embodiments of NMT may be used to assess the progression of neurodegenerative disorders such as Parkinson's disease, Duchenne Muscular Dystrophy (DMD), Multiple Sclerosis (MS), Amyotrophic Lateral Sclerosis (ALS) and many other debilitating conditions. The progression of such disorders can be tracked by performing assessments of neuromuscular control at different times, enabling assessment of the effects of treatments (including rehabilitation programs). Furthermore, Neuro-Modulation Therapy may be applicable for treatment of some neurodegenerative disorders to at least retard the progress of these conditions. Similarly, NMT may be used to assess whether the user is suffering from a mild cognitive impairment such as a concussion. Furthermore, embodiments of the method enable assessment of the extent to which the user is suffering cognitive impairment by comparing the differences in neuromuscular control obtained when the user is under a varying cognitive loading. Embodiments of systems and apparatus for implementing embodiments of NMT and assessing neuromuscular control are also described.

According to a first aspect there is provided a method of Neuro-Modulation Therapy (NMT) comprising:
instructing a user, and/or using an apparatus to guide a user, to perform a plurality of exercises involving at least one target musculoskeletal joint, wherein the plurality of exercises comprises one or more feedforward exercises;
providing a representation of one or more target parameters for at least one of the plurality of exercises wherein the at least one of the plurality of exercises includes at least one feedforward exercises, wherein the representation is provided for one or more representation time periods over a duration of the respective exercise, and during the respective feedforward exercise the representation is suppressed for at least a portion of the one or more representation time periods over the duration of the respective exercise;
measuring one or more target parameters for one or more capture time periods whilst the user is performing one or more of the plurality of exercises; and
providing a feedback representation to the user using the measured one or more target parameters.

That is each exercise has a duration which comprises one or more representation time periods. As discussed below these do not need to span the entire duration so that a particular portion of the exercise can be targeted. The representation time periods are the nominal time(s) when the representation is to be displayed subject to the requirement that when performing a feedforward exercise the representation is suppressed (e.g., hidden, occluded or not represented) during at least a portion of the representation time periods. That is during a feedforward exercise the representation will not be represented for the entire duration of the representation time periods and instead they represent the nominal times during which the target parameters may be represented. The suppression portion may be one or more suppression time periods, and these are a subset of the representation time periods. As discussed below the suppressed portion may be changed from exercise to exercise. The representation time periods can thus be defined as one or more time periods during the duration of an exercise when a representation of one or more target parameters may be provided. A feedforward exercise may be defined as an exercise during which a representation is provided for one or more target parameters where the representation is suppressed during one or more suppression time periods within one or more representation time periods. In the context of this specification feedforward is a label for a type of exercise and a feedforward exercise could equivalently be labelled a partially suppressed target representation exercise. The capture time periods are the time periods during which the target parameters are actually measured. These do not have to the same time periods as the representation time periods, although they may be the same. The capture time periods will typically include at least some of the suppression time periods or portions of the suppression time period.

In one form, the user is instructed to attempt to match the one or more target parameters in real time for the one or more representation time periods and each exercise comprises one or more actions, and each representation time period is the time to perform at least one complete action.

In one form, the plurality of exercises further comprises at least one feedback exercises, wherein during a feedback exercise the representation is provided for all of the one or more representation time periods over the duration of the respective exercise. That is in contrast to feedforward exercises there is no suppressed portion. In the context of this specification feedforward and feedback are labels for exercises which distinguish between whether suppression occurs or not during representation time periods. We could equivalently label feedback and feedforward exercises as target representation exercise and partially suppressed target representation exercises.

In one form, the method further comprises performing an assessment of the neuromuscular control of the at least one target musculoskeletal joint during one or more exercises of the plurality of exercises comprising:
determining one or more differences between the one or more target parameters and the respective measured parameter over the one or more capture time periods.

In one form, the assessment is used to trigger a change in the portion of the one or more representation time periods that the representation is suppressed during a feedforward exercise to change a phase, a duration or a complexity of the suppression of the representation.

In a further form, the assessment may comprise using the one or more differences to determine one or more accuracy measures, wherein the one or more accuracy measures are compared with one or more predefined trigger thresholds to determine when to trigger the change in the portion of the one or more representation time periods phase that the representation is suppressed.

In a further form the one or more differences are compared with one of more predefined difference thresholds, and the one or more accuracy measures are an estimate of the percentage of time during a respective exercise that the one or more differences are within one or more difference ranges, wherein the one or more difference ranges are defined by the one or more difference thresholds.

In a further form, the method further comprises:
generating and electronically reporting an assessment of the neuromuscular control of the at least one target musculoskeletal joint.

According to a second aspect there is provided a method of assessing neuromuscular control of at least one target musculoskeletal joint comprising:
instructing a user, and/or using an apparatus to guide a user, to perform one or more exercises involving at least one target musculoskeletal joint;
providing a representation of one or more target parameters for at least one of the one or more exercises during one or more representation time periods, wherein the user is instructed to attempt to match the target parameter in real time;
measuring the one or more target parameters as a function of time for one or more capture time periods whilst performing the one or more exercises; and
determining one or more differences between the one or more target parameters and the respective measured parameter over the one or more capture time periods;
generating and electronically reporting an assessment of the neuromuscular control of the at least one target musculoskeletal joint using the one or more differences.

In one form, the assessment may further comprise using the one or more differences to determine one or more of a measure of smoothness of a motion, an error summary, and a control summary comprising one or more ranges of the target parameter and/or one or more joint angle ranges where the user lacks control of the at least one target musculoskeletal joint.

In one form, determining one or more differences may comprise calculating one or more error measurements by comparing the one or more target parameters and the respective measured parameter over the one or more capture time periods, and the method further comprises performing a statistical analysis of the one or more errors to characterise where the user lacks control of the at least one target musculoskeletal joint.

In one form, each exercise comprises one or more actions wherein each action is either a dynamic action comprising activating one or more muscles associated with the at least one target musculoskeletal joint to move the at least one target musculoskeletal joint over one or more joint angle ranges or a static action comprising activating one or more muscles associated with the at least one target musculoskeletal joint whilst holding the at least one target musculoskeletal joint in a static position at a fixed joint angle.

In one form, the target parameter(s) is varied over at least one of the one or more representation time periods. This may either be during a single exercise, or over a plurality of coordinated exercises performed during the one or more representation time periods. In another form, the target parameter(s) is held constant during at least one of the one or more representation time periods. Again, this may be during a single exercise, or over a plurality of coordinated exercises performed during the one or more representation time periods. In one form, each exercise may comprises moving a plurality of musculoskeletal joints including at least the target musculoskeletal joint over a defined range of movement and/or activating a specific muscle or muscle group associated with the at least one target musculoskeletal joint.

In one form, a representation of the target parameter(s) as a function of time is provided to the user. This may be via one or more of a visual apparatus, an audible apparatus, and a haptic apparatus. This may provide a representation of the target parameter as a function of time during each exercise. The visual representation may be a target level, target curve, numerical value, a shape with a variable property such as size and or colour. The visual apparatus may be a chart, a slider, a gauge or other representation displayed on a computer monitor, a tablet screen, a cell phone screen, a wearable glasses screen or some other visual apparatus. An audible apparatus may be a speaker, such as speaker of a headphone, portable speaker, computing apparatus, tablet, or smart phone, such that an audible volume or pitch is used to represent the target parameter such that a change in volume or pitch is used to indicate to the user how close to the target parameter. A haptic apparatus may use a predefined strength and or frequency of haptic to represent the target parameter.

In one form the method may further comprise providing a representation of the measured parameter as a function of time (including in real-time) as a charted comparison against the target parameter or as an instantaneous measurement of the parameter either in isolation or overlaid on or with the visual representation of the target parameter to represent the difference between the target and measured value. In another form the difference between the target parameter and the measured parameter may by represented by an audible pitch or volume change, or change in frequency and/or amplitude of haptic feedback.

In one form, the target parameter(s) may vary predictably such as according to a periodic function such as a sinusoidal function, a sawtooth or triangular function, or polynomial function, a parametrical function or any other predictable way, or it may vary unpredictably. The target parameter(s) may vary predictably, and the representation is suppressed for one or more suppression (i.e. gap) time periods. In one form the feedback representation comprises a representation of the measured target parameter in real-time. The target parameter(s) may be displayed for a short time window in advance of a current time, or it may vary without the user's prior knowledge to test the user's ability to track the parameter and the response time. In the case of a visual representation, it may be partially or fully occluded (suppressed) from view. In the case of an audible target parameter there may be a gap in the audible output for audible target parameter presentation (i.e., suppression of the audio signal). In one form the one or more exercises comprises a plurality of exercises, and for each sequential exercise in the plurality of exercises the representation is suppressed according to a sequence of suppressions. The user may be instructed to perform each exercise separately and may also be instructed to perform other exercises i.e., the one or more exercises comprises the plurality of exercises following the suppression sequence, along with other exercises. Each suppression in the sequence may comprise a different time period. For example, the suppression time gap may be constant, but the location or phase range of the suppression varies. In one form the sequence of suppressions may be a progression of suppressions from none through to complete suppression in a predetermined sequence, or vice versa. That is, each suppression in the sequence of suppressions comprises a progressively longer suppression (or gap) time period or an increase in complexity, or vice versa. The partial suppression or progressions of suppression may be configured such that a future target value is shown after a suppressed (gap) time period so as to require the user to predict the amount of force required to meet the further target value. The plurality of exercises may be divided into a plurality of sessions each comprising one or more sets (or trials, or reps) of one or more exercises. Over time, the exercises in a session may transition from all or mostly feedback exercises (i.e., at least 80%) in a session to all or mostly feedforward exercises (i.e., at least 80%) in a session, and the amount of suppression and complexity of suppression in feedforward exercises is also increased over time, or vice versa. The amount of suppression and complexity of suppression increases until the representation of the target parameter is 100% suppressed leaving only a measured representation of the target parameter during the respective exercise, and in further exercises the measured representation is then also suppressed for the duration of the respective exercise. In one form, the representation is a sinusoidally varying force curve having a curve period, and increasing the amount of suppression and complexity of suppression in feedforward exercises over time comprises beginning with suppression of a central region of the sinusoid curve for a first time period, wherein the first time period is less than 20% of the curve period, followed by lengthening the first time period, followed by suppression of the turning points, followed by suppression of increasingly larger percentages of the curve time period until the curve is 100% suppressed. An increase in complexity may comprise selecting a period of time in which the target parameter transitions a turning point and/or subjecting the user to an increased cognitive load.

The plurality of exercises may be arranged as a program, such a rehabilitation program, treatment program or exercise program, in which the plurality of exercises are divided into a plurality of sessions where each session comprises one or more sets of one or more exercises. exercise During a reference session an assessment of neuromuscular control may be performed to determine one or more ranges of the target parameter and/or one or more joint angle ranges where the user lacks control of the at least one target musculoskeletal joint, and then in subsequent sessions the one or more sets of one or more exercises the user is instructed to perform, or is guided by an apparatus to perform, are each selected using the determined one or more ranges of the target parameter and/or one or more joint angle ranges where the user lacks control of the at least one target musculoskeletal joint. Further an assessment of the neuromuscular control of the at least one target musculoskeletal joint may be performed in a reference session and again in one or more subsequent sessions to assess one or more of a progress of a treatment, a progress of a rehabilitation program, a progress of a development program, a progress of a performance optimisation program, a measure of the progression of a neurological disorder and to assess whether the user is suffering from a mild cognitive impairment. For example, assessing the progression of neurodegenerative disease may be performed by measuring the change in neuromuscular control over time, such as by performing the method during a reference session to obtain a reference set of differences or a reference assessment of neuromuscular control, and then repeating the method at a later session (i.e., a late time) to compare the measured differences or measured assessment of neuromuscular control with those obtained in the reference session in order to assess the change. In one form, at least one set of exercises during a session has a higher degree of difficulty compared to another set of exercises during the same session to impose a higher cognitive load on the user, and assessing whether the user is suffering from a mild cognitive impairment is based on comparing the differences obtained when the user is under a higher cognitive loading.

In one form, the method may further comprise measuring a target joint angle for the at least one target musculoskeletal joint as a function of time and determining the one or more ranges of the measured target parameter and/or one or more joint angle ranges comprising determining the one or more joint angle ranges corresponding to the one or more parameter ranges where the user lacks control of the at least one target musculoskeletal joint. The determination of joint angle may be via geometrical calculations based on the apparatus involved or it may include synchronous video analysis of the user's motion captured by an image sensor during the plurality of exercises. In this embodiment the method further comprises using one or more image sensors, such as one or more cameras, to record the motion of the user and using motion capture software executing on a computer apparatus to perform motion capture and analysis of the user exercises. The software may be configured to create a representation of the user's ability to track the target parameter and determine deviations from the target parameter over time. The computer program product may be an app which is downloaded onto a user computing apparatus which establishes a communications link with other apparatus such as portable load cell apparatus.

In one form, the method may further comprise the preliminary step of determining a maximum achievable value of the target parameter as a reference value of the user and the target parameter is varied as a percentage of the measured maximum achievable reference value. By way of example, the maximum achievable value may be the maximum strength of the user, minimum strength of the user or some other statistically derived or relevant strength metric if the parameter of choice was a force measurement. It would be obvious to someone of ordinary skill in the art that this would be applicable to other parameters, such as, but not limited to, a range of movement, extent of movement, fluidity of movement (such as gait analysis), speed of movement, change of direction, type of muscle contractions (such as concentric or eccentric), muscle fatigue, total amount of exercise, smoothness or jerkiness of control, accuracy, length of viewing window, suppression (e.g., occlusion) of parts of the desired path, derivatives of displacement, a relative reference of a parameter, or a combination of these and their corresponding maximum achievable or statistically derived values. The target parameter may then be varied over the range of the maximum achievable value. This may be over the full range from 0 to 100% of the measured maximum achievable value (and allowing some tolerance to allow for the user improving their performance above the 100% measured reference level). In this way all subsequent measurements of the degree to which a user can control the joint or joints under investigation can be related to the maximum achievable value of the given parameter during that particular assessment. In some embodiments the maximum achievable value may then be used as a baseline value to quantify some aspect of the progression of musculoskeletal control over time. A lower range may also be set, and the target parameter is then varied from the lower range to the maximum achievable value. The lower range may be determined from measurements used to determine the maximum achievable value, such as a percentage (e.g., 10%, 25%, etc) or it may be a determined from an estimate of the range of control, such as from a previous assessment of the range of control, or it may be a predetermined value.

In an alternative form, the method may further comprise the preliminary step of estimating a maximum achievable reference value of a given parameter for the user based on one or more of physiology, age or sex of the user, and varying the target parameter over the range from a lower limit (e.g., 0, 10%, 20% etc) to an upper limit value determined from the estimated maximum achievable reference value. In some embodiments this may be 100% of the estimated maximum achievable reference value or it may be a greater value (e.g., 110% or 120%) to allow some extension above the 100% estimated reference level to account for users who exhibit greater neuromuscular control than the average of their demographic.

In an alternative form, the method may further comprise the preliminary step of looking up a historical measurement of a maximum achievable value of a given parameter for the user and varying the target parameter over the range from a lower limit (e.g., 0, 5%, 10%, 20%) to an upper limit value determined from the historically measured maximum achievable parameter. In some embodiments this may be 100% of the estimated maximum achievable reference value or it may be a greater value (e.g., 110% or 120%) to allow some extension above the 100% historically measured value to account for users who exhibit improved neuromuscular strength and/or control.

In a further form, for a first group of one or more sets of one or more exercises, the target parameter varies unpredictably, and the target parameter level is displayed for a short time window in advance of the current time, and for a subsequent group of one or more sets of one or more exercises, the target force level varies predictably and/or is displayed for a long time window in advance of the current time.

In a further form, a plurality of parameters is measured during an exercise, wherein analysis of the plurality of simultaneously targeted parameters is used to provide extra insight into the musculoskeletal system and the specific compound motion defined by the exercises. In yet another form additional measures may be extracted from the measured target parameters including subsets of the data, statistics on repetitive exercises, comparisons between the target parameter and the measured parameter, statistics on selected subsets of the data (for example the extension phase or even the acceleration phase of the extension phase of an extension/flexion exercise) and other measures.

In one form, measuring the target parameter(s) may include measuring a plurality of parameters during both dynamic and static control of a joint or joints. By way of a non-limiting example, dynamic control may be applying a constant force to an instrument while undergoing a defined series of exercises.

One example of this may be applying constant or varying force (or torque) to a cycling machine by keeping the legs moving in a constant cycling motion. This may also be via changing the direction and speed of pedalling—quickly in the forward direction, slowing to a controlled stop and increasing speed in the reverse direction and vice versa. By way of another non-limiting example, static control may be applying a constant or varying force to a force plate with the joints of the leg maintained in a fixed position.

In one form, measuring the target parameter further comprises measurement of one or more parameters of subsets of the measurements of one or more exercises performed repetitively over a period of time to determine a measure of stamina and the lack of control induced by tiring of the neuromuscular system. Such a time series indicating a decline in performance may indicate both cognitive and/or physiological fatigue. In another embodiment, the method includes a static measurement of one or more parameters over time to determine a measure of stamina and the lack of control induced by tiring of the neuromuscular system. Fatigue in general is a useful parameter to monitor potentially yielding greater insight due to local factors such as reduced motor unit recruitment and reduced rate coding (motor unit firing rates). Fatigue would therefore result in difficulty holding a steady state and having greater variability in target accuracy. This may also cause central effects as the gamma loop sensitivity may be changed affecting the corticospinal and cortical drive systems (via the cerebellum). In a one form, the target parameter may be a force exerted against a force plate sensor during a static measurement or some dynamic motion, a speed of movement, an acceleration or some other measurement of motion. Similarly (or additionally), the target parameter may be positional accuracy of one or more joints while under constant or varying load and while static or during some exercise or motion. The representation of the measured parameter may also be provided in real-time to provide real-time feedback to the user whilst performing the exercise (i.e., they may be shown the target parameter and the actual measured parameter).

In doing so the user is provided with a representation of their error or lack of control which creates a feedback mechanism so the user can adjust their neuromuscular control to reduce their error. Analysing the difference between the target parameter and the measured parameter as a function of time during the static measurement or exercise provides insight into the muscle groups and/or joint angles where the user lacks control of the at least one target musculoskeletal joint. Furthermore, the type of error signal, the frequency, the control loop delay, the relative ability to control in either increasing or decreasing parameter level, all provide insight into the lack of control and its possible mechanisms. This leads to greater knowledge for a clinician to target a rehabilitation programme to the specific neuromuscular issue.

In one form the one or more exercises are divided into a reference session and one or more subsequent sessions, and the assessment of the neuromuscular control comprises at least comparing the one or more differences determined in a last session of the one or more subsequent sessions with the one or more differences determined in a reference session to assess where the user is suffering from a mild cognitive impairment or to measure the progression of a neurological disorder. Assessing whether the user is suffering from a mild cognitive impairment and the extent to which the user is suffering cognitive impairment can be performed by comparing the differences in neuromuscular control obtained at two different time points or when the user is under a varying cognitive loading, assuming the user is in substantially the same state of physical health at the two different time points. Thus, in one form the at least one set of exercises during a session has a higher degree of difficulty compared to another set of exercises during the session to impose a higher cognitive load on the user, and assessing whether the user is suffering from a mild cognitive impairment is based on comparing the differences obtained when the user is under a higher cognitive loading. The cognitive loading need not be muscular in nature.

In a further form, the reference session provides a historical baseline measure and is obtained from the user performing the same assessment when not suffering from a mild cognitive impairment or at an early stage of cognitive decline such as after initial diagnosis. With cognitive decline a baseline may be taken at any stage of decline as the decline process is typically monotonic.

In one form applying cognitive loading may be achieved by asking the user to perform a mental cognitive task. This may comprise counting backwards from 100 by 7s (or some other combination of numbers so the user can't game the system by learning a sequence). In another form the alternate method may be achieved by asking the user to draw a given scene, an example of which may be drawing a clock face with a given time shown. In another form the alternate method may be asking the user to perform certain pattern recognition and matching tasks. In yet another form, the alternate method may be by changing the colour of the defined patterns of the representations of target parameter on the visual display or adding multiple different patterns to the visual display. Any number of cognitive loading techniques may be applied as the alternate method, and it is also possible to determine whether the user is truly under cognitive loading during these tasks by comparing their performance from session to session and cross correlating with the measure of neuromuscular control. Mild cognitive impairment can therefore be assessed on the basis of the degree to which factors such as arthrogenic muscle inhibition or neuromuscular control of a target joint are impacted by cognitive loading.

In one form, instructing the user further comprises playing one or both of an audible sequence and a haptic sequence whilst the user is performing an exercise, and the method further comprises replaying the one or both audible sequences and haptic sequences when the user is not engaged in a cognitively demanding task. In one form replaying the one or both audible sequences and haptic sequences is performed when the user is asleep (e.g., passive memory reactivation). The audible and/or haptic sequences may also be played along with a visual representation of the target and/or measured target parameter to create a linkage between visual and audible cues. In one form replaying the one or both audible sequences and haptic sequences is performed when the user is asleep in order to reactivate a past memory to enhance neuromuscular control.

In one form, the one or more exercises are performed by the user by using a NMT apparatus comprising a resistive element, and a sensing apparatus configured to measure the one or more target parameters as a function of time for the one or more capture time periods whilst performing the one or more exercises and a computing apparatus is configured to perform the steps of instructing the user, providing a representation of a target parameter, providing a feedback representation and determining one or more differences and generating and reporting an assessment. The computing apparatus is in communication with the sensing apparatus which provides the measurements of the one or more target parameters.

In one form the computing apparatus is a mobile computing apparatus comprising a display apparatus which is configured to display the one or more target parameters as a function of time and may be configured to receive measurements in real-time. The mobile computing apparatus may be a personal computer, a laptop, a tablet a smartphone, smart glasses, a dedicated microcontroller-based device or other application specific computing device. The computing apparatus may be integrated with the extension apparatus, or it may be operatively connected to the sensing apparatus via a wired or wireless communications link and be configured to display the feedback representation using the display apparatus.

In one form, the target parameter is force, and the force is measured by one or more load cells which are connected to one or more resistive components. The one or more load cells may be integrated into the NMT apparatus. The load cells may be portable load cells which wirelessly transmit measured force data to the computing apparatus and are adapted with attachment points to attach one end of the one or more resistive elements to one or more attachment points on the NMT extension apparatus. The user may be instructed to exert one or more forces on the load cells, and this may be represented as a visual graph of one or more forces versus time by the display apparatus of the computing apparatus. Similarly, the user may be instructed to apply the given force by one or more audio signals. The force may be represented by an audio tone wherein the frequency of the audio signal is representative of the required level of force (such as increasing frequency or higher pitch signifying the user to increase their exerted force). It would be readily apparent to those skilled in the art that any number of visual graphical displays, audio techniques, haptic feedback or other forms of instruction/feedback may be applied to instruct the user as to the required exertion force levels.

In another form, the computing apparatus is further configured to upload data comprising at least the assessment of the neuromuscular control to an external data storage location. In one form the computing apparatus further comprises an external interface adapted to provide connection to other computing resources such as an external computer or cloud storage and processing resources via any commonly used interface or protocol. In this case data associated with a given user's assessment may be uploaded to an external storage location (for example but not limited to an external database, a cloud storage apparatus, or some form of archive). This allows the system to maintain a longitudinal record of a given user's performance over time. Data archiving and mining then allows summary statistics, progress, compliance, and other data to be derived for a given patient, a given cohort of patients or for any other demographic subset of patients.

In one form the sensing apparatus comprises a computer vision system configured to capture and measure a joint angle as an exercise is performed. The computer vision system may be configured to capture location and/or pose data of the at least one target musculoskeletal joint, and one or more associated limbs whilst performing an exercise, and the computing apparatus uses the location and/or pose data, with the synchronous force measurements to estimate applied torque as a function of time during the exercise. In one form the computing apparatus is further configured to generate one or more plots representing the range of at least one target musculoskeletal joint movement in three dimensions, and the associated level of control.

In one form the sensing apparatus comprises one or more digital goniometers each configured to measure a joint angle when worn by the user. In one form the sensing apparatus comprises a force sensing apparatus comprising a force sensor, an inertial measurement unit, and communications module which is configured to wirelessly transmits measured force data and position data from the Inertial Measurement Unit (IMU) to a computing apparatus to estimate the applied force and joint angle as a function of time whilst performing the exercise. The sensing apparatus may comprise multiple sensing apparatus including combinations of the above.

In one form, using an apparatus to guide a user comprises attaching an end effector of a collaborative robot (cobot) to the user and wherein the end effector comprises a force measurement apparatus, and the cobot is programmed to follow a predefined path during an exercise and the user is instructed to activate one or more muscles associated with a at least one target musculoskeletal joint during the exercise to provide a reaction force, and the cobot comprises one or more force measurement apparatus (or force sensors) to measure the reaction force applied by the user whilst performing the exercise.

In another form, a user is attached to a force measurement apparatus which itself is rigidly attached to an end effector of a cobot. The user is instructed to perform one or more actions (i.e., movements along a predefined path) wherein the cobot is instructed to provide a programmed resistance profile to the user's motion, as measured by the force measurement apparatus. According to this embodiment, the programmed resistance profile may be a constant force, a force dependent on joint angle, a smoothly varying force (such as a sinusoid) or an arbitrarily changing force.

In a further form the cobot is a 6 degrees of freedom cobot configured to move the at least one target musculoskeletal joint through a full range of multiplanar joint movements of the at least one target musculoskeletal joint or the cobot is configured to simultaneously move a combination of multiple joints including the at least one target musculoskeletal joint in a multiplanar movement, during which the user provides a reaction force which is measured by the cobot.

In a further form, wherein the force data and joint angle data whilst performing an exercise is stored, and the cobot is configured to replicate a previously performed exercise for which stored force data and joint angle data is available, and the force data and joint angle data during the replicated exercise is compared to the stored force data and joint angle data to assess a change in the control of the at least one target musculoskeletal joint,

In a further form, the cobot stores or determines a threshold movement and force envelope for an exercise, and generates an alert if the measured force and/or location is outside of the threshold movement and force envelope for an exercise.

In a further form, the cobot is configured to maintain the force measurement apparatus perpendicular to a point of contact with the user whilst performing the one or more exercises.

In one form the one or more exercises may comprise a set of simultaneous exercises to be performed by multiple musculoskeletal joints, with each simultaneous exercise having a separate target parameter as a function of time.

In a further form the method further comprises generating one or more plots representing the range of at least one target musculoskeletal joint movement in three dimensions, and the associated level of control.

In a further form, the one or more exercises comprise multiple combinations of multi-planar joint movements, and generating one or more plots representing the range of at least one target musculoskeletal joint movement in three dimensions, and the associated level of control, and indicating ranges associated with performing functional tasks using the at least one target musculoskeletal joint.

In a further form, the method further comprises repeating the method for a contralateral musculoskeletal joint to the at least one target musculoskeletal joint, and generating a comparison of the associated level of control of the at least one target musculoskeletal joint, and the contralateral musculoskeletal joint.

In a further form the method may further comprise determining a rehabilitation plan based on the one or more force ranges where the user lacks control of the at least one target musculoskeletal joint, and periodically repeating the method to assess the improvement of the at least one target musculoskeletal joint.

In one form the plurality of exercises comprises one or more balance exercises. The one or more balance exercise may comprise the user remaining motionless in a first pose, and the one or more target parameters comprise the deviation from an initial position. The first pose may comprise a standing position on one leg, a standing position on both legs, a seated position, or a kneeling position. The first pose may be performed whilst the user is on a surface which is able to move in pitch, roll and/or yaw, and/or whilst the user is watching a moving visual field. The user may be instructed to remain motionless in the first pose for a first period of time with their eyes open, and then to remain motionless in the first pose for a second period of time with their eyes closed. The one or more balance exercises may also comprise one or more swaying exercises where the user is instructed to follow a sway pattern in which the user sways in a predefined path at a predefined rate, and the target parameter is the deviation from the sway pattern.

A variation of the NMT method may also be used to assess balance and comprises:
instructing a user, and/or using an apparatus to guide a user, to remain motionless for one or more time periods;
measuring one or more target parameters as a function of time for one or more capture time periods during the one or more time periods;
determining one or more differences between the one or more target parameters and the respective measured parameter over the one or more capture time periods; and
generating and electronically reporting an assessment of the user's balance using the one or more differences.

A representation of the one or more target parameters may also be provided during the one or more time periods. Any of the forms and variations described above in relation to the first aspect may be used in this assessment method.

According to a third aspect there is provided a computer program product comprising instructions for causing a processor to perform the method of the first or second aspects.

According to a fourth aspect a Neuro-Modulation Therapy (NMT) system is provided comprising:
a neuromuscular therapy apparatus comprising at least a resistive element;
a sensing apparatus comprising at least one sensor configured to measuring one or more target parameters when a user is using the neuromuscular therapy apparatus;
one or more output apparatus configured to output a representation of one or more of the one or more target parameters;
a computing apparatus comprising at least one processor, a memory, and a communications interface, wherein the communications interface is configured to receive a measurement of the one or more target parameters from the sensing apparatus and at least one processor is configured to control the one or more output apparatus and wherein the memory comprises instructions for configuring the processor to perform the method of the first of second aspects.

The system may comprise the sensor apparatus, load cells, and cobot arrangements as described above.

According to a fifth aspect there is provided a Neuro-Modulation Therapy (NMT) apparatus comprising:
at least one resistive element;
a sensing apparatus comprising at least one sensor configured to measuring one or more target parameters when a user is using the NMT apparatus, wherein the sensing apparatus is configured to provide a measurement of the one or more target parameters to a computing apparatus comprising at least one processor, a memory, and a communications interface, wherein the computing apparatus is operatively connected to or integrates one or more output apparatus configured to output a representation of the one or more target parameters and the memory comprises instructions for configuring the processor to perform the method of the first or second aspect.

According to a sixth aspect there is provided a Neuro-Modulation Therapy (NMT) kit comprising:
the NMT apparatus of the fifth aspect; and
a computer program product comprising instructions for causing a processor to perform the method of any one of the first or second aspects.

### BRIEF DESCRIPTION OF DRAWINGS

Embodiments of the present disclosure will be discussed with reference to the accompanying drawings wherein:

FIG. 1 is a schematic representation of a human body showing the three orthogonal planes used in medical imaging shown at 10;

FIG. 2A shows the main bone groups at 20 and FIG. 2B shows the main muscle groups at 40;

FIG. 3A is a schematic representation of a typical, healthy motor neuron pool shown at 60 indicating the plurality of individual motor units contained therein and FIG. 3B is a schematic representation of the action potential of the healthy motor neuron pool during an exertion event at 70;

FIG. 4A is a schematic representation of a typical motor neuron pool of a person suffering from arthrogenic muscle inhibition shown at 80 and FIG. 4B is a schematic representation of the action potential of the arthrogenic muscle inhibited motor neuron pool during an exertion event at 90;

FIG. 5A is a schematic representation of the major muscle groups of the thigh associated with extension of the lower leg at 100, the groups shown at rest and FIG. 5B is a schematic representation of the major muscle groups of the thigh shown during exertion of raising the leg against a resistance at 110;

FIG. 6 is a schematic representation of the neurological network of the cerebellum at 120;

FIG. 7 is a schematic representation of the spinal-reflex neurological pathways associated with movement of the lower leg at 160;

FIG. 8A is a sagittal plane schematic representation of a Neuro-Modulation Therapy apparatus, configured to employ resistance training of the knee joint, according to an embodiment and FIG. 8B is a sagittal plane schematic representation of leg press gym equipment adapted for use as an extension apparatus according to an embodiment;

FIG. 8C is a perspective view of an embodiment of a Neuro-Modulation Therapy apparatus configured to employ resistance training of the knee joint using a slidable pedal, and FIGS. 8D and 8E are corresponding top and side views;

FIG. 8F is a sagittal plane schematic representation of a Neuro-Modulation Therapy apparatus containing an active resistive element, configured to employ resistance training of the knee joint, according to an embodiment;

FIG. 9A is a schematic representation of a neuro-modulation apparatus in the form of a conventional stationary or exercise bike according to an embodiment; FIG. 9B is a schematic representation of a neuro-modulation apparatus in the form of a pedal set according to an embodiment;

FIG. 9C is a side view of a pedal set with a crank arm of fixed length according to an embodiment; and

FIG. 9D is a side view of a pedal set with a crank arm of variable length during a rotation according to an embodiment;

FIG. 10 is a sagittal plane functionally equivalent schematic representation of an embodiment of an extension apparatus wherein only the functionally equivalent elements are presented according to an embodiment;

FIG. 11 is a schematic representation of a force measurement apparatus according to an embodiment;

FIG. 12 is a sagittal plane schematic representation of a portable neuro-modulation apparatus being used in an office environment according to an embodiment;

FIG. 13A is a representation of a portable extension apparatus according to an embodiment, and FIG. 13B is a schematic representation of a portable extension apparatus according to an embodiment;

FIG. 14 is a schematic representation of a computing apparatus to which a sensor of a neuro-modulation extension apparatus connects and delivers information according to an embodiment;

FIG. 15A is a schematic representation of a defined pattern presented to the user according to an embodiment;

FIG. 15A is a schematic representation of a defined pattern presented to the user according to an embodiment, and FIG. 15B is a schematic representation of the defined pattern of FIG. 15A shown at a later point according to an embodiment;

FIG. 16A is a schematic representation of a defined pattern presented to a user according to an embodiment, FIG. 16B is a schematic representation of the user's measured replication of a defined pattern during a capture period; FIG. 16C is a schematic representation of the user error in replicating the defined sinusoid shown in FIG. 16A during the capture period, and FIG. 16D is a schematic representation of the absolute error (i.e., modulus of the error shown in FIG. 16C—although not to the same scale) according to an embodiment;

FIG. 17 is a series of visual representations of a sinusoidally varying defined pattern of target force shown on visual display apparatus in which different regions of the pattern are suppressed (occluded) in each visual representation according to an embodiment;

FIG. 18 is a series of visual representations of a sinusoidally varying defined pattern of target force shown on visual display apparatus in which different regions of the pattern are suppressed (occluded) in each visual representation according to an embodiment;

FIG. 19 is a schematic representation of the segmentation of a sinusoidal target force into the four quadrants according to an embodiment;

FIG. 20 is a schematic representation of the progression over time of an embodiment of NMT in which treatment progresses from feedback exercises to feedforward exercises;

FIG. 21 is a schematic representation of several days of a Neuro-Modulation Therapy programme according to an embodiment;

FIG. 22 is a flowchart of a Neuro-Modulation Therapy method according to an embodiment;

FIG. 23 is a flowchart of a method of assessing neuromuscular control according to an embodiment;

FIG. 24 is a flowchart of a Neuro-Modulation Therapy based rehabilitation, development or performance optimisation method according to an embodiment;

FIG. 25A is a schematic representation of an electromyography study of an exemplary subject suffering arthrogenic muscle inhibition according to an embodiment;

FIG. 25B by comparison is a schematic representation of an electromyography study on the same exemplary subject of FIG. 25A after a course of rehabilitation according to an embodiment;

FIG. 26A is a sagittal plane schematic representation of a bicep curl extension apparatus applied to the elbow joint according to an embodiment and FIG. 26B is a sagittal plane functionally equivalent schematic representation according to an embodiment;

FIGS. 27A, 27B, and 27C are sagittal plane schematic representations of a bicep curl extension apparatus applied to the elbow joint in which the shoulder joint is fixed to create a purely hinge-like action of the elbow joint according to an embodiment such that in FIGS. 27A and 27B the proximal member is kept in a vertical position and in an horizontal position, respectively, and in FIG. 27C the proximal member is allowed to move such that the wrist traces out a straight line in the sagittal plane according to an embodiment;

FIG. 28A is a partial schematic representation of an extension apparatus for treating the elbow configured to allowing movement in two degrees of freedom according to an embodiment, FIG. 28B is a schematic representation of a human hand holding the grip bar of handle shown in FIG. 28A, and FIG. 28C is a schematic representation of the distal end of the distal member having been rotated by roughly 90 degrees according to an embodiment;

FIG. 29A is a schematic representation of a sagittal plane extension apparatus adapted to treat the wrist in flexion and extension according to an embodiment, FIG. 29B shows a representation of a wrist extension using the extension apparatus of FIG. 29A, and FIG. 29C shows a sagittal plane schematic representation of the extension apparatus of FIG. 29A for flexion and extension of the wrist according to an embodiment;

FIG. 30 is a schematic representation of an extension apparatus accommodating the full range of motion of the wrist according to an embodiment;

FIGS. 31A and 31B are sagittal plane schematic representations of an extension apparatus applied to the ankle joint showing two extremes of the ankle motion in flexion such that ankle motion is purely in the sagittal plane, according to an embodiment;

FIG. 32 is a functionally equivalent schematic representation of a multi-planar neuro-modulation extension apparatus according to an embodiment;

FIG. 33 is a schematic representation of a cobot according to an embodiment;

FIG. 34A is an axial plane view of a user undergoing a static joint test in a cobot-based neuro-modulation apparatus according to an embodiment, and FIG. 34B is a near-sagittal plane view of the static joint test of FIG. 34A according to an embodiment;

FIGS. 35A through 35D are schematic representations of a first person view of an augmented reality neuro-modulation visualisation apparatus in which FIGS. 35A, 35B and 35C are augmented views at a first, second and third time point, respectively, of a hand of the user holding a position aid and tracing a first curve in which the view at each time point also shows representations of the hand at earlier time points, and FIG. 35D is an augmented view of a sinusoidal path with central occlusions similar to the view in FIG. 16A according to an embodiment;

FIGS. 36A through 36D are schematic representations of a first person view of an augmented reality neuro-modulation visualisation apparatus in which the user's hand may be obscured with FIGS. 36A, 36B and 36C are augmented views at a first, second and third time point, respectively, of a hand of the user holding a position aid and showing a past time path and a future time path wherein in FIG. 36B the hand is also occluded, and FIG. 36D is an augmented view of a sinusoidal path with central occlusions similar to the view in FIG. 16A and in which the hand is occluded according to an embodiment;

FIG. 37 is a schematic representation of a post-surgical total knee arthroplasty according to an embodiment;

FIG. 38 is a schematic representation of an externally attached digital goniometer according to an embodiment;

FIG. 39 is a schematic representation of an exoskeleton configured as a NMT apparatus according to an embodiment;

FIG. 40 is a schematic representation of the push-pull power cycle required to pedal a bicycle from the perspective of a single leg according to an embodiment;

FIG. 41 is a schematic representation of the data delivered by an exemplary connected pedal from a neuro-modulation apparatus according to an embodiment;

FIG. 42 is a schematic representation of a maximal performance calibration data capture according to an embodiment;

FIG. 43A is a schematic representation of a user display during a single leg neuro-modulation training session of a training programme according to an embodiment;

FIG. 43B is a schematic representation of a user display during a dual leg neuro-modulation training session of a training programme according to an embodiment;

FIG. 43C is a schematic representation of a user display during a dual leg neuro-modulation training session of a training programme that focuses only on the push power cycle for both legs according to an embodiment;

FIG. 44 is an exemplary user display of neuro-modulation training programme adapted to optimising performance of a series of muscles according to an embodiment;

FIG. 45 is a schematic representation of a mirror therapy according to an embodiment;

FIG. 46 is a schematic representation of a neuro-modulation apparatus combining the features of mirror therapy and a virtual or augmented reality headset according to an embodiment;

FIG. 47 is a schematic representation of a whole body neuro-modulation exercise using a virtual or augmented reality headset according to an embodiment;

FIG. 48 is a plot of the percentage of time of a trial that the error was in each of three error bands for 8 trials forming a single Neuro-Modulation Therapy session for a patient using a neuro-modulation apparatus according to an embodiment;

FIG. 49A is a schematic representation of an apparatus for characterisation of balance and Neuro-Modulation Therapy to improve balance, shown to characterise balance in only the sagittal plane and FIG. 49B is a schematic representation of an apparatus for characterisation of balance and Neuro-Modulation Therapy to improve balance;

FIG. 50 is a plot of the fore-aft component of a defined pattern used in characterisation of balance and Neuro-Modulation Therapy to improve balance;

FIG. 51 is a representation of a series of Lissajous figures;

FIG. 52A is a schematic representation of a circular defined sway pattern for characterisation of balance and Neuro-Modulation Therapy to improve balance;

FIG. 52B is a schematic representation of the fore-aft component of a circular defined sway pattern for characterisation of balance and Neuro-Modulation Therapy to improve balance;

FIG. 52C is a schematic representation of the left-right component of a circular defined sway pattern for characterisation of balance and Neuro-Modulation Therapy to improve balance;

FIG. 53A is a schematic representation of a circular defined sway pattern and the measured real-time position of a user undergoing characterisation of balance and Neuro-Modulation Therapy to improve balance;

FIG. 53B is a schematic representation of the fore-aft and left-right orthogonal components of a circular defined sway pattern and their respective measured real-time position for characterisation of balance and Neuro-Modulation Therapy to improve balance;

FIG. 54A is a schematic representation of an occluded circular defined sway pattern for characterisation of balance and real-time feed-forward motor learning in a Neuro-Modulation Therapy to improve balance;

FIG. 54B is a schematic representation of the fore-aft component of an occluded circular defined sway pattern for characterisation of balance and real-time feed-forward motor learning in a Neuro-Modulation Therapy to improve balance;

FIG. 54C is a schematic representation of the left-right component of an occluded circular defined sway pattern for characterisation of balance and real-time feed-forward motor learning in a Neuro-Modulation Therapy to improve balance; and

FIG. 55 is a schematic representation of an occluded circular defined sway pattern and the measured real-time position for characterisation of balance and real-time feed-forward motor learning in a Neuro-Modulation Therapy to improve balance.

In the following description, like reference characters designate like or corresponding parts throughout the figures.

### DESCRIPTION OF EMBODIMENTS

Embodiments of methods, systems, and apparatus for performing Neuro-Modulation Therapy, and associated assessment of neuromuscular control of a musculoskeletal joint, will now be described with reference to the figures. Neuro-Modulation Therapy, which for convenience may be abbreviated to NMT, covers a range of applications. This includes treating conditions such as AMI, rehabilitation therapies, or applications where there has been damage and NMT is used to restore the injured joint or neuromuscular system to a former condition and level of function, or to at least improve from the injured state even if full functionality cannot be restored. For example, NMT may be used to assist in recovery from surgical and related treatments including TKA, Cartilage repair, Synvisc injections (synthetic viscous fluids injected into a joint), Cortisone injections, etc. NMT also broadly covers therapy to achieve a higher state of function that was previously held prior to the therapy. This is broader than rehabilitation and may be performed by a patient to manage a condition as well as by a person seeking to develop or enhance their capabilities. For example, a child with Cerebral Palsy may use NMT to help them manage their condition but would not strictly be classed as rehabilitation. A child or amateur athlete may also use embodiments of NMT described herein to accelerate the development of motor skill function, providing a more targeted, faster and better way of reaching a desired capability. Development applications may thus include performance optimisation or performance enhancement, for example by elite athletes who may use embodiments of NMT to perform highly relevant targeted motor skill training to fine tune their neuromuscular control of specific joints and neuromuscular systems to achieve a higher probability of accuracy in an outcome-based task (i.e., to achieve optimum performance). It will thus be understood that methods of providing NMT, and associated systems and apparatus, as described herein may be used for a wide range of applications including rehabilitation, development, and performance optimisation applications. In the following discussion it is to be understood that the term NMT will be used broadly to refer to any of these applications.

Embodiments of NMT broadly comprise performing a series of feedforward exercises (i.e., actions) in relation to at least one target musculoskeletal joint or muscles that engage the sensorimotor system whilst performing the exercise. The series of exercises may also include feedback exercises, and the ratio of feedback to feedforward exercises as well as the sequencing may be varied over the course of the treatment The exercise could target a single musculoskeletal joint, or several musculoskeletal joints for example angle, knee and hips. The user is instructed to perform an exercise along with any performance instructions such as attempt to match one or more target parameters, and whilst the user is performing the exercise, one or more target parameters are represented and measured. During feedback exercises a representation of a target parameter is provided to the user as they perform the exercise for the duration of the exercise and in feedforward exercises the representation is suppressed (e.g., occluded, hidden or silenced) for at least a portion of the exercise. The representation may be a visual representation, an audio representation, a haptic representation, or some other representation or combination. The instruction to the user may be a visual, audio or haptic instruction, or some combination, and may include an explicit instruction to match the target parameter. Suppression refers to one or more time periods during which the representation of the target parameter is not presented to the user. These suppression time periods are time gaps in the representation of the target parameter. During suppression the target parameter(s) has a known value which the user is trying to match. However, as the target is not represented then the user has to actively estimate how to perform the exercise during the suppression time period. In some embodiments the suppression creates a gap in the representation of the target parameter and the thus feedforward refers to the requirement of the user to estimate the amount of control of the target musculoskeletal joint over the suppression timer period in order to match the target parameter at the end of the suppression time period which is a forward time point. Feedforward is also used as a contrasting term to feedback where the user is shown the representation for all of the representation time periods. The exercises may comprise one or more actions including static actions such as holding a joint in a specific position or angle, applying a constant force, or remaining motionless, as well as dynamic actions such as moving a joint over a range, or a series of actions, including repeated (cyclic) series of actions. The terms actions and exercises will be used interchangeably. That is, a reference to performing an exercise is equivalently a reference to performing one or more actions (and one or more exercises is equivalent to performing one or more actions). The instruction may be provided prior to starting the exercise and optionally whilst the user performs the exercise, e.g., the next action required if the exercise comprises a sequence of actions, or when during an exercise a target parameter is to be matched. The user may be instructed to perform the exercise on an instrumented apparatus which is configured to measure the target parameter, or the user may use a system comprising one or more sensors configured to measure the target parameter whilst the user performs the exercise. As outlined above a representation of the one or more target parameters may be provided to the user whilst they perform the exercises. They may also be provided with a feedback representation, either during the exercise or at the end of an exercise. The feedback representation is based on measurements of the target parameter obtained during the exercise (i.e., during capture time periods). The capture time periods may be the entire duration of the exercise (i.e., constant sampling of measured for the duration of the exercise), or some portion. The capture time periods will include at least the time period during which the representation is suppressed in a feedforward exercise. The capture time periods may be the same time periods as the representation time periods, or they may be different time periods. For example, if the target parameter was force, we may continuously sample force during the capture time period to generate a time series of force measurements. The feedback representation may be a representation or summary measure of the measurements (i.e., no calculation of the difference between the target and measured parameter) or the representation may be of a parameter or summary measure obtained from the measurements, including those based on calculating differences between the measured and target parameter. In some embodiments the user may be guided by an apparatus, such as a cobot, to perform the exercise and the apparatus is further configured to measure the target parameter whilst the user performs the exercise. The user may also be provided with instruction on how to perform the exercise, such as to resist a guided movement or to attempt to follow a predefined action or a target parameter(s), and as outline above, a representation of the target parameter(s) may be provided to the user whilst the user performs the exercise.

Neuromuscular assessments of the neuromuscular control of a target musculoskeletal joint may also be performed as part of Neuro-Modulation Therapy or as a standalone assessment. During assessment the method additionally comprises determining the accuracy with which the user performs the exercise, for example, by determining a difference between a target and measured value of the target parameter (or parameters). The accuracy may then be reported to the user and/or a clinician. During standalone assessments the exercises may comprise solely of feedback exercises, solely of feedforward exercises, or a combination of feedback and feedforward exercises. When performed as part of Neuro-Modulation Therapy an assessment may be performed to determine a baseline measurement and/or to assess progress during the Neuro-Modulation Therapy.

Neuromuscular assessments may be used to determine when to trigger progression of the treatment, such as introduction of feedforward exercises, or progression of suppression such as to increase (or decrease) the complexity of the exercise. Triggering a change may comprise changing the portion of the representation time periods that the representation is suppressed during a feedforward exercise to change a phase, a duration or a complexity of the suppression of the representation. The triggering of progression may be automated, or used by a clinician to make a progression decision. In some embodiments the NMT assessment may be a measure of the smoothness of motion, an error summary or a control summary obtained by using the differences (or deviations) between the target parameter and measured parameter for the duration of an exercise, or a set of exercises, and may be used to determine progression. This may be performed by analysing the distribution of the differences (deviations or errors) between the target parameter and measured parameter such as comparing with predefined thresholds or ranges, or by performing a temporal, spectral, or statistical analysis of the distribution. In one embodiment the assessment comprises using the differences to determine one or more accuracy measures which may be compared with one or more predefined trigger thresholds to determine when to trigger the change in suppression (i.e., the change in the portion of the representation time periods that the representation is suppressed). The differences may be compared with predefined difference thresholds and may be used to define difference ranges. The accuracy measure may then be an estimate of the percentage of time during a respective exercise that the differences are within one or more of the difference ranges.

In one embodiment, the one or more representation time periods and the one or more capture time periods are both the duration of the exercise, and the force is repeatedly measured (i.e., sampled) by an apparatus for the duration of the exercise. The difference between the target force and measured force is then calculated at each measurement (or sampling) point. That is a time series of force measurements are transformed to a time series of differences. These differences may be normalised or converted such as to a percentage error differences: % error difference=100%×[target−measured]/target. Then each percentage error difference may be compared to a difference threshold (e.g., 5%, 10%, etc) and the percentage of time during the exercise that the percentage error was less than the difference threshold, or within a difference range, calculated as the accuracy measure(s). This accuracy measure may then be compared with a trigger threshold, such as 90% to determine whether to trigger a change such as a progression in complexity. For example, if the difference was less than 10% for at least 90% of the duration of the exercise (i.e., difference threshold of 10% and a trigger threshold of 90%) we may automatically trigger a progression in complexity. Assessments may also be performed to assess when to decrease complexity, for example by using a second trigger threshold to indicate a decrease in accuracy. For example, if the differences were less than 10% for at least 50% of the duration of the exercise, then we may trigger a decrease in complexity (i.e., difference threshold of 10% and a second trigger threshold of 50%). To be clear an accuracy measure may be compared to both the first trigger threshold (trigger an increase in complexity) and the second trigger threshold (trigger a decrease in complexity). These may be programmed into a control apparatus of a NMT apparatus in which case the trigger thresholds may automatically trigger a change in suppression for subsequent exercises.

Other more complex or multifactor assessments of the error could be used to measure the smoothness of a motion (or exercise), or to generate an error summary or control summary (i.e., the ranges of the target parameters and/or one or more joint angle ranges where the user lacks control of the target musculoskeletal joint). One or more of these may be electronically reported and/or used to trigger progression of suppression, for example by defining multiple thresholds values and/or error ranges (green/good=difference<4.25%, orange/moderate=4.25-7.5%; red/poor≥7.5%) and determining the percentage of time in each difference range. Thus, rather than just determining the percentage of time the error difference is in the green/good band and using this to assess smoothness or triggering of progression, more complex triggering conditions taking into account the percentage of time in the orange/moderate error and/or red/poor bands may be used. In other embodiments the assessment could also look at absolute deviations rather than use percentages, or assess smoothness by performing a temporal, spectral or statistical analysis of the differences, or by analysing first and second derivatives of the differences, or performing outlier analysis, etc.

To assist in understanding embodiments of NMT (and neuromuscular assessment), we first focus on rehabilitation applications. Whilst many rehabilitation programs work on building or regaining strength, they are too generalised, and they often fail to accurately measure or improve control of the joint. In some cases, they even work on the wrong muscles/movements. Often the loss of control is limited to a particular range of exertion force, timing, coordination or to particular movements/joint angles. This loss of control is not adequately assessed by existing methods and systems, leading to incomplete recovery or to the recovery plateauing at a level below the optimum musculoskeletal control capacity because the rehabilitation program is not addressing the underlying cause of the loss of joint control. In contrast, embodiments described herein are configured to assess and quantify neuromuscular control to allow the development of more effective treatment and rehabilitation programmes and thus improve the recovery of the patient.

Existing rehabilitation approaches fail to consider that following injury and surgery the afferent signalling environment has significantly changed. For example, surgery itself generates trauma to the skin, capsule, graft sites, blood vessels, nerves, and bone as a result of cutting, sewing, drilling, debriding and removing. These interventions also immediately and permanently alter the morphology and biomechanics of the joint. For example, in ACL reconstructive surgery it is rare to achieve exact surgical placement of a tendon autograft. Similarly, knee replacement surgery can result in permanent leg length changes. These changes to the pre-surgical joint alignment and mechanics typically affect more than just the joint, and the rest of the body must also suddenly adapt to the post-operative state. In particular these changes create a monumental challenge for the central nervous system to instantly adapt to.

We thus propose neuromuscular assessment methods that focus on determining the range of parameters where the user lacks control of the target musculoskeletal joint, and which can also be used as the target parameters for rehabilitation exercises in a NMT based rehabilitation program where a user performs feedback and feedforward exercises in relation to the target parameters. This directs focus on the where the user lacks control and/or the new post injury afferent signalling environment and can thus lead to better rehabilitation outcomes. Embodiments may also be used for treating Arthrogenic Muscle Inhibition. AMI is a major cause of weakness, muscle wasting and poor motor control in rehabilitation from injury and surgical interventions as well as in ageing and sedentary populations. We hypothesise that the inhibition observed in AMI is due to the mismatch between the current state of afferent information and that which was expected by the brain, created from past experience. The brain's motor cortex is wired by years of afferent information which forms the baseline afferent information. When an injury or joint insult suddenly occurs, including surgical interventions, the new or altered afferent joint information doesn't match the brain's model of how the joint is supposed to respond to efferent signals, or commands. As a result, the brain inhibits motion because the feedback it receives from the joint is inconsistent with its expectation of the motion (i.e., the baseline afferent information). Functional magnetic resonance imaging studies of altered brain activity have shown that participants with a history of injury utilise greater activation of frontal lobe cortical areas responsible for motor planning and neurocognition. Physiologically, these frontal lobe areas have direct connections to cortical areas responsible for efferent motor drive (i.e., the motor cortex) and are able to influence descending motor output. We hypothesise that the increases in frontal lobe activity are reflective of a need for higher neurocognitive attention and processing during muscle contraction, likely as a compensation for loss of trusted sensory input coming from the injured joint. That is, the brain is likely forced to rely on other sensory information such as vision or cerebellar input to regulate motor function.

We thus propose that AMI is not purely reflexive in origin, as is commonly believed, but is likely an adverse neuroplastic adaptation to the changed sensory environment. That is, these chronic alterations to the afferent signalling from the injured joint drives further cortical/nervous system reorganisation and which unfortunately creates a neural environment less favourable to optimal muscle contraction and thus presents as AMI. Rehabilitation may therefore be assisted by embodiments of the NMT methodology described herein which focus on assessing musculoskeletal control and performing repeated exercises which are designed to accelerate the retraining of our neuroplastic brain to adapt to the new signalling environment in order to restore control and functionality. Further we propose that the same rehabilitation programs used to treat AMI can be used to monitor the progression of, and provide treatment for, neurodegenerative disorders and mild cognitive impairments, and assist athletes or other develop or enhance skills. We will refer to these various embodiments collectively as Neuro-Modulation Therapy (NMT) and will describe equipment for providing NMT referred to as NMT apparatus.

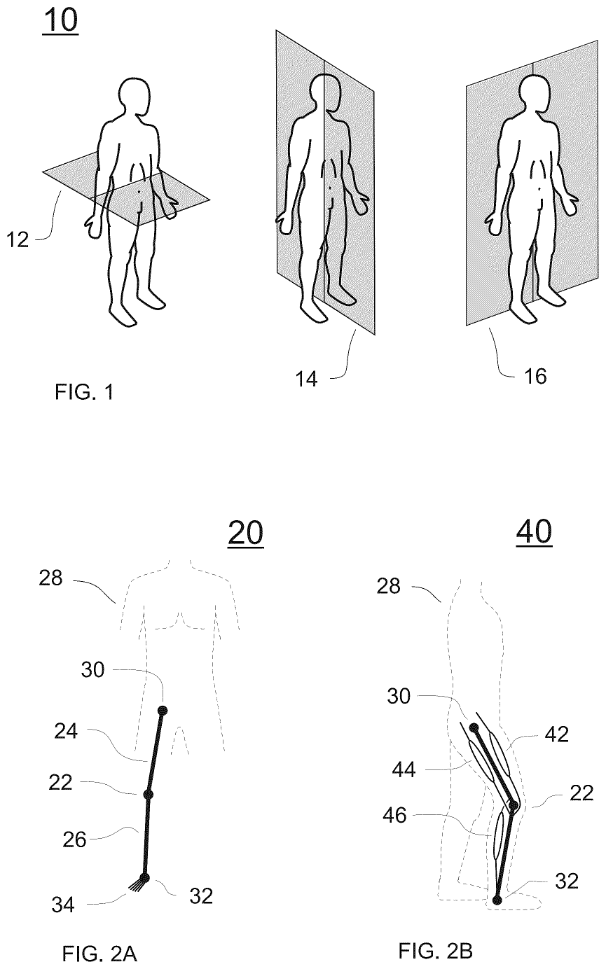

To further assist with understanding the embodiments it is helpful to first discuss in detail the musculoskeletal structure of the human body, and how the nervous system and brain provide neuromuscular control to control body movements. To simplify the discussion, it is prudent to define principal planes of movement with respect to a notional representation of the body. FIG. 1 is therefore a schematic representation of the three orthogonal planes used in medical imaging shown at 10. The transverse or axial plane 12, the sagittal plane 14 and the coronal plane 16 will be used throughout this disclosure to simplify the description of embodiments.

Control of a musculoskeletal joint typically involves coordination of the nervous system and the musculoskeletal system. The degree of musculoskeletal control can be characterised as applying the right force, to the right action at the right time at each of a plurality of joint angles. Impairment or reduction of normal function can be caused by many factors, including injury, surgery, ageing and diseases affecting the neuromuscular system. These impairments present as a range of clinical conditions including loss of control of one or more musculoskeletal joints. Often the loss of control is limited to a particular range of exertion force or joint angle. Whilst embodiments are disclosed in terms of a target musculoskeletal joint, it is to be understood that in a clinical setting patients may present with issues surrounding a joint, ligament, tendon, muscle, muscle group or other issue that impacts the loss of control of a given musculoskeletal Joint and treatment of all the above are considered to be within the scope of the present disclosure. Fluid, controlled motion requires the coordination of many muscle groups across a plurality of joints and while much of the present disclosure is taught by reference to a given joint in isolation (the target joint), this is to simplify the discussion and act as a reference point for a larger system. Embodiments as described herein therefore apply to rehabilitation and improved movement of an isolated joint, as well as to a group of related joints such as a whole limb, through to overall improvements in movement of the entire body. That is, embodiments as described herein are to be understood to relate to methods, apparatus and systems for all levels of muscular coordination.

The loss of control of a given body part can be characterised by the person's inability to apply a required control force over a defined range of motion, specific to that body part. The loss of control may be due to a multitude of factors. A loss of control may be due to localised impairment such as loss of sensing cells or functional cells. It may also be the result of a loss of neuromuscular control involving the brain and/or central nervous system issuing inappropriate signals to the target functional musculoskeletal group or adjacent functional group due to a loss of feedback information or other signals from the region or due to the brain issuing inappropriate compensatory signals in response to an injury to the site. In this latter case, the loss of control may be the brain continuing to implement an inappropriate learnt behaviour which can be compensated for by appropriate retraining. In the context of this specification a loss of control will be understood to include any loss of control, including but not limited to, a loss of neuromuscular control.

Embodiments of the method of assessing neuromuscular control are applicable to a wide range of body parts and joints including but not limited to shoulder, elbow, finger, hip, knee, back etc in which the exertion force of related muscles and/or a range of joint movement can be measured over a period of time. While the embodiments are applicable to any musculoskeletal structure or group of structures in the body the exemplary structure chosen for the initial description is the knee joint. However, it is to be understood this is simply to aid in understanding the embodiments of the various methods, apparatus and systems for assessing neuromuscular control and developing and implementing rehabilitation programs. That is, the embodiments described can be adapted, modified and extended for other joints beyond the knee joint. Accordingly FIG. 2A is a coronal view schematic representation of the main skeletal structure of the human leg at 20. The knee joint 22 connects extended proximal 24 and distal 26 members. Proximal member 24 (the femur) is attached to the body 28 by the hip joint 30 and the distal member 26 (which is comprised of the tibia and fibula) is terminated at the ankle joint 32 with a simplified view of the bones of the foot shown at 34 for clarity. FIG. 2B is a sagittal view schematic representation of the main muscle groups of the human leg at 40 wherein similar elements from previous figures are similarly numbered. The quadriceps group 42 on the anterior side of the femur 24 and the hamstrings group 44 on the posterior of the femur 24 act against one another to control the elevation of the femur 24 in sagittal plane. Other smaller muscle groups, which are not shown, act to control the lateral motion of the femur. The calves 46 on the posterior of the tibia act to control the position of the lower leg or the angle that the tibia makes with respect to the femur in the sagittal plane. None of the other soft tissue are shown but in order for proper functioning of the various joints it is appreciated that smaller muscles, ligaments, tendons, nerves and other soft tissues are present in the human leg.

Musculoskeletal control may be defined for the purposes of this disclosure as the ability to apply the right force, to the right action at the right time for a given joint. Proper functioning is then seen as maintaining fluid motion of a joint through the required angles in the required period of time and under varying loads. Conversely it may be defined as the ability to exert a required force under a given set of related circumstances. The degree of control may therefore be characterised by the ability to perform a task whereby the joint under investigation is at a given angle and under a defined force loading.

Normal functioning of a joint or muscle group may be impaired due to an injury or surgical intervention for some form of disease. In the case of the exemplary knee joint discussed previously, atypical sporting injury is anterior cruciate ligament rupture which is often surgically reconstructed. Similarly total knee arthroplasty is a common procedure with millions of such surgeries performed every year. In both of these cases rehabilitation programmes include exercises to strengthen the surrounding musculature, typically comprising resistance training. In the case of the exemplary knee joint, resistance training may include the exercise known as squats. In the early stages of recovery, a user may not be able to lift their own body weight so more gentle or assisted forms of exercise may be more suitable. This might include the use of a Pilates Wunda Chair or Pilates Reformer in which the resistance is provided by springs that increase in resistance with extension. Alternatively, this may include the use of gym equipment such as a leg press machine, in which the user's body weight is lifted along an inclined ramp or a series of adjustable weights are lifted via a non-elastic cable and pulley system. In all of these rehabilitation exercises a progression of increasing resistance is used as the user regains strength in muscle groups surrounding the joint; the objective being to reach maximum possible strength. Unfortunately, arthrogenic muscle inhibition remains a seemingly insurmountable limiting factor in any rehabilitation program. In AMI, an inhibited motor neuron pool exists, leaving less fully functional motor units available to activate muscles and produce force. A reduced number of available functional motor units also results in earlier onset of fatigue because the same healthy motor units are repeatedly recruited during repetitive exercises. This presents clinically as weakness, muscle wasting and poor motor control lacking neuromuscular adaptation that would be expected in a healthy untrained state.

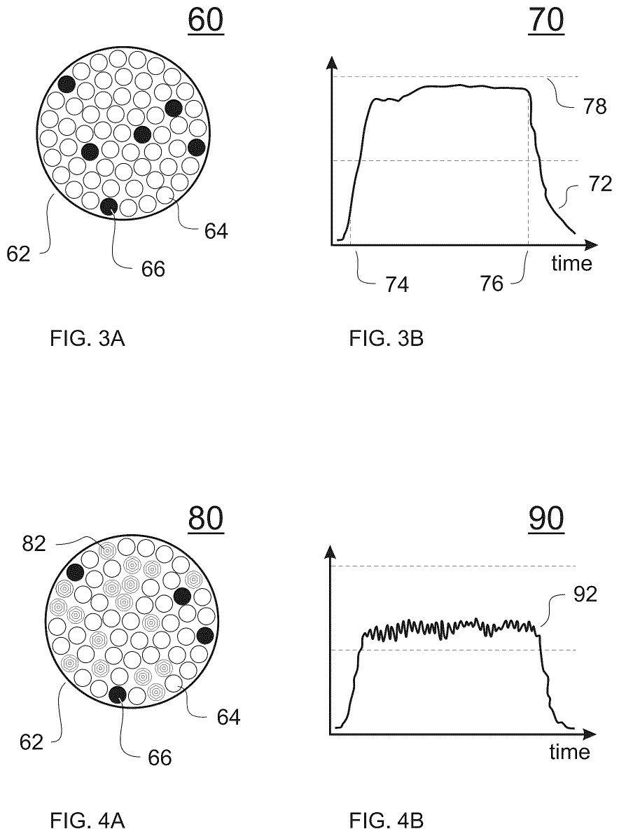

FIG. 3A shows a normal, healthy motor neuron pool under excitation at 60. Motor neuron sheath 62 retains the bundle of motor neurons, including a vast majority of actively engaged motor units 64 and a few healthy reserve motor units 66. Motor neurons bundle 60 is in a state of maximum exertion wherein almost all of the motor units are active, and a small minority of motor units are in what is considered the reserve state. They are not errantly unresponsive. Under normal conditions less than the full complement of motor units are actively engaged in what would be considered a normal full exertion attempt. This is the body's way of protecting itself from unnecessary daily overexertion. In extreme circumstances these reserve motor units can be recruited into action when high levels of adrenalin are present, and this would be akin to the superhuman strength that people have been reported to possess when trying to move a heavy object that has trapped a loved one.

FIG. 3B is a schematic representation of the action potential of resultant voluntary muscle force generated from the motor neuron bundle 60 at 70. The action potential of voluntary muscle generated force 72 is shown as a function of time from the onset of an exertion event 74 until the end of the maximal exertion event 76. The maximal possible resultant voluntary muscle force output action potential is represented by intensity 78 which under normal maximal exertion is not achievable due to the small number of motor units in reserve at any point in time. For the normal, healthy motor neuron pool 60 it can be seen that action potential 72 increases as the motor units turn on and the maximal resultant voluntary muscle force output action potential is relatively consistent across the exertion event.

By comparison FIG. 4A shows an arthrogenic muscle inhibited motor neuron pool under excitation at 80 wherein similar elements from previous figures are similarly numbered. Under maximal exertion there are fewer actively engaged motor units 64 and a similar small number of reserve motor units 66. In stark contrast to the healthy motor neuron of 60 however the arthrogenic muscle inhibited motor neuron pool has a large number of inhibited motor units 82. The ratio of inhibited motor units 82 to active motor units 64 determines the degree to which maximal total voluntary output is reduced. Similarly FIG. 4B is a schematic representation of the resultant voluntary muscle force output from arthrogenic muscle inhibited motor neuron bundle 80 at 90. The resultant voluntary muscle force output 92 shows the maximal possible exertion is significantly diminished as would be expected from a reduced number of active motor units operating normally. The maximal value of resultant voluntary muscle force output 92 across the exertion event is also shown to be quite erratic. This demonstrates the variability in maximal exertion force over time during the exertion event and is consistent with the typical physiological presentation of AMI. Arthrogenic muscle inhibition is then a major cause of weakness, muscle wasting, inability to control a steady state of force output and poor motor control in rehabilitation from injury and surgical interventions as well as in ageing and sedentary populations.

Despite the goal to restore quadriceps strength and function during rehabilitation, many individuals continue to present with lingering quadriceps deficits for months to years after surgery. Incomplete voluntary activation—the inability to adequately activate the quadriceps muscle completely during a contraction—is a commonly attributed source of quadriceps weakness after ACL reconstruction. Incomplete voluntary activation can occur due to under-recruitment of the motor units within the quadriceps muscle and/or due to suboptimal firing of the recruited motor units, called rate coding. This persistent quadriceps weakness has been associated with abnormal knee biomechanics, increased joint contact forces, co-activation of quadriceps and hamstrings, poor patient-reported function and decreased functional performance in individuals with ACL reconstruction. Strangely, quadriceps weakness is not limited to the ACL reconstructed leg (or the leg that underwent total knee arthroplasty). Quadriceps weakness and voluntary activation failure are common in individuals with ACL reconstruction and are often observed bilaterally after the surgery as is well documented in the literature. This suggests a neurological bilateral inhibition process rather than something localised within the injured, reconstructed or surgically altered joint.

Diminished voluntary activation is well documented in the literature for both the reconstructed and the non-reconstructed legs relative to healthy control legs (i.e., the legs of the uninjured control group study participants) and this bilateral inhibition has been attributed to the observed bilateral weakness after unilateral ACL injury and reconstruction. Recent evidence also points to the contributions of neural alterations that occur following ACL reconstruction. For example, alterations in spinal-reflex and corticospinal excitability have been reported following ACL injury and reconstruction and these alterations have been associated with quadriceps weakness after ACL reconstruction. Spinal-reflex excitability is typically measured using the quadriceps Hoffmann reflex (H-reflex), which provides an estimate of the proportion of the motor-neuron pool that can be voluntarily activated. The H-reflex can be influenced by both pre- and post-synaptic pathways, and decreased H-reflex excitability is considered to contribute to poor quadriceps voluntary activation after ACL injury or surgery. Following injury and surgery, changes in neurophysiology associated with lost ACL mechanoreceptors appear to influence the excitability of the corticospinal pathways. Consequently, the disrupted corticospinal pathways may suppress the neural drive to the quadriceps muscle and induce quadriceps weakness and activation failure.

Despite all of this evidence pointing to neuromuscular factors driving arthrogenic muscle inhibition and post-injury/surgery weakness and lack of control, prior art treatment protocols focus almost entirely on strength. Squat exercises and leg raise exercises are often prescribed as part of the rehabilitation programme for anterior cruciate ligament recover or after total knee arthroplasty. Recovery is often deemed to be strength symmetry between the injured knee and the uninjured knee to within 5%. This is problematic for several reasons, not least of which is that of bilateral inhibition and weakness.

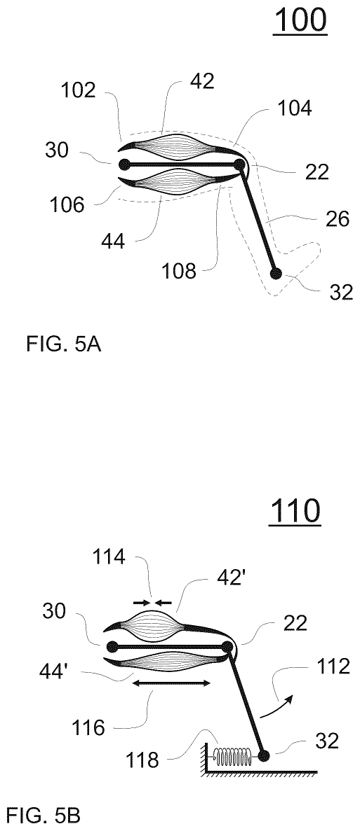

In order to advance the discussion on neuromuscular control of the exemplary knee joint it is instructive to highlight the major muscle groups driving the biomechanics of the knee. FIG. 5A is therefore a simplified sagittal plane schematic representation of the musculoskeletal system of the human leg at 100 showing the quadriceps 42 and hamstring 44 in their rested state, where similar elements from previous figures are similarly numbered. Quadriceps 42 is connected to the hip, not shown, via tendon 102 and to the tibia 26 by tendon 104. Hamstring 44 is connected to the hip via tendon 106 and to the tibia 26 via tendon 108. FIG. 5B is a simplified sagittal plane schematic representation of the musculoskeletal system of the human leg undergoing a lower leg raise 112 at 110 where similar elements from previous figures are similarly numbered. In raising the lower leg 112 the quadriceps 42′ are considered the agonist muscle group and are therefore contracted 114 to initiate this movement while the hamstrings 44′ are the antagonist and are lengthened 116 in an inhibitory process. Raising the lower leg like this is akin to a squat exercise, particularly if the motion is resisted by a spring 118, weight or other form of resistance. Conversely, to lower the leg against some resistance holding it up (not shown in FIG. 5B, as might be the case in gym exercise equipment, the hamstring 44 acts as agonist while the quadriceps 42 become the antagonist. The process is an exact mirror of the former discussion surrounding raising the lower leg. The nervous system acts to contract the hamstring 44 while the quadriceps are reciprocally inhibited and lengthen.

There are two main aspects of neuromuscular control, and both have been implicated in reduced precision of motor control following an injury or surgical intervention—corticospinal pathways of the central nervous system and spinal-reflex pathways of the peripheral nervous system. Corticospinal pathways can be considered to be the main highway of information up and down the spinal column whereas spinal-reflex pathways are like the side roads that transmit information from the spinal column to and from the periphery of the nervous system.

Planning, control and execution of voluntary movements originate in the motor cortex, a region of the frontal lobe of the cerebral cortex. These ‘command’ signals originating in the cerebral cortex pass along the upper motor neuron being routed through the thalamus, midbrain, pons and medulla before entering the spinal cord. The upper motor neuron passes through the corticospinal tract in the spinal column until it reaches the relevant vertebra and prepares to exit the spinal cord, where it synapses with a second neuron on the ventral horn of the spinal cord. The second neuron is the lower motor neuron which exits the spinal cord to ultimately synapse with motor units in the skeletal muscle of interest. [00175]‘Command’ signals are only part of the neuromuscular control system. The trajectory of a given movement is monitored in real time against the desired or commanded motion. Error correction feedback systems then fine tune the trajectory of a movement via secondary motor control systems.

Again, there are two feedback pathways that influence trajectory error correction—corticospinal and spinal-reflex. The corticospinal feedback system is largely modulated in the cerebellum, a major feature of the hindbrain of all vertebrates that plays a particularly important role in voluntary motor control. It may also be involved in some cognitive functions such as attention and language as well as emotional control such as regulating fear and pleasure responses, but its movement-related functions are the most extensively established. The human cerebellum does not initiate movement, but contributes to coordination, precision and accurate timing. The cerebellum itself receives input from sensory systems of the spinal cord and from other parts of the brain and integrates these inputs to regulate and fine tune motor activity. To put this into context, the afferent input to the cerebellum is 40 times greater than the output, expressing the extent of organisation and modulation of large amounts of sensory information into coordinated voluntary motor outputs. The cerebellum motor functions can resemble a conductor coordinating an orchestra, making sure the brass, woodwind, string and percussionists are playing in harmony. Without the cerebellum, motor function would be disorganised as is seen in erratic ataxic movement.

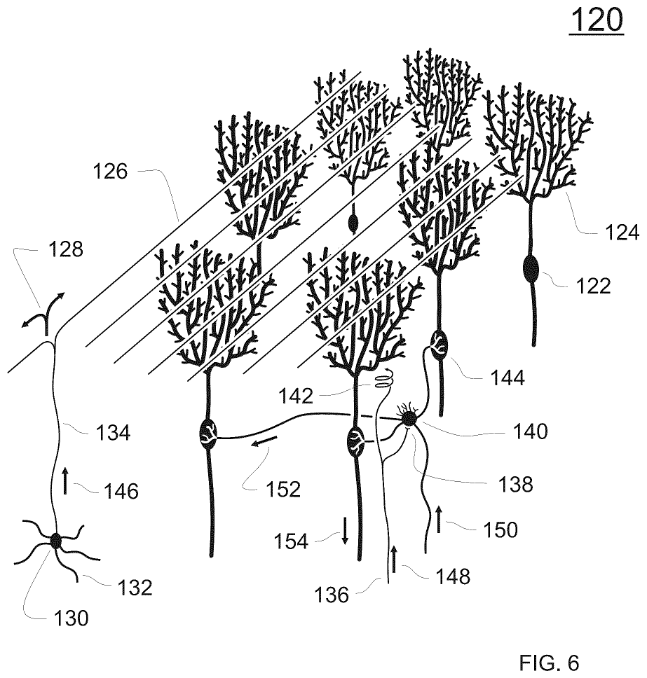

In addition to its direct role in voluntary motor control, the cerebellum is necessary for several types of motor learning, most notably learning to adjust to changes in sensorimotor relationships. Structurally, the cerebellum contains only a few key cell types and structures, organised in distinct layers. FIG. 6 is a schematic representation of the signal processing structure of the cerebellum at 120. Purkinje cells 122 are a unique type of neuron specific to the cerebellar cortex. They are remarkable and instantly recognisable for their massive, intricately branched, flat dendritic trees 124 giving them the ability to integrate large amounts of information and learn by remodelling their dendrites. In the three-layered cerebellar cortex, the bodies of Purkinje cells make up the middle Purkinje cell layer, while their dendritic trees make up the outermost layer. Purkinje cells play a major role in the cerebellar circuit's motor coordination, providing fine tuning or course correction of a movement in progress.

Purkinje cells are arranged in flat sheets perpendicular to the folds of the cerebellum through which orthogonal ‘Parallel Fibres’ 126 carry information 128 aggregated by Granule cells 130 deep in the cerebellum. Granule cells 130 receive information from a plurality of Mossy Fibres 132 and have axons 134 that branch this into the network of Parallel Fibres 126. Climbing Fibres 136 carry afferent information about the actual motion of the body and ascend the cerebellum synapsing 138 with Basket Cells 140 and subsequently wrapping themselves 142 around the dendritic trees 124, synapsing many times as they climb 142 through the dendrites. Basket cells 140 make synaptic connections to a plurality of Purkinje cells 144 as well as connections across a subset of the Parallel Fibre network (not shown for simplicity). Information 146 about the required or expected motion of a body part, received from the Mossy Fibres, is transferred across the multitude of Parallel Fibres and multiplexed across the Purkinje cells' dendritic trees, each Parallel Fibre making hundreds or thousands of synaptic connections as they pass through the layers of dendrites. This is analogous to a massive switchboard-like matrix architecture facilitating highly parallel processing. Climbing Fibres 136 and their associated Basket Cells 140 carry information 148 and 150 respectively from a range of sensory systems including the vestibular system (responsible for providing information about motion, head position and spatial orientation), the reticular formation (a complex network of brainstem neurons that serve as a major integration and relay centre), the superior colliculus (for incorporating environmental stimuli and coordinating both eye and head movements associated with gaze) and a range of other sensory systems. The expected motion information 146 is compared to the actual motion information 148 and 150 resulting in inhibitory signals 152 being sent to both Purkinje cells and the synaptic connections between parallel fibres and the dendrites themselves (not shown for simplicity). The result is inhibition of the drive 154 to the motor system, resulting in course correction of motion. The entire output of the Purkinje cells then gets routed via the deep cerebellar nuclei (not shown for simplicity) to the ventrolateral nucleus of the thalamus, where it feeds into the motor cortex's ongoing actions and hopefully smooths any error out of the motion.

The large dendritic trees of the Purkinje cell are thought to be critical to the course correction process; they receive complicated inputs from the massive array of Parallel Fibres and integrate them into a single representation of what the Purkinje cell “thinks” the current motion should be. This is then compared to the actual motion derived from afferent signals conveyed to the Parallel Fibre-Purkinje cell synapses via Climbing Fibres and to the Purkinje cells themselves by Basket cells; an error correction signal then modifies efferent drive. Each Purkinje cell receives input from up to 200,000 parallel fibres and thus indirectly from a million or more mossy fibres. The vast array of parallel fibres allows motor signal from the arms, legs, trunk and head to be all coordinated together to produce meaningful voluntary movement. The cerebellum therefore exists as signal processing system that aggregates commands from across the central nervous system to course correct motion via inhibition of either agonist or antagonist muscle groups as required.

The brain as a whole is highly neuroplastic at birth. In many vertebrates, neurons undergo extensive rewiring during postnatal development, removing synapses from an initially over connected network. This process, known as synapse elimination, occurs in the central nervous system and peripheral nervous system. One of the most striking examples of synapse elimination in the central nervous system occurs in the cerebellum, where connections between Climbing Fibres and Purkinje cells are modified. This phenomenon has been studied extensively in rodents, where shortly after birth, multiple Climbing Fibres innervate Purkinje cells. By the end of the third postnatal week in rodents, only one Climbing Fibre innervates each Purkinje cell. This parallels the synapse elimination in the peripheral nervous system, which occurs between motor axons and muscle fibres at the neuromuscular junction. Perinatally, approximately 10 motor axons innervate each muscle fibre in a muscle but almost immediately after birth axons begin removing synapses from some muscle fibres.

It was thought that neuroplasticity declines significantly as we age but the cerebellum in particular has proven to remain highly neuroplastic. Climbing Fibres are now considered to convey ‘teaching’ signals instrumental to synaptic plasticity and learning in the cerebellar cortex throughout life. Climbing Fibres modify the connection strength of synapses between Parallel Fibres and the dendritic tree network. Several theoretical models have been developed to explain sensorimotor calibration in terms of synaptic plasticity within the cerebellum. At least four key principles of cerebellar action have been identified as important: feedforward processing, divergence and convergence, modularity and finally plasticity.

Signal processing in the cerebellum is almost entirely feedforward—that is, signals move unidirectionally through the system from input to output. The cerebellum is a highly deterministic system. Signals enter the circuit, are processed by each stage sequentially and then leave. The cerebellum provides a quick and clear response to the input of any particular set of stimuli. Feedforward motor control occurs in the planning of an action. When dropping a weight that is held with an outstretched arm, the ankle and hip compensate for the anticipated shift in the body's centre of gravity upon release. This action occurs subconsciously but is a feedforward process because it occurs before the event to place the body in the right position for the event or action. In the human cerebellum, information from 200 million ‘Mossy Fibre’ inputs is expanded to 40 billion ‘Granule’ cells, which drive signals along ‘Parallel Fibres’ that then converge onto 15 million ‘Purkinje’ cells. Because of the way that they are lined up longitudinally, the 1000 or so Purkinje cells belonging to a given processing function may receive input from as many as 100 million Parallel Fibres and focus their own output down to a group of less than 50 so called ‘Deep Cerebellar Nuclei’ cells. Thus, the cerebellar network receives a modest number of inputs, processes them through its very extensive, highly parallel and rigorously structured internal network and sends out the results via a very limited number of output cells—divergence and convergence. The cerebellar system is functionally divided into more or less independent modules, which probably number in the hundreds to thousands. All modules have a similar internal structure but different inputs and outputs. Different modules share input from Mossy Fibres and Parallel Fibres but in other respects they appear to function independently—the output of one module does not appear to significantly influence the activity of other modules.

Perhaps most importantly for motor learning is that the synapses between Parallel Fibres and Purkinje cells as well as the synapses between Mossy Fibres and Deep Nuclear cells are both susceptible to modification of their strength. In a single cerebellar module, input from as many as a billion Parallel Fibres converges onto a group of less than 50 Deep Nuclear cells. The influence of each Parallel Fibre on those nuclear cells is adjustable. This arrangement gives tremendous flexibility for fine-tuning the relationship between the cerebellar inputs and outputs and it is this plasticity that the methods described herein (and associated apparatus and systems) seeks to harness.

Years of neural system reinforcement and Purkinje cell dendritic remodelling builds a hard-coded map within the cerebellum of the neural sensory signals that correspond to each desired motion. This is often referred to as the ‘internal model’ of motor function. If a desired motion returns sensory information 148 and 150 that is inconsistent with the hard-coded expectation of that desired motion 146, the cerebellum acts as an inhibitory brake on the cerebellum's motor output. It is probable therefore that when an injury or surgical intervention results in changes to the body's sensory system an inhibitory process also occurs within the cerebellum that is consistent with processes of arthrogenic muscle inhibition. Moreover, there appears to be a significant reduction in the excitability of the corticospinal pathways after injury or surgical intervention. This appears to be responsible for the reduction in strength observed and can be directly attributed to a mismatch between the altered sensory input and the hard-coded cerebellar mapping of desired motion to sensory input. Musculoskeletal rehabilitation therefore needs to focus on methods of enhancing neuroplasticity and efficiently retraining the brain to a new normal of altered sensory signalling.

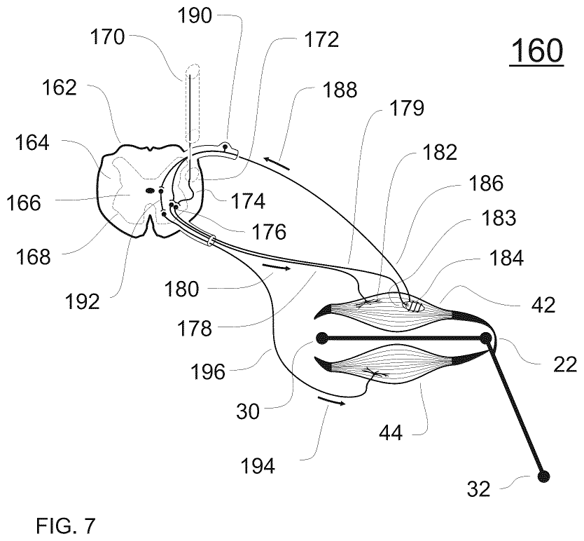

The second major component of the neuromuscular system includes the peripheral nervous system, the spinal-reflex pathways. FIG. 7 is a schematic representation of the spinal-reflex pathways associated with control of the human leg at 160 in a resting state with a sagittal plane representation of the leg where similar elements from previous figures are similarly numbered. An axial plane slice of spinal cord 162 corresponding to the motor control of quadriceps 42 and hamstrings 44 contains areas of ascending and descending corticospinal pathways 164 often referred to as white matter and local spinal-reflex pathways 166 often referred to as grey matter with boundary between the two regions 168. In order for the quadriceps 42 to contract and raise the lower leg as discussed by way of FIG. 5B, quadriceps 42 must contract and hamstrings 44 must be inhibited and lengthen.

Cortical drive initiates voluntary contraction of the quadriceps via an efferent signal from the central nervous system that travels down the lateral corticospinal tract 170 to the relevant slice of the spinal cord 162 wherein it traverses the boundary 172 between the lateral corticospinal tract and subsequently enters the grey matter 166. The distal end of the upper motor neuron 174 synapses with both the proximal end of the lower or alpha motor neuron 178 which innervates extrafusal skeletal muscle to generate force-producing contractions and the proximal end of the gamma motor neuron 179 which innervates intrafusal muscle fibres. Alpha motor neuron 178 transmits the efferent signal 180 towards the quadriceps 42 where the neuromuscular junction or synapse 182 excites each corresponding extrafusal multi-fibre, single motor unit within the quadriceps 42 to contract. If a high proportion of the available motor units within the agonist quadriceps 42 were to be suddenly excited, with a corresponding inhibitory lengthening of antagonist motor unit within the hamstring 44, then a sudden and rapid, maximal force leg raise could occur. If, however, the person wanted to raise their lower leg in a slow and controlled action aimed at accuracy or specifically targeted response, a more complex and coordinated process occurring between the corticospinal command signals of the central nervous system and the feedback system of the spinal-reflex system would become apparent.

Skeletal muscles are not just motor actors, they contain within the skeletal belly their own proprioceptive sensory system. In FIG. 7, the quadriceps 42 contain their own sensory system called the muscle spindle 184 enclosed in a sheath (spindle), surrounded by extrafusal muscle fibres 182, connected to motor neurons 179 and sensory neurons 186 that communicate with the spinal cord. The muscle spindles contain intrafusal muscle fibres that are much smaller than extrafusal fibres, hence their role isn't to generate force-producing movements but instead to sense the quadriceps 42 muscle contractile state. The intrafusal fibres run parallel to the extrafusal muscle fibres. The main sensory afferent neurons of muscle spindles, known as type Ia and II afferent fibres, provide sensory signals about the intrafusal muscle fibre length and the rate of change of length. When type Ia and II neurons are activated, an afferent signal is sent into the dorsal horn 190 of the spinal cord to link up to a gamma motor neuron 179 in the anterior horn of the spinal cord. The efferent signal passing along the gamma motor neuron 179 produces a contraction of the intrafusal fibres. The sensory afferent Ia and II neuron and efferent gamma motor neuron form a loop called the gamma loop or gamma spindle system. This gamma spindle system allows muscle spindles to be a central component in the regulation of muscle contraction state. Even in the muscle spindle resting state, described as muscle tone, the gamma spindle system is in a constant state of operation. When a quadriceps muscle 42 is stretched, the change in length is transmitted to the intrafusal muscle fibres within the spindle that contain it, being subsequently stretched. This in turn activates the muscle spindle sensory afferent type Ia and II fibres that transmit a signal 188 in through the dorsal horn of the spinal cord to link up to the gamma motor neuron. The efferent gamma motor neuron activates the intrafusal muscle fibres to relax and lengthen to maintain equilibrium with the quadriceps muscle. Likewise, the opposite process occurs when the quadriceps muscle contracts and shortens. The muscle spindle intrafusal fibres become lax and again the sensory afferent type Ia and II fibres respond by sending an action potential to link to the gamma motor neuron to contract and shorten the intrafusal fibres. This process retains the sensory sensitivity within the muscle.

Alpha motor neurons 178 at the neuromuscular junction 182 innervate extrafusal muscle fibres 183 that generate force-producing contractions, whereas gamma motor neurons 179 innervate intrafusal muscle fibres within the spindle 184 to regulate muscle spindle sensitivity. Gamma motor neurons, through the muscle spindle system, modulate the activity level of alpha motor neuron activation. When an alpha motor neuron activates the agonist quadriceps 42 muscle to contract the type Ia and II afferent fibres within the corresponding muscle spindle are activated. This in turn links up to an inhibitory interneuron 192 within the spinal cord to inhibit the alpha motor neuron activation of the opposing hamstrings antagonist muscle 44. The result is the hamstrings reciprocally relaxing by reducing the contraction of the muscle opposing the quadriceps. This is termed Reciprocal Inhibition. Reciprocal inhibition allows a synchronized and coordinated response to smooth and controlled movement as well as opposing stiffness. The finely-tuned dynamic interplay between afferent signalling, alpha/gamma motor neuron signalling and reciprocal inhibition to opposing muscles is critical to produce a voluntary, targeted response that allows smooth and efficient neuromuscular operation.

Unfortunately, a problem exists with reciprocal inhibition in subjects with anterior ligament reconstruction and total knee arthroplasty. It is widely reported via EMG studies that these subjects experience co-activation or co-contraction of the quadriceps and hamstring muscles that cause knee stiffness and stiff-legged walking as a protective strategy. In addition, gamma motor neurons which regulate the gamma spindle system are known to be modulated from supraspinal centres such as the cerebellum within the central nervous system and hence become a source of arthrogenic muscle inhibition. This is evidenced in studies that have found altered corticospinal and spinal reflexive excitability in subjects with anterior cruciate ligament damage (Rush et al. Assessment of quadriceps corticomotor and spinal-reflexive excitability in individuals with a history of anterior cruciate ligament reconstruction: a systematic review and meta-analysis. Sports Med. 2021 51(5): 961-990.).

In summary, we propose that injury or surgery results in altered sensory input and resultant changes within the supraspinal centres of the cortex and cerebellum impact the cortical drive and interact locally within the gamma spindle system. The finely-tuned system has now lost sensitivity and its ability to apply the right force, to the right action, at the right time is now in jeopardy. If this poor functioning system is left unattended, it is likely that a less than optimal system will continue without resolve. This will have a considerable impact on a patient's function, and we believe it is not surprising that at least 1 in 5 are unsatisfied with their total knee arthroplasty.

An afferent signal 188 from the muscle spindle is transmitted to the spinal cord 162 via the sensory neuron 186, the cell body or dorsal root ganglion 190 of which is located outside of the spinal cord. Sensory neuron 186 transmits the afferent feedback signalling 188 to motor neuron 178, via a neurotransmitter exchange between the presynaptic and postsynaptic cells 176 in a feedback process that regulates the overall efferent signal which drives contraction of the quadriceps 42 and allows controlled, smooth and coordinated motion of the lower leg.

The process of providing fine motor control for contracting the quadriceps 42 must be matched by a corresponding inhibitory lengthening of the hamstring 44. A given muscle can only apply force in one direction, contraction; there is no lengthening force. Contraction and lengthening of opposing muscles are the result therefore of increasing and decreasing the overall efferent signals respectively. Muscle spindle 184 provides feedback afferent information 188 about the actual state of quadriceps contraction. This information undergoes a phase inversion via interneuron 192; a feedback induced increase in the efferent signal 180 results in a decrease in the efferent signal 194 delivered to hamstring 44 via the hamstring lower motor neuron 196. In this simplified FIG. 7 we do not show the hamstring's upper motor neuron exiting the lateral corticospinal tract 172 or its complementary muscle spindle sensor for simplicity nor do we show feedback to the quadriceps via a similar interneuron pathway. The inventors do not intend to discuss synchronous hamstring inhibitory lengthening from a corticospinal perspective, but this process will be readily apparent to one of ordinary skill in the art. Only one alpha motor neuron 196 is shown for the hamstring but muscle spindles, type Ia and II motor neurons and gamma motor neurons are present although not shown for simplicity. Beta motor neurons (not shown) also play a part in the innervation of both intrafusal and extrafusal muscle fibres, but their action is beyond the scope of this discussion.

It must be stressed that this is a simplified discussion of the neuromuscular pathways of the spinal-reflex system. There are countless reflex pathways that provide both excitatory and inhibitory reflex control of the lower leg flowing into each slice 162 of the spinal cord from sensory neuromuscular junctions in other major muscle groups. The important point to note for this discussion is that both excitation and inhibition of opposing muscle groups must work synchronously and in tandem for smooth neuromuscular control.

There is a growing body of evidence to suggest that individuals with ACL reconstruction exhibit alterations in both corticospinal and spinal-reflex excitability and these alterations may contribute to poor quadriceps function after ACL reconstruction. Specifically, there appears to be a significant reduction in the excitability of the corticospinal pathways which is compensated for, to a limited extent, by an increased excitability of the spinal-reflex pathways. Arthrogenic muscle inhibition is acknowledged to be one of the primary barriers to recovering full strength after injury or surgical intervention as has been well documented in the exemplary case of ACL injury or total knee arthroplasty.

We believe that AMI is consistent with both the error control inhibitory mechanism of the cerebellum and reduced corticospinal excitability, particularly in light of the way the cerebellum's motion processing system works. After injury or surgical intervention, the aggregate afferent sensory feedback from the plurality of muscle spindles 184 received by the cerebellum does not match the expected response from a given motion command initiated by the motor cortex. This is akin to a pattern matching state machine. Altered afferent signalling from Climbing Fibres cannot match the patterns pre-programmed into the Parallel Fibres to Purkinje cell dendritic trees synapse connections. The normal feedforward processing runs into an error state with the pre-programmed ‘logic’ and inhibits the output of Purkinje cells resulting in the overall reduction of corticospinal excitability observed. AMI can thus be understood as the body's natural protective mechanism against further injury in response to the mismatch between received and expected afferent information, resulting in co-contraction of opposing muscles around a joint to limit motion, causing dynamic joint stiffness and affecting fine motor control. This could also be described as a weaker core motor function response but an increased peripheral motor response. Framed another way, greater motor emphasis is given to the fine motor feedback process. Rather than providing small adjustments to the gross corticospinal drive, the spinal-reflex pathways' adjustment process becomes dominant, resulting in jerky stop-start action of the drive to agonist motor units, presumably by amplified exaggerated inhibitory braking action of the antagonist muscle group. This underscores the importance of targeting both the spinal-reflex and corticospinal pathways in rehabilitation to normalise quadriceps function after ACL injury or reconstruction.

Arthrogenic muscle inhibition is a major cause of weakness, muscle wasting and poor motor control in rehabilitation from injury and surgical interventions as well as in ageing and sedentary populations. Motion must be considered a closed loop neuromuscular ‘system’. A command is sent to move a limb through a defined path via efferent signals. Afferent information provides feedback about the actual motion relative to the commanded motion and the motor cortex (as well as other higher functions) provides adjustments to the commands in order to improve and fine-tune motor control. As discussed above, when an injury, surgery or joint insult suddenly occurs, the new or altered afferent joint information doesn't match the brain's model of how the joint is supposed to respond to efferent signals, or commands. We hypothesise that the brain responds by inhibiting motion as a protective mechanism; causing co-contraction of opposing muscles around a joint to limit motion and seeking other input such as vision or cerebellar input to regulate motor function, resulting in dynamic joint stiffness, jerkiness, AMI and a lack of control. Furthermore, the protective mechanism is observed to present bilaterally, even with unilateral injury as previously discussed.

Rehabilitation must therefore focus on retraining the brain to expect new afferent information flow in response to the motion commanded of a chronically altered joint. Just like learning to walk for the first time, the neuromuscular system must learn how to operate the new joint morphology. We must train the brain to recognise this new normal. We thus propose a method of assessing neuromuscular control of a target musculoskeletal joint. This provides benchmarking and enables assessment of progress in a rehabilitation program. This information can be used to design a rehabilitation program which we will refer to as Neuro-Modulation Therapy (NMT), that we consider offers a pathway to recovery of function. NMT is a process that appears to prime the brain and promote neuroplasticity to unlock corticospinal drive. This in turn activates previously inhibited motor units, resulting in greater overall motor unit recruitment, a significant increase in strength and a subsequent greater degree of fine motor control. NMT therapy utilises repeated exercises (or actions) based on the embodiments and variations of the method of assessing neuromuscular control. In some embodiments NMT therapy uses feedback exercises and feedforward exercises using an instrumented exercises apparatus, which we will refer to as a NMT apparatus or NMT system. The feedback exercises allow the user to calibrate their effort on the equipment with the feedforward exercises then requiring them to predict the required effort to meet a target effort. Rather than just repeating exercises to regain strength, this approach may force the brain to pay attention to the sensory feedback and thus allowing the brain to relearn control of the target joint. Interestingly unilateral Neuro-Modulation Therapy appears to downregulate the effects of arthrogenic muscle inhibition bilaterally. That is, Neuro-Modulation Therapy of one limb has a positive effect on reducing AMI on both that limb and the similar contralateral limb.

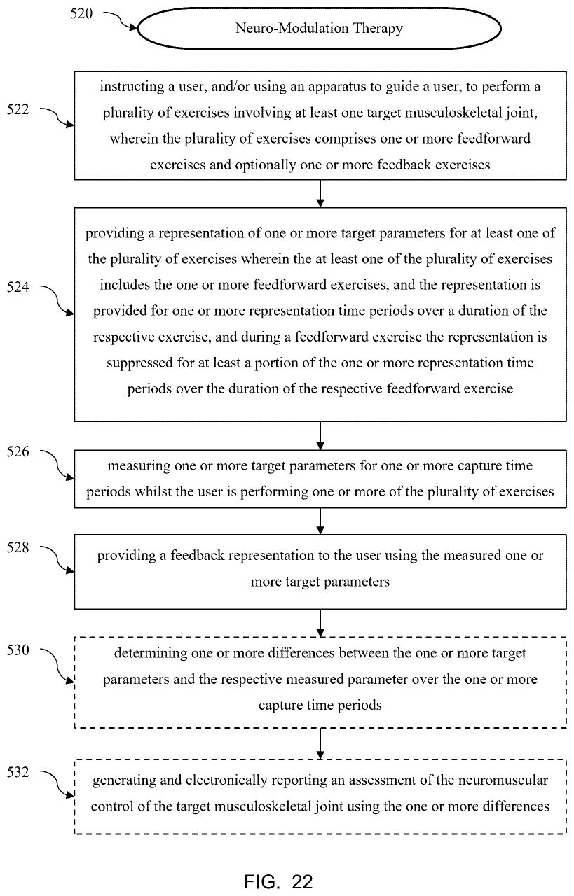

FIG. 22 is a flowchart of an embodiment of Neuro-Modulation Therapy 520. The user, at 522, is instructed, and/or an apparatus is used to guide the user, to perform a plurality of exercises involving at least one target musculoskeletal joint. These exercises comprise at least one or more feedforward exercises and may optionally comprise one or more feedback exercises. The user, at 524, is also provided a representation of a target parameter for at least one of the plurality of exercises wherein the at least one of the plurality of exercises includes the one or more feedforward exercises. The representation is provided for one or more representation time periods over a duration of the respective exercise, and during a feedforward exercise the representation is suppressed for at least a portion of the one or more representation time periods over the duration of the respective exercise. The representation time period(s) may be the duration of the exercise, or a portion of the duration of the exercise. For example, a user may be instructed to perform an exercise for a duration, such as 30 seconds. During a feedback exercise, if performed, the representation is provided for all of the one or more representation time periods over the duration of the respective exercise. That is, and in contrast to the equivalent feedforward exercise, the representation is not suppressed. One or more target parameters are measured, at 526, for one or more capture time periods whilst the user is performing the exercises, and a feedback representation is provided to the user, at 528, using the measured target parameter(s). The capture time periods may be the same as the representation time periods or may be different time periods. The user may be instructed to attempt to match the target parameter in real time for the representation time period.

Further each representation time period may be the time to perform at least one complete exercise, i.e., one set or one cycle, or the representation time period may be the duration of the exercise (e.g., three sets of an exercise, or 30 seconds of continuous activity). The exercise may comprise one or more actions and the user may be instructed to perform a single exercise, or multiple repeats of an exercise (i.e. repeat the one or more actions N times), or to continuously perform an exercise for a duration of time (i.e., keep repeating the one or more actions). The use of representation and capture time periods gives flexibility in how the treatment is provided based on the exercises being performed. For example, representation time period may be the entire duration of the exercise, but the capture period is shorter such as excluding an initial warm up period, and/or warm down period. For example, in a 30 second duration exercise the warmup period may be 5 seconds such that the representation time period is 30 seconds and the capture time period 5-30 seconds or 5-25 seconds (warm up and warm down periods). The representation time period could also exclude warm up and/or warm down periods to allow the user to start performing an exercise or using the equipment before being shown the representation to try and follow. For example, in a 30 second duration exercise the representation time period may be 5-25 seconds. In another example the exercise may be a long duration with representation and capture periods starting after an extended time period when fatigue may have set in. For example, the exercise may be a duration of 300 seconds, with a representation time period of 60 seconds spanning 200-260 seconds. In some embodiments a second warm-up period may be provided before the start of the capture period to allow the user to adjust their actions to match the representation before their performance is measured. For example, in a 30 second duration exercise, the representation time period may be 5-30 seconds and the capture period may be 10-30 seconds. Warm down periods may also be provided. In some embodiments multiple representation time periods may be used during the duration, for example from 0-15 seconds then 20-30 seconds with a 5 second gap. Similarly multiple capture periods may be used which may be the same or different than the representation time periods. In the case of a repetitive exercise with a fixed frequency, the capture time periods may be synchronised with a particular phase of the exercise, and the duration may be some integer number of periods of the exercise. For example, if the exercise is matching a sinusoid force varying at a frequency of 0.3 Hz for 30 seconds, two capture time periods may be used each synchronised to the zero-phase point (x-axis crossing point) and each be 3 periods long (10 seconds). The feedback representation may be provided during or after the exercise, and may be representation of the measurement, or a representation derived from the measurement such as based on a comparison with the target parameter, and provide feedback to the user to assist them in matching the target parameter during a current or future exercise.

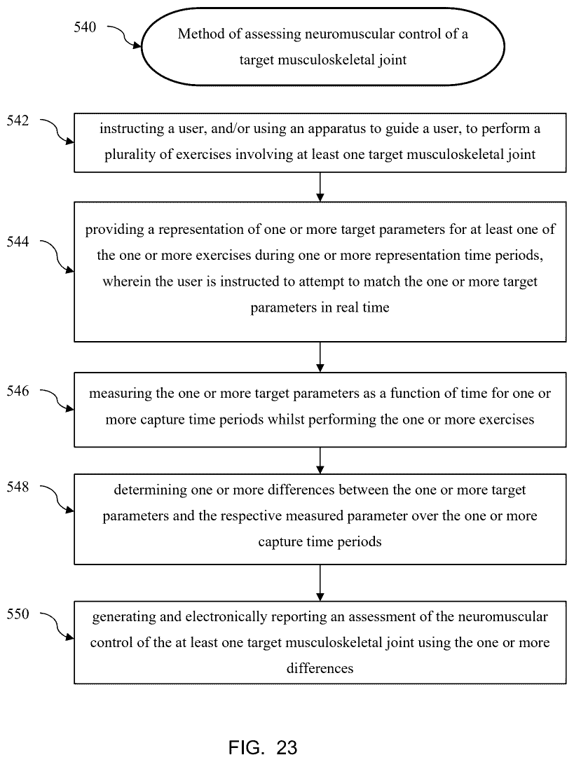

In some embodiments an assessment of the neuromuscular control of the target musculoskeletal joint during one or more exercises may be performed for example by determining one or more differences between the target parameter and the measured parameter over the one or more capture time periods. The assessment or the differences may be used to trigger a change in the portion of the of the one or more representation time periods that the representation is suppressed during a feedforward exercise to change a phase, a duration or a complexity of the suppression of the representation. For example, if the user is demonstrating improved control the complexity may be increased, and if the user is demonstrating a reduction in control the complexity may be decreased. The assessment or differences may also be used to assess smoothness of the exercises. In some embodiments the assessment of the neuromuscular control of the target musculoskeletal joint may be electronically reported 532, for example by providing a representation to the user or a clinician, or an electronic document such as an electronic report (e.g., Word, PDF, excel file) may be generated and provided to the user and/or clinician, such as by being sent over email or stored on an electronic storage apparatus (e.g., hard disk, cloud storage, website) requiring the user to login/authenticate to gain access to the report. Additionally, assessment of the neuromuscular control of the target musculoskeletal joint may be performed as a standalone method, and thus FIG. 23 shows a flowchart of a method of assessing neuromuscular control of at least one target musculoskeletal joint 540. In this embodiment the user, at 542, is instructed, and/or a user is guided, to perform one or more exercises involving at least one target musculoskeletal joint. The user is provided with a representation of a target parameter, at 544, for at least one of the exercises during one or more representation time periods. The user is instructed to attempt to match the target parameter in real time. The target parameter is measured as a function of time, at 546, for one or more capture time periods whilst the user is performing the exercises. We then determine, at 548, one or more differences between the target parameter and the measured parameter over the one or more capture time periods and generate and electronically report an assessment of the neuromuscular control of the at least one target musculoskeletal joint using the one or more differences at 550. Reporting may be a representation on a screen during and/or after completion of the exercise, or it may be an electronic document provided to the user and/or clinician, including being sent electronically or stored electronically and requiring the user to login/authenticate to gain access to the report.

Each exercise is either a dynamic exercise or a static exercise, and each may comprise one or more actions, or repeating the same exercise for a duration of time. A dynamic exercise may comprise activating one or more muscles associated with a target musculoskeletal joint to move the target musculoskeletal joint over one or more joint angle ranges. The number of joint angle ranges will typically depend upon the joint and nature of the impairment or injury. For example, a knee joint is a hinge joint allowing movement in one dimension and thus there is only one joint angle range. In contrast a shoulder allows movement of an arm in 3 dimensions, and thus there may be one, two or three joint angle ranges, depending upon the exercise specified. A static exercise may comprise activating one or more muscles associated with the target musculoskeletal joint whilst holding the target musculoskeletal joint in a static position at a fixed joint angle. For example, the user could be instructed to activate the muscles at different force levels whilst the joint is held at a fixed joint angle.

The user may be instructed to a perform a single exercise, a plurality of coordinated exercises or a set of exercises, each of which may be a single exercise which is repeated, or multiple exercises including coordinated exercises following an ordered sequence. In some embodiment the user will be instructed to use a particular apparatus (a neuro-modulation apparatus, or more succinctly, a NMT apparatus) to perform the exercise. This may be based on existing gym apparatus which has been suitably instrumented as described herein or may be a purpose-built apparatus as described herein. In some embodiments a cobot or other similar apparatus may be used which is configured to guide the user to perform an exercise. In these embodiments the user may be further instructed on what action to do in response and the cobot (or other apparatus) can be used to measure the target parameter. For example, the user may be instructed to grip an end effector which follows a particular path, and the user is instructed to resist the movement.

A representation of a target parameter is provided for at least one of the one or more exercises during one or more representation time periods. This may be a static (constant) value, or a dynamic value and the user is instructed to attempt to match the target parameter level in real time. For example, the representation may be a number, shape or curve, displayed on a display apparatus of a computing apparatus 212, and/or a sound level from an audible apparatus such as a speaker or headphone, and/or haptic feedback from a haptic apparatus. The target parameter may be periodically varying force, such as sinusoidal force varying at a specific frequency and amplitude for the duration of the exercise. The target parameter may be a force, a joint or limb position, a joint angle, an acceleration or any other similar parameters relating to movement of the target joint. A sensing apparatus may be used to measure, sample, or capture the value of the parameter at a point in time and to transfer this value, or a representation of the value, to a computing apparatus. For example, the sensor may obtain samples at specific times, or continuously sample at a sampling rate. The sensing apparatus may perform on-board processing of signals prior to sending to the computing apparatus. For example, in the case of measuring force, a force transducer or load cell may be configured to capture a measurement, or a series of measurements at a sampling rate, and transfer the measurements of the force to the computing apparatus 212. The steps 524 and 528 may be combined by showing a representation of the target parameter and the feedback representation such as the measured parameter as a function of time, and optionally a representation of the difference between the target parameter and the measured parameter as a function of time. In one embodiment the representation may be a charted comparison which displays both the target parameter along with, or compared to, the measured parameter in real-time or as an instantaneous measurement of the target parameter either in isolation or overlaid on or with the visual representation of the target parameter to represent the difference between the target and measured values.

In another form the difference between the target parameter and the measured parameter may by represented by an audible pitch or volume change or change in frequency and/or amplitude of haptic feedback. The representation time periods, and the capture time periods maybe the same time periods, partially overlapping time periods, for example the representation time periods may be a subset of the measurement time periods or the time periods may be distinct time periods. In some embodiments the measurement time period may be a single continuous time period whilst multiple representation time periods may be used, at least some of which are within the continuous time period.

It would be readily apparent to one of ordinary skill in the art that the parameter may be a parameter such as force, but it could also be a position, a joint angle, an acceleration or any relevant parameter than can be measured using apparatus as described herein. Further the target value need not be a static value but may be allowed to vary in amplitude, frequency, phase, or a parametric curve such as a sinusoid, sawtooth, polynomial, etc, and may be displayed as a time series/curve representation in which the x axis is time, and the y axis is the value of the target parameter. As will be discussed below, in some embodiments part of the curve or time series may be suppressed or occluded. Determining the difference between a target parameter and measured parameter may comprise calculation of an error value at a point in time, or over a time range, and analysis may comprise performing mathematical or statistical analyses of the error values.

In assessment methods (at steps 532 and 550) we may generate and report an assessment of the neuromuscular control of the target musculoskeletal joint using the one or more differences. This may be performed by using the one or more differences to determine one or more ranges of the target parameter and/or one or more joint angle ranges where the user lacks control of the target musculoskeletal joint. In one form assessment may comprise performing a statistical analysis of the one or more errors to characterise where the user lacks control of the target musculoskeletal joint. In some embodiments predetermined thresholds and/or statistical/computational methods may be used to assess where the user lacks control, for example where it exceeds or crosses one of the predetermined thresholds. In one form reporting an assessment of the neuromuscular control may include displaying the differences, such as a charted comparison discussed above on a screen. A software module executing on the computing apparatus may be configured to collect measured differences and generate the assessment, including perform the statistical analysis and/or estimate the ranges where there is a loss of control. Multiple difference values may be processed to determine the ranges.

Generating the differences and reporting the assessment may be performed for a single exercise or for a set of exercises. That is, the user could perform several different exercises, or the same exercise repeatedly, and the assessment reported after all exercises are completed. Assessment could be performed on each exercise, or each action in an exercise, and only reported at the end. The report may be displayed on a display apparatus or written to an electronic file such as a spreadsheet or pdf document, and stored on an electronic storage apparatus. Access control restrictions such as a password, or storage on password-controlled apparatus or site, may be used to prevent unauthorised access to the report. The report may be sent to the patient or another person such as a clinician, or sent to a computer storage including cloud storage apparatus. In some cases, the report may be an internal report comprising a value generated by a software module and which is reported to another software module or the processor, or it may be stored in a memory or written an electronic file until all exercises are performed. Determining range where the user lacks control may also take into account additional information such as the user's medical history and any prior injury or surgical information. For example, a prior injury or surgery may have permanently limited a range.

Embodiments of NMT and assessment of neuromuscular control may be varied or expressed slightly differently. For example, in one embodiment providing a representation of the target force level, measuring the force and determining the differences could equivalently be considered the steps of determining an accuracy with which the user performs each of the one or more exercises and the assessment may comprise providing real-time feedback to the user on the accuracy. Further instances of the above method may form the basis of a NMT based rehabilitation, development program or performance optimisation method which comprises repeating the above method, or variations of the above method, at multiple different times. This may be performed as sets of multiple exercises (each of which may include multiple actions), and a session may comprise multiple sets of exercises, with one or more sessions conducted on different days. The sets or sessions may comprise assessment exercises, where the range over which the user lacks control is assessed and reported, as well as feedback and feedforward treatment exercises, where the target parameter is selected based on the range where the user lacks control (from a previous assessment). In the treatment exercises, a feedback representation is provided to the user of the measurement of the target parameter. In some exercises (or embodiments) this may be a representation of the measured value or a summary representation of the measured values, and no direct comparison is made with the target parameter. That is the step of determining any differences between the measured and target parameter may be optionally omitted. Similarly, if a difference is measured, any assessment or reporting of the assessment may be optionally omitted. The differences or assessment of the differences may be used to trigger a change in the complexity of the exercises. The feedback representation may comprise displaying the difference between the measured and target parameter, rather than determining the one or more ranges of the target parameter and/or one or more joint angle ranges where the user lacks control of the target musculoskeletal joint. That is only minimal (e.g., at the start and/or end of a session) or even no assessment of neuromuscular control may be performed during an NMT session (or sessions). In some embodiments the differences may be calculated periodically, such as during one session per week, or on demand, and used to determine when to trigger a change in the complexity of the exercises, such as in a review session with a clinician. For example, a user may perform NMT therapy at their home, with periodic review sessions with a supervising clinician to determine whether to make changes to the therapy. In some embodiments the differences may be used by the computing (or control) apparatus of the NMT apparatus to automatically change the complexity of the exercises in response to a trigger condition being met. This allows automation of the NMT. In some embodiments the trigger conditions may be preset and then reviewed and adjusted by a clinician over the course of the therapy.

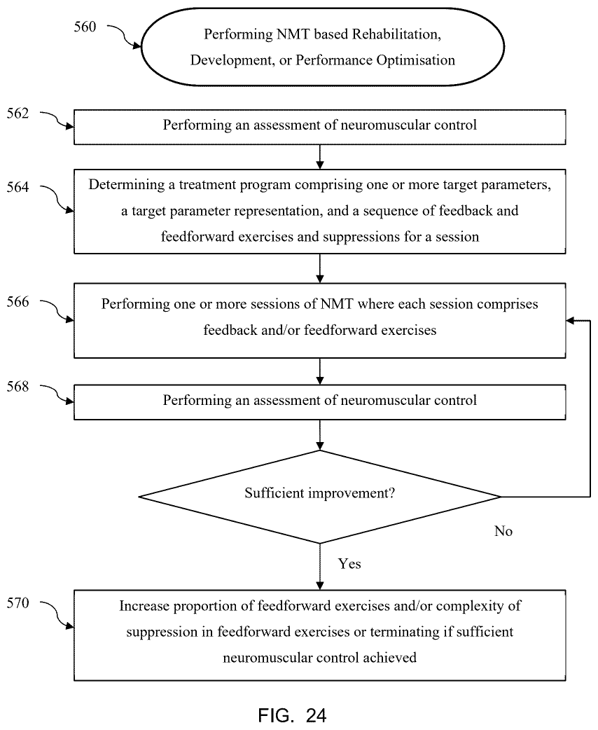

This is illustrated in FIG. 24 which is a flowchart, of performing, at 560, a Neuro-Modulation Therapy based rehabilitation, development or performance optimisation method according to an embodiment. We begin by performing an assessment of neuromuscular control 562, such as according to the method 540 illustrated in FIG. 23. We then determine, at 564, a treatment program comprising one or more target parameters, a target parameter representation, and a sequence of feedback and feedforward exercises and suppressions for a session, or for multiple sessions. These may be designed to target where there is poor control of the target musculoskeletal joint based on the assessment at 562. In some embodiments a sequence of suppressions to be used over a plurality of sessions may be determined.

We then perform, at 566, one or more sessions of NMT where each session comprises feedback and/or feedforward exercises. A further assessment of neuromuscular control is performed at 568 followed by checking whether sufficient improvement has been achieved. If not, we return to 566 and perform additional sessions of NMT, otherwise we proceed to 570 and either increase proportion of feedforward exercises and/or complexity of suppression in feedforward exercises, or terminating if sufficient neuromuscular control achieved.

Exercises may be further grouped into feedback and feedforward exercises or sets of exercises (each of which may be one or more actions). For example, once a neuromuscular assessment has been performed to determine where the user lacks control, the target parameter may be selected to vary over the range where the user lacks control or over some component of this range in order to direct the user to focus attention on the range of motion where there is a lack of control. Each set may comprise a different exercise or the same exercise with a different range of the target parameter or even different target parameters. Similarly, the different sets may each be identical, or each set may be different, or some sets may be repeated. The sets may be organised into sessions. The choice of exercise, and what range of the target parameter to use in a set may be guided by an initial assessment of neuromuscular control, or it may be based on performance in previous sets, and updated or revised as the user progresses.

For example, a treatment method, or treatment exercise, may comprise step 522 of instructing a user to perform one or more exercises and providing feedback to the user on the measured one or more target parameters (step 528). This may be real-time feedback where a representation of the measurement is displayed in real time, including traced over the target parameter in real time, or displayed as a difference between the measured and target values is displayed in real time. The representation may also be a summary representation provided after the exercise, or after a series of exercises are performed, such as an average accuracy or percentage of time within a threshold value of the target. The representation might also be a classification-based representation, e.g., red, orange, or green traffic light indicator, based on the accuracy or difference between the target and measured values of the target parameter (for example percentage of time during an exercise that the error is within a predefined error band). In the case of feedforward treatment exercises, the representation of the target parameter is suppressed for at least a portion of the exercise, and may even be suppressed during the entire exercise, such that whilst performing the feedforward exercise the user has to predict, or estimate, the required amount of force or control during the suppressed portion to match the target parameter. In some embodiments the suppression may be configured such that only a future target value or a future set of target parameter values are displayed, for example by displaying a curve with one or more portions occluded, or by displaying a target value to be reached at a specific point in an action (e.g., a peak value). The feedback representation of the target parameter may be provided whilst the target parameter is suppressed, or both the target parameter and feedback representation may be suppressed at the same time. As will be discussed further below, feedback exercises can progress to feedforward exercises.

As noted above, a Neuro-Modulation Therapy (NMT) apparatus or system may be used to implement the method. These NMT apparatus may be custom built apparatus, or an exercise/gymnasium apparatus which have been suitably modified to enable measurement of neuromuscular control for example by integrating a resistive element and a sensor apparatus, such as a load cell. Musculoskeletal control can be characterised by the ability to deliver the required force at each of a plurality of defined joint angles within a co-ordinated manner. Thus, whilst strength alone is not the primary determinant of rehabilitation progress, resistance strength training equipment and techniques may be modified for use as NMT apparatus as by adding a resistive element, or an extension component with a resistive element, along with a sensor or sensing arrangement. The extension component may be an elastic resistance band, spring or similar component. The sensor or sensing arrangement may be used to measure the force applied by a user and the sensor may include force transducers, strain gauges, load cells and accelerometer-based sensors. The force may be measured as a function of time (i.e., a time series of measurements may be obtained by sampling the force measurements at a sampling rate, which may be a fixed sampling rate, or it may be varied depending upon the exercise being performed) and for a given joint angle or a range of joint angles to allow determination of the degree of neuromuscular control. In other embodiments, the sensor or sensing arrangement may be non-force based, such as angular sensors or positional sensors (i.e., that measure a point in 3D space) or even remote sensing technologies such as computer vision based methods configured to remotely measure joint angles during exercises to allow determination of the degree of neuromuscular control.

It would be readily apparent to one of ordinary skill in the art that extension and flexion refers to an exercise of certain joints such as the knee. However, embodiments of the method may be used for assessment of all joints and musculature. Therefore, extension and flexion apparatus shall be understood to include other joint movements and their respective apparatus—abduction, adduction, inversion, eversion, rotation and any joint movement.

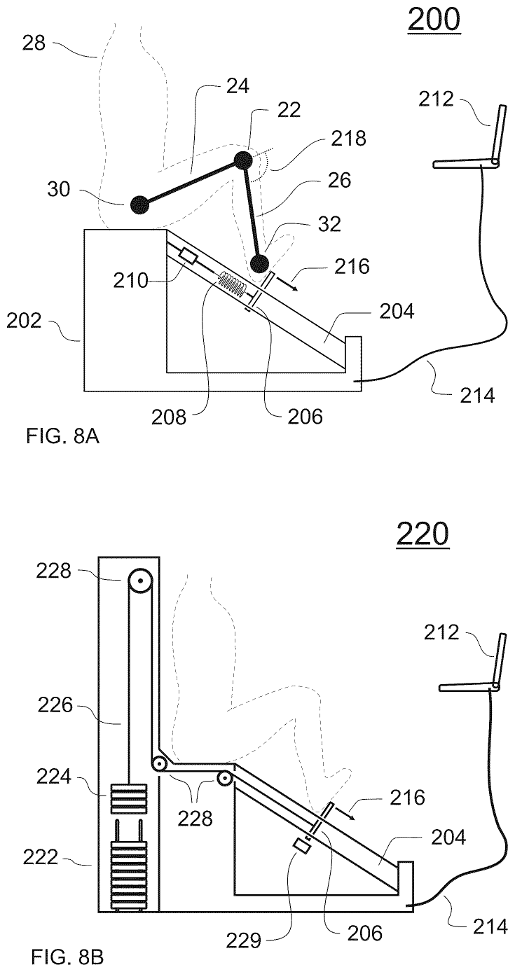

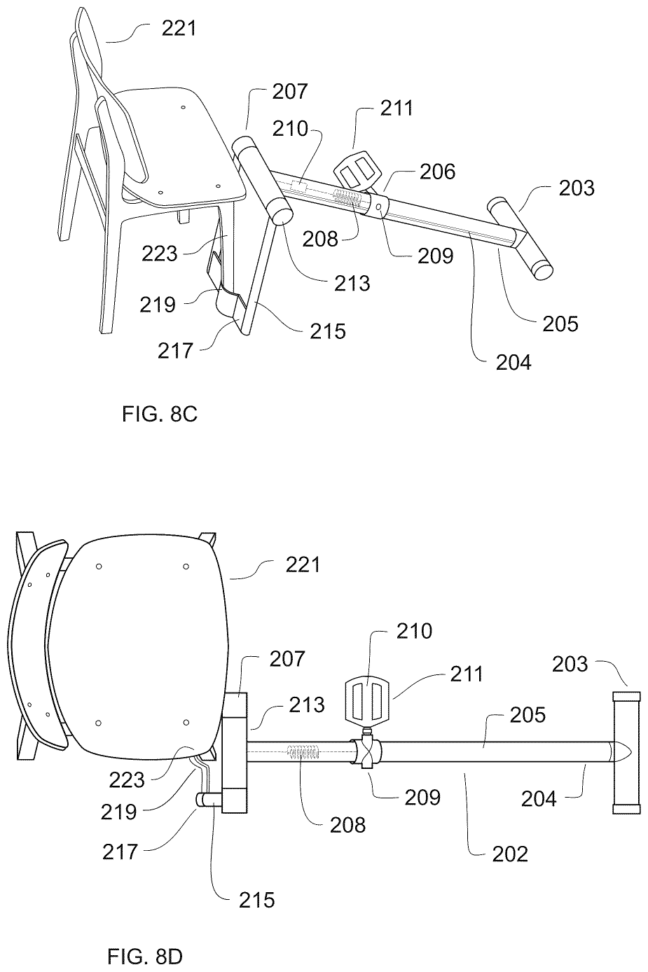

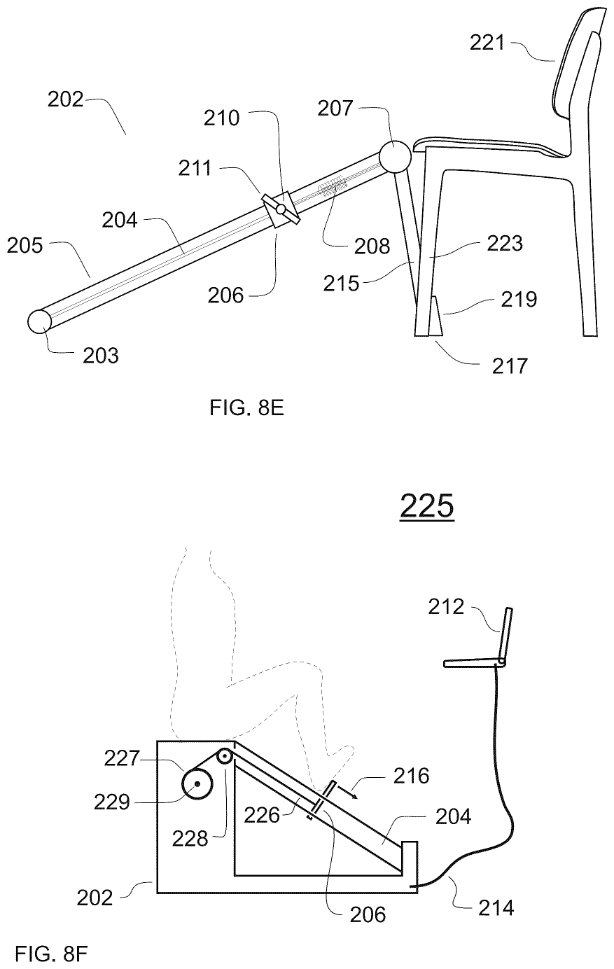

FIG. 8A is a sagittal plane schematic representation of an embodiment of a Neuro-Modulation Therapy apparatus at 200, configured to employ resistance training of the knee joint where similar elements from previous figures are similarly numbered. NMT apparatus comprises, in a first part, an extension apparatus 202 which consists of a linear guide rail 204 along which a sliding carriage 206 is free to move. Sliding carriage 206 is attached to a resistive element 208 which is also rigidly attached to a sensing arrangement such as a force measurement apparatus 210 which is itself rigidly attached to the apparatus 202 proximally to the seated position of the user 28. Furthermore, the NMT apparatus may comprise or integrate, in a second part, a computing apparatus 212 connected to the force measurement apparatus 210 of the extension apparatus 202 via a data cable 214. Alternatively, a NMT system may comprise the NMT apparatus 202 and a computing apparatus 212 wherein the computing apparatus is configured to analyse the data from the force measurement apparatus 210 according to embodiments of the methods described herein. The computing apparatus may be connected to the sensor apparatus via a wired connection, such as via a data cable 214 or via a wireless connection. Alternatively, the NMT system may comprise the NMT apparatus 202, and software modules configured to execute on a computing apparatus 212 and implement embodiments of the methods described herein. Multiple computing apparatus may be used, such as a first computing apparatus configured to calculate differences and/or analyse difference measurements and a second computing apparatus to display results (e.g., differences or the ranges where the user lacks control). Resistive element 208 is represented as a spring in the schematic representation. This may be provided by a coiled wire spring as explicitly shown in FIG. 5B as an extension spring 118. FIG. 5B may be rearranged and the resistive element provided via a compression spring, a torsion spring, a leaf spring or some other form of spring as would be readily apparent to one of ordinary skill in the art. Similarly, other forms of resistive element may be provided including but not limited to an elastic material such us rubber as would be the case with a TheraBand™ or some form of elastic rope in the form of a bungee cord or shock cord. In this embodiment the first part of the NMT apparatus is referred to as an extension apparatus. This refers to the functional exercise of extending the distal member 26 of the leg and is the opposite of flexion. Throughout this disclosure the first part of the NMT apparatus will be referred to as an extension apparatus for consistency of discussion. However, it would be readily apparent to one of ordinary skill in the art that NMT may be applied to other joints in the body.

The knee is a tightly constrained joint with only flexion and extension possible. The elbow is able to move in flexion and extension but has one more degree of freedom, allowing pronation and supination of the distal end of the distal member. The wrist allows flexion and extension but also includes lateral movements known as abduction and adduction. The ankle is more complex still with the motions defined as plantarflexion, dorsiflexion, inversion and eversion. The hip is a ball and socket joint with motion defined as flexion, extension, abduction, adduction, internal rotation, external rotation and circumduction. The human shoulder is the most mobile joint in the body. This mobility provides the upper extremity with a tremendous range of motion such as adduction, abduction, flexion, extension, internal rotation, external rotation and 360° circumduction in the sagittal plane. To round out the major joints of the body, the spine can move in flexion, extension, rotation and lateral flexion. These movements occur as a combination of rotation and translation in the sagittal, coronal and axial planes.

It would therefore be a complex and lengthy task to describe the application of Neuro-Modulation Therapy with regard to each and every possible joint movement. Therefore, throughout this disclosure the first part of a neuro-modulation apparatus will be referred to as an extension apparatus notwithstanding its potential application to other joint movements—abduction, adduction, inversion, rotation and any joint movement disclosed or otherwise—each being readily apparent to one of ordinary skill in the art. However, it will be generally understood that irrespective of the specific joint, the apparatus is configured to allow the target joint to be measured at a joint range or move over a certain joint range or joint ranges (i.e., in 1, 2 or 3 dimensions).

In the above example embodiments, the resistive elements are all passive elements. In some embodiments the resistive elements may be active resistive elements. In this case the resistive element may be a pneumatic apparatus or hydraulic apparatus that can be adapted, with the use of suitable sensors and/or the use of a suitable feedback control loop, to deliver either a constant force or a variable force across the range of motion as required. In a preferable embodiment an electric motor is used as an active resistive element. In such an embodiment the electric motor could be further adapted to include force or torque sensors to deliver a desired force profile across a range of motion. In another embodiment, the force may be measured by a force sensing apparatus comprising a force sensor, an Inertial Measurement Unit (IMU) and communications module. In this embodiment the IMU tracks three dimensional position and orientation (posture) data, which is wirelessly transmitted with force data to a computing apparatus to estimate the applied force and joint angle as a function of time whilst performing the exercise.

Rehabilitation often focuses on the measurement of strength, but this is highly dependent on joint angle, shown as angle 218 in FIG. 8A. Historically most knee strength research selects a 90 degree joint angle due to the ease of measurement at this angle. However, the knee is in its most loose packed and unstable in a mid-flexion position between 30 to 60 degrees. This is consequently the same range which patients with a ruptured anterior cruciate ligament will suffer instability. Total knee replacement patients also report mid-flexion knee instability and weakness at a joint angle of roughly 45 degrees. Thus, in some embodiments the testing and treatment of these instabilities is performed at the mid-flexion point. As the user 28 extends their leg, the sliding carriage 206 moves along the guide rail 204 as shown in 216, the knee joint 22 is made to move through a range of angles 218, which preferably includes the mid-flexion instability. Additionally, as would be recognised by someone of ordinary skill in the art, the spring 208 lengthens and the force applied to the sliding carriage 206 on the user 28 increases accordingly by Hookes' law—the force applied by a spring is proportional to its extension. Force is a proxy for the position of the carriage 206 and hence the extension of the leg of the patient 28. In this way, measured force and measured position of the carriage are providing similar information about the extension or position of the patient's leg. The exemplary joint is therefore exercised across a range of force conditions and a range of angles.

Force sensing apparatus 210 transmits force measurements via data cable 214 to computing apparatus 212. In one embodiment the data cable 214 is a Universal Serial Bus (USB) cable which provides both power to the force measurement apparatus 210 and a digital data connection between force sensing apparatus 210 and computing apparatus 212. It would be readily apparent to one of ordinary skill in the art that other forms of data cable may be used in other embodiments, including but not limited to Firewire, Thunderbolt, Ethernet, CAN bus, I2C or any of the RS-4xx protocol busses. Furthermore, data cable 214 may be operable via copper or fibre optic physical layers. Alternatively, the data connection embodied in the presently disclosed data cable 214 may be provided via a wireless apparatus using transports and protocols including but not limited to WiFi, Bluetooth, Zigbee, Thread, LTE and GSM cellular networks. Force measurements may be captured at a predefined sampling rate including rates such as 1 kHz or more, or at low rates such as 20 Hz, 1 Hz, 50 Hz, etc. The sampling rate may be fixed or variable and may be set based on the capabilities of the sensing apparatus or sensor, speed of the communication link, processing power, or complexity of the exercise. For example, a controller could vary the sampling rate to increase data capture during more complex actions.

Using the extension apparatus 202, the exemplary knee joint 22 is restricted to move only in the sagittal plain and thus operate in one degree of freedom. Further embodiments wherein the joint is operable in more than one degree of freedom will be readily apparent to one of ordinary skill in the art and these embodiments are therefore anticipated in the present disclosure.

The sensing apparatus may be a force measurement apparatus (or force sensing device) 210 configured to measure the force applied by a user as a function of time whilst the user is performing an exercise. A range of force measurement apparatus may be used including without limitation force transducers, strain gauges and load cell based technologies. In some embodiments these force measurement apparatus measure forces (or strain) at sample rates of 1 kHz or more. The load cell may also be incorporated into any of the force-loading apparatuses as an integrated neuromuscular characterising apparatus. The load cell may be connected to the computing apparatus by a wired or wireless communications link. In one embodiment the input apparatus comprises a portable load cell apparatus, comprising a load cell with a wireless communications interface configured to wirelessly transmit measured force data to a computing apparatus. This may be a portable load cell apparatus as described in PCT application PCT/AU2019/000078 filed on 24 Jun. 2019 and claiming priority to provisional application AU 2018902252 filed on 22 Jun. 2018, the content of which is hereby incorporated in its entirety. In one embodiment the portable load cell apparatus comprises an attachment arrangement, such as a pair of hooks allowing the load cell apparatus to be temporarily attached to and added in-line to the force transfer components of an exercise apparatus. In one embodiment the portable load cell apparatus is adapted to temporarily attach, or be removably attachable, to an exercise apparatus and in yet another a load cell is permanently integrated. The load cell may be configured to measure a force under dynamic conditions whereby the joint or adjacent joints are moving. The extension apparatus may incorporate rigid or stiff materials to maximise the force transmitted to the load cell and would be analogous to the case of the load cell being attached to a fixed point. In the case of a fixed point attachment or an extension apparatus with stiff/rigid materials the load cell would be measuring forces under static conditions, namely the force applied by the user at a fixed joint angle or series of joint angles. It would also be evident to one of ordinary skill in the art that the force may be applied in extension or compression as would be required of the specific exercise or rehabilitation programme. It would also be evident that this would be applicable to both the flexible extension apparatus, the rigid extension apparatus and the fixed attachment point cases.

In one embodiment the load cell is integrated within the extension apparatus or any force-loading apparatus for the purpose of measuring a specific action, specific exercise, or specific actions in an exercise, within an occupational, gym, exercise, clinical or rehabilitation setting. The extension apparatus may include a plurality of features that allow integration of the load cell such that the system can perform measurements of neuromuscular control for a specific physiologic action in a range of joint angle positions. The load cell may or may not be easily removable from the extension apparatus. For example, the load cell may be integrated into a thigh squeezing, adduction style ‘extension apparatus’ to allow specific measurements around the hip. This also could be incorporated into a hand grip apparatus for measuring neuromuscular control of the finger, fingers, hand grip or other joint. The hand grip apparatus could also incorporate the wrist such that both hand and wrist can be assessed at the same time or in isolation. Likewise, a chair-desk or occupational setup could incorporate an extension apparatus into its physical space to measure trunk and neck neuromuscular control at periodic intervals both as prophylactic and as a rehabilitation interaction in a specific occupation.

In one embodiment the attachment points of the portable load cell are adapted to mate with a plurality of accessories. Accordingly, a convenient attachment point would be a simple hook that would allow quick and reliable attachment of a plurality of accessories. Measuring a task-specific output of neuromuscular control is important for people who play sport or conduct specific tasks within the workplace. For example, a baseball player or cricketer is required to throw a ball of certain size, shape, texture, grip and weight. Measuring the execution of a throwing action via a ball attachment to the load cell will capture the whole-of-body action that imparts force onto the ball and the resultant end outcome. This can be simply attached and detached via a hook and loop (Velcro) method. Similarly, a manufacturing line worker maybe employed to undertake a repetitious action of using a specific hand tool to assemble parts. For example, the worker uses a mechanised wrench to apply a nut to a bolt whereby the user applies force to control the tool from rotating in the hand. A measure of hand control may be used as a guide of neuromuscular fatigue and hence reduce the effects of a repetitive stain injury. In this particular case, a sensor within the hand tool may monitor the hand tool for parameters such as rotational inertia as the bolt reaches the desired torque setting. The end outcome of measuring the neuromuscular performance of the specific hand tool via an attachment will be important to capture the repetitive manner of human inefficiencies and potential impact on repetitive overload injuries. This also could be in the form of a trunk attachment to measure the neuromuscular control for people suffering back pain within a seated arrangement. For example, the user is instructed to extend and release their low back into the chair against a resistance a plurality of times as a way to measure neuromuscular control after sitting for an extended period of time to examine the effects of postural control. It would be readily apparent to those skilled in the art that the embodiments described herein are readily applicable to the determination of any neuromuscular process, be it control deficiency or targeted objective.

Attachments enable various movements and functions at target joints and can be incorporated into whole body motion. A handle of different length can be used that allows a multitude of both isolated and combined joint motion. A static motion or dynamic motion could be used for the following attachments. For example, a swivel handle will allow the joint motion of wrist flexion/extension, or ulnar/radial deviation or circumduction. Likewise, this could be applied to the elbow to allow flexion/extension, shoulder flexion/extension or internal/external rotation or abduction/adduction or circumduction. A handle could also be used for functional activities such as hitting and striking, useful for sport endeavours or occupations. The handle could be extended in one direction to allow an offset used for hammering attachments that target elbow and wrist supination/pronation motion. A sling or wrist harness could replace to replace the handle. A plate could be used as an alternative to push against to allow some of the motions previously mentioned. Other attachments such as a dart to target fine finger and wrist motion can be used. A spear attachment such as that used in javelin could provide advances to improve whole upper arm and trunk motion used in throwing sports. A ball attachment could allow all of the handle joint motions with the added benefit of being more functional to throwing and pitching, used in sporting pursuits. This further could be enhanced by using a ball connected to a handle via a chain attachment, such as that used in hammer throwing. This could be a disc attachment such as that used in discus. For the trunk motions of rotation, flexion/extension, lateral flexion or combinations of these, a handle to grip or a harness attached to the body would allow an important attachment to achieve these motions. For the lower limb, a pedal, plate, ankle harness or strap or sling could allow motions of hip flexion/extension, abduction/adduction, internal/external rotation, circumduction, knee flexion/extension and ankle plantarflexion/dorsiflexion or inversion/eversion as well as circumduction. Someone skilled in the art will appreciate that a combination of whole-body movements can be applied using these attachments to improve function and that any attachment currently used in gym or rehabilitation settings will allow for various alternatives to achieve recovery or performance. It would be readily apparent to those of ordinary skill in the art that the load cell is merely one form of force measuring apparatus. Other types of force measurement apparatus may be interchangeably used in other embodiments including, but not limited to, load cells, force sensitive resistors, strain gauges, piezoelectric sensors, inertial measurement units and accelerometers.

It would be readily apparent to one of ordinary skill in the art that spring 208 is representative of any resistive element, including but not limited to a spring, an elastic element such as a TheraBand™ or other elastic material. In another embodiment the extension apparatus is designed such that the spring 208 is substituted for an apparatus that maintains a constant force regardless of the position of the sliding carriage. An example of this would be an attachment wherein extension of the leg would raise an attached weight. In another embodiment therefore, FIG. 8B is a sagittal plane schematic representation 220 of leg press gym equipment adapted for use as an extension apparatus according to an embodiment where similar elements from previous figures are similarly numbered. Leg press equipment 222 includes a set of weights 224 connected to sliding carriage 206 by non-elastic cable 226 via a series of pulleys 228. Motion of sliding carriage 206 along the guide rail 204 occurs with constant force applied by the suspended weight 224. In this embodiment the position of the sliding carriage 206 is measured by position sensor 229. Position sensor transmits information about the position of sliding carriage 206 via data cable 214 to computing apparatus 212. In one embodiment the position sensor 229 is a linear position encoder; a sensor, transducer or reed-head attached to the sliding carriage 206 paired with a scale attached to the guide rail 204 that together encodes position. The sensor reads the scale in order to convert the encoded position into an analog or digital signal, which can then be decoded into position by computing apparatus 212. The encoder can be either incremental or absolute. It would be readily apparent to one of ordinary skill in the art that other forms of position sensing are achievable including using a shaft encoder on one of the pulley axles and all such are therefore anticipated in the present disclosure. Furthermore, in one embodiment the system can provide positional information at sample rates of 1 kHz or more.

It would be readily apparent to one of ordinary skill in the art that a number of combinations of apparatus and measurement sensor technology may be equivalently substituted into different embodiments of NMT apparatus, and all are to be considered interchangeable and functionally equivalent within the intent of the present disclosure. It would also be readily apparent to one of ordinary skill in the art that other forms of equipment may be adapted for use in other embodiments, such as but not limited to, resistance training systems, therapeutic apparatus and standard exercise/gymnasium apparatus.

FIGS. 8C, 8D and 8E illustrates another embodiment of an NMT extension apparatus 202 similar to those shown in FIGS. 8A and 8B and configured for use with a chair 221. This embodiment is a portable NMT apparatus configured for home use. FIG. 8C shows a perspective view configured for treatment of the right knee, and FIGS. 8D and 8E show corresponding top and side views. The user may use a suitable chair 221 at their own home, or a chair 221 may be provided with the apparatus 202. The apparatus 202 comprises a base 203, a central shaft 205 and a pivotable support arrangement 207. The pivotable support leg arrangement 207 comprises an upper mounting bar 213, two legs 215 and a chair locating connector 217. A sliding carriage 206 is mounted or located around the central shaft 205 and has two opposing pedal mounts 209 each configured to receive a pedal 211. The sliding carriage is connected to a resistive element 208 located internally within the shaft. In this embodiment the resistive element is an extension spring anchored at the top of the central shaft where it meets upper mounting bar 213 and is shown as a dashed line in FIG. 8C. The shaft 205 is provided with two linear guide side 204 located on opposite sides of the shaft and receive and guide the sliding carriage 208, for example the sliding carriage may comprise a pair of T-pieces in which the shaft is received in a gap between the rails 204. The spring is biased to pull the pedal towards the upper mounting bar 213 (i.e., top of the shaft 205) such that as the patient 28 sits on the chair 221 they can push the pedal towards the base to extend the spring 210, and can also apply a resistance force to the pedal as it is pulled back towards the upper mounting bar 213 by the spring 210 (as it compresses returning from the extended position). As the pedal slides up and down the shaft, the internal resistance spring is extended and compressed. The base 203 rests on the floor and is a T shaped piece, and may be a heavy piece and/or comprise high friction feet to prevent movement of the base during use.

The upper mounting bar 213 receives the upper end of the shaft 205 (distal with respect to the base) and the upper ends of the two legs 215 are pivotally mounted to the ends of the upper mounting bar 213. The chair locating connector 217 connects the base ends of the two legs 215 and has a central chair locating portion 219 which in this embodiment is a curved section which matches the profile of the shaft 205 such that when the pivotable support arrangement is folded against the shaft 205 the extension apparatus 202 is substantially flat and compact for storage and transport. In other embodiments the chair locating portion 219 could have another profile or shape. The legs 215 are configured to pivot with respect to the upper end of the shaft 205, and the rotational range of the legs 215 with respect to the shaft may be limited to a range, such as 0-25°. The pivot mounts for the legs may be configured such that the legs can be locked at any desired angle in the range, locked only at the minimum (0°) and maximum (25°) ends of the range, or at specific increments over the range (e.g., 5° increments).

In one embodiment the support legs fold out and are locked at the maximum range (25°) to create a 25° incline with the ground. A chair leg 223 is located in the central chair locating portion 219 of the chair locating connector 217, preventing the chair 221 (and the patient 28 not shown) from sliding away from the apparatus when the patient uses the apparatus. The pivotable support leg arrangement 207 can also be pivoted and folded up to allow the apparatus to be used flat with the patient position supine on the floor or in a bed. As outlined above, the pedal 211 slides down and up the shaft 205 as the patient extends their leg, stretching the internal resistance spring 210. The pedal 211 is mounted to the sliding carriage 206 by a pedal mount 209 which is configured to allow the pedal 211 to freely rotate as the leg extends forward to maintain a neutral 90° angle with the ankle. In this embodiment the pedal 211 comprises an integrated force sensor 210, such as a power meter, and further comprises a communications interface configured to communicate with computer 212, for example to send measured force data. The communications interface may be a wireless communications interface (e.g., Bluetooth, or ANT+), or a wired interface such as USB connector. The pedal 211 may be mounted on either side of the shaft 205 to provide therapy to either leg. For example, to provide therapy to the right knee, the pedal 211 is mounted on a left side of the shaft 205 (viewed from above) and the right chair leg 233 is received in the central chair locating portion 219. Then to provide therapy to the left knee, the pedal 211 is moved to the right side of the shaft 205, and the left chair leg 233 is received in the central chair locating portion 219.

Alternatively, the sliding carriage 206 may be designed or configured such that the pedal 211 can rotate or flip from one side of the shaft 205 to the other side, or two pedals 211 could be permanently mounted to both sides of the shaft 205.

FIG. 8F illustrates another embodiment of an NMT extension apparatus at 225 where similar elements from previous figures are similarly numbered. In an embodiment the resistive element is an active element or electric motor 227. Sliding carriage 206 is attached to electric motor 227 by non-elastic cable 226 via a pulley 228 (although the use of a pulley is not necessary in other embodiments).

Motion of sliding carriage 206 along the guide rail 204. In this embodiment the position of the sliding carriage 206 is measured by a shaft encoder 229 in motor 227. Position sensor transmits information about the position of sliding carriage 206 via data cable 214 to computing apparatus 212 as in previous embodiments. This embodiment has advantages over the passive resistive element. Using a passive resistive element requires a specific set point of parameters for each patient. Force is proportional to extension, so a given spring provides a given force range over a specific extension. Users with long legs reach a higher force than users with shorter legs and accommodating both can result in a compromise in resolution or dynamic range of the force measurement system. The use of an active resistive element allows independent configuration of force and extension.

In practise, the NMT extension apparatus would undergo a simple calibration cycle that would configure the system for each user. The user would be seated, and the system would:
a) Determine the maximum leg extension of the user. The user is asked to extend their leg as far as possible while the device measures the maximum extension (either via a direct positional measurement or by the force measurement).
b) Determine the minimum leg extension of the user. The user is asked to retract their leg as far as possible while the device measures the maximum extension (either via a direct positional measurement or by the force measurement); and
c) Determine a calibration factor that maps the user's range of extension to the system's required range of motion.

As a user progresses through NMT, their range of movement may increase, and a different force profile may be required. These parameters can be dynamically adjusted much more easily with the embodiment of FIG. 8F.

Resistance training systems include but are not limited to multi-station gyms, pulley systems, cable systems such as lat-pulldowns, cable rows, knee extension machines, hamstring curls, hip flexion/extension/abduction/adduction, calf raises, bench press, bunch pull, pec deck machines, shoulder press, shoulder pulls, shoulder abduction/adduction/extension/flexion machines, triceps extension, bicep curls, arm curls, wrist curls, trunk rotations, trunk flexion/extension machines, smith machines and leg presses. Therapeutic apparatus includes but are not limited to Pilates equipment including reformer, Cadillac, Trapeze, Ladder Barrel, Spine Corrector, Pilates Chair and Pilates Ring. Force plate measurement equipment, all forms of force resistive apparatus and devices not limited to ForceFrames, Nordic exercise apparatus, jumping mats and various forms of resistance bands. Standard exercise/gymnasium apparatus include but are not limited to manual and motorised treadmills, body-weight supported treadmills, elliptical, rowing machines, upright and recumbent bikes, spin bikes, under-desk bikes, stairmasters, steppers, stair climbers, stepmills, arc trainers, ski ergometers, arm ergometers, punching bags, climbing ropes, rope apparatus such as battle ropes, skipping ropes, various forms of resistance bands and body weight systems such as TRX Suspension systems. These may be suitably adapted to include sensing technologies which can be operatively connected to a computing apparatus to reconfigure the apparatus as a NMT apparatus.

In other embodiments the joint angle may be measured using a computer vision system as a proxy for position. For example, the user could wear markers over the joint and on the proximal and distal members to allow a computer vision system to capture the motion of the markers and provide estimates of the joint angle function as a function of time—in the case of the exemplary knee, the knee joint 22, the femur 24 and the members comprised of the tibia and fibula 26 of FIG. 2A. Indeed, computer vision systems are becoming increasingly sophisticated and any sensors, apparatus or systems capable of identifying the joint angle or position of the sliding carriage 206 may also be used. In yet other embodiments a digital goniometer or inertial measurement units (IMU) worn by the user provides real time information about the position or motion of the exemplary knee and its associated members.

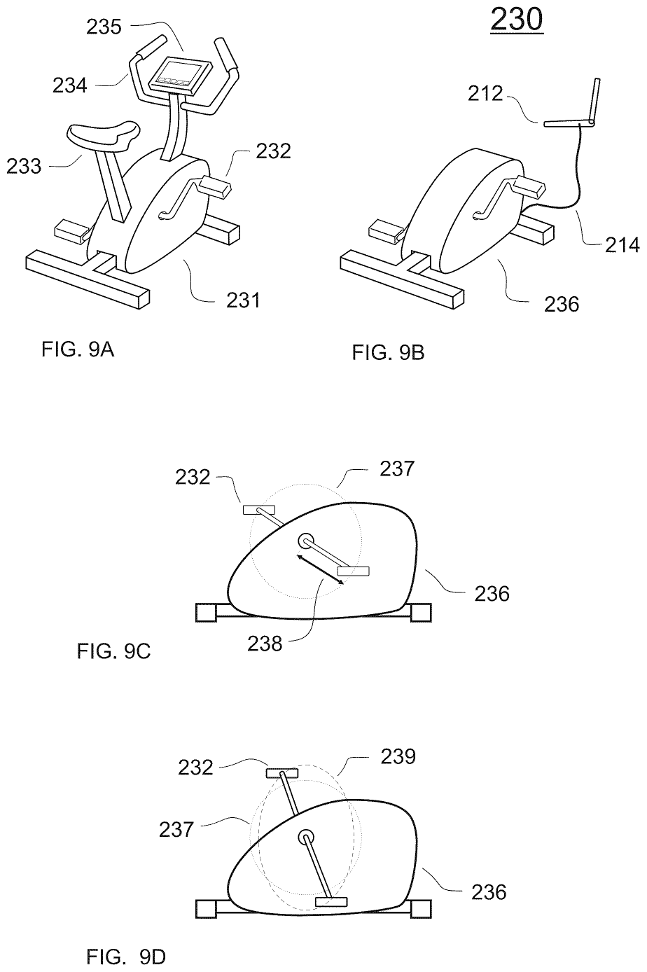

It would be readily apparent to one of ordinary skill in the art that the neuro-modulation apparatus may take the form of other typical exercise equipment. FIGS. 9A to 9D are therefore a representation a stationary or exercise bike adapted to perform the function of a neuro-modulation apparatus at 230 where similar elements from previous figures are similarly numbered. FIG. 9A is a schematic representation of a neuro-modulation apparatus in the form of a conventional stationary or exercise bike 231 which contains pedals 232, a seat 233 upon which the user can sit, handlebars 234 which a user can use to steady themselves and a computing apparatus 235. Pedals 232 drive a shaft to which a resistance element 208 (not shown) and a force sensor 210 (not shown) is attached, which are configured to determine the torque required to rotate the pedals. The variable resistive element 208 may be a friction apparatus, a geared system, an electromechanical system, an electromagnetic system or some other form of resistive element such that the resistance element 208 and force sensor 210 are functionally equivalent to a resistive extension apparatus such as shown in FIG. 8A. In an alternative embodiment the force sensor 210 is integrated in the pedal 232, or the pedal shaft (that is screwed into or retained in the crank shaft, and about which the pedal body rotates). The force sensor may comprise a load cell, a battery and wireless communications module (e.g., Bluetooth or ANT+) such as described in PCT/AU2019/000078, or it may comprise a pedal power meter such as those manufactured by SRM (https://onlineshop.srm.de/) including the X-power Flat pedal range, Favero electronics (https://cycling.favero.com/en) including the Assioma range, or Garmin (https://www.garmin.com) including the Garmin Vector range. A pedal power meter may be used in one or both pedals.

In an embodiment the resistive element is an inductive element or electric motor which can provide a constant or variable force against which the user pushes the pedals and also senses the force applied or angular velocity of the pedals. In some embodiments the sensing element is a shaft encoder which provides information as to position and hence the time taken for the pedals to rotate through a certain angle. Computing apparatus 235, which itself may be operably connected to another computing apparatus or cloud computing resources, may contain a display apparatus and a program or software module for instructing the user, providing feedback to the user or any of the other functions taught of a computing apparatus in this disclosure. Computing apparatus 235 may additionally contain one or more cameras or imaging sensors as a source configured to capture imagery or video of a user using the neuro-modulation apparatus. Neuro-modulation apparatus 231 and its inherent extension apparatus may be operated at constant angular velocity, at a variable and well defined angular velocity, in one direction or both directions (pedalling forward and reverse) and any combination thereof. Not all populations are able to operate a stationary exercise bike. Ageing populations for example may not have the balance required to sit up on the seat 233. Thus, another embodiment of the bicycle style neuro-modulation apparatus may be provided in FIG. 9B as a pedal set unit 236 which can be placed in front of a user's chair. Pedal set neuro-modulation apparatus 236 may be functionally equivalent to the body of the stationary exercise bike 231 and may be operably connected to a computing apparatus 212 by a data cable 214 or alternatively via a wireless apparatus (not shown). Computing apparatus 212 may also be optionally connected to cloud computing resources and retain all of the features of the neuro-modulation apparatus 231, except the seat, handle bars and embedded display apparatus.

These embodiments of the neuro-modulation apparatus, 231 and 236, have the advantage that the pedals 232 may incorporate restraining foot straps, foot slots or preferably cleats that create a direct and consistent connection between the user's foot (your shoe) and the pedal. This means the user can push and pull on the pedal all the way through the pedal stroke. Cleats also keep the user's feet in a solid and consistent position, providing reliable alignment of the middle of the foot through the centre of the pedal where measurement is applied and also avoiding the need to reposition the user's feet every few pedal strokes. Such an arrangement allows the user to apply dynamic control of neuro-modulation apparatus on both the down stroke and up stroke of each leg, working different muscle groups in the process. In yet other embodiments a force sensing is contained in one or both pedals 232 such that one or both pedals are capable of measuring the dynamic forces in both push and pull phases of a cycle, utilising cleats to attach the user's shoes to the pedals 232.

A full 360 degree pedal cycle is described as follows from the perspective of a single leg. Beginning at the top of the stroke, the pedal is driven forward and downward, hereinafter referred to as the push power phase. At the bottom of the pedal stroke, the user's foot reaches the lowest point of the cycle, and the leg is at its greatest extension. The user then begins to lift their foot rearwards and towards the top of the pedal stroke, hereinafter referred to as the pull power phase, flexing their leg towards the top of the stroke, at which point the leg is at the point of greatest flexion. It would be readily apparent to one of ordinary skill in the art that at any given phase of the pedal cycle, the user is engaging slightly different muscle groups. This is particularly the case when using cleats or a similar form of foot restraining apparatus. The neuro-modulation apparatus, 231 or 236, is therefore operable using a single leg at a time to isolate specific muscle groups. Alternatively, the user can operate the neuro-modulation apparatus using only 180 degrees of range. In this scenario, beginning at the top of the stroke, the pedal is driven forward and downward. At the bottom of the pedal stroke, the user's foot reaches the lowest point of the cycle, and the leg is at its greatest extension. The user then begins to lift their foot forwards and towards the top of the pedal stroke. Yet again this engages slightly different muscle groups. Furthermore, for a given cycling motion, continuous forward motion, continuous backwards motion or changing direction, a subtle change in the user's posture will change the muscle groups that are engaged during each phase of the cycling motion. For example, a seated posture behind the pedal set apparatus 236, a standing posture directly above the pedal set apparatus and a squatting position directly above the apparatus 236 will each engage slightly different muscle groups during the session. Finally, the user can operate the neuro-modulation apparatus, 231 or 236, with both feet in a continuous forward motion, continuous backwards motion or changing direction periodically or randomly as the control software dictates in any given neuro-modulation training session.

FIG. 9C is a schematic representation of the pedal set neuro-modulation apparatus 236 showing the circular path 237 taken by the pedals 232 when the crank arm is a fixed length 238 where similar elements from previous figures are similarly numbered. This describes the path taken by the pedals in a standard bicycle or stationary exercise bike. In some of the embodiments the length of the crank arm, defined as the distance between the axle and the pedals, may be varied to increase the range of joint motion. If the length 238 of the crank arm is increased, the knee undergoes a wider range of motion during one rotation. The crank arm length can be set to a desired length prior to an exercise. Thus over the course of a session, or several sessions of NMT, the crank arm length may be manually adjusted and set to a desired length, for example to incrementally increase the range of motion. An adjustable crank length 238 can be implemented using a variety of mechanical arrangements. In one embodiment the crank arm comprises multiple apertures along the crank arm, and a support comprises a removable retaining pin that may be received into any one of the apertures. The crank arm can thus be adjusted by removing the pin, sliding the crank arm relative to the support to set the crank arm length 238, and then reinserting the pin into a different aperture to retain the crank with the new length. The pin may be a spring loaded pin biased to drive the pin into an aperture, which can be adjusted using a button arrangement that temporarily drives the pin out of the aperture (and counteracts the bias force). Other arrangements such as two part arm where a first part has an internal cavity which receives a second part which is slidable with respect to the first part, and which can be fixed at either a nominated point or at any points along a range through the use of screw fasteners or similar fasteners. Embodiments may be based upon adjustable cranks such as those made by Hase Bikes (https://hasebikes.com/en/special-accessories/), SRM (https://onlineshop.srm.de/ergometer/). In another variation the crank arm may comprise multiple pedal receiving apertures such that whilst the crank arm may be of a fixed length or size, the relative distance from the axle to the pedal (or axle mount point to pedal mounting point) is variable, which as noted above is our definition of crank arm length. In one embodiment the crank arm is triangular plate structure which comprises an axle mounting point at the apex, and multiple apertures in the distal end of the plate (base) into any of which a pedal may be inserted (and retained) such that each aperture effectively creates a different crank arm length. In another embodiment the crank arm comprises multiple pedal receiving apertures along its length to allow the pedal to be mounted at various distances with respect to the axle and thus provide an adjustable crank arm length. Embodiments may be based upon those manufactured by Fouriers (https://www.fouriers-bike.com/en/), or Trek (https://www.trekbikes.com/).

FIG. 9D is a schematic representation of a pedal set neuro-modulation apparatus 236 with a variable crank length during one rotation where similar elements from previous figures are similarly numbered. In this arbitrary representation, the crank length is at a maximum when the crank is vertical and at a minimum length when the crank is horizontal with the pedals tracing out an elliptical path. The length of the crank arm may be varied between exercise sets or dynamically during a given exercise. The orientation of the major and minor axes of the ellipse may be adjusted arbitrarily to any phase angle relative to the orientation shown in FIG. 9D. Taken to its extreme, if the length of the minor axis of the ellipse approaches zero then the path of the pedals approaches a purely linear motion, approximating the motion of the linear slide neuro-modulation apparatus taught in FIG. 8A. The length of the crank arms may be adjusted in such a manner as to provide an elliptical path that has a fixed phase relationship to the coronal plane. The length of the crank arms may be adjusted in such a manner as to provide an elliptical path that has a monotonically increasing or decreasing phase relationship with the coronal plane, that is, the major axis of the ellipse changes its relationship with the coronal plane in a consistent way on each successive revolution. The length of the crank arms may be adjusted to provide some other non-elliptical pedal path or may even be adjusted independently of one another on the left and right pedals. It would be readily apparent to one of ordinary skill in the art that changing the length of the crank arm is equally applicable to the stationary bike 231 or the pedal set 236 neuromuscular apparatus.

In another embodiment the pedal set neuro-modulation apparatus 236 may be adapted for upper limb use by swapping out the foot pedal 232 for a hand grip or other type of human interface to allow use in rehabilitation of the upper limbs such as after shoulder, elbow or wrist injury or surgery. Furthermore, the pedal spindles to which the pedals 232 and hand grips are attached may be of an adjustable length, allowing for different body anthropologies such as wide shouldered or pelvis. Increasing the axle and/or spindle length will also allow targeted Neuro-Modulation Therapy in greater shoulder and hip abduction ranges. This could also allow an additional elliptical motion and change the neuromuscular combinations around a joint. Thus, the length of both the crank arm and the axle/spindle could be unilaterally adjusted to move away from mirrored pedal motion to further enhance neuromuscular adaptation. The pedal set neuro-modulation apparatus 236 may furthermore be provided as a desk top unit to allow a user to sit in front and use it for upper body rehabilitation.

In yet another embodiment, the pedals 232 of the neuro-modulation apparatus 231 or any of the pedals, grips or other end-effectors of the pedal set neuro-modulation apparatus 236 (generically described herein and throughout this disclosure as pedals 232) are operatively connected to the computing apparatus 235 or another computing apparatus, wherein connectivity may be by any of the wireless or wired communication protocols taught in this disclosure, hereinafter described as connected pedals. Connected pedals 232 are adapted to measure parameters of their motion in time and transmit to at least one of the computing apparatus such parameters, including the position of the connected pedals and the force exerted on them using any combination of strain gauge, gyroscope, inertial sensor, accelerometer or any other apparatus or devices which may provide a stream of information on the position, motion and/or the forces exerted on the connected pedals 232. The parameters may be transmitted to a computing apparatus for display to the user in real time. The use of connected pedals (including grips or other end-effectors) is anticipated across the breadth of this disclosure as would be readily apparent to one of ordinary skill in the art.

Furthermore, the neuro-modulation apparatus 231 and 236 may incorporate a headphone jack or some wireless apparatus, including Bluetooth, for connecting headphones, earbuds or other audio equipment. The audio equipment may be adapted to provide a user with instructions, music or other audio sounds to enable it to perform other aspects, including sleep memory activation, as taught throughout this disclosure.

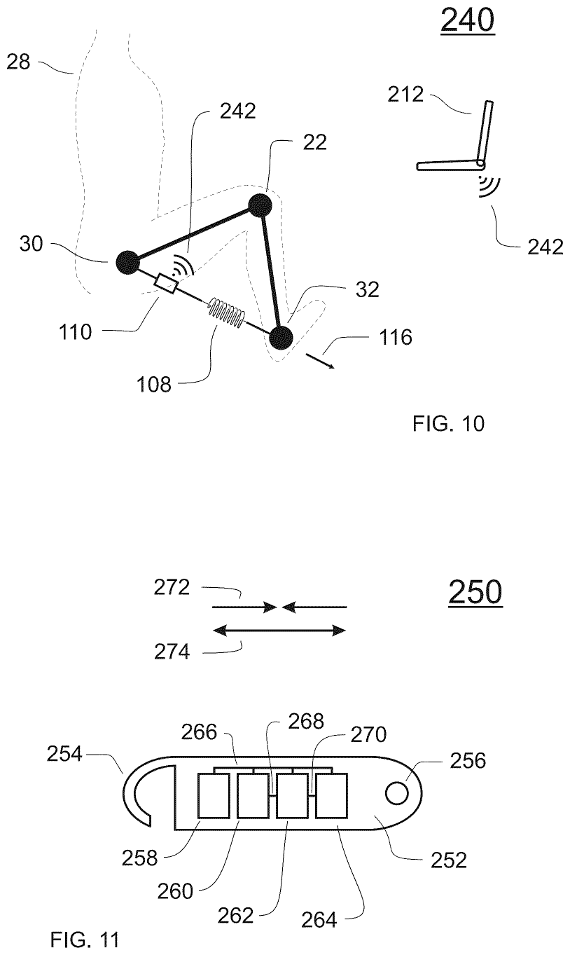

FIG. 10 is a sagittal plane functionally equivalent schematic representation 240 of an embodiment of an extension apparatus wherein only the functionally equivalent elements are presented and where similar elements from previous figures are similarly numbered. In an ‘equivalent-circuit’ sense, the hip 30 and ankle 32 are connected via resistive element 208 and a rigidly-coupled measuring apparatus 210, such as a force measuring apparatus, which in this embodiment is shown connected to computing apparatus 212 via a wireless protocol 242 (although it may be connected over a wired connection). In this configuration the hip 30 is the reference point and the knee 22 and ankle 32 are constrained to move only within the sagittal plane. Under these conditions the main operable muscle groups are the gluteals (not shown), quadriceps 42, the hamstrings 44 and calves 46. In one embodiment, resistive element 208 of FIG. 10 is replaced by a rigid coupling such that the user is unable to extend their leg and no motion 116 is possible. In this embodiment application of force against the apparatus provides a static measure of force at a fixed joint angle. In this way, assessment of both the user's neuromuscular control and maximum strength at each joint angle can be made by slight adjustment of the seating position or length of the rigid coupling. The force measuring apparatus 210 of FIG. 10 is rigidly coupled to the resistive element 208 such that force sensor apparatus measures the force applied against the resistive apparatus by the user at any given time. FIG. 11 is a schematic representation of a force measurement apparatus 252 at 250 which has attachment points 254 and 256, a battery 258, a load cell transducer 260, a computing apparatus 262 and a communication apparatus 264. Attachment points may be hooks 254 which allow force measurement apparatus to be inserted in-line with the force path of a resistance element or other exercise equipment or a hole 256 that allows attachment via a bolt or any other apparatus. Power supply bus 266 provides power from battery 258 to transducer 260, computing apparatus 262 and communication apparatus 264 that provides data to an external apparatus. There exists a measurement channel 268 that allows computing apparatus 262 to determine the instantaneous force on transducer 260 and a communication bus 270 that connects the computing apparatus 262 to the communications apparatus 264. Connection via bolt hole 256 would provide a rigid connection for the force measurement apparatus would allow measurement of forces under compression 272 or under tension 274, while connection via the hook 254 would limit measurements to tension 274. The force measurement apparatus 252 may contain connection via only hooks 254, only bolt holes 256, a combination as shown in FIG. 11 or any combination of other types of attachment apparatus as would be readily apparent to one of ordinary skill in the art. Communication apparatus 264 may be a wireless communications apparatus such as Bluetooth or any of the wireless or wired communication protocols taught in this disclosure.

High resolution measurement of these parameters may allow better identification of the mal-adaptive changes such as unwanted jerk in injured subjects, with the intent on improving the smoothness and restoring a healthy functioning nervous system. To this end the sensors contemplated in this disclosure maybe configured to provide high resolution determination of force, position or any other of the physical parameters at a high temporal resolution, such as by sampling at rates in excess of 100 Hz or even in excess of 1 kHz.

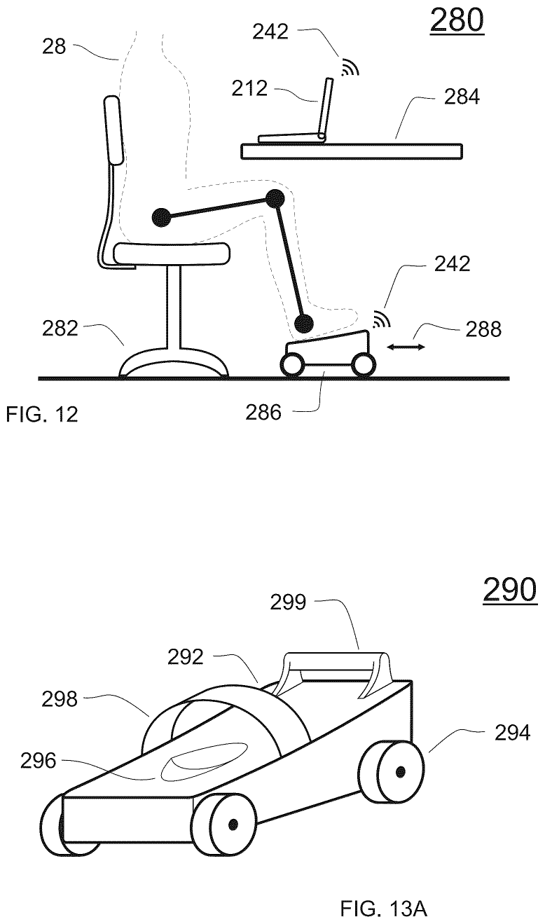

Repetition is beneficial for neuroplasticity and thus it is desirable that the rehabilitation treatment is conducted as separate sets, or sessions, of exercises which are performed multiple times over the course of weeks or months, for example each day, every second day, or once a week over the course of the treatment. However, it is not always practical for a user to have access to the large NMT equipped exercise equipment of FIG. 8 or 9. FIG. 12 is therefore a sagittal plane schematic representation of a portable neuro-modulation apparatus being used in an office environment at 280. User 28 is seated on office chair 282 at office desk 284 (shown without legs for simplicity) upon which computing apparatus 212 is placed. Continuing with the exemplary case of the knee, the user 28 places their foot on the extension apparatus component 286 of a portable neuro-modulation apparatus and extension of the leg results in extension apparatus component 286 moving fore and aft 288 in the sagittal plane.

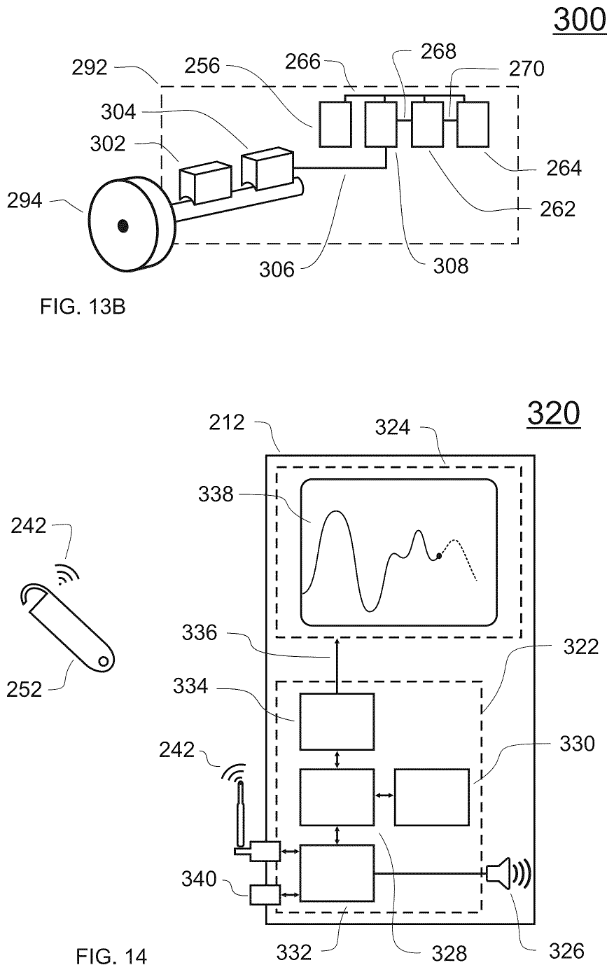

FIG. 13A is a representation of a portable extension apparatus at 290. Portable extension apparatus 292 has wheels 294. In one embodiment portable extension apparatus may have a wedged profile which may additionally be slightly concave or contoured in some suitable manner. Portable extension apparatus 292 may additionally have one or more depressions 296 to facilitate location of the heel for better grip or a profile that accommodates the palm of the hand. Extension apparatus 292 may optionally include one or more straps 298 that allow the foot to be securely located during use. Extension apparatus 292 may optionally include a handle 299 to enable upper limb use. FIG. 13B is a schematic representation of a portable extension apparatus at 300 where similar elements from previous figures are similarly numbered. Portable extension apparatus 292 contains a plurality of wheels 294, the axles of which are connected to at least one friction apparatus 302 and at least one shaft encoder 304. Friction apparatus 302 may be optionally applied to one or more of the wheels and is used to deliver an adjustable amount of friction to the axle such that the user can adjust the force required to move the apparatus. Shaft encoder 304 measures the rotation of the wheel, thereby providing a measure of the distance the portable extension apparatus moves in a given amount of time. Shaft encoder 304 provides, via bus 306, a signal to transducer 308, which in this case is adapted to read the output of a shaft encoder before further processing and transmitting data to an external computing apparatus.

Portable extension apparatus 286 can be used for therapy of the knee, quadriceps and hamstrings as shown in FIG. 12. If the portable extension apparatus 286 is rotated by 90 degrees, then its motion is in the coronal plane the apparatus would be used for therapy of the hip, exercising the hip rotator muscle groups. Similarly portable extension apparatus 286 could be used on the desktop 284, either in the sagittal plane moving the arm fore and aft or in the coronal plan moving the arm left and right. It would be readily apparent to one of ordinary skill in the art that portable extension apparatus 286 can be used in a variety of ways to treat a variety of muscle groups and these embodiments are therefore anticipated in the present disclosure. It would also be readily apparent to one of ordinary skill in the art that in addition to use in the office environment depicted in FIG. 12, the portable extension apparatus 286 can be used in a home office, at the dinner table, in front of the television, in a seated position, in a standing position, in a prone position, against a wall or any other suitable position and location that allows unrestricted motion in the desired plane and these embodiments are therefore anticipated in the present disclosure.

The exercise and force sensing extension apparatus as shown in its reduced, equivalent-circuit form in FIG. 10 is only one part of the Neuro-Modulation Therapy system which aims to retrain the mismatch between afferent and efferent signals in a damaged or surgically altered joint such as the knee. NMT consists of a specific range of exercises and a defined protocol, based on embodiments discussed herein, to prime the brain for neuroplasticity. NMT opens a window of opportunity where a short-term depression of the inhibitory function allows excitatory cells to create new neural connections. This allows the brain to efficiently rewire itself to recognise the new afferent signals from an altered joint under motion. NMT creates an environment conducive to neuroplasticity; the range of exercises then condition the brain to a new set of expectations from the altered afferent signalling under a defined efferent motion command.

When first learning a motor task, movement is often slow, stiff and easily disrupted without attention. This is similar to the physiological presentation of arthrogenic muscle inhibition associated with a joint injury or recovery from surgical intervention. With practice, execution of a motor task becomes smoother, there is a decrease in limb stiffness and muscle activity is performed without conscious effort.

The neuroanatomy of memory is widespread throughout the brain. The pathways important to motor memory include the motor and somatosensory cortices but the main area involved in motor learning is the cerebellum. Studies of cerebellar-dependent motor tasks show that cerebral cortical plasticity is crucial for motor learning. The basal ganglia also play an important role in memory and learning, in particular relating to stimulus-response associations and the formation of habits. Importantly, the basal ganglia-cerebellar connections are thought to increase with time when learning a motor task. Hebb's rule states that “synaptic connectivity changes as a function of repetitive firing”. That would suggest that the high amount of stimulation coming from practicing a movement would cause the repetition of firing in certain motor networks, presumably leading to an increase in the efficiency of exciting these motor networks over time. This would assist the formation of these new neural representations within the motor cortex by up regulating neurotropic factors that could enhance the survival of the newer neural maps formed due to the skilled movement training. Mere repetition though is not enough. Functional issues resulting from an injury or surgical intervention are largely treated as a strength deficiency. Little consideration is given to the lack of control. Prior art rehabilitation focuses on repetitive strength training and quantification of strength is the primary measure of efficacy—all of which have largely proven ineffective. Embodiments teach a methodology for creating the preconditions for neuroplasticity and thereby retraining the brain to accommodate the new, altered afferent signalling delivered by a joint that has been altered in some way, through inflammation, damage to soft tissue, via a completely new, surgically altered geometry or some other apparatus.

As previously stated, neuromuscular control can be considered as applying the right force, to the right action, at the right time. The degree of neuromuscular control can then be characterised by the ability to smoothly deliver a required varying force across a range of defined joint angles or in the case of the exemplary equivalent-circuit of FIG. 10, across a range of leg extensions 116. This would typically be determined by measuring the progression of a movement under load; measuring at least one parameter such as the position, force or angle as a function of time, wherein an individual suffering from AMI would typically exhibit jerky movement under load. It would be readily apparent to one of ordinary skill in the art that derivatives of position such as velocity and acceleration or combinations are equivalent parameters and these can also be substituted, but not limited to these variables.

Neuromuscular control is a complex system of feedforward command, cortical drive, motor neuron signalling, sensory feedback, cerebellar processing and error correction, combined with spinal-reflex feedback pathways. Arthrogenic muscle inhibition is a short circuit in that process preventing accurate application of the right force to the right action at the right time—this must be overcome in any treatment regime. Embodiments teach a method, referred to in this disclosure as Neuro-Modulation Therapy, of priming the neurological pathways, postulated to occur largely in the cerebellum, for neuroplasticity thereby reducing arthrogenic muscle inhibition and allowing restoration of function.

Embodiments of NMT may utilise a combination of feedback and feedforward mechanisms that appear to accentuate the body's capacity for motor learning plasticity and accelerate the reorganisation of the body's internal model of movement within its environment. Neuro-Modulation Therapy primes the body to assimilate the ‘new normal’ of post injury or surgical intervention afferent joint information into its internal model of the world. Parallel Fibre to Purkinje cell synapses has been shown to be especially plastic under certain conditions and this appears to be part of the mechanism for reducing arthrogenic muscle inhibition. Repetition is a precondition for reinforcing the strength of synaptic connections. NMT therefore utilises a series of repetitive exercises which, crucially to the process, involve a combination of feedback and feedforward training; initially begin as feedback training but progress to feedforward training.

Neuro-Modulation Therapy is a system that utilises an extension apparatus including but not limited to those disclosed in FIGS. 8 through 10. The method requires a user to replicate a defined pattern of force (position, velocity or some other target parameter) versus time using the extension apparatus. In one embodiment the defined pattern is a simple, repeating pattern although other more complex repeating or indeed non-repeating patterns may be used. Referring to the exemplary embodiment of FIG. 8A, the defined pattern is presented to the user as a visual graph of force versus time by computing apparatus 212 which the user must replicate using the extension apparatus 202. Force sensing apparatus 210 measures the force applied by the user which is transmitted to the computing apparatus 212 and displayed in real time, in this preferred embodiment, via a wired communication protocol. The method of presenting the defined pattern to the user can be a visual apparatus, audible apparatus, haptic apparatus, tactile apparatus or other apparatus using a single sense or plurality of senses. A visual representation will typically be used as it can easily be implemented on a display apparatus of a computing apparatus, but the present disclosure is not limited to such visual representation.

FIG. 14 is a schematic representation of a computing apparatus 212 at 320 to which the sensor 210, represented here by the embodiment 252 of FIG. 11, of a neuro-modulation extension apparatus connects and delivers information according to an embodiment wherein similar elements from previous figures are similarly numbered. It must be noted that sensor 210 may measure force, position, angle or some other parameter relating to the motion of the muscle groups of interest. Computing apparatus 212 may be a desktop computer, a laptop computer, a tablet computer, an iPad, an embedded computer attached to the extension apparatus, a cell phone or other computing apparatus. Computing apparatus 212 contains a processing apparatus 322 and a first output apparatus 324 and a second output apparatus 326. Processing apparatus 322 comprises at least one processor 328, at least one memory 330, at least one I/O (input/output) controller 332 and at least one graphical processing unit 334 wherein the processing apparatus 322 executes software instructions to provide a representation of a predefined pattern to at least one of a plurality of output apparatus or devices. In this exemplary embodiment first output apparatus 324 is a visual display apparatus that receives data from graphics controller 334 across a graphics bus 336 and displays a target force as a function of time during the exercise, which in this embodiment is shown graphically as a chart of force versus time 338. First output apparatus 324 may be integrated with the processing apparatus 322 as shown. Conversely the first output apparatus 324 may be separate from the processing apparatus 322 but operatively connected as a functional unit. The first output apparatus may also be an augmented or virtual reality headset. Computing apparatus 212 may optionally contain a second output apparatus 326 adapted to provide an audible representation of the predefined pattern. The sensor 210 of a neuro-modulation extension apparatus communicates with the I/O controller 332 of computing apparatus 212 via a communication interface 242. In this embodiment communication interface 242 is shown schematically as a wireless communications interface but it would be readily apparent to one skilled in the art that such communication channels may be via wireless, wired, optical of any other communications interface and protocol such as, but not limited to USB, Ethernet, Bluetooth, BLE, WiFi, Zigbee, IrDA, fibre-optic link etc. Computing apparatus 212 may further contain an external interface 340 that allows connection to other computing resources such as an external computer or cloud storage and processing resources via any commonly used interface or protocol. This may be achieved via Ethernet, WiFi or other suitable interface.

The first output apparatus 324 may also be used to display instructions on the exercise(s) or actions to be performed, including displaying a video of the required exercise (i.e., sequence of one or more actions), as well as the start and stop time of the assessment time period and other status information (such as repetition number, session ID, etc). The target force level may be displayed as an icon such as a ball, bar, or polygon on a force-time curve, a bar chart or some other graphical representation of the defined pattern. The second output apparatus 326 alternatively may be used to provide information or instructions to the user. The second output apparatus 326 in FIG. 14 may be an audio apparatus in which the audible amplitude (sound pressure level), pitch (frequency) or other audible property changes to indicate the target level. A haptic output apparatus (not shown) may also be used in which the amplitude and frequency of haptic feedback is controlled. Output may be provided simultaneously on multiple output apparatus including any combination of visual, audible and haptic feedback.

The user may be instructed to exert a force on the load cell, and this is represented as a visual graph of force versus time. Similarly, the user may be instructed to apply a given force by an audio signal. The force may be represented by an audio tone wherein the frequency of the audio signal is representative of the required level of force (such as increasing frequency or higher pitch signifying the user to increase their exerted force). It would be readily apparent to those skilled in the art that any number of visual graphical displays, audio techniques, haptic feedback or other forms of instruction/feedback may be applied to instruct the user as to the required exertion force level.

The computing apparatus may further contain an external interface adapted to provide connection to other computing resources such as an external computer or cloud storage and processing resources via any commonly used interface or protocol. In this case data associated with a given user's assessment may be uploaded to an external data storage location (for example but not limited to an external database, a cloud storage apparatus or some form of archive). This allows the system to maintain a longitudinal record of a given user's performance over time. Data archiving and mining then allows summary statistics, progress, compliance and other data to be derived for a given patient, a given cohort of patients or for any other demographic subset of patients. Such summary statistics may be applied to a range of applications. Statistical analysis of the performance of a plurality of treatment programmes can be performed to determine the most effective. The process can be applied to analysis of the performance of a given orthopaedic joint replacement for example. This would allow the rehabilitation and long term longitudinal performance of joint replacement surgery. The analysis may also be applied to determine the performance of surgeons with regard to the rehabilitation and long term health outcomes of their patients. Such analysis may also be applied to develop actuarial tables for insurance purposes. Thus, embodiments may comprise statistical analysis of both the short term and long term effects of a variety of neuromuscular treatment and rehabilitation programmes and their interplay with injury and surgical outcomes.

In some embodiments, the target force level may vary unpredictably, and the target force level is displayed for a short time window in advance of the current time. This ensures the user must watch and react to what they see rather than predict and this prevents the user getting into a rhythm where they may be able to compensate for an injury. For example, in many cases the exercise (e.g., flexion and extension of the exemplary knee) will be repeated several times and the repeated exercises could have unpredictable frequency and/or amplitude variations (e.g., the target parameters for specific actions in an exercise may be varied). In some embodiments the user only sees enough of the curve for an immediate reaction to the changing force-time profile. The unpredictable element is such that it prevents learning by the user, so that they cannot fall into a rhythmical pattern. In some embodiments the target force level is suppressed for a period of time, such that the user is shown the immediate target during a first or current time period and a future target level at a later time period with a time gap between the two time periods. This is a feedforward approach which requires the user to predict the amount of force to apply during the time gap period in order to follow a suppressed or obscured target curve and/or to match the future target level and the end of the time gap period.

Exercises that require a user to execute varying force versus time, or position versus time in the case of an extension exercise such as a leg press, can promote skill learning and motor proficiency as the basis of creating a memory movement ‘blueprint’, to be recalled at a later time. In some embodiments a uniform sine wave consistently repeating identical amplitude and frequency used as a target force profile allows a limb to move through a reliable and consistent range of motion. Using this movement blueprint as the basis of motor memory variations can be applied and the measured accuracy of reproduction can be used to assess motor control and adaptability.

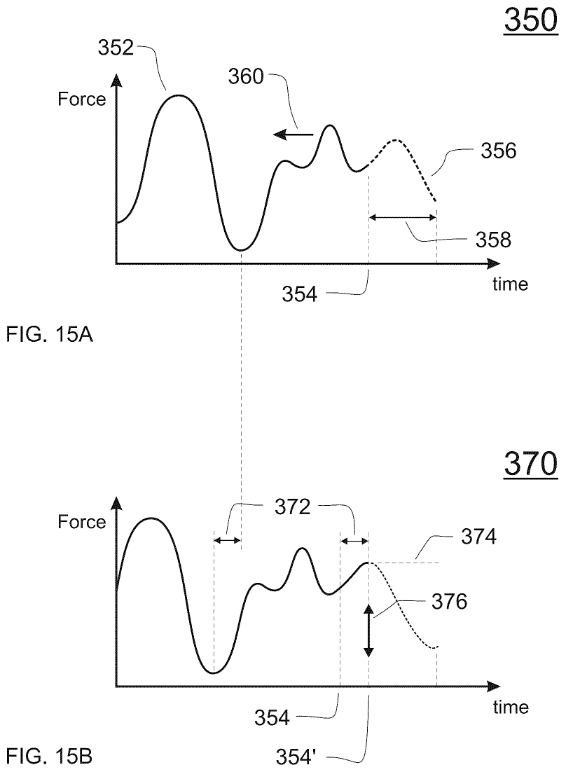

FIG. 15A is a schematic representation of a defined pattern presented to the user at 350 wherein similar elements from previous figures are similarly numbered. Visual display apparatus 324 of FIG. 14 displays a graph of a target force as a function of time that a user is required to replicate on the extension apparatus. Force as a function of past time 352 is shown prior to the present time 354 and force as a function of future time 356 is shown for a limited future time window 358. The length of this future time window 358 may be adjusted based on the complexity of the exercise (or actions) and/or the capabilities of the user to respond to the exercise (e.g., due to the degree of impairment or fatigue).) In some embodiments the future time window is between 0.1 second and 5 seconds. The future time window may be varied as the exercises are performed; for example, the time window may be extended as the complexity increases or over time for extended sessions in which fatigue may occur. In some embodiments the display is a scrolling display with the curve of force versus time 352 and 356 scrolling 360 such that the present time 354 stays at a fixed horizontal point on the display. FIG. 15B is therefore a schematic representation of the defined pattern of FIG. 15A shown at a later point in time 354′ at 370 wherein similar elements from previous figures are similarly numbered. A period of time 372 has passed since the representation of target force shown in FIG. 15A such that the new present time is given by the formula, time(354′)=time(354)+time(372). The target force 374 at the new present time 354′ (which is later than 354) is at the same horizontal location on the screen. The target force at the present time is therefore seen on screen to move only in the vertical axis 376 with the passage of time, the vertical extent of the target force 374 representing how much force the user is required to exert on the extension apparatus at any given time.

In some embodiments, the target force may be allowed to vary predictably (i.e. constant frequency/period) such as but not limited to a sinusoid for at least for some portion of the session or exercise. Each user will have different capabilities and impairments and thus in one embodiment the neuro-modulation apparatus and system may be used to perform an assessment (or characterisation) of neuromuscular control which may include a measurement of the maximum strength of the user which may be recorded as a maximum strength reference value. The target force may then be varied over the range from 0 to 100% of the measured maximum strength value or some other range as desired. The frequency of the periodic function may be in the range of 0.1 to 0.5 Hz (period of 6.6 seconds to 2 seconds). In some embodiments a frequency of 0.3 Hz is used. Frequencies in this range correspond to common movement speeds for most joints. Frequencies below 0.1 can be too slow such that it can be difficult for an impaired person to perform a smooth movement and they concentrate on not moving too fast, and is atypical from normal joint movements (and thus may be useful in retraining normal movements. Frequencies above 0.5 Hz represent rapid movements, and patients may become focused simply on completing the movement in the allocated time over performing the movement accuracy (i.e., the rate may be too fast to enable them to cognitively focus on the accuracy). The frequency may be preset, for example 0.3 Hz, or an assessment may be performed on the appropriate frequency for a particular patient to determine the capabilities or range. Frequencies outside of the 0.1 to 0.5 Hz range may be used, but an assessment should preferably be performed on the patient. Further the frequency range for a specific joint may vary from the range of 0.1 to 0.5 Hz. Again, deviation from this default range can be determined through assessment trials on patients.

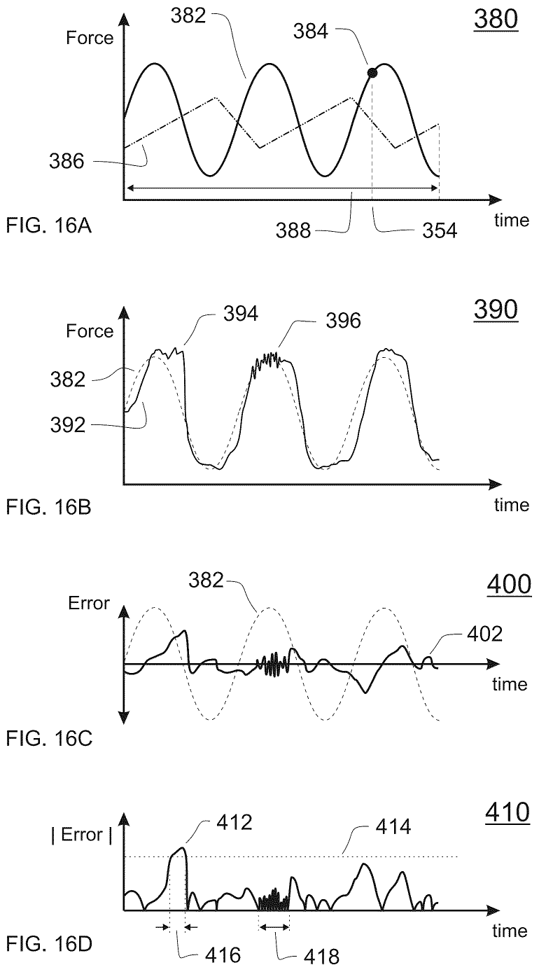

FIG. 16A is a schematic representation of a defined pattern 382 presented to the user at 380 wherein similar elements from previous figures are similarly numbered. Visual display apparatus 324 presents a sinusoidal defined pattern 382. A dot 384 is displayed to show the actual instantaneous force applied by the user on the extension apparatus as measured by sensor 252. As the sinusoidal target force pattern 382 scrolls to the left on the screen the target force traces a sinusoid on the vertical axis at the present time 354. The dot 384 would also trace out a perfect sinusoid in the present time axis 354 if the user was perfectly delivering the required force. The dot 384 provides real time feedback to the user about the actual force applied. The user can then compare the real time actual force applied to the target force which is the vertical separation of the dot 384 from the scrolling curve 382 and apply some force correction in order to make the dot 384 track the curve 382 more smoothly and accurately. This will be subsequently referred to as real-time-feedback throughout the course of this disclosure. It would be readily apparent to those of ordinary skill in the art that other periodic or non-periodic defined patterns may be used. A sawtooth target force 386 is shown for comparison where the force increases and decreases linearly between target extremes, shown here with differing rates of change. The frequency of a periodic or repeating pattern can be altered, the rise time and fall time of the pattern can be altered, the shape of the curve can be altered. There are infinite variations possible within the scope and intent of the present disclosure. However, a sinusoid or similar slowly varying target force, especially at the turning points, is particularly advantageous as the slow change from increasing to decreasing force (extension to flexion of the leg in the exemplary embodiment of FIG. 10) is difficult to achieve even for a health neuromuscular system. Slow changes in force therefore highlight any deficiencies in neuromuscular control.

In one embodiment of neuro-modulation assessment or therapy the user attempts to follow the target force curve for a defined period of time, a capture period 388, while the neuro-modulation apparatus records the measured force applied by the user. In the case of exemplary sinusoid shown in FIG. 16A, the capture period is roughly 2¾ sinusoid cycles (or 5.57π radians). In other embodiments the capture period may be the duration of the exercises, such as 10, 20, 30, 40, 50, 60 seconds or more. In practice there will be some deviation from perfect replication of the target force, even for someone with a healthy neuromuscular system. FIG. 16B is a schematic representation of the results of capture period 388 at 390 wherein similar elements from previous figures are similarly numbered. After completion of a capture cycle, the sinusoidal defined pattern 382 is displayed on the visual display 324 along with the measured force 392 applied by the user. In this exemplary diagram, the user lags the target force on both increasing and decreasing force with poor control at the turning points and a strong linear stroke to ‘catch up’ rather than slowly increasing and decreasing force. Some erratic response is also shown at the second peak of the sinusoid. Following the first peak of the sinusoid the response lag 394 is particularly evident while around the second peak of the sinusoid some erratic response 396 is observed. This is quite normal for someone suffering arthrogenic muscle inhibition. When a contracting muscle is lengthened while under tension, an eccentric muscle contraction results that utilises less motor units per force unit compared to a concentric contraction where the muscle shortens under tension. Subsequently greater error is more likely when the primary muscle is aiming to control eccentrically. Quickly changing direction on the apex of the curve utilises a deceleration concentric action followed by a miniscule pause at the peak and subsequent eccentric action on the neuromuscular system. This highly challenging action is akin to a functional change of direction activity used on a sports field or for a pedestrian to avoid a collision with a car.

FIG. 16C is a schematic representation of the user error in replicating the defined sinusoid 382 during the capture period 388 at 400 wherein similar elements from previous figures are similarly numbered. Error 402 is calculated as the difference between the target force 382 and the actual measured force applied by the user, shown as 392 in FIG. 16B. The target force 382 is shown as an overlay on FIG. 16C only for reference with respect to visual alignment. The amplitude and phase of the error 402 along with a determination of the smoothness of the error leads to characterisation of the degree of neuromuscular control. For example, neuromuscular control is greater if the error curve 402 is smooth and slowly changing compared to an error curve that showed large amplitude variations with irregular or sharp changes in amplitude. The sign of the difference may enable an analysis of whether lack of control is limited to particular movement of the muscle or joint, such as extension versus flexion.

FIG. 16D is a schematic representation of the absolute error 412 (i.e., Modulus of the error 402) at 410, not to the same scale as the error in FIG. 16C. In one embodiment a predetermined threshold error value 414 is used to determine the range of motion 416 where a user lacks control, or the errors may be used to perform an assessment of the smoothness of the action, or they may be processed to determine summary measures that may be used to guide further treatment (e.g., a change in complexity of exercises). This analysis may be used to determine a range of force values for which the error exceeds a threshold, a range of joint angles, the frequency of user correction or other parameters of interest. It would also be apparent to one of ordinary skill in the art that other types of error analysis may be of value. In one embodiment, a threshold difference value may be set, such as 5% or 10%, and the percentage of time during the exercise that the difference was below the difference threshold is determined, and this is compared to a trigger threshold. This difference measurements may be normalised, or converted, to a percentage error difference: % error=100%×|target−measured|/target. Then each percentage error difference may be compared to a difference threshold (e.g., 10%) and the percentage of time during the exercise that the percentage error was less than the difference threshold may be calculated as an accuracy measure. This accuracy measure may then be compared with a trigger threshold, such as 90% to determine whether the motion is smooth and thus whether to trigger a change such as a progression in complexity. In one embodiment, a Fourier analysis of the absolute error signal is conducted with the threshold set at a certain frequency threshold. Under these circumstances the region of erratic behaviour 418 is identified as exceeding the frequency limit. In another embodiment the threshold value 414 may be obtained by taking a median, average or other central measure or statistic (i.e., trimmed means, robust means, etc) of the threshold force values when the error exceeds the threshold. Alternatively, the threshold values could be ranked and the 75th percentile (or other percentile) value used. Multiple force or error ranges may be defined based on a spread in threshold force values. For example, a measure of the spread of values, such as an interquartile range or standard deviation may be used to determine whether the threshold values should be split into separate groups (and thus ranges). Error bands may be defined and the percentage of time during an exercise that the percentage error is within each error band determined. For example, error bands may be defined such as green/good=difference<4.25%, orange/moderate=4.25−7.5%; red/poor≥7.5%. Other ranges may be used, and more bands may be used, or a single threshold (e.g., 5% or 10% error) may be used to define two bands (e.g., good/bad). Clustering approaches or other pattern matching, or informatics approaches could also be used to identify multiple force ranges of interest. In another embodiment a region of interest may be identified by analysis of overshoot, undershoot, hysteresis, smoothness or other parameters of interest wherein some form of analysis of the error 402 or 412 would exceed a given threshold (or indeed analysis of the measured force 392). All forms of analytical or statistical analysis performed on the raw signal 392, the error 402 or absolute error 412 are anticipated in the present disclosure.

In another embodiment the defined sinusoidal pattern 382 may be a stationary curve visible at all times and in its entirety while the measured force 392 applied by the user is drawn in real time as an overlay (with the dot 384 moving simultaneously in both vertical and horizontal planes). In yet another embodiment the user may be presented with a representation of the error signal 402 rather than displaying two curves and would actively control the error to remain as close as possible to zero.

The degree of neuromuscular control can therefore be characterised and quantified by the methods taught in the present disclosure. Furthermore, repetitive application of a periodic and cyclical defined pattern such as the sinusoid of FIG. 16A creates a motor memory blueprint via real-time-feedback. Studies have demonstrated that learning a new motor task changes the sensitivity of fast feedback responses (i.e., reflexes) but that it also modifies feedforward (i.e., voluntary) motor commands. Embodiments may therefore harness the interplay between feedback and feedforward motor learning mechanisms increasing neuroplasticity for accelerated motor learning.

Internal models are a well-established concept in self-initiated motor function. These models represent a neural map of the afferent environment that allow voluntary control mechanisms to compensate for the complex mechanical properties of the limbs, delayed and noisy sensory feedback and to adapt to changes of the body or the environment. Less appreciated is that internal models also apply to fast feedback responses, which have to compensate for the same factors when countering external perturbations. Modern theories of motor control, based on optimal feedback control, also posit that motor behaviour is achieved via the sophisticated manipulation of sensory feedback. Under this class of model, it is no wonder that arthrogenic muscle inhibition limits control and recovery from injury or surgical intervention. Sensory information does not match the model of joint motion, so inhibition shuts down the system as a protection mechanism. However, this class of model suggests that bidirectional transfer between feedforward and feedback control is expected because feedforward motor commands and transcortical feedback responses are part of the same control system implemented in common neural circuits. The cerebellum, which is richly interconnected with the primary motor cortex is strongly implicated in multi-joint coordination during both feedforward control and feedback responses and have long been hypothesised to house the computations related to internal models.

Embodiments therefore teaches a method of harnessing the duality of feedback and feedforward motor learning. Real-time-feedback has been discussed as the mechanism for learning the motor skill, which in one embodiment requires a user to apply a sinusoidally varying defined pattern of force to a neuromuscular extension apparatus in synchrony with a defined pattern's target force. Feedforward motor learning is achieved by suppressing parts of the defined pattern and forcing the user to ‘gap-fill’ using a combination of feedback and feedforward control mechanisms as they recall the learnt movement blueprint, taught during real-time-feedback, and apply it to a known gap in the defined pattern. This will be subsequently referred to as real-time-feedforward throughout the course of this disclosure. Feedback can be considered a form of calibration where the user is directly presented, i.e., observes, the outcome of their efforts in real time, and can learn how much effort is required, or how to adapt their effort, to match the target parameter. This then primes them for feedforward sessions where the user must estimate how much effort is required to achieve an instructed target value at a future time point, for example by suppressing or occluding a curve of the target parameter, and any measured values for some portion of the exercise (i.e., the measurement or capture time periods).

Graduated, increasing suppression encourages a greater reliance on estimating and gap-filling movement proficiency, hence greater reliance on feedforward capabilities. As feedback and feedforward work in tandem, a shift from high-feedback and low-feedforward to low-feedback and high-feedforward creates greater emphasis on predictive error processing. Although the present disclosure teaches the many benefits of a progression from feedback-dominant to feedforward-dominant exercises, this need not occur in a monotonic sequence. Embodiments thus teach that various combinations of feedback-dominant and feedforward-dominant exercises are beneficial. Embodiments thus teach a method of utilising both real-time-feedback and real-time-feedforward motor learning, called Neuro-Modulation Therapy (NMT). Neuro-Modulation Therapy creates a highly neuroplastic environment and allows rapid training of internal models, which is particularly advantageous in an environment of new-normal afferent information associated with injury or surgical intervention.

Curve suppression variations can test different aspects of movement skill. For example, occluding the nearly linear sections of a sinusoid where momentum has already been generated can be regarded as a simple task to gap-fill. Using leg press exemplary extension apparatus as shown in FIG. 10, suppression (occlusion) of the upward part of the sinusoid involves a gradual increase of force over time that uses a concentric muscle contraction. Suppression (occlusion) of the downward part of the sinusoid involves gradually letting go of force over a period of time which uses a different eccentric muscle contraction. It is widely known that eccentric muscle contractions use less motor units per unit of than concentric muscle contractions; more force is exerted per motor units on the eccentric muscle group which would therefore be subject to greater load per unit and subsequent fatigue. By comparison, maintaining neuromuscular control when the turning point of a sinusoid is suppressed (occluded) is a much more a complex task involving a change of direction; predictive deceleration to a momentary isometric state (static position) followed by an acceleration task, all in a short period of time from memory of the occluded pattern. Depending upon which section of the turning point this undertaken, the muscle actions undergo a sudden contraction change from either eccentric muscle contraction to momentary static isometric contraction to concentric muscle contraction or concentric muscle contraction to momentary static isometric contraction to eccentric muscle contraction. When the amount of visible curve information is reduced through suppression (occlusion) or minimising the view window, the subject is required to increase their prediction through greater reliance on feedforward mechanisms. Injured subjects utilise greater visual and cognitive processing than non-injured, so occluding visual information challenges their mal-adaptive system, especially under real-time constraints. This was seen in our unpublished study, where ACL injured athletes performed more poorly on their ACL injured side, noting greater error scores compared to their non-injured side when occluding the visual curve. Fatigue would affect the smoothness of movement and result in higher error. Fatigue may also affect the excitatory/inhibitory balance, reducing the cortical drive and subsequent motor unit output. Curve occlusion and the progression thereof is an important parameter in developing a suitable treatment regime for any given joint or muscle group on any given patient. The decision making for complex tasks such as turning point occlusion also creates more cognitive load than simple tasks. The longer a test is delivered in a given cycle or aggregate cycles, the greater the level fatigue which may affect the measured error. Repeated tests with insufficient rest periods challenge both local muscle fatigue and the central nervous system including but not limited to cognitive load.

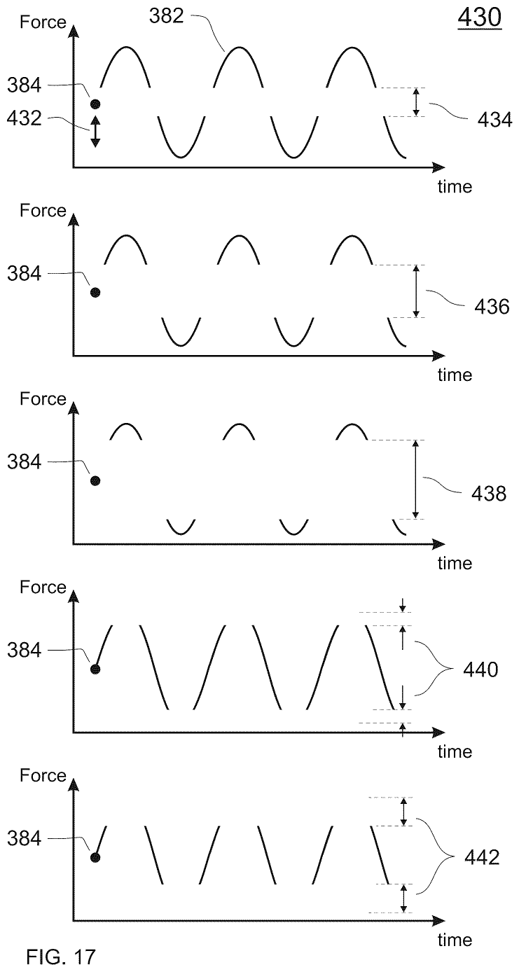

FIG. 17 is a series of visual representations of feedforward exercises where the target parameter is a sinusoidally varying target force shown on visual display apparatus 324 in which different regions of the pattern are suppressed (occluded) in each visual representation according to an embodiment at 430. The dot 384 represents the instantaneous force applied to the extension apparatus by the user in real time, which moves only in the vertical axis 432 and the present time is shown near the left of the graph with a long future time window visible. The series of visual representations comprises a centrally occluded 25% window 434; a centrally occluded 50% window 436; a centrally occluded 75% window 438; a 25% occlusion to the turning points 440 (i.e., 12.5% from either end); and a 50% occlusion to the turning points 442. In alternative embodiments, an audibly presented representation of target force may be used whereby amplitude, pitch or some other parameter is used to represent a change of target force can also be adapted to indicate suppression whereby suppression is indicated by silence, a constant unique tone or some other identifiable sound in an otherwise varying sound field (i.e., to suppress the representation of the target level). In some embodiments where any combination of visual, audible and haptic feedback is used, suppression may be performed on one or two feedback types, but provided on another feedback type. For example, when the visual curve is being suppressed (or occluded), the audio and/or haptic feedback may continue to be provided.

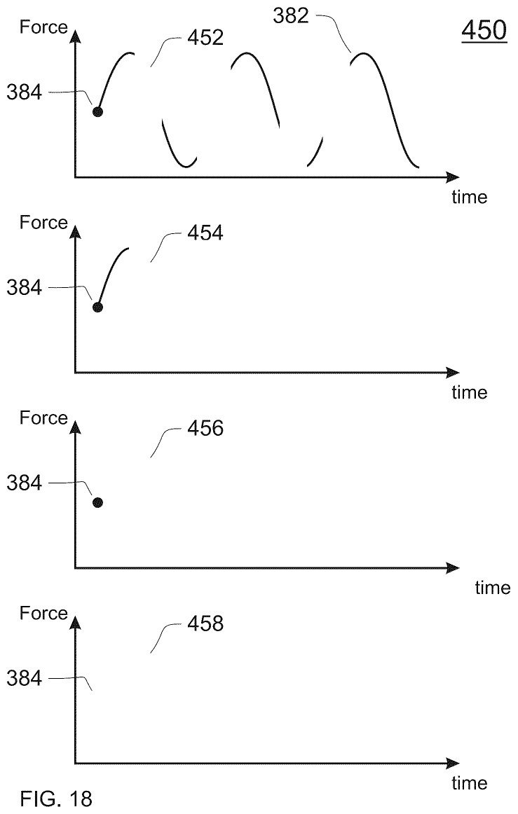

FIG. 18 is a series of visual representations of a sinusoidally varying defined pattern of target force shown on visual display apparatus 324 in which different regions of the pattern are suppressed (occluded) in each visual representation according to an embodiment 450. The first plot comprises a random occlusion 452 and the second plot comprises a small future time window 454. This may be a very short fixed period or variable period. Shortening the future time window reduces cognitive load because it reduces the amount of visual information that needs to be processed. Neuro-Modulation Therapy uses a method of beginning with a full sinusoid (continuing with this preferred embodiment) for real-time-feedback training and then subsequently transitioning to real-time-feedforward with ever increasing occlusion windows. As the user advances in the therapy, it may be appropriate to completely suppress or occlude the sinusoid 456, as shown in the third plot showing only the dot 384 which represents the user applied force (i.e., complete suppression of the representation of the target parameter whilst displaying the feedback representation). After a period of therapy is it surprising how well the user can replicate the sinusoid in both amplitude and frequency with only the dot 384 providing feedback even if the sinusoid is completely suppressed or occluded. Finally, both the sinusoid 458 and the dot 384 may be suppressed or occluded as shown in the last plot. For reference the non-occluded sinusoid 382 is shown in FIG. 16A.

Error analysis of the difference between measured and target parameter may be used to determine when to change or progress treatment, for example to when to introduce suppression (occlusion) and/or when to change the parameters of the suppression such as the percentage of suppression (e.g., 10%, 25%, 50% 75%) and location (e.g., phase angle range) of the suppression in order to change (e.g., to increase) the complexity of the task. In one embodiment one or more thresholds and/or one more error ranges (or error bands) may be defined, and the percentage of time during an exercise that the difference error is above/below a threshold, and/or within each error band may be calculated. For example, a single threshold error level may be defined, such as 10% to define good and bad error bands (0-10%=good>10%=bad). Similarly, two thresholds could be defined to create three error ranges or error bands which could be labelled good, middle, and bad; or green, orange, red (i.e., traffic light representation). In one embodiment the error thresholds are 4.25% and 7.5%, but other threshold values could be used such as 5% and 10%, or 10% and 25%. Additional thresholds could also be used, e.g., 3, 4 or 5 thresholds to define 4, 5 and 6 error bands. Change in complexity could be automated. In some embodiments the transition could be based on local averages and measures of stability or reduced variance. For example, a measure of the variation of the time in one or more error ranges, such as the good or lowest error range may be calculated over the previous n trials (e.g., n=3, 4, 5, 6, etc). If this variation is small (<5% or <10%), then this may indicate the patient is no longer being challenged by the exercise task, and thus a progression of complexity may be triggered. Similarly, if the increase in the red/bad range is stable, this may indicate the patient is not able to adjust to the increase in complexity, or is becoming fatigued, and a decrease in complexity may be triggered. Alternative and more complex criteria could be used such as a significant change in the magnitude of the time in a particular error band combined with a variation measure may be used to trigger a change. Appropriate trigger rules could be programmed into the control apparatus (computer) to control the triggering of a change in the suppression in a session to further automate the therapy. In some embodiments a default progression in suppression may be programmed for one or more session, which may then be varied in real time according to stored trigger rules that take into account the performance of the user.

Other aspects of the target force pattern can be manipulated to measure changes in the level of neuromuscular control. In one embodiment partial occlusion of the entire curve may be achieved by changing the transparency of the curve on screen or via the contrast between the curve and the background. In another embodiment his can take the form of a ‘blinking’ occlusion where the entre visual representation of target force is alternately visible and invisible at a variety of frequencies and/or duty cycles. In another embodiment the dot 384 that represents the real time force applied by the user can be displayed differently. The dot 384 may be visually present, absent, have different levels of transparency or contrast with the background. The frequency of the repeating target force pattern can be increased or decreased to challenge cognitive processing. The higher the frequency, the greater the cognitive load, which challenges injured users; greater cognitive load and less autonomic function compared to uninjured counterparts. At higher frequencies, tighter curves present greater emphasis on complex skills that involve quicker changes of direction, further challenging users. The direction that the curve moves across the screen may affect the user's ability to accurately replicate the target force. The curve can move from left to right, right to left, top to bottom or bottom to top. The ideal curve may also be represented by a Lissajous figure where target force is represented in the horizontal axis and force applied by the user is represented in the vertical axis (or vice versa). A circle is an exemplary Lissajous figure of parameters a=1, b=1 and phase=pi/2 and a straight line inclined at 45 degrees to the horizontal is a Lissajous figure of parameters a=1, b=1 and phase=0. The curve may also travel in a simulated 3 dimensional space as depicted on screen of with a virtual reality presentation.

FIG. 16C teaches a method of assessing the error 402, being the difference between a target force 382 and a force applied by a user 392, thereafter discussing the different types of error 416 and 418 relate to the differing exemplary user applied force error profiles 394 and 396 respectively. Error distribution, the form of the error (including overshoot and undershoot among other things), amplitude of the error, the phase relationship of the error to the target force, the frequency components and other characteristics of error provide information to a clinician that can be applied to optimising NMT or improving functional therapy design. The software application contained within the NMT apparatus and system displays error to the user (and/or the clinician as appropriate throughout this disclosure). It may be beneficial to observe the measured force applied overlaid on the target force as depicted in FIG. 16B. It may also be beneficial to observe effort as depicted in exemplary FIGS. 16C and 16D, for example where error may be presented as a percentage of target force or some other parameter. Other forms of error display would be readily apparent to one of ordinary skill in the art and are anticipated in the present disclosure.

Error can also be classified into bands of ranges that help the user quickly identify their progress after a test, both in terms of the value of the error and the segment of the target force profile. An exemplary band of errors may be represented by the categories ‘Good’, ‘Fair’ and ‘Poor’ and may also include colour coding such as green, orange and red respectively, but other categories may be suitable under different circumstances. The error distribution can also be arranged according to the type of cohort. For example, athletes with higher level of motor skill may require tighter tolerance on these error bands than the general population or those in early rehabilitation after injury or surgical intervention. The bands used in an unpublished athletic cohort study performed by the inventors were green 0%-4.25%, orange 4.26%-7.50% and red>7.50%. This classification can help users remain motivated to improve error and ultimately increase the volume or dosage potency of NMT. The error ranges, and percentage of time in each range may be used to determine when to trigger a change in the suppression, such as increase in the complexity of the suppression. An increase in errors could also be used as a trigger to reduce the complexity. Other error ranges including two ranges (good/bad) using single threshold value such as 2.5%, 4.25%, 5%, 10%, 20% or 25%, or more than three ranges may be used. Assessments or calibration experiments may be performed for specific joints to determine the threshold values appropriate to a specific joint.

Embodiments teach that suppression (occlusion) places a higher reliance on prediction and feedforward mechanisms. Comparing the occluded and non-occluded error profiles provides insight into a subject's feedforward and feedback mechanisms. Injured subjects display increased reliance on areas such as the cerebellum, which assist in predicting and reducing error in motor tasks. Therefore, occlusion using feedforward provides insight into the cerebellum. In a yet to be published study, injured subjects performed worse than healthy control subjects during turning point occlusion tests.

Physiologically, there are differences in the way muscles behave under concentric (shortening) and eccentric (lengthening) muscle contractions. Eccentric muscle contractions use less motor units per unit of force and are thereby subject to higher forces per motor unit activated. This can result in rapid fatigue of the motor units, especially in subjects suffering arthrogenic muscle inhibition. Furthermore, anterior cruciate ligament reconstruction and total knee replacement recipients have difficulty ‘letting go’ of quadriceps and hamstring muscles (eccentric contractions), leading to decreased control during deceleration tasks, which was confirmed in our yet to be published study.

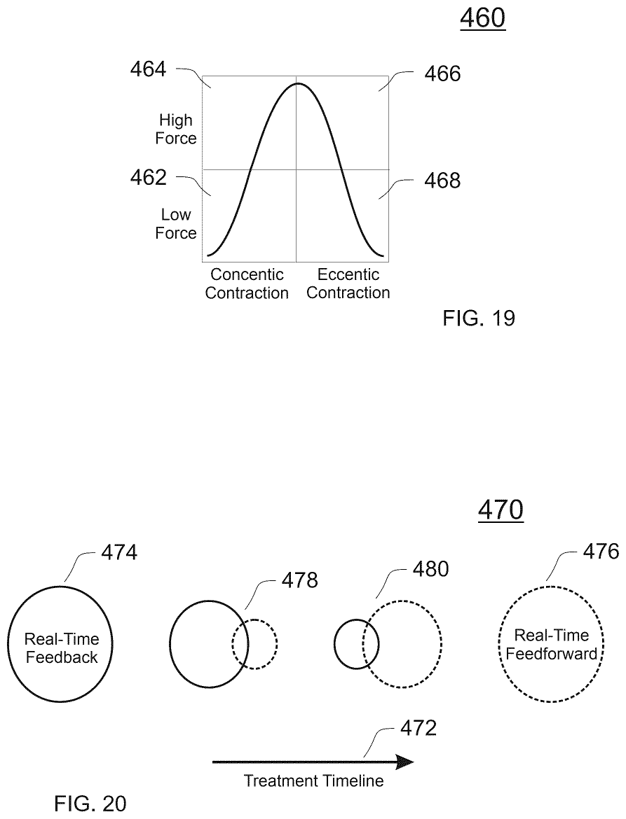

Segmenting the exemplary sinusoidal target force into quadrants provides further insight beyond concentric and eccentric muscle contractions. FIG. 19 is a schematic representation of the four quadrants at 460. Quadrant one 462 depicts a concentric contraction at low force. Quadrant two 464 depicts a concentric contraction at high force. Quadrant three 466 depicts an eccentric contraction at high force. Quadrant four 468 depicts an eccentric contraction at low force. Categorising a full extension and flexion cycle using the quadrant approach provides a way to map force ranges to the number of motor units recruited. For example, quadrant three 466 represents a high-force eccentric contraction which would likely suffer earlier onset fatigue than a quadrant one 462 low-force concentric contraction as a result of arthrogenic muscle inhibition impacting the fewer available motor units. When error analysis is categorised under this quadrant model, insights may be gained into the design and progression of NMT or rehabilitation; too high an error may limit the benefits of increasing cortical drive and subsequent decreased intracortical inhibition. Embodiments therefore provide a method of characterising error using segmentation by quadrant, although it would be readily apparent to one of ordinary skill in the art that segmenting error in other ways is anticipated within the scope of the present disclosure.

Using the results of quadrant analysis for progressing and improving Neuro-Modulation Therapy allows targeted therapy aimed at users' neuromuscular control deficits. Our studies have identified that total knee replacement recipients experience greater error in the low force and eccentric contraction quadrant 468 when compared to low force and concentric contraction quadrant 462. This is consistent with patients having difficulty letting go of force in a controlled manner due to the co-contraction state of both the quadriceps and hamstrings muscles as extensively reported in the literature. Specifically spending more time training within this quadrant using Neuro-Modulation Therapy offers a pathway to improving a patient's capacity to perform a controlled ‘release’. Therefore, improving the error score has the ability to impart greater emphasis on the deficit and allow the total knee replacement recipient to improve their functional status during activities such as walking. It is noted that total knee replacement patients immediately improve their functional quality of gait (unprompted) after Neuro-Modulation Therapy, reducing limping and displaying limb movement during walking that mimics their non-operative leg, resulting in greater optimised bipedal motion. Likewise, quadrant 466 displaying a summarised greater error due to high eccentric muscle contraction and fatigue may indicate that the total knee replacement recipient may have greater difficulty traversing down stairs using an eccentric muscle contraction and be subject to higher risk of falls. Fatigue however can be improved with Neuro-Modulation Therapy targeting the quadrant 466 to extend the onset of fatigue limiting onset and hence reduce the probability of falls. With reference to the exercise bike variant of the neuro-modulation extension apparatus 231 presented at FIG. 9A, the sinusoidal defined pattern represented in FIG. 19 may be replicated by pedalling forwards to follow the curve during quadrants 462 and 464 and to pedalling backwards during quadrants 466 and 468 wherein pedalling speed would be akin to the rate of change of the slope of the defined pattern. Other variations of this approach would be readily apparent to one of ordinary skill in the art and are anticipated in the present disclosure.

In some embodiments of NMT, a progression from feedback to feedforward motor learning is used to teach the new signalling environment and thus improve motor control. FIG. 20 is a schematic representation of the progression of treatment in time 472 in an embodiment of Neuro-Modulation Therapy at 470. Therapy begins exclusively with real-time-feedback motor learning 474 (feedback exercises). Using the exemplary sinusoidal predefined pattern by way of example, the user would undergo a plurality of cycles of the capture period 388 presented with the non-occluded target force curve 382 shown in FIG. 16A. Analysis of the error 402 or absolute error 412 shown in FIGS. 16C and 16D determines when the user is ready to progress to the next stage in the therapy; the introduction of suppression of parts (or all) of the target force curve 382. In this embodiment we will refer to the suppression as visual occlusion, or simply occlusion (that is, references to occlusion throughout this disclosure should be understood to more generally refer to suppression). Treatment may progress to the inclusion of a small amount of occlusion 478. In some embodiments, the user would undergo a plurality of exercise cycles of the capture period presented with a slightly occluded target force curve such as a small window of centrally occlusion 434 as shown in the top plot of FIG. 17. Central occlusion is easier for the user to gap-fill than turning point occlusion because it merely requires continuation of an existing motion rather than anticipation of a change in direction.

When the user is ready to progress, the window of central occlusion may be increased sequentially 436 and then 438 as shown in FIG. 17 as treatment progresses from 478 to 480 representing a larger real-time-feedforward contribution to motor learning. As motor control improves treatment progresses to occlusion of the turning points, firstly with minimal occlusion of the turning points 440 as shown in FIG. 17 (e.g., 5%, 10%, 20%, 30%, 40%, 50%). This is much more challenging for the user as it involves precise control of deceleration, accurate timing of the turning point and precise control of acceleration in the opposite direction in order to be at the right place on the curve (force, position or another parameter) when the curve comes out of occlusion. Again, as motor control improves treatment progresses to larger windows of occlusion at the turning points 442 as shown in FIG. 17 (e.g., 10%, 20%, 30%, 40%, etc.) until the curve is almost completely occluded (e.g., 90%, 95%, 99%, not shown in the figures). In this way the treatment timeline alternates between 478 (or even 474) and 480 in the middle of the treatment timeline; minimal central occlusion through maximal central occlusion and then minimal turning point occlusion maximal turning point occlusion. However, there is a general progression from greater emphasis on real-time-feedback 478 to greater emphasis on real-time-feedforward 480 as the difficultly of occlusion increases. That is the NMT comprises a series of sessions of exercises, each comprising a plurality of exercises, wherein over time, the plurality of exercises in a session transition from mostly or all (e.g., at least 80%) feedback exercises in a session to mostly or all (e.g., at least 80%) feedforward exercises in a session, and the amount of suppression and complexity of suppression in feedforward exercises is also increased over time. Complexity refers to the aspects such as difficulty and cognitive loading in performing the exercise including predicting the time and effort required to match the target force curve. In some embodiments the user may be instructed to perform cognitive tasks whilst performing exercises as discussed herein. The instruction may be aural or visual such as task displayed on a screen.

As the treatment timeline progresses and neuromuscular control improves the user may be presented with no defined pattern to follow 456 (i.e., complete or 100% suppression) and rely almost entirely on motor memory of the amplitude and frequency of the exercise (e.g., the sequence of actions in an exercise). At this stage the user would still be provided with a feedback representation about the measured force, position or other parameter applied by the user although with no defined pattern to follow 456 the user is entirely in real-time-feedforward. The therapy may continue to progress to removal of the measured force feedback, the user being presented with a completely blank screen 458, noticing the absence of dot 384. This would be full real-time-feedforward 476. This demonstrates the interplay of real-time-feedback and real-time-feedforward motor learning in the Neuro-Modulation Therapy taught in the present disclosure.

In some embodiments, NMT comprises multiple sessions over a period of time such as hours, days, weeks, months or longer, each session may begin with several cycles of the non-suppressed pattern (i.e., feedback exercises). Introducing a short period of feedback motor learning at the beginning of a session refreshes the neural memory system and encourages the interplay of feedback and feedforward motor systems, improving the speed of motor learning. That is each session may comprise one or a few sets of feedback exercises followed by a larger number of feedforward exercise sets, or a session may comprise feedback sets interspersed between larger numbers of feedforward sets. Assessment of control and associated reporting may be provided at the end of each exercise, each set of exercises, or the end of the sessions.

The effect of the NMT is that it leads to updating of the internal model of motor function based on the new-normal afferent information and thus the effects of arthrogenic muscle inhibition subside. Motor response in the altered joint matches the newly updated internal model so there is no need for inhibitory protection and thus control improves. Neuro-Modulation Therapy is believed to create a window of time in which the motor learning function is in a state of high neuroplasticity. Fine tuning of the internal model can then occur when sensory experience is mapped to a known or planned motor function. In other words, the neural pathways are primed for optimal learning. Surprisingly this window can be opened with just minutes of Neuro-Modulation Therapy and lasts for several hours. In a yet to be published study, the inventors have found that the window extends for several days after a single session of targeted Neuro-Modulation Therapy. That is, benefits can be obtained rapidly in as little as a single session or only a few sessions, with repeated sessions further reinforcing and improving control outcomes. Neuro-Modulation Therapy can be thought of as opening a window through which traditional functional treatments can be efficiently mapped into new motor function skill. Once the window is open, traditional function-based therapeutic protocols should be employed for maximum benefit.

An example of an early Neuro-Modulation Therapy programme and primed functional exercise integration for one week is as follows. A simple example will be explained using a sinusoid with a frequency of 0.1 Hz to 3 Hz with exercise trials lasting 30 seconds, the same length of rest breaks between trials of 1 minute and evenly separated into 3 times a day (morning, early and late afternoon) programme. Once the Neuro-Modulation Therapy has been completed, the primed neuromuscular system enhances the integration into a self-directed quality functional movement that is relevant to the patient.

This enables mapping quality functional movement into the central nervous system. The target representation is a visually displayed sinusoidally varying force curve and thus references to un-occluded refer to feedback exercises and occluded refers to feedforward exercises. In the following exemplary programme, ‘flipped’ refers to changing the phase of the exercise. For example, a typical exercise would involve the user increasing their applied force as the sinusoid increases (or moves toward the top of the screen). Conversely, a ‘flipped’ exercise would involve the user decreasing their applied force as the sinusoid increases (or moves toward the top of the screen). The one week program may comprise:
Day 1: (Force range=2-8 kg·f). 3 trials of un-occluded, 3 trials of 25% centrally occluded. 3 trials of 50% centrally occluded. 1 trial of force un-occluded flipped. Functional movement training=walk 500 steps.
Day 2: (Force range=2-8 kg·f). 3 trials of un-occluded, 3 trials of 25% centrally occluded. 2 trials of 50% centrally occluded. 1 trial of 75% centrally occluded. 1 trial of force un-occluded flipped. Functional movement training=walk 550 steps.
Day 3: (Force range=2-8 kg·f). 3 trials of un-occluded, 2 trials of 25% centrally occluded. 2 trials of 50% centrally occluded. 2 trials of 75% centrally occluded. 1 trial of force un-occluded flipped. Functional movement training=walk 600 steps.
Day 4: (Force range=2-8 kg·f). 3 trials of un-occluded, 1 trial of 25% centrally occluded. 2 trials of 50% centrally occluded. 2 trials of 75% centrally occluded. 1 trial of 25% corners occluded. 1 trial of force un-occluded flipped. Functional movement training=walk 650 steps.
Day 5: (Force range=2-8 kg·f). 3 trials of un-occluded, 1 trial of 25% centrally occluded. 1 trial of 50% centrally occluded. 2 trials of 75% centrally occluded. 2 trials of 25% corners occluded. 1 trial of force un-occluded flipped. Functional movement training=traverse 20 stairs.
Day 6: (Force range=2-8 kg·f). 3 trials of un-occluded, 1 trial of 25% centrally occluded. 1 trial of 50% centrally occluded. 1 trial of 75% centrally occluded. 1 trial of 25% corners occluded. 2 trials of 50% corners occluded. 1 trial of force un-occluded flipped. Functional movement training=traverse 30 stairs.
Day 7: Rest

The above week-long program can then be repeated over the course of several weeks or months. This is further illustrated in FIG. 22 which is a flow chart 520 of a Neuro-Modulation Therapy method according to an embodiment. The method comprises choosing a target parameter, such as force, and a curve function such that the force varies as a function of time. For example, this may be as a sinusoid curve, sawtooth, or any of the waveforms or functions taught or contemplated in the present disclosure. An associated sequence of occluded (suppressed) curves is then determined. These may comprise a curve with a central occlusion, a turning point occlusion or any combination of the types of occlusion (suppression) taught or contemplated in the present disclosure. That is, for each subsequent cycle of neuromuscular control characterisation, the user is presented with the next sequential occluded curve representation. In some embodiments an initialisation feedback step is performed in which the user is instructed to perform an exercise (or sequence of actions) to match the target parameter curve (i.e., with no added suppression or occlusion). This is followed by repeated execution of feedforward exercises. The suppressed or occluded portions may follow a predetermined (or predefined) sequence. That is, we start with a first exercise wherein the target parameter follows the current curve in the sequence of occluded curves, in this case the first curve. We then progressively present the next occluded curve in the sequence until all have been presented. Each occluded curve in the sequence may be presented one or more times. Assessments may be performed on the occluded curves or at various time points in the sequence.

The method taught by way of FIG. 23 may include one, or be a set of multiple cycles, of characterisation of neuromuscular control. Furthermore, if more than one set of characterisation of neuromuscular control is required, they need not be conducted in the one session. They may be conducted over the course of a day, multiple days or longer. In this case progression of the predefined sequence of occluded curves need not be strictly enforced in a linear fashion. In some embodiments each session comprises at least one initialisation feedback exercise using a non-occluded curve before proceeding to feed-forward exercises using occluded curves. This allows the user to refresh the real-time-feedback pathways in advance of engaging the real-time-feedforward pathways. In some embodiments the session may start with feed-forward exercises (i.e., without an initial feedback exercise).

In some embodiments the output apparatus may be a haptic apparatus which provides haptic feedback in which the frequency or intensity of haptic feedback changes to indicate a target level or the difference between the target level and the measured level.

In yet other embodiments the second output apparatus 326 in FIG. 14 may be an audio transducer in which the audible amplitude (sound pressure level), pitch (frequency) or other audible property changes to indicate the target level, or the difference between the target level and the measured level (the error). In some embodiments, the computing apparatus may also include an audio interface, which may include a text to speech converter, for example to provide an audio instruction to the user of the requested exercise and/or to notify them of the beginning and end of the session. The audio interface can also be used to provide other audio cues or information to the user. The audio interface may include one or more speakers or be operatively connected to one or more speakers, headphones, ear buds or other form of sonic transducer either via wired connection or wireless connection. In one embodiment an audio transducer, functionally equivalent to second output apparatus 326 of computing apparatus 212 as shown in FIG. 14, is adapted to provide an audible representation of the predefined pattern wherein pitch is used to indicate the target force. Either increasing or decreasing pitch may be used to indicate an increasing target force (or another parameter). The audio transducer would either replace the first output apparatus 324 of FIG. 14, or act as an additional output apparatus operating in parallel, to provide information about the required force that the user would be required to replicate on the neuro-modulation apparatus (such as extension apparatus). Feedback to the user about the measured force may or may not be provide to the user by visual apparatus, audible apparatus or other apparatus. In all other respects the Neuro-Modulation Therapy would be the same as previously disclosed. Using an audible apparatus has additional benefits. Memory activation during sleep using sound has been investigated as a way to consolidate a learnt motor skill. Some studies have demonstrated enhanced motor performance after audible activation of motor networks during sleep thereby using target memory reactivation to augment learning in a clinical context. Studies have shown improved, selective precise control of specific muscles, improved efficiency/accuracy of motion and reaching a target location more quickly.

Neuro-Modulation Therapy may therefore optionally include memory activation of motor learning during sleep. In some embodiments a treatment method includes simultaneously using both the first output apparatus 324 and the second output apparatus 326 of computing apparatus 212 as shown in FIG. 14 to simultaneously represent the sinusoidal defined pattern 382 as shown in FIG. 16A while using the neuro-modulation apparatus. In some embodiments the first output apparatus provides a visual representation of the defined pattern, such as the sinusoid 386 of FIG. 14, and the second output apparatus provides an audible representation of the defined pattern as previously disclosed, the visual and audible representations being synchronised. Preferably as the curve on screen moves upward the pitch of the audible signal increases to indicate an increase in the target force although other combinations are possible. Subsequent to using the neuro-modulation apparatus, memory activation occurs overnight or during napping by replaying the same audible signal that was played during use of the neuro-modulation apparatus. In this way the neural patterning created while using the neuro-modulation apparatus is activated while asleep. In all other respects the Neuro-Modulation Therapy would be the same as previously disclosed.

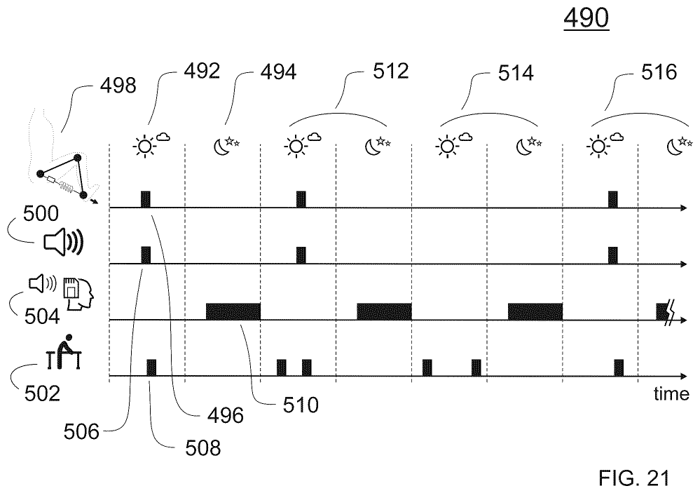

FIG. 21 is a schematic representation of several days of a Neuro-Modulation Therapy programme at 490. A day is depicted as two periods; daytime 492 defined here as 6 am to 6 pm and night-time 494 defined as 6 pm to 6 am. Sessions using a neuro-modulation apparatus, indicated by a block 496, are shown in neuro-modulation apparatus timeline 498. Similarly, sessions using an audible apparatus to provide a sinusoidal defined pattern to the user are shown in audible timeline 500. Sessions of functional therapy are similarly shown on the functional therapy timeline 502. Sessions of audible memory activation are shown in audible memory activation timeline 504.

In some embodiments both visual apparatus and audible apparatus are used to provide a sinusoidal defined pattern to the user, however only the audible apparatus is necessary for memory activation and neuro-modulation apparatus timeline 498 may optionally not include visual apparatus. For the purpose of simplifying this exemplary embodiment the neuro-modulation apparatus timeline 498 is assumed to include the visual apparatus and use of audible apparatus is shown separately on the audible timeline 500.

On the first day of therapy 492 the user undergoes a session 496 using the neuro-modulation apparatus 498 and an accompanying and synchronised session 506 presenting the sinusoidal defined pattern to the user via the audio apparatus, the audio signal having been synchronised to the visual in the manner taught in the present disclosure. Neuro-Modulation Therapy creates a window of neuroplasticity for motor learning, and it is advantageous to begin functional therapy as soon as possible.

If possible, a session of functional therapy 508 is conducted shortly after completing the first session 496 of using the neuro-modulation apparatus 498. A session of audible memory activation 510, nominally depicted as roughly 10 pm through 6 am in exemplary FIG. 21, is conducted while the user sleeps on the night 494 of the first day of Neuro-Modulation Therapy. Audible memory activation involves playing the sinusoidal audio defined pattern to the user repeatedly on an infinite loop at audible but non-waking sound pressure levels (for example 40-50 dB). The audio signal played during audible memory activation session may be played in the background while you sleep using the user's cell phone, iPod, Bluetooth speaker, connected headband speaker or other audio reproduction equipment at a suitable sound pressure level.

Day two 512 of the Neuro-Modulation Therapy programme shown as encompassing both daytime and night-time periods depict the user undergoing a session of functional therapy 502 in the morning and again shortly after using the neuro-modulation apparatus 498. The timing of functional therapy is not critical because Neuro-Modulation Therapy creates a window of neuroplasticity that persists for several days. The exemplary functional therapy timeline 502 merely demonstrates that functional therapy should continue whenever possible, but a Neuro-Modulation Therapy programme should ideally include a session of functional therapy 502 shortly after each session of using the neuro-modulation apparatus 498. Some time on day two 512 another session of using the neuro-modulation apparatus 498 is conducted with simultaneous use of using a synchronised audible apparatus 500. Audible memory activation 504 continues overnight on day two 512 of therapy, as it would ideally (but not mandatorily) do on every night of the programme. Use of the neuro-modulation apparatus 498 need not occur every day as the neuroplasticity window lasts for several days. This is depicted by the somewhat arbitrary choice of not using the neuro-modulation apparatus 498 on day three 514 of therapy. While it is not necessary to use the neuro-modulation apparatus every day, it may be beneficial to use the neuro-modulation apparatus in the morning (to kick start the body for the day) and again for several sessions throughout the day for best results. Functional therapy 502 and audible memory activation 504 should continue whenever possible throughout the course of the therapy and this is depicted as two sessions of functional therapy and overnight audible memory activation on day three 514. Use of the neuro-modulation apparatus 498 resumes on day four 516 of therapy to refresh the neuroplastic response followed by functional therapy 502 and continuation of overnight audible memory activation 504.

This embodiment teaches the use of synchronous visual and audible apparatus while using the neuro-modulation apparatus and using audible apparatus during sleeping memory activation. Embodiments also anticipate using only the audible apparatus while using the neuro-modulation apparatus and audible apparatus during sleeping memory activation. Combinations of this methodology are anticipated in the present disclosure.

The therapy taught by way of FIG. 21 is exemplary of a general Neuro-Modulation Therapy regardless of whether audible memory activation is utilised. Specifically, if a given course of Neuro-Modulation Therapy does not include audible memory activation, the discussion of FIG. 21 is a suitable description of the therapy programme if the audible memory activation timeline 504 is removed from the discussion. In other words, Neuro-Modulation Therapy includes use of a neuro-modulation apparatus 498, use of a visual apparatus 324 and/or an audible apparatus 326 (or any other apparatus taught or contemplated in the present disclosure) of providing a target force (or other parameter taught or contemplated in the present disclosure) followed by sessions of functional therapy 502. Furthermore, numerous studies have shown the benefits of sleep in reinforcing and improving new motor skills. Sleep immediately after learning a new motor skill has been shown to significantly improve motor skill retention. This can occur after a 90 minute period of sleep. It is therefore advantageous to perform neuro-modulation up to and/or immediately prior to a period of sleep. If one is to augment this with sleep memory activation the benefits may be multiplicative.

Together a neuro-modulation apparatus, comprising all of its constituent parts as disclosed (or any of the variants taught or contemplated within the present disclosure) including the extension apparatus 202, the computing apparatus 212, software, procedures and algorithms contained therein and any of the elements taught in this disclosure comprise systems and methods of measurement or characterisation of neuromuscular control, enhancing neuroplasticity particularly with regard to motor learning, reducing the effects of arthrogenic muscle inhibition, rehabilitation following injury or surgical intervention, performance optimisation and training the body to improve neuromuscular control of a given joint, structure or muscle group.

The system may be configured to measure the target parameter such as the force exerted, or position, acceleration, joint angle or any of the other parameters taught or contemplated in the present disclosure, by a user as a function of time. Variation of the target parameter may include form (sinusoid, sawtooth or other form), amplitude, frequency, phase, suppression or any other parameter taught or contemplated in the present disclosure. In particular the extension apparatus measures the force whilst the user is performing an exercise using a neuro-modulation apparatus attempting to apply the target force in real time. The force measurement apparatus 210 communicates the force measurements in real time for real time display on the display apparatus of computing apparatus 212. The force measurement apparatus 210 may be integrated into the extension apparatus or it may be configured to be removably attached to the extension apparatus. Calculation of the error, that is the difference between the target and measured parameter, may include any mathematical or statistical analyses taught or contemplated in the present disclosure.

Embodiments have been disclosed with respect to an exemplary knee joint however it is applicable to any joint, muscle, tendon, ligament or other structure in the body that relies on motor control. The knee is one of the simplest joints in the body as it is to a large extent constrained in one plane. Although embodiments disclose the assessment of an exemplary knee, it is not assessing the knee in isolation. It would be readily apparent to one of ordinary skill in the art that the methods taught via the equivalent circuit of FIG. 10 also exercise and therefore assess the hip (albeit in-plane) and to a lesser extent the ankle. If the user has normally functioning knees, then operating the hip joint with the single plane extension apparatus of, for example, FIG. 8A is a good proxy measure of hip function in at least the in-plane dimension and possibly in all dimensions. Fluid motion in one plane implies control in the other planes to some extent. The same cortical drive and cerebellum processes apply. That is, even in the case of more complex joints such as the hip or shoulder with movement in multiple dimensions embodiments which provide assessment in a single plane or dimension can be used to provide information on the respective joint.

The elbow is a much more complex joint than the knee. It consists of two separate articulations defined by firstly the trochlear notch of the ulna and the trochlea of the humerus and secondly the head of the radius and the capitulum of the humerus. This gives the joint an additional degree of freedom. Although it can move in very much the same hinge-like manner as the knee (such as when someone does a bicep curl exercise) the distal segment of the arm or forearm is able to rotate around its axis.

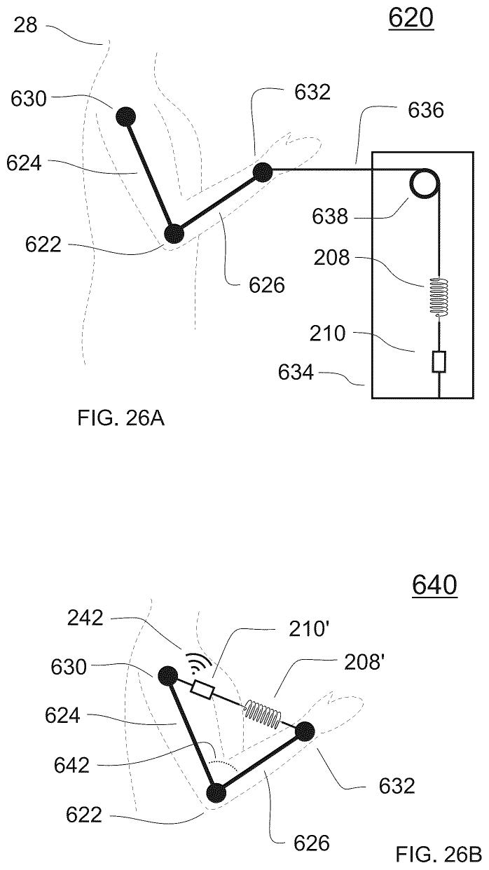

The present discussion teaches the use of embodiments in treating conditions of the elbow within a single plane NMT in much the same manner as the hinge-like treatment of the knee. FIG. 26A is a sagittal plane schematic representation of a bicep curl extension apparatus applied to the elbow joint at 620 where similar elements from previous figures are similarly numbered. The elbow joint 622 connects extended proximal 624 and distal 626 members. Proximal member 624 (the humerus) is attached to the body 28 by the shoulder joint 630 and the distal member 626 (which is comprised of the radius and ulna) is terminated at the wrist joint 632. Bicep curl equipment 634 includes a force measurement apparatus 210 which is rigidly attached to the bicep curl equipment 632 at the distal end and rigidly attached to a resistive element 208 at the proximal end. The proximal end of the resistive element 208 is connected to the user by a non-elastic cable 636 via a pulley 638. Assuming that the user maintains a fixed position of the wrist 632 or the connection point between the wrist 632 and cable 636 is somehow rigid, perhaps via a wrist-strapped glove, then the motion of the elbow joint in the sagittal plane closely resembles that of the knee. Bicep curl extension apparatus 634 is preferably configured with communication apparatus, computing apparatus, software applications and all of the features taught in prior embodiments of neuro-modulation apparatus in any combination. FIG. 26B is a sagittal plane functionally equivalent schematic representation of the embodiment of the bicep curl extension apparatus 634 at 640 wherein only the functionally equivalent elements are presented and where similar elements from previous figures are similarly numbered. In an ‘equivalent-circuit’ sense, the shoulder 630 and wrist 632 are connected via resistive element 208′ and rigidly-coupled, force measuring apparatus 210′, which in this embodiment is shown connected to computing apparatus (not shown) via a wireless protocol 242. In this configuration the shoulder 630 is the reference point and the elbow 622 and wrist 632 are constrained to move only within the sagittal plane. Furthermore, in this equivalent circuit resistive element 208′ is a compression spring element wherein the resistance is greatest when the internal elbow angle 642 is at a minimum. The biceps muscle is used to compress the equivalent circuit spring in a concentric contraction. When releasing against the spring, an eccentric muscle contraction is used by the biceps muscle.

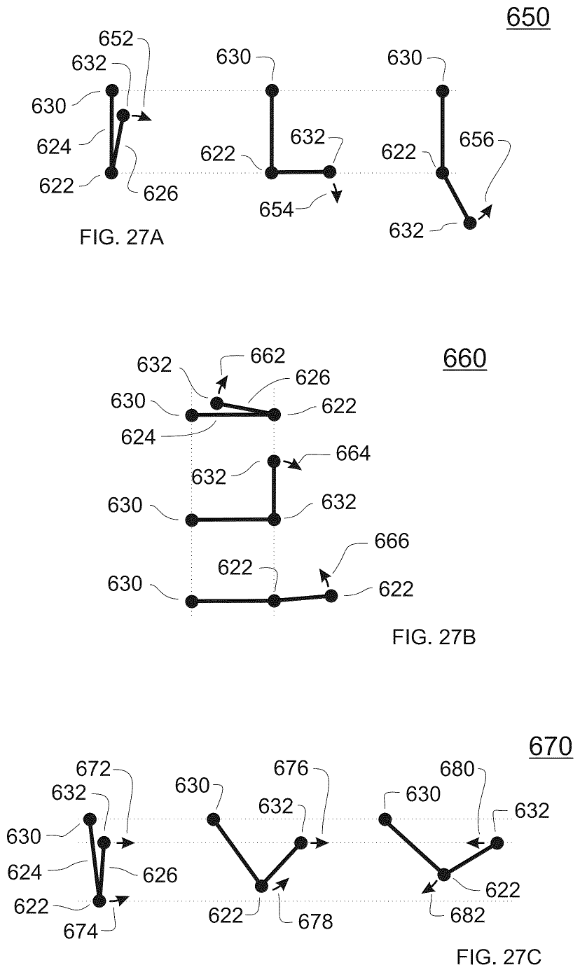

In order therefore to isolate the motion of the elbow into a purely hinge-like action it is preferable to ensure that the shoulder joint (and the wrist for that matter) is fixed throughout the range of motion required by the defined pattern of motion taught in this disclosure. This is achieved by keeping proximal member 624 in a fixed geometry with respect to the rest of the body 28. FIG. 27A is therefore a schematic representation of an embodiment at 650 wherein similar elements from previous figures are similarly numbered as a sagittal plane schematic representation of a bicep curl extension apparatus applied to the elbow joint. In this embodiment the user is either standing or in an upright seated position and the proximal member 624 is vertical. The wrist (or distal end 632 of the distal member 626) is preferably fixed to the extension apparatus by a strap or some other restraint. Beginning from left to right in the figure with the arm retracted (a minimum internal elbow angle 642) the arm is extended in an eccentric biceps contraction with the wrist tracing out an arc 652. As the eccentric contraction continues, the internal elbow angle 642 reaches 90 degrees and the wrist continues to trace out its arc 654. Once the arm reaches its full extension with the internal elbow angle 642 reaching its maximum near 180 degrees the user begins a concentric biceps contraction and the wrist begins to trace out an arc 656. FIG. 27B is similarly a schematic representation of one embodiment at 660 wherein similar elements from previous figures are similarly numbered. In this embodiment the user is either standing, seated or lying down with the proximal member 624 is horizontal. The wrist (or distal end 632 of the distal member 626) is preferably fixed to the extension apparatus by a strap or some other restraint. Beginning from top to bottom in the figure with the arm retracted (a minimum internal elbow angle 642) the arm is extended in an eccentric biceps contraction with the wrist tracing out an arc 662. As the eccentric contraction continues the internal elbow angle 642 reaches 90 degrees and the wrist continues to trace out its arc 664. Once the arm reaches its full extension with the internal elbow angle 642 reaching its maximum near 180 degrees the user begins a concentric biceps contraction and the wrist begins to trace out an arc 666.

In both of these cases, FIGS. 27A and B, the motion and therefore the Neuro-Modulation Therapy is isolated to the elbow joint, quite unlike the embodiments taught for the exemplary knee where both the knee joint and the hip joint (and possibly the ankle joint) were in motion to track the defined pattern. Embodiments may therefore be adapted to improving neuromuscular control of an isolated joint or an entire limb. Someone of ordinary skill in the art would appreciate that given the motion of the wrist 632 traces out an arc, the resistance element of the extension apparatus undergoes non-linear motion and this needs to be accounted for in analysis of the measurements. In order then to complete the discussion on elbows it is worth considering another case where the motion of the wrist is linear with respect to the extension apparatus.

FIG. 27C is therefore a schematic representation of one embodiment at 670 wherein similar elements from previous figures are similarly numbered. In this embodiment the both the elbow and the shoulder will be in motion in order to ensure the wrist traces out a straight line in the sagittal plane. The wrist (or distal end 632 of the distal member 626) is preferably fixed to the extension apparatus by a strap or some other restraint. Beginning from left to right in the figure with the arm retracted (a minimum internal elbow angle 642) the arm is extended in an eccentric bicep contraction across the elbow with the wrist tracing out a straight path 672 and the elbow therefore tracing out an arc 674. As the eccentric biceps contraction continues the internal elbow angle 642 reaches 90 degrees, the wrist continues to trace out its linear path 676 and the elbow its arc 678. Once the arm reaches its full extension with the internal elbow angle 642 reaching its maximum near 180 degrees the user begins a concentric biceps contraction across the elbow to return the arm to the starting position. The wrist begins to retrace its linear path 680 and the elbow its arc 682. Further, embodiments allow the wrist to trace out a path in a plane other than the sagittal including the coronal plane, the axial plane or indeed in three dimensions and an apparatus for this will be taught later in this disclosure.

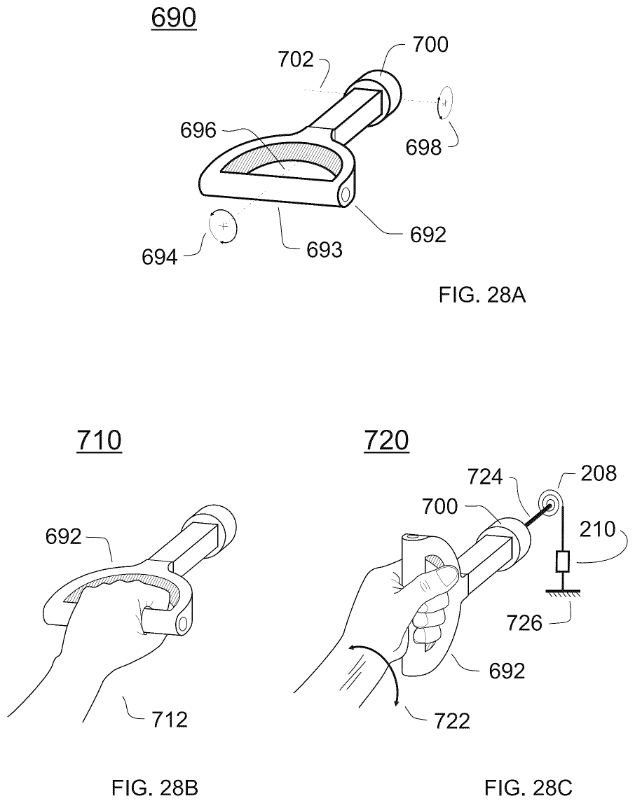

As stated, the elbow is much more than a simple hinge-like joint. The second degree of freedom allows nearly a 180 degree rotation of the distal end of the distal member 626 around its axis. FIG. 28 is a partial schematic representation of an extension apparatus for treating the elbow, particularly in the second degree of freedom. FIG. 28A is a schematic representation of the end effector of the apparatus for treating the elbow at 690, however this particular extension apparatus may be further adapted for treatment of the wrist, as will be disclosed in due course, but for the purpose of FIG. 28A and the present embodiment the wrist is assumed to be in a fixed position. Handle 692 is gripped by the hand around the grip bar 693 and the apparatus has two degrees of freedom. Handle 692 is attached to the extension apparatus such that it is able to be rotated 694 around its long axis 696, preferably via a rotary bearing 700. Handle 692 may furthermore be rotated 698 (or perhaps more aptly, pivoted) around a perpendicular axis 702. FIG. 28B is a schematic representation at 710 of a human hand 712 holding the grip bar 693 of handle 692 wherein similar elements from previous figures are similarly numbered. The forearm or distal member 626 is placed on a table or similar resting surface such that it is coaxially aligned with the long axis 696 of the handle and the elbow is preferably rigidly located with respect to the handle 692. This ensures that rotational motion of the distal end of the distal member 626 is entirely controlled by the elbow joint rather than via a contribution from other joints such as the shoulder.

FIG. 28C is a schematic representation at 720 of the distal end of the distal member 626 having been rotated 722 by roughly 90 degrees wherein similar elements from previous figures are similarly numbered. Handle 692 is therefore rotated around its long axis 696, assuming for the moment that the perpendicular axis 700 is fixed. Handle 692 contains a rigid extension 724 coaxially aligned with the long axis 696 to which the proximal end of a resistive element 208 is attached. In a preferable embodiment resistive element is a torsional spring adapted to provide a variable force as the handle 692 is rotated around its long axis 696. The distal end of resistive element 208 is rigidly attached to a force measurement apparatus 210 which is subsequently rigidly attached to the extension apparatus. In this way, rotation of the handle 692 against the resistive element results in a measurable force. The present apparatus is preferably configured with communication apparatus, computing apparatus, software applications and all of the features taught in prior embodiments of neuro-modulation apparatus in any combination.

It would be readily apparent to one of ordinary skill in the art that other forms of resistive element would be functionally equivalent as taught throughout this disclosure and each should be considered operably replaceable in the present embodiment where appropriate. It would also be readily apparent to one of ordinary skill in the art that a positional sensor may be used instead of the force measurement apparatus 210—as was the case with positional sensor 229 of FIG. 8B. In such an embodiment a rotary shaft encoder may be used to directly measure the angular displacement of the handle during rotation rather than using a force measuring apparatus as a proxy for rotation. It would furthermore be readily apparent to one of ordinary skill in the art that a constant frictional force may be applied in the rotary bearing 700 or using an ancillary frictional resistive apparatus.

The wrist joint has an additional degree of freedom, flexion-extension, radioulnar deviation and rotation, allowing movement of the hand in three dimensions. The normal functional range of wrist motion is 73 degrees of flexion, 71 degrees of extension, 19 degrees of radial deviation, 33 degrees of ulnar deviation, 140 degrees of supination and 60 degrees of pronation (A study on the measurement of wrist motion range using the iPhone 4 gyroscope application. Kim T S, et al. Ann Plast Surg. 2014 August; 73(2):215-8). The present embodiment of FIG. 28A anticipated treatment of the wrist and an initial discussion will be limited to motion only in the sagittal plane—flexion and extension.

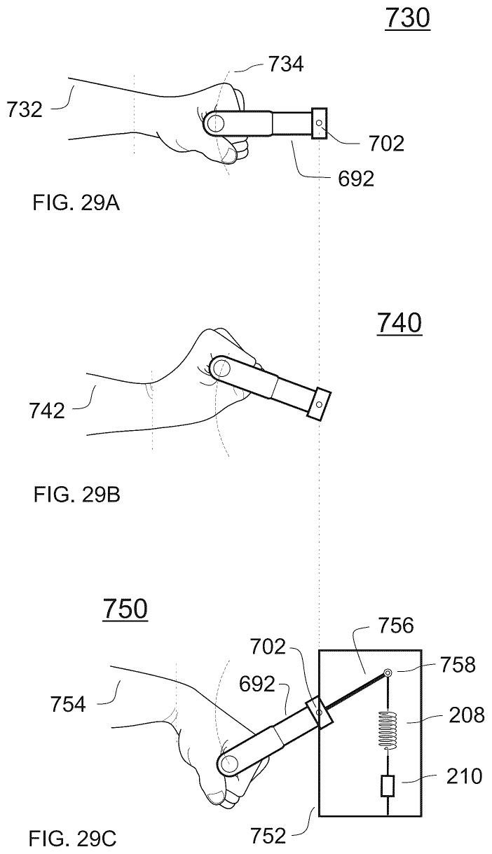

FIG. 29A is therefore a schematic representation of a sagittal plane extension apparatus adapted to treat the wrist in flexion and extension at 730 wherein similar elements from previous figures are similarly numbered. The wrist is shown gripping handle 692 in neutral position 732. Handle 692 is allowed to pivot around axis 702, perpendicular to the plane of the page in FIG. 29, but axis 696 is fixed such that there is no rotation 694 of the handle 692. In this way the grip of handle 692 traces an arc 734 in the sagittal plane. FIG. 29B is a schematic representation at 740 of the wrist in extension 742. It is important to note that in order for the wrist to flex or extend the distal member 626 must be allowed to slide fore and aft in the sagittal plane along the table or supporting surface rather than the elbow being fixed as was the case with the rotational motion disclosed by way of FIG. 28. Finally, FIG. 29C is a sagittal plane schematic representation of the extension apparatus 752 for flexion and extension of the wrist at 750. The wrist is shown in flexion 754 with the handle 692 pivoted around the bearing of the hinged axis 702. Handle 692 includes a rigid extension 756 coaxially aligned with the long axis 696 to which a hinge 758 is attached at the distal end. Hinge 758 is also rigidly attached to a resistive element 208, the distal end of which is rigidly attached to a force measurement apparatus 210 which itself is subsequently rigidly anchored to the extension apparatus 752. The present apparatus is preferably configured with communication apparatus, computing apparatus, software applications and all of the features taught in prior embodiments of neuro-modulation apparatus in any combination. It is readily apparent to one of ordinary skill in the art that resistive element 208 and force measurement apparatus 210 may be substituted with similarly functional sensors, apparatus or devices as taught throughout this disclosure.

The extension apparatus 752 of FIG. 29C exercises the wrist in flexion and extension however the wrist joint is also able to move the hand laterally and indeed in circles via a combination of flexion-extension and radioulnar deviation. In order to fully assess the functionality of the wrist joint an extension apparatus must accommodate this full range of motion. One such method would be to replace the hinged axis bearing with a gimbal bearing. Such a gimbal bearing would indeed allow the long axis 696 of the handle 692 to move within a solid angle from the perspective of the bearing however this would result in the axis of the grip bar 693 becoming non-orthogonal to the plane of the page as viewed in FIG. 29. This would completely change the dynamics of the wrist during and the whole arm would need to move to accommodate the changing geometry. This confounding issue can be overcome with the incorporation of a universal joint or a ball joint in the handle which would allow independence of the grip bar 693 and long axis 696 geometry during use.

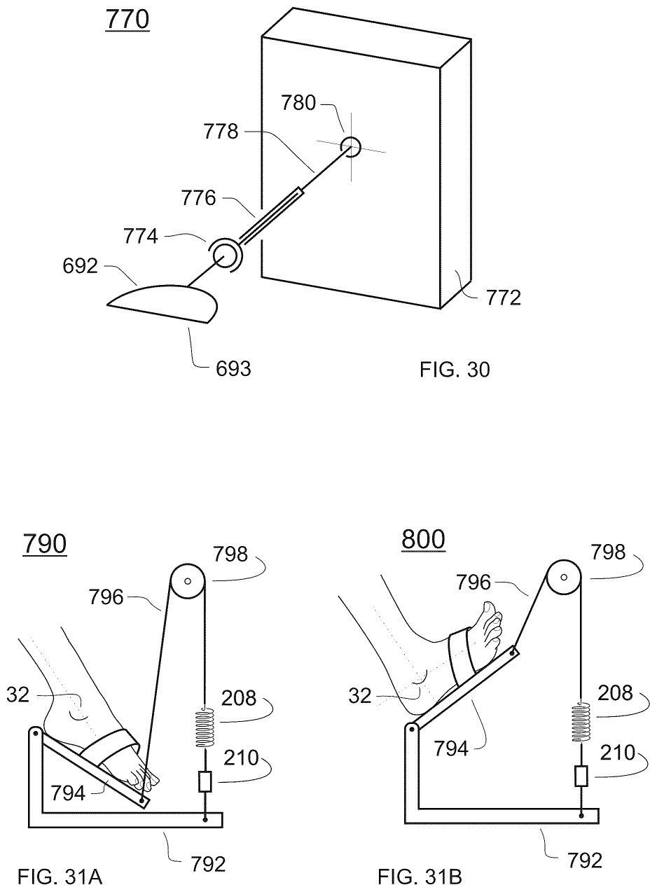

Therefore FIG. 30 is a schematic representation of an extension apparatus accommodating the full range of motion of the wrist wherein similar elements from previous figures are similarly numbered. Extension apparatus 772 comprises a handle 692 and its associated grip bar 693, shown in the neutral wrist position described by way of FIG. 29A, which is rigidly attached to an articulated arm comprising a ball joint 774 and a sliding extension tube 776. The distal end of the sliding tube assembly passes through a gimbal bearing 780 or similar bearing structure which itself it rigidly attached to the body of the extension apparatus 772. Such an assembly allows the grip bar to be moved in 3-dimensional space as the wrist joint is articulated through its complete range of motion. The sliding extension tube allows for the variable length of the ball joint 774 to gimbal bearing 780 distance as the wrist joint is articulated. The ball joint 774 may optionally be a universal joint or any other articulated joint offering a similar range of motion. It would be readily apparent to one of ordinary skill in the art that the internal mechanism of the extension apparatus 772 would be similar to that of other apparatuses taught in the present disclosure with the obvious extension that it would need to map force and or position in orthogonal dimensions and possibly with the use of one or more resistive elements similar to resistive element 208. The present apparatus is preferably configured with communication apparatus, computing apparatus, software applications and all of the features taught in prior embodiments of neuro-modulation apparatus in any combination. It would also be readily apparent to one of ordinary skill in the art that the apparatuses of FIGS. 28, 29 and 30 may be combined such that the one apparatus accommodates NMT of the wrist and rotation 722 of the distal end of the distal member 626.

It will be readily apparent to one of ordinary skill in the art that embodiments of NMT are adaptable to all musculoskeletal functional groups. FIG. 31A is a sagittal plane schematic representation of an extension apparatus applied to the ankle joint at 790 wherein similar elements from previous figures are similarly numbered. Extension apparatus 792 comprises a hinged foot plate or foot retaining apparatus 794 to which a non-elastic cable 796 is attached. Non-elastic cable 796 passes around a pulley 798 and is attached to a resistive element 208. In the present embodiment the resistive element is a spring, but it may be any of the resistive elements taught in this disclosure. Resistive element 208 is rigidly attached to a force measurement apparatus 210 which itself is attached to the extension apparatus 792. FIG. 31A demonstrates the ankle joint 32 at the extension extreme of its range of motion. FIG. 31B is a sagittal plane schematic representation of the other extreme of ankle motion, flexion, in an extension apparatus at 800 wherein similar elements from previous figures are similarly numbered. The ankle motion represented in FIG. 31 is purely in the sagittal plane. The present apparatus 792 is preferably configured with communication apparatus, computing apparatus, software applications and all of the features taught in prior embodiments of neuro-modulation apparatus in any combination. It would be readily apparent to one of ordinary skill in the art that all of the teachings of the present disclosure apply to this preferred embodiment of the ankle extension apparatus, including the application of mechanisms to enable the full range of 3-dimensional motion for the ankle joint. An example of such would be an embodiment wherein the foot retaining apparatus 794 is attached to the body of the extension apparatus by a ball and socket joint centrally located beneath the foot retaining apparatus 794 with a pair of force or position measuring apparatus adapted to determine the motion of foot plate in orthogonal planes.

It would be readily apparent to one of ordinary skill in the art that the hip joint has more degrees of freedom than the knee joint. Treating the hip across its full range of motion would therefore require an apparatus that can measure force versus time (or position or any of the other measured variables taught in this disclosure) in three dimensions whereas the knee only requires one dimensional command and measurement. Embodiments have taught apparatuses for assessing neuromuscular control and performing Neuro-Modulation Therapy on peripheral joints, but these are equally valid for the ball-and-socket joints of the hip and shoulder. The shoulder joint is more akin to a golf ball sitting on a tee whereas the hip is more of a true ball-in-socket joint. The shoulder joint is made up of the head of the humerus which rests on the glenoid fossa of the scapula the two being held together only by the 4 muscles that make up the rotator cuff. The hip joint by comparison comprises the head of the femur which fits into the acetabulum of the ilium.

The general principles of NMT have been taught by reference to an exemplary knee joint. The general principles have been constrained largely to in-plane, linear, extension-flexion motion. Those principles have been extended to other joints which have more degrees of freedom leading to the present discussion of the hip and shoulder joints. It would therefore be readily apparent to one of ordinary skill in the art that an apparatus may be and indeed is intended to be constructed using a combination of the various embodiments taught in the present disclosure to enable coordinated three dimensional neuromuscular assessment and Neuro-Modulation Therapy.

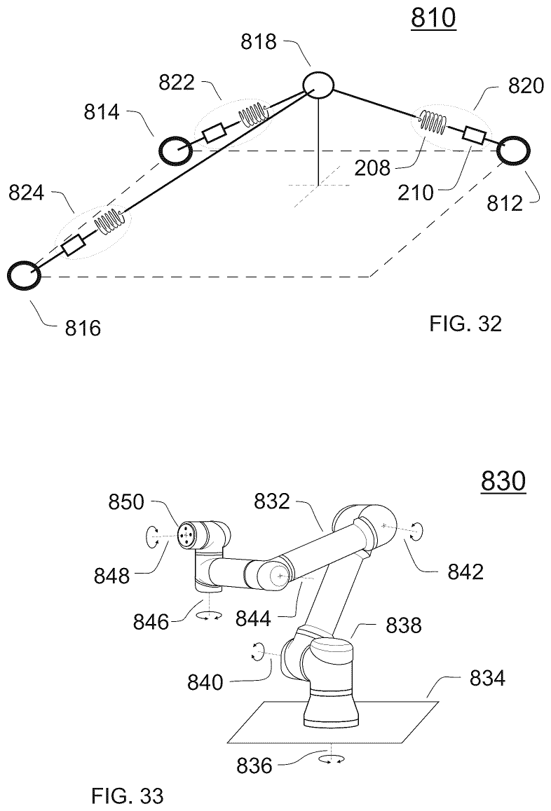

In an embodiment a user is attached to a neuro-modulation extension apparatus comprising a plurality of orthogonal extension apparatuses, each extension apparatus taking the form of the functionally equivalent circuit of FIG. 10. FIG. 32 is therefore a functionally equivalent schematic representation of a multi-planar neuro-modulation extension apparatus at 810 wherein only the functionally equivalent elements are presented and where similar elements from previous figures are similarly numbered. First reference point 812, second reference point 814 and third reference point 816 define the reference points required to triangulate the position of the user attachment point 818 of the extension apparatus. In an ‘equivalent-circuit’ sense, each of the reference points 812, 814 and 816 are connected to the user attachment point 818 by a resistive and force measuring element 820, 822 and 824 respectively, each of which is comprised of a resistive element 208 and a force measurement apparatus 210. The resistive element 208 and the force measurement apparatus 210 can comprise any of the variants taught throughout this disclosure. In this way the user attachment point 818 can be monitored in three dimensional space to enable the full range of motion to be captured, analysed and subsequently rehabilitated. Similarly, the joints of the spinal column are complex with significant changes across the articulating functional groups, cervical, thoracic and lumbar and more subtle changes across members of these groups. There is a vast range of motion possible for the range of joints in the human body. It is therefore preferable to have a single extension apparatus for assessing neuromuscular control and for providing Neuro-Modulation Therapy, wherein the single extension apparatus can assess a plurality of joint movements—abduction, adduction, inversion, rotation and any joint movement disclosed or otherwise.

In some embodiments, a multi-axis articulated arm may be used which offers many advantages, particularly in terms of flexibility to accommodate the motion of any joint and in any configuration. A multi-axis articulated arm is suitable for incorporation of an active resistive element associated with each axis or degree of freedom of the arm with an electric motor being a preferred active resistive element. In such an embodiment the electric motor could be further adapted to include force or torque sensors on one or more of the axes to deliver a desired force profile across a range of motion.

A cobot, or collaborative robot, is a robot intended for direct human-robot interaction within a shared space or where humans and robots are in close proximity. Cobot applications contrast with traditional industrial automation robot applications in which robots are isolated from human contact. Cobot safety is built on lightweight construction materials, rounded edges, limitations on speed of movement, force and most importantly the use of feedback sensors and software systems. A typical cobot is comprised of a series of arm segments, each articulated with respect to the previous and configured with an electric motor in the elbow between each segment. FIG. 33 is a schematic representation of a typical cobot at 830. Cobot 832 is attached to a table or work surface 834. The whole cobot 832 is operable to rotate around first axis 836, perpendicular to the plane of the table 834 and driven by internal electric motor 838. Cobots typically have right angle elbow joints such as that defined by the first axis 836 and second axis 840 although other angles are possible. Second axis 840 is driven by its own internal electric motor (not shown), as are all of the elbow joints. Third axis 842, fourth axis 844 and fifth axis 846 complete the 5 degrees of freedom of the articulating arm and sixth axis 848 provides a sixth degree of freedom for rotating an ‘end-effector’ (which may be a gripping ‘hand’, suction cup or some other form of physical cobot interface) attached to mounting plate 850. Cobots contain control software that ensures their safety while working with humans and this software is able to control the position, speed, force and orientation of the end-effector. It is also able to determine the force imparted on the end-effector and log all of the data to its attached computing apparatus.

A cobot could then be used with one or more specific end-effectors designed for attachment of different body parts to enable one apparatus to be flexibly configured to perform neuromuscular assessment or therapy of any body part. Cobots provide almost unlimited flexibility for neuromuscular assessment and therapy. Cobots can be used with or without computer vision and the robotic arms can utilise force feedback mechanisms to capture neuromuscular assessment measurements through any range or position of motion. Identifying where in the multi-directional range of a joint motion a patient loses neuromuscular control is critical to unlocking targeted interventions that personalise efficacious rehabilitation and manage instability after injury or surgical intervention. Using robotic arms in cobots has the advantage over existing exercise equipment for the ability to ideally position and measure the full, high resolution three-dimensional nature of a joint or joints and provide the user with a meaningful, targeted intervention exercise in the area it is most needed. Cobots allow the force transducers to be ideally placed perpendicular to the body part or direction of motion. Computer vision may be optionally used to measure the body parts and hence calculate accurate torque measurements at any point in the humanly usable space. Alternatively, a plurality of cobots may be simultaneously used to locate a plurality of reference points on the user's body which can subsequently be used to calculate position, angle, torque or any other relevant parameter. This allows targeting of rehabilitation for the precise conditions for which a patient displays musculoskeletal control deficits. Stored neuromuscular profiles for each patient, measured by the cobot, allows rehabilitation to be exactly replicated and compared at any point in time. This is important when commencing again from the last session and especially when the patient has had a period of time away from rehabilitation. This is particularly valuable for periodic follow-up examinations by clinicians such as at the one-year total knee arthroplasty following-up examination to determine if a patient requires further musculoskeletal control rehabilitation. This may also be used with an elite athlete who arrives at a tournament wishing to dial in their own, personal neuromuscular profile, parameters and training regimen. In one embodiment, the cobot measures the user's range of motion and assesses their neuromuscular control with no applied force by the cobot's electric motors—essentially freely moving—with this performance compared to the same motion profile under load.

Pilates is an exercise regime that focusses on strength, flexibility, core stability, posture, muscle control and breathing. Pilates comprises is a combination of exercises to challenge the user through consistency of repeated exercise movements that involve repetitions of an exercise. While patients benefit from this form of exercise, including those in rehabilitation, there is no way of knowing precisely where the patient is losing musculoskeletal control, nor is there a way of specifically targeting exercise at the correct joint ranges for the deficits. The use of cobots with the Pilates method enables the exercised to not only be replicated but enhanced through identifying and targeting the musculoskeletal control deficits. This creates a value-based Pilates approach, particularly in light of the feedback and feedforward neuroplastic therapies taught in the embodiments, which encourages easy progression through cobot programmes with clearly defined targets and end points, in a shorter period of time than traditional therapies.

In some embodiments, a neuro-modulation extension apparatus comprises one or more cobots which are used to guide the user in performing an exercise, provide support during the exercise and/or to measure the force and joint pose (position/orientation) data whilst performing an exercise. Cobots may thus be used for neuromuscular assessments, therapy, and development applications. Cobots may take the form of a robotic arm, such as an articulated arm comprising multiple arm segments connected by rotary joints and ending with an end-effector. In some embodiments the end effector includes one or more force measurement apparatus such as one or more load cells, strain gauges or torque sensors. The end-effector may be flat, a curved plate, a beam, a hook, a cuff, a handle, a foot plate, a gripping arrangement or some other effector configured to attach to the user, be gripped by the user or otherwise engage with a body part of the user. The end-effector may incorporate an additional resistive element 208 and/or force measuring apparatus 210 as described throughout the present disclosure. The end-effector may also be configured as a support, including a grip, cuff, handle, curved plate or other support to support the user whilst performing an exercise. The end-effector may comprise additional components such as accelerometers, an inertial measurement unit or other sensors which are configured to allow the user's pose/posture (three dimensional position and orientation) to be determined. The cobot may have a plurality of Degrees of Freedom (DOF) depending upon the number of arm segments and joints.

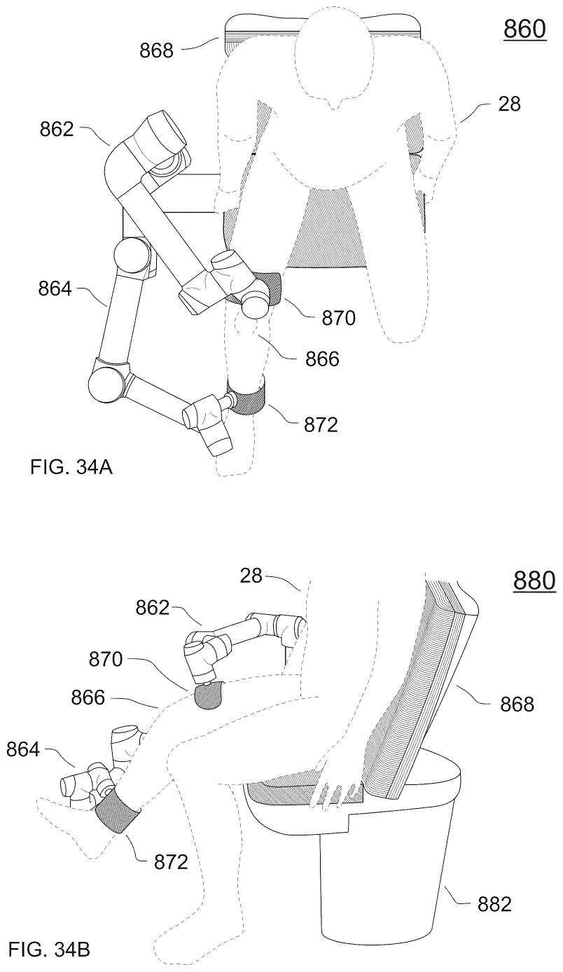

FIG. 34A is an axial plane schematic representation of a user undergoing a static joint test in a cobot-based neuro-modulation apparatus at 860 wherein similar elements from previous figures are similarly numbered. A first cobot 862 is employed to stabilise the user and a second cobot 864 is used as both a resistive element and a force measurement apparatus to collect force data from user 28 while they perform a set of exercises to assess or therapeutically treat a knee joint 866 (or 22 from FIG. 2). The display of a computing apparatus (not shown) provides the user a defined pattern 382 and the actual force 392 exerted by the user during the exercises as disclosed by way of FIG. 16, or indeed any of the force profiles taught throughout this disclosure. In this embodiment the first cobot 862 and the second cobot 864 are each mounted to a support apparatus 868 which is also used to support the user 28 whilst they perform an exercise. In this embodiment the target musculoskeletal joint is the knee joint 866 and the first end effector 870 (of the first cobot 862) is a curved plate to stabilise the quadriceps muscles to locate the knee joint 866 whilst the user performs the requested exercise. The second end effector 872 (of the second cobot 864) comprises a cuff that goes around the user's leg just above the ankle joint and incorporates a force measurement apparatus to measure the force exerted by the user when performing an exercise.

While the present embodiment discloses the use of two cobots, it would be readily apparent to one of ordinary skill in the art that the embodiment is functionally equivalent to one cobot with two arms and has been written in terms of two cobots to simplify the discussion. In fact, one or more first cobots may be employed to stabilise one or more limbs or parts of the body and one or more second cobots may be employed to measure and/or resist the motion of one or more body parts with respect to the plurality of first cobots.

It would be readily apparent to one of ordinary skill in the art that the subtle and complex motion of the spine can also be assessed utilising a plurality of first cobots to stabilise the torso and a plurality of second cobots to resist and measure the subtle changes in the torso given suitable restraining and force applying end-effectors. FIG. 34B is a near-sagittal plane view of the static joint test of FIG. 34A at 880 wherein similar elements from previous figures are similarly numbered. In this embodiment the support apparatus 868 comprises a base portion 882, to which the cobots 862 and 864 are mounted and a reclinable chair portion 868. The base portion 882 can be used to house associated power supplies, controllers and communications for driving the cobots and communicating the measured data to a computing apparatus such as 212 which may be operatively attached to the apparatus or integrated into the apparatus.

In one embodiment the cobot is programmed to follow a predefined path during an exercise and the user is instructed to activate one or more muscles associated with a target musculoskeletal joint during the exercise to provide a reaction force. The cobot may comprise one or more force measurement apparatus to measure the reaction force applied by the user whilst performing the exercise. For example, the user may sit in a support chair and the end-effector may be a cuff attached to the ankle of the user while the cobot follows a path to move the knee joint over a defined range. The user may then be asked to resist the motion of the cobot and provided with a target level of (resistance) force to apply to the cuff during the movement (which is measured by a force measurement apparatus in the cuff). In one embodiment the cobot contains enough degrees of freedom to be configured to move the target musculoskeletal joint through the full range of possible multi-planar joint movements. In another embodiment the cobot is configured to simultaneously move a combination of multiple joints including the target musculoskeletal joint in a multi-planar movement. In each case the user provides a reaction force which is measured by the cobot. In other embodiments the user may be asked to volitionally move the target musculoskeletal joint through a full range of multi-planar joint movements of the target musculoskeletal joint, by manipulating (e.g., pushing or pulling) the end effector which is used to measure the applied force. In one embodiment the force data and joint angle or pose data whilst performing an exercise are stored and the cobot is configured to replicate a previously performed exercise for which stored force data and joint angle or pose data is available. The force data and joint angle or pose data during the replicated exercise is compared to the stored force data and joint angle data to assess a change in the control of the target musculoskeletal joint.

In one embodiment, the system could be used to study how overall force is generated from multiple joints/muscle groups to detect abnormal compensatory behaviour. For example, if an injury or surgical intervention occurred to a specific joint this may cause adjacent joints to compensate for the deficit in the short term, with the compensation becoming a new (but comparatively abnormal) normal, over time. Retraining would then be required to return to the pre-injury or surgical intervention state.

Consider an exemplary exercise such as extending the leg in a single leg press functional movement. The cobot-based system could be configured to individually measure each associated joint (hip extension, knee extension and ankle planar-flexion) and compare this to a complete extension combination movement of the hip/knee/ankle all working at once. Comparing this complete vs individual data would allow the identification of relative contribution of each joint to the overall dysfunction (or functional deficit) and the specific joint angle or the force range over which the deficit occurs.

In a further form, the cobot stores or determines a threshold movement and force envelope for an exercise and generates an alert if the measured force and/or location is outside of the threshold movement and force envelope for an exercise. These envelopes may be determined by theoretical estimates or via aggregation of data from a cohort of people. This may be further refined by determining functional envelopes for specific cohorts which may include different age based cohorts, athlete cohorts, disease state cohorts and indeed may result in disease state progression metrics such as a biomarker or validated Clinical Outcome Assessment (COA). In these cases, the cobot may follow a predefined path and define the envelope based on applying a fixed radius around the end-effector so that movement along the path defines a volume. Similarly upper and lower threshold force ranges (around the target) may be defined for each point or segment along the path. The method includes methods for determining statistically relevant metrics for each cohort. If a user deviates outside of this envelope they may be issued with an alert, or the system may flag the data for further follow-up testing which may include a focus on regions where deviations from the envelope have occurred.

In one embodiment, the cobot is configured to maintain the force measurement apparatus perpendicular to a point of contact with the user whilst performing the one or more exercises. This may be predetermined based on the desired path and/or exercise. Alternatively, a feedback system may be used such as using one or more sensors or with the extension of a computer vision system to actively determine pose and adjust the end effector location.

In one embodiment a computer vision system is used to capture location and/or pose data of the target musculoskeletal joint, and one or more associated limbs whilst performing an exercise. In some embodiments a computer vision system is used with a cobot, and the computer vision system may estimate the pose of the user with respect to the end-effector. A computing apparatus (operatively coupled to or forming part of the computer vision system) uses the location and/or pose data with the force measurements to estimate applied torque as a function of time during the exercise. The computer vision system may be a marker based system or a marker-less system which use multiple cameras, LiDAR sensors, pose information from the cobot (if present) and/or and image processing to determine the joint angle or pose of the target joint and/or end-effector location. The computer vision system may be a motion capture system. The computer system may be used to estimate the torque by estimation (or measurement) of the limb length, contact point, forces and perpendicular transducer position. The force data and the joint angle or pose data may then be used to generate one or more plots representing the range of target musculoskeletal joint movement in three dimensions and the associated level of control. In one embodiment where the exercises comprise multiple combinations of multi-planar joint movements, the plots may represent the range of target musculoskeletal joint movement in three dimensions and the associated level of control. The plots may be three dimensional plots or maps with projections against orthogonal axes providing a measure of the range of force-motion in each dimension. The plots may also be used to indicate ranges associates with performing functional tasks using the target musculoskeletal joint.

In all of these embodiments, the cobot may be configured to provide a programmed resistance profile against which the user moves—in particular moves the end-effector. In some embodiments these resistance profiles are the active analog of the passive resistive element 208 of earlier embodiments, against which the user moves their body part and the user-imparted force (as measured by the force sensors in the cobot) is the analog of the force measurement apparatus 210 of earlier embodiments. Similarly, the cobot may provide a constant force across the range of movement and provide a measure of position in an analog of the positional sensor 229. Indeed, the flexibility of the cobot may provide for any combination of the embodiments taught in the present disclosure.

It would be readily apparent to one of ordinary skill in the art that the cobot-based neuro-modulation extension apparatus taught in the present disclosure is intended to be adapted to the motion of every joint or groups of joints in the body for neuromuscular assessment, reduction of arthrogenic muscle inhibition, heightening neuroplasticity, rehabilitation of injury or surgical intervention, development of motor skill function, performance optimisation or any of the other applications taught in the present disclosure. To be clear, whilst FIG. 34 shows the use of a cobot to perform static and dynamic joint tests, for example to perform a neuromuscular assessment, the cobot may be configured as a neuro-modulation cobot apparatus (or system) for therapeutic or development/performance applications. According to these embodiments the user is presented with a defined pattern of actions, which may target one joint in isolation or a plurality of joints and may define motion in one plane or in three dimensional space. The user is then provided with a range of feedback exercises along the define path (as taught by way of the single plane motion of FIG. 16) and a series of feed-forward exercises (as taught by way of the single plane motion of FIGS. 17 and 18). In this way the cobot represents a highly flexible neuro-modulation therapeutic apparatus or system. Similarly in development and performance enhancing applications the pattern of exercises may be used to further develop or refine existing skills of the user. The pattern may be stored and repeated at later time points to exactly replicate an earlier test to allow an assessment of progress or a comparison with earlier state. Artificial intelligence (AI) systems could measure progress against stored normative data. If a deviation from the normative data is detected a “red flag” may be raised for additional intervention assessment. A cobot may also be used to map musculoskeletal control and/or deficit in three dimensions (3D) e.g., hip joint 6 DOF. A comparison of left and right sides could be performed, and data could also be compared against other data measurements like MRI, CT scan, x-ray images to create a passive versus active 3D neuromuscular control picture of a joint of interest. A cobot can also be used to measure numerous combinations of complex multiplanar joint movements and to compare these against functional tasks the patient has pain or difficulty with. The cobot could be used as a Pilates replacement, for example with the addition of data capture and graduated load based on deficits measured in real-time. Cobots are also well suited to repeated repetitious neuromuscular learning using graduated progression of load/complexity.

It would be readily apparent to one of ordinary skill in the art that the cobot neuro-modulation apparatus may include a plurality of apparatus or devices for providing the defined pattern to the user, including visual apparatus, audible apparatus and haptic apparatus. Combinations of these apparatus may be used simultaneously according to any of the methods taught throughout this disclosure. It would therefore be apparent that sleep memory activation is intended to be applied within cobot-based neuro-modulation therapies.

In some embodiments a computer vision or object tracking system is used with the extension apparatus to determine the pose (location and orientation) of the user's joints and limbs and/or the pose, i.e., location and orientation, of the cobot end effectors whilst the user performs requested exercises. An audio/visual display system may also provide a visual representation (i.e., display) to the user of the defined pattern and the measured force levels and any associated instructions. The computer vision system may comprise a plurality of cameras in known locations around the apparatus. Multiple cameras may be co-located to provide binocular or depth of field information or may be used to provide triangulated information. Alternatively, LiDAR (light detection and ranging) sensors may be used to map the three dimensional position of the user. LiDAR is a method of determining the range (distance) of a target or object using a laser to measure the time of flight for the reflected light returned to the receiver. Cameras may also operate in visual or infrared ranges and may include IR emitters/illumination to assist with object tracking. Optical and LED markers may also be used to assist with pose estimation. Additionally, the cobots may comprise pose sensors (e.g., accelerometers, gyroscopes, or an inertial measurement unit) to provide additional pose information. These embodiments incorporating video or computer vision provide a third person view of the user.

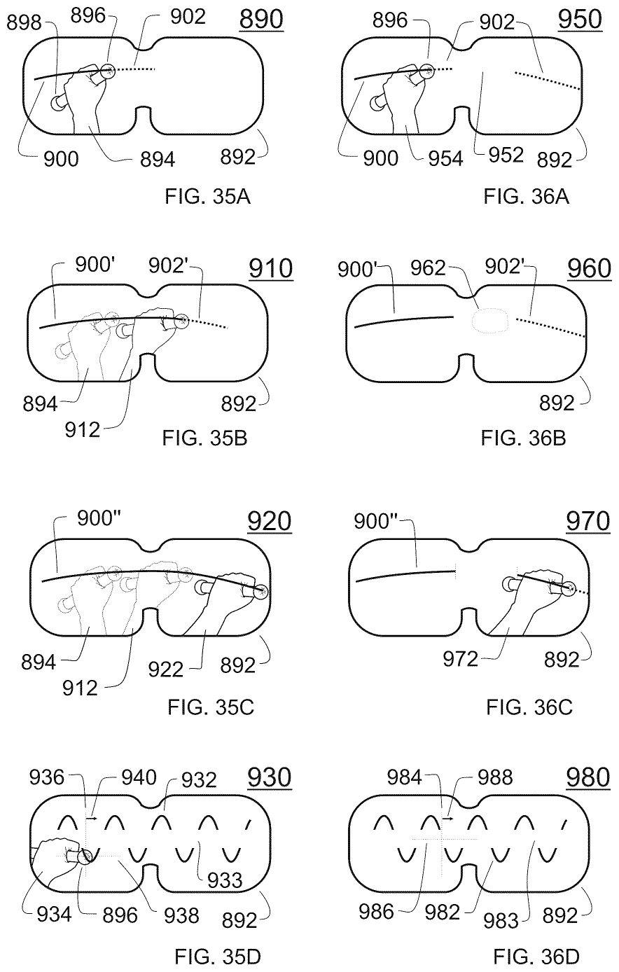

Other embodiments are designed to provides the user with a first person view of the neuro-modulation apparatus. FIG. 35A-D is therefore a schematic representation of a first person augmented reality neuro-modulation visualisation apparatus. FIG. 35A is a schematic representation of the user's first person view while using an augmented reality headset at a first time at 890. Augmented reality headset 892 provides a transparent view of the real world upon which computer graphics can be overlaid. At a first time the user's hand 894 is visible at a first position, holding a positional aid comprising a first marker 896 and a second marker 898. A computer generated curve 900 is overlaid on the view of the user's hand 894, representing the defined pattern or path (equivalent to the required force 382 of FIG. 16) that the user must trace with the positional aid's first marker 896. A first curve 900 is the path already traversed and a second curve 902 is the future path to be traversed wherein the transition point between the solid ‘past time’ first curve and dotted ‘future time’ second curve moves to the right with the passage of time and the user is attempting to ensure that the first marker 896 maintains steady progression with the present time transition between the first and second curves respectively. This task may be conducted with the user's hand connected to a resistive extension apparatus, without any extension apparatus or with a weighted positional aid, such as a dumbbell. Third and fourth computer generated curves (not shown) may also be applied which show the path through which the second marker 898 must be traced in space, in which case the user's hand must trace not just a translational movement across the field of view but translation plus a potential rotational component. FIG. 35B is a schematic representation of the augmented reality headset 892 at a second time at 910 wherein similar elements from previous figures are similarly numbered. The location 894, at a first time, of the user's hand is shown for reference only and with the passage of time the user's hand has moved to a new position 912 at a second time, with the first marker 896 tracing the defined path 900 in synchrony with the transition between the first and second curves 900′ and 902′ respectively. FIG. 35C is a schematic representation of the augmented reality headset 892 at a third time at 920 wherein similar elements from previous figures are similarly numbered. The location 894, at a first time, of the user's hand and the location 912, at a second time, of the user's hand are shown for reference only and with the passage of time the user's hand has moved to a new position 922 at a third time, with the first marker 896 continuing to trace the defined path 900″. In some embodiments the augmented reality headset may be configured with a camera system to record the path of the hand via location of the first and second markers 896 and 898 respectively within the field of view. Preferably the camera system is a stereoscopic camera system employing at least two separate sensors a fixed distance apart to triangulate similar pixels from their respective 2-dimensional image planes allowing depth perception of the imagery and mapping the position of the first and second markers in 3-dimensional space.

In some embodiments the defined path is periodically suppressed (occluded) forcing the user to adapt in predictive real-time feedforward as previously taught. In this way the motion of the user's hand tracing the defined pattern or path 900 is functionally equivalent to the defined pattern 382 of FIG. 16A and indeed the defined path 900 may be any path including a sinusoidal pattern. FIG. 35D is therefore a schematic representation of the augmented reality headset 892 with a sinusoidal defined path at 930 wherein similar elements from previous figures are similarly numbered. Sinusoidal pattern 932 is periodically occluded 933 as was taught in the real-time feedback and real-time feedforward embodiments of FIG. 17, FIG. 18 and FIG. 20. The user's hand 934 is seen tracing out the sinusoidal pattern 932 with the first marker 896 at horizontal position 936 and vertical position 938 within the field of view while moving from left to right 940. Embodiments therefore provide a method of using real-time feedback and real-time feedforward neuro-modulation with an augmented reality headset including obscuring part of the defined path. It would be readily apparent to one of ordinary skill in the art that the variations of real-time feedback and real-time feedforward neuro-modulation taught throughout this disclosure are intended to be applied to this embodiment.

In addition to obscuring the defined path, the augmented reality headset has the ability to obscure the view of the user's hand. Therefore, FIG. 36A is a schematic representation of the augmented reality headset 892 at a first time at 950 wherein similar elements from previous figures are similarly numbered. Past time defined path 900 and future time defined path 902 are shown with a portion of the future time path 902 occluded 952. The user's hand 954 is seen tracing out the position of the first marker 896 along the defined path. FIG. 36B is a schematic representation of the augmented reality headset 892 at a second time at 960 wherein similar elements from previous figures are similarly numbered. In this embodiment the virtual reality headset 892 totally obscures the real view of the user's hand but still provides a visualisation of the defined path 900′ and 902′. At the second time the position of the first marker 896 is somewhere in a region 962 in the occluded part of the defined path and the user's hand has therefore also been occluded. This is a highly real-time feedforward state. FIG. 36C is a schematic representation of the augmented reality headset at a third time at 970 wherein similar elements from previous figures are similarly numbered. At the third time the user is back in the non-occluded part of the defined path 900″ and the user's hand 972 is subsequently visible once more. It would be readily apparent to one of ordinary skill in the art that either the defined pattern or the user's hand or indeed both may be occluded in the real-time feedforward sections of the Neuro-Modulation Therapy. In some embodiments the defined path and/or the user's hand are periodically occluded forcing the user to adapt in a predictive, real-time feedforward state as previously taught. FIG. 36D is therefore a schematic representation of the augmented reality headset 892 with a sinusoidal defined path at 980 wherein similar elements from previous figures are similarly numbered. Sinusoidal pattern 982 is periodically occluded 983. In contrast to FIG. 35D, the user's hand is in an occluded part of the sinusoidal pattern 982 with the first marker 896 at horizontal position 984 and vertical position 986 while moving from left to right 988. Embodiments as described herein provide a method of using real-time feedback and real-time feedforward Neuro-Modulation Therapy with an augmented reality headset including obscuring part of the defined path and/or obscuring the user's body.

It would be readily apparent to one of ordinary skill in the art that augmented or virtual reality techniques are adaptable for and intended to be used with various embodiments taught throughout this disclosure.

As a further example we consider application of a cobot based system to the swing of a tennis racquet. In this case the cobot would have an end effector that resembled the handle of a tennis racquet. The cobot would be programmed to provide resistance to motion in a way that simulates the three axis moment of inertia of the given user's preferred tennis racquet and may even simulate speed of motion and racquet angle dependent air resistance. In this way the user should not be able to tell the difference between swinging the cobot end effector and the real tennis racquet. The user's natural swing may be analysed and captured by the cobot's three dimensional motion sensing. Deviations from the ideal swing may then be presented on the output apparatus and an idealised path may be presented as a defined pattern disclosed as elements 352 and 356 of FIG. 15A for the user to replicate. The output apparatus may take any form disclosed or suggested in the present disclosure including augmented or virtual reality headsets. The user may be presented with the defined patterns at various speeds. Furthermore, the user may be presented with occlusion of the defined patterns creating a transition from purely feed-back to feed-forward motor training pathways. In this way, all of the methods, systems and apparatuses as described herein would be applicable to training coordinated body movement, as a whole or in part. Embodiments therefore allow the remapping of neural pathways associated with motion during sporting activities. That is, it may be used to rewrite muscle memory, improving performance. Embodiments thus have application to sports training and training coordinated body movement for athletes, dancers or for any persons required to perform tasks that require fine motor control. It would be readily apparent to one of ordinary skill in the art that a virtual reality embodiment is possible, wherein a computer generated world is used rather than an augmented reality view and a user is provided with some computer generated task to perform while undergoing neuro-modulation assessment or therapy. It would also be apparent that other aspects as disclosed herein are intended to be applied to these embodiments, such as but not limited to, sleep memory activation.

Embodiments discussed above are directed towards capture of motion (including precision capture) using external measurement apparatus including extension apparatus, cycling apparatus and virtual reality apparatus among others. All of these apparatuses are external measures of the motion of the body, principally disclosed in terms of the motion of the knee for the sake of clarity (noting that similar apparatus may be used for other joints). However modern implants and joint replacements are beginning to incorporate motion sensing apparatus which are typically used for step counting and simplistic gait analysis. The present disclosure teaches that these apparatus, may be further adapted for use as a data source for the presently disclosed applications. That is, the measurement of a target parameter may be performed by a sensor integrated in an implant, or by a surgically implanted sensor.

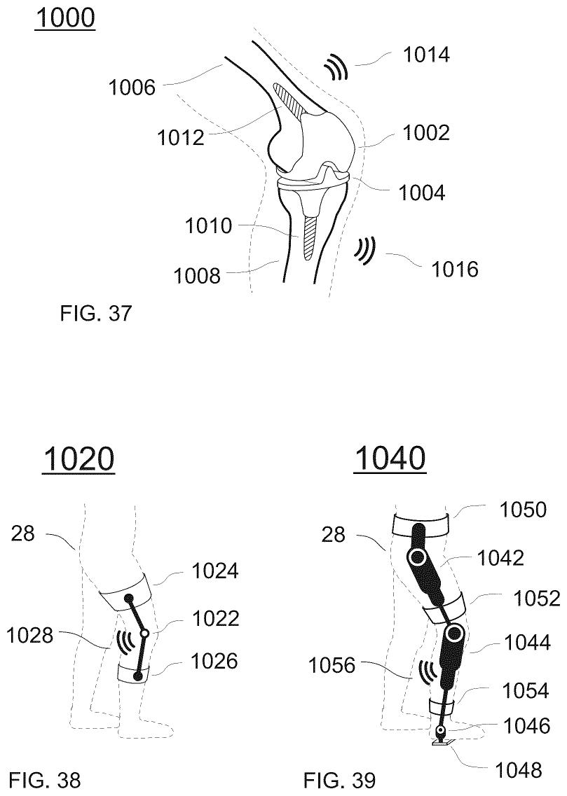

FIG. 37 is a schematic representation of a post-surgical total knee arthroplasty at 1000 according to an embodiment. The artificial knee is generally composed of three components, the femoral component 1002, the tibial component 1004 and the patella (not shown for clarity). During surgery, the end of the femur 1006 is excised and the femoral component 1002 is attached. The end of the tibia 1008 is also excised and the tibial component or the stemmed tibial tray 1004 is attached. The bearing surface between these two components is a plastic insert (not shown for clarity) which is typically a polyethylene layer that is attached to the stemmed tibial tray 1004. The stem 1010 of the tibial tray may incorporate a motion sensing apparatus or sensor apparatus. This may include an Inertial Measurement Unit (IMU) and communications module. In this embodiment the IMU tracks data comprising the three dimensional position and orientation of the stemmed tibial plate, or the lower leg. The IMU may contain any combination of gyroscope, inertial sensor, accelerometer or any other sensor apparatus or device which may provide a stream of information on the position or motion of the tibial tray 1004. The data is wirelessly transmitted to an external computing apparatus. In another embodiment a motion sensing apparatus 1012 or sensor apparatus could be incorporated into the femoral component 1002. Information about the motion of the femoral component 1002 and the tibial component 1004 are transmitted wirelessly 1014 and 1016 respectively to an external computing apparatus. In this way the user is able to utilise the neuro-modulation methodology—defined patterns, real-time feedback and real-time feedforward therapies, sleep memory activation, etc.—taught in the present disclosure using only the motion sensing apparatus embedded in the joint replacement apparatus. In this way a user can utilise the embodiments with traditional gym equipment such as conventional exercise bikes, elliptical trainers, rowing machines, weights equipment, stair-climbers or even walking up a flight of stairs in synchrony with a specifically designed defined pattern.

In a similar way embodiments may use any external apparatus of monitoring the motion of the body. A neuro-modulation system is therefore able to be created with a simple, body worn, digital goniometer that provides measurement of motion which can be combined with the other aspects as described herein. FIG. 38 is a schematic representation of an externally attached digital goniometer at 1020 wherein similar elements from previous figures are similarly numbered. Digital goniometer 1022 is adapted to measure the knee joint angle of a user 28, being attached between a thigh cuff 1024 and a calf cuff 1026. Digital goniometer 1022 contains a wireless apparatus 1028 for transmitting a high resolution time series comprising range of motion information to an external computing apparatus wherein analysis as described herein may be performed.

The robotics industry is investing heavily in exoskeleton development. A robotic exoskeleton is a wearable robotic unit controlled by computers to power a system of motors, pneumatics, levers or hydraulics to restore locomotion, aid in movement or increase load carrying capacity. FIG. 39 is a schematic representation of an exemplary exoskeleton configured as a NMT apparatus according to an embodiment at 1040 wherein similar elements from previous figures are similarly numbered. at 1040. User 28 is wearing a lower limb (leg) exoskeleton comprised of a first actuator unit 1042 for the movement of the thigh, a second actuator unit 1044 for movement of the lower leg and a third actuator unit 1046 for movement of a foot plate 1048. The proximal end of the first actuator 1042 is attached to a waist strap or cuff 1050 and the distal end of the first actuator 1042 is attached to a thigh cuff 1052. The first actuator thus controls and aids in motion of the hip joint. The proximal end of the second actuator 1044 is attached to the thigh cuff 1052 and the distal end of the second actuator 1044 is attached to the calf or ankle cuff 1054. Motion of the second actuator 1044 thus aids in motion of the knee joint. The proximal end of the third actuator is attached to the calf or ankle cuff 1054 and the distal end is attached to the foot plate 1048. Motion of the third actuator 1046 thus controls the placement and angle of the foot plate 1048. Such an exoskeleton is analogous to a wearable cobot as described above. In this way, information about the motion of the exemplary leg can be captured and transmitted to an external computing apparatus via a wireless protocol 1056. Accordingly, an exoskeleton can be used to perform neuromuscular assessments, and provide NMT or development. In these embodiments the actuators are configured to simultaneously provide a programmable resistance across a range of motion and to measure forces, positions and timing of the motion. The exemplary exoskeleton of FIG. 39 suggests in-plane articulation of the knee (sagittal plane articulation of the hip and knee) but it would be readily apparent to one of ordinary skill in the art that such an exoskeleton may offer multiple degrees of freedom in each joint such that it approximates the natural range of motion of the human body. When combined with any of the other teachings of the present disclosure and deployed across substantially all of the body, the exoskeleton may be configured to perform as a whole body neuro-modulation apparatus. In particular it would be readily apparent to one of ordinary skill in the art that the exoskeleton neuro-modulation apparatus may be combined with augmented reality embodiments and sleep memory activation embodiments taught in the present disclosure. A neuro-modulation exoskeleton apparatus would then be applicable to rehabilitation of any joint following injury or surgical intervention. Further it may be used for developmental or performance enhancing applications by users seeking to further develop or refine a skill. The natural range of motion afforded by such an exoskeleton neuro-modulation apparatus is perfectly suited to Neuro-Modulation Therapy of a range of joints as taught throughout the present disclosure. Such an exoskeleton neuro-modulation apparatus is also suitable for highly targeted neuro-modulation training to optimise motion and performance in a range of applications including both amateur and professional sports.

The seemingly simple action of throwing a ball is anything but simple to master. The ability to repeatedly throw a baseball requires complex coordination of a vast range of muscles to sequentially produce a chain of event towards the desired amount of force imparted on the ball. Each body segment is required to produce enough force for the next sequential body part to utilise the generated force and build upon it, transferring force summation into an elevated final state. Proficiency involves the correct sequence and timing of all the muscles involved in throwing. Starting from the larger and heavier body parts, to finishing the throwing action using the smaller and lighter parts of the wrist and fingers, the ball is released efficaciously. Anyone skilled in the art will appreciate complex mastery of the throwing action. During the wind-up phase, the large leg muscles of the hip and knee transfer weight backwards, preparing and prepares the upright posture muscles of the trunk. The cocking phase allows the throwing arm to be left behind in a stretched out externally rotated shoulder position whilst the opposite large leg muscles thrust forward to generate momentum towards the desired target, all the while whilst creating a stable base to support the throwing arm action. The coordinated trunk muscles hold the 24 spinal segments upright as well as generate rotation in the transvers axis to drive the throwing shoulder forward. This acceleration phase takes the shoulder from an extreme external rotation to violently internally rotating it at over 7000 degrees per second using a highly refined concentric muscle contraction of primarily the rotator cuff, pectorals and latissimus dorsi. Once the ball is released with optimum speed, the throwing motion requires violent deceleration and the opposing force generation muscles act eccentrically to slow the motion during final follow-through.

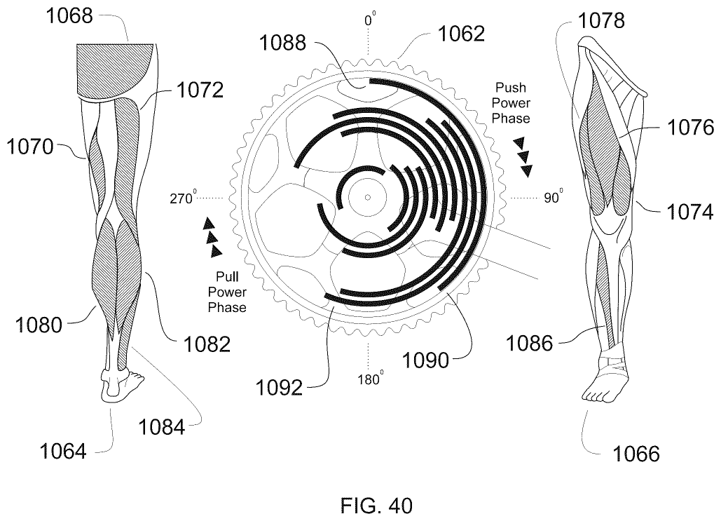

Utilisation of an exoskeleton allows mapping the user's natural action in all its detail. Such an exoskeleton may be further adapted for neuro-modulation training utilising combinations of the methodology taught throughout this disclosure. An exemplary training regime for throwing a ball may begin with the exoskeleton performing a motion capture of the user's natural throwing action. Analysis of the user's action may highlight technical deficiencies as well as aspects of the motion that may suggest risk of possible injury. Neuro-modulation training may incorporate a representation of the user on screen while wearing a neuro-modulation exoskeleton. The neuro-modulation exoskeleton provides real time feedback as to the user's actual motion and compares this against the defined pattern, which in this case is the action of throwing the ball. The method may begin with a ‘slow motion’ demonstration of the correct technique, which may be only marginally slower than the user's natural action or it may be substantially slower. The user is presented with a representation on screen or via a virtual or augmented reality headset demonstrating the ‘defined pattern’ action that they must accurately replicate. Embodiments described herein teach the beneficial use of occlusion in training a user to perform a specific motion. As the user progresses through the training programme, certain combinations of feedback and feedforward occlusion exercises are conducted, all the while progressing towards the perfect technique and speed of action. This leads back to the prior discussion of the neuro-modulation pedal set as disclosed with reference to FIG. 9A and the application of neuro-modulation to performance optimisation and training, even in the absence of injury or surgical intervention. Pedalling a bicycle may appear to be a simplistic exercise but efficient pedalling requires precise sequential control of many muscle groups. FIG. 40 is a schematic representation of the push-pull power cycle required to pedal a bicycle at 1060 from the perspective of a single leg. The top of the stroke is designated zero degrees and the crank and chain drive 1062 is depicted in the push power phase, rotating clockwise. The push power phase of the cycle begins at zero degrees with the leg at its greatest flexion, moves through 90 degrees and extends to 180 degrees with the leg at its greatest extension. The pull power phase assumes the cyclist is cleated into the pedals and begins at 180 degrees, moves through 270 degrees and back to zero degrees.

The key muscles involved in the cycling motion are highlighted on the rear musculature of the leg 1064 and the front musculature of the leg 1066. Their respective activation during the pedalling cycle is shown by the concentric arcs superimposed over the crank and chain drive 1062. The key muscles in this process are the Gluteus maximus 1068, the Semimembranous 1070, the Biceps femoris 1072, the Vastus medialis 1074, the Rectus femoris 1076, the Vastus lateralis 1078, the Gastrocnemius medialis 1080, the Gastrocnemius lateralis 1082, the Soleus 1084 and the Tibialis anterior 1086. A smooth and efficient cycling action requires each of these muscles to deliver power at the right phase of the cycle. The outermost concentric arc denotes the portion of the cycle during which the Gluteus maximus 1068 is actively engaged with each successively smaller concentric arc corresponding to the next respective muscle disclosed above until the last muscle in the list, the Tibialis anterior 1086, corresponds to the innermost concentric arc. Beginning at the top of the stroke (substantially zero degrees), the gluteus maximus 1068 is engaged (the beginning of the outermost concentric arc 1088) and continues to be engaged until about 140 degrees into the cycle 1090. The next respective muscle, the Semimembranous 1070, is engaged over the range denoted by the arc with the second largest radius 1092, from about 45 degrees through about 200 degrees. Most of the key muscles are seen to be engaged during the push power phase but some are engaged during the pull power phase. The important point to note is that each muscle plays its part for a specific part of the cycle and the sequencing of each muscle is important for the efficiency of the cycle.

The use of a connected pedal, that is a pedal comprising one or more sensors and a communications module, is particularly applicable within the context of a detailed understanding of the push power and pull power phases of the pedalling cycle. Thus, in some embodiments a connected pedal is used to capture information about the forces involved during each pedal's cycle. The data is transmitted to a computing apparatus in real time for display to the user. FIG. 41 is therefore a schematic representation at 1100 of the data delivered by an exemplary connected pedal 232 from a neuro-modulation apparatus 231 or 236. Connected pedals can measure a range of parameters such as force and motion in multiple axes concurrently. For simplicity, FIG. 41 is a synchronous representation of a selection of orthogonal components of the force exerted by the user on the pedals. The vertical component of force 1102 exerted by the first leg and the vertical component of force 1104 exerted by the second leg are shown. The temporal origin (t=0) of every graph 1105 corresponds to initiation of the push power phase of the first leg or zero degrees 1106 with respect to the push-pull power cycle (FIG. 40) of the first leg. The push power phase for the first leg passes through 90 degrees 1108 and ends at 180 degrees 1110. In this representation, positive force corresponds to exerting a force downwards towards the ground and negative force corresponds to exerting a force upwards. The pull power phase for the first leg begins at 180 degrees 1110, passes through 270 degrees 1112 and ends at 360 degrees (or zero degrees of the next cycle) 1114 completing one full cycle of the first leg.

Beginning at time t=0, corresponding to zero degrees 1106 for the first leg (graph 1102), the user typically applies a rapidly increasing force downward with broad, high power transmission across the majority of the push power phase. The peak in force is likely right translated and force decreases rapidly to zero at 180 degrees 1110. The force exerted by the typical user during the pull power phase is narrower and at far lower peak force as would be evident by observation of the concentration of muscle engagement shown in the concentric arcs of FIG. 40. In fact, in a lot of cases the user does not exert any force during the pull power phase. The whole cycle is repeated again from 1114. The force exerted by the second leg of a typical user (graph 1104) is substantially the same as that exerted by the first leg, but when the graphs are time aligned, the curve representing the force exerted by the second leg 1104 is phase shifted by 180 degrees with respect to that of the first leg. At time t=0, the second leg 1104 is beginning the pull power phase at 180 degrees 1110. The horizontal component of force 1116 exerted by the first leg of a typical user is much smaller in magnitude than the peak force exerted in the vertical plane 1102 (figures not to scale). It is also 90 degrees out of phase with respect to the force exerted in the vertical plane by the nature of circular motion. While far less powerful than the vertical component of force, particularly with respect to the push power phase, accurate engagement of the muscle groups responsible for rearward horizontal force (at 180 degrees) and forward horizontal force (at zero degrees) are a necessary component of an efficient pedalling action. An optimal power cycle, such as the exemplary vertical component of force 1102 of FIG. 41, is presented to the user as a scrolling ‘defined pattern’ and the user is required to match the instantaneous pedal force applied with the target defined pattern. Occlusion of the defined pattern is then utilised in order to harnesses the neuro-modulation methodology's feedback and feedforward mechanisms that engage the neuroplasticity of the motor cortex. While the optimum power cycle may not be anywhere near a sinusoid, it is possible to map the maximal force that a user is able to exert throughout the power cycle onto a sinusoid. This allows the user to attempt to maintain full power around the cycle while visualising a simple, target force, defined pattern on the display. In order to create this mapping, the user must perform a maximal performance test or calibration whereby the system captures their maximal force exerted throughout the power cycle over at least one resistance setting on the neuro-modulation apparatus 231 or 236.

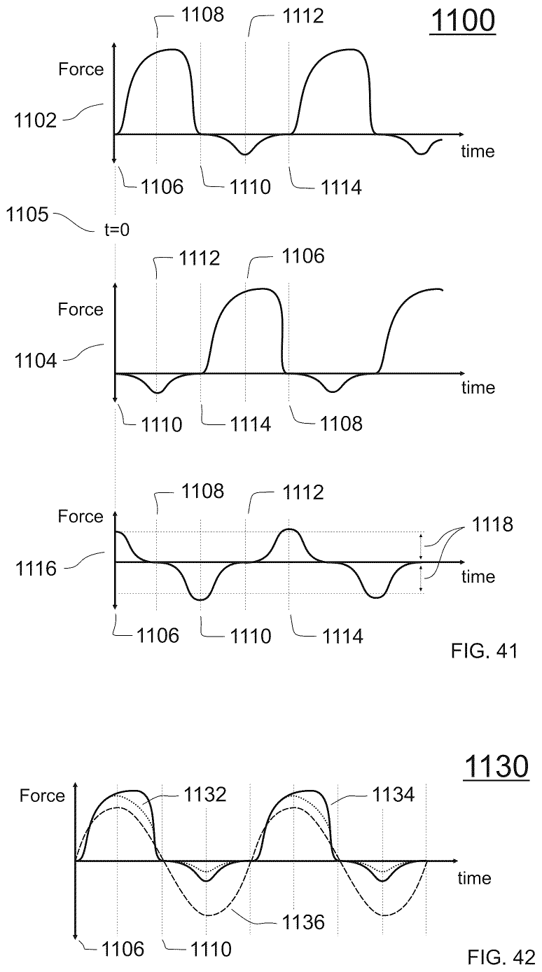

FIG. 42 is therefore a schematic representation of a maximal performance calibration data capture at 1130 where similar elements from previous figures are similarly numbered. The user's maximal effort is captured over a plurality of power cycles and averaged to provide a maximal effort curve 1132. Data for each leg would be captured and processed in a similar manner. It would be readily apparent to one skilled in the art that other forms of filtering or statistical processing are possible and indeed anticipated in the present disclosure. The user's maximal effort curve 1132 for each leg is then compared to the optimal power cycle curve 1134. In this exemplary schematic representation, the peak of the push power phase for the optimal power cycle 1134 is seen to be right translated while the user's performance in this phase is left translated suggesting sub-optimal technique and possibly a muscle imbalance or injury leading to a relative weakening of that part of the push power phase. Similarly, the user's pull power phase is seen to be substantially lower in magnitude to the optimal performance.

The user's statistically processed maximal effort curve 1132, or alternatively the optimum power cycle curve 1134, is then mapped to a periodic function such as a sinusoid 1136. This mapping may additionally be modified to take into account the user's relative weakness (or possible strength) in various phases of the power cycle. For each part of the power cycle where a user's performance is sub optimal, the user must adjust their exertion during that part of the power cycle in order to match the target defined pattern during a neuro-modulation session. During the suggested deficiency i.e. the latter part of the push power phase, between 90 degrees 1108 and 180 degrees 1110 in FIG. 42, the user must exert greater relative force in order to match the defined pattern. In this way, the dominant muscles during that part of the push power phase, namely the Semimembranous 1070, the Biceps femoris 1072, the Gastrocnemius medialis 1080 and the Gastrocnemius lateralis 1082 would be worked hard than normal in order to reach the ideal cycling form. Thus, in one embodiment a performance enhancement (or optimisation method comprises a training methodology that functionally targets discrete muscle groups in order to optimise their performance as described herein. This methodology has the effect of ‘balancing’ the relative strength of each muscle group in order to optimise overall performance.

Neuro-modulation training may begin with a target force at some reasonable percentage of the user's maximal output, increasing in force towards the user's maximal output over time as the user improves their performance, wherein the target would be to create the most efficient, optimal form. It may also be advantageous to operate this form of neuromuscular training at slower crank speeds than a typical cycling workout session. Neuro-modulation is most effective when there is enough time for feedback and feedforward processing. This may be achieved with negligible resistance whereby the user focuses on perfecting their slow motion technique. It may also be achieved at moderate or high resistance where the user focuses on the right relative muscle exertion throughout the cycle, again at slower than normal speeds, at more typical cycling loads. Training at these sub-maximal output levels makes neuro-modulation training ideal for the warm-up and cool-down phases of a training session. This turns the warm-up and cool-down into a science driven, targeted, functional, and performance optimisation part of the session rather than what might otherwise be seen as just filling time.

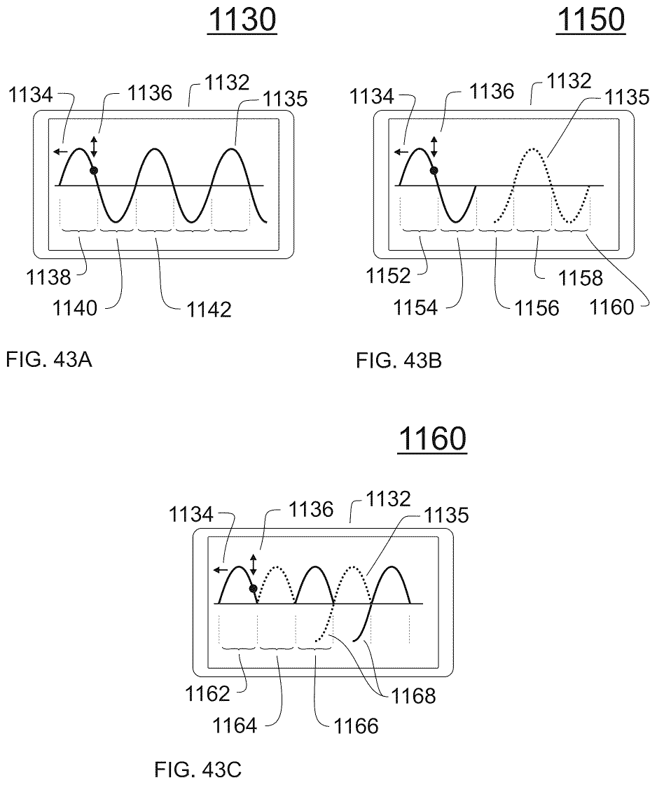

It is important to note that with the data coming from the connected pedals it is possible to monitor not only the average angular momentum of the drive shaft, which represents the aggregate force generated by both legs in their complementary push-pull power phases, but the individual forces applied to each pedal, in both push and pull. This allows simultaneous, independent analysis of both legs throughout the whole of their power cycles. This opens up some interesting opportunities for presentation of the real-time data to the user. FIG. 43A-C is therefore a schematic representation of a user display during a neuro-modulation training session. FIG. 43A is a schematic representation of a single leg neuro-modulation training programme at 1130, that is, a programme that is focused entirely on optimising the performance of one leg, perhaps a weak leg, during a normal bipedal cycling action. According to an embodiment, the display apparatus 1132 displays a scrolling 1134 sinusoidal defined pattern 1135. The user's actual applied force is shown in real time as a dot that moves 1136 in the vertical plane. The push power phase 1138 is followed by the pull power phase 1140 which is followed in turn by the push power phase 1142 for the single leg being trained (and so on). FIG. 43B is a schematic representation of a dual leg neuro-modulation programme at 1150 where similar elements from previous figures are similarly numbered. According to an embodiment, the display apparatus 1132 displays a scrolling 1134 sinusoidal defined pattern 1135. The user's actual applied force is shown in real time as a dot that moves 1136 in the vertical plane. The push power phase 1152 of a first leg is followed by the pull power phase 1154 of the first leg. The neuro-modulation then switches to the second leg but because the two legs are 180 degrees out of phase then system needs to provide a gap and lead in period 1156 for a second leg. Subsequently the user follows the push power phase 1158 for the second leg and the pull power phase 1160 of the second leg. The defined pattern then switches back to a focus on the first leg's push power phase with a similar break and lead in display. In this way, both legs are sequentially trained via an alternating focus on each leg according to embodiments of NMT. FIG. 43C is a schematic representation of a dual leg neuro-modulation programme at 1160 that focuses only on the push power cycle but for both legs where similar elements from previous figures are similarly numbered. According to an embodiment, the display apparatus 1132 displays a scrolling 1134 sinusoidal defined pattern 1135. The user's actual applied force is shown in real time as a dot that moves 1136 in the vertical plane. The push power phase 1162 of a first leg is followed by the push power phase 1164 of a second leg, followed again by the push power phase 1166 of the first leg, and so on. In this way the user alternates focus on the push phase of each leg. Optionally a lead in curve 1168 is displayed for each leg as a way of preparing to transition the focus from one leg to the other. In a similar way the system may focus only on the pull power cycle for both legs in the same exercise. The defined patterns may include suppression or occlusion via any of the methods taught in the present disclosure and suppression (or occlusion) may also be by apparatus of a ‘blink’ occlusion of the entire pattern with varying duty cycle and mark-space ratio.

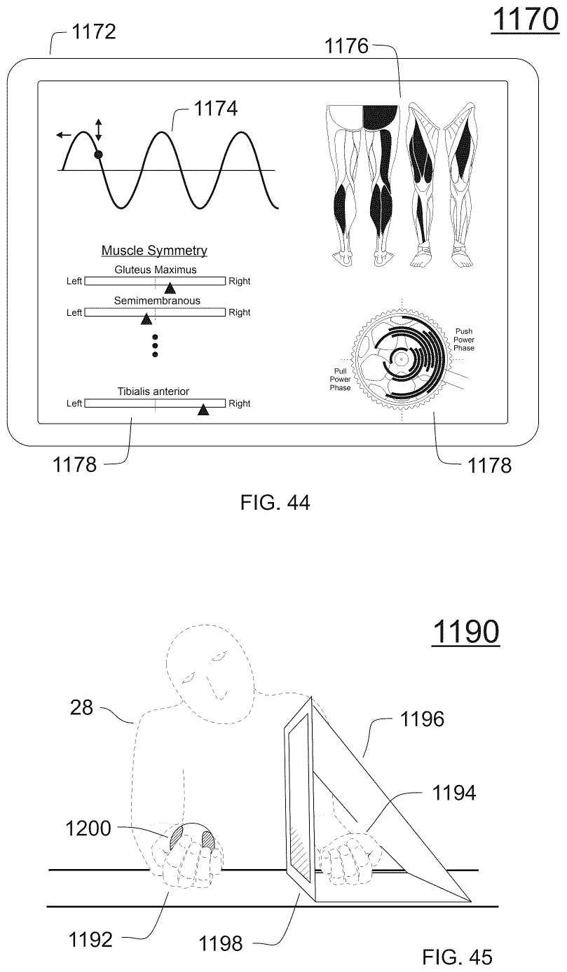

An objective of neuro-modulation training is to optimise (or at least improve) performance and focus on specific muscle groups that may be injured or deficient in some way, or which the user simply wishes to enhance or further develop. Body symmetry is also important in athletic performance and the system is suitable for assessing symmetry and training to develop a more symmetrical musculature. FIG. 44 is therefore an exemplary user display of neuro-modulation training programme adapted to optimising performance of a series of muscles at 1170. Display apparatus 1172 displays the neuro-modulation defined pattern 1174, which in this case is focused on a single leg as disclosed via FIG. 43A. Optionally the display may include a muscle group graphic 1176. As the user progresses through the programme, successive muscle groups are ‘coloured in’ as the user reaches a desired level of control for that muscle. The objective of the programme is to reach a target level of control for each muscle group. The display may optionally include a muscle timing display 1178 that shows which muscles have reached the target level of control and therefore which phases of the power cycle the user needs to focus on to complete the programme. The display may optionally include a display of muscle symmetry 1178 which shows the degree of muscle symmetry. Other forms of display may include a relative strength measure for a given leg or both legs to highlight the phases of the power cycle that a user needs to focus on. This may be suitable for a warm-up or cool-down training programme where peak efficiency, performance or symmetry are desired as a lead into the workout or as a final cool-down exercise.

It would be readily apparent to one of ordinary skill in the art that this technique is equally applicable to deficiencies across the full power cycle and applies whether a pedal is used for the lower limbs in a range postures as previously disclosed, hand grips are used for upper limb training or indeed any type of end effector. The same techniques apply to any neuro-modulation apparatus envisioned in this disclosure.

Some people who suffer from neurodegenerative diseases such as Parkinson's disease, motor neuron disease and others will be unable to operate a pedal-set style of neuro-modulation apparatus. Indeed, some conditions such as stroke leave patients with varying degrees of paralysis. Upper limb paresis occurs in up to 80% of stroke patients. Six months after stroke, two-thirds of patients are not able to perform activities of daily living using their paretic hand and often do not return to work.

Mirror therapies have found favour recently in stroke rehabilitation. Mirror therapies target the brain's mirror neurons in an effort to trigger neuroplastic cortical responses promoting reorganisation and functional recovery. Motor skills can also be learned through observation, a process linked to the mirror neuron system. Mirror neurons are a class of brain cell that respond similarly when we perform an exercise (or action) and when we witness someone else perform the same exercise (or action). The mirror neurons activate and fire accordingly when observing motor behaviour. It has been proposed that mirror therapy, which has been shown to lead to functional improvements in some stroke patients, facilitates cortical reorganisation—neuroplasticity. FIG. 45 is a schematic representation of a mirror therapy at 1190 wherein similar elements from previous figures are similarly numbered. User 28, who may be a stroke patient, someone with a neurodegenerative disorder or someone recovering from an injury or surgical intervention, is observing a first fully functional hand 1192 while a second, injured or paralysed hand 1194 is hidden from view behind a screen 1196. The user is looking at the reflection of the first hand 1192 in a mirror 1198 oriented in such a way as to provide the appearance that the image of the first hand 1192 is actually the second hand 1194. This fools the brain into thinking that it is observing both hands at once and that the reflected motion of the first hand is actually the second hand moving. When the user squeezes a ball 1200 held in the first hand 1192, the brain perceives the second hand 1194 to be squeezing a similar ball. This has the effect of activating mirror neurons and provides therapeutic benefit via neuroplasticity.

Stroke typically presents with unilateral paralysis or paresis as a result of brain trauma to the contralateral side. This often results with the user having substantially unaffected motion on a first side of the body and paralysis or paresis on the second side. Paralysis or paresis may be in the form of facial muscle weakness, upper limb weakness, lower limb weakness, muscle spasticity, permanent muscle contractures or any combination of the above, among other issues. The user may undergo neuro-modulation assessment or therapy of either their exemplary stroke affected hand, or their exemplary non-stroke affected hand (wherein hand may be substituted in this context for limb, arm, leg, foot or any other stroke affected body part) according to any of the methods taught in the present disclosure.

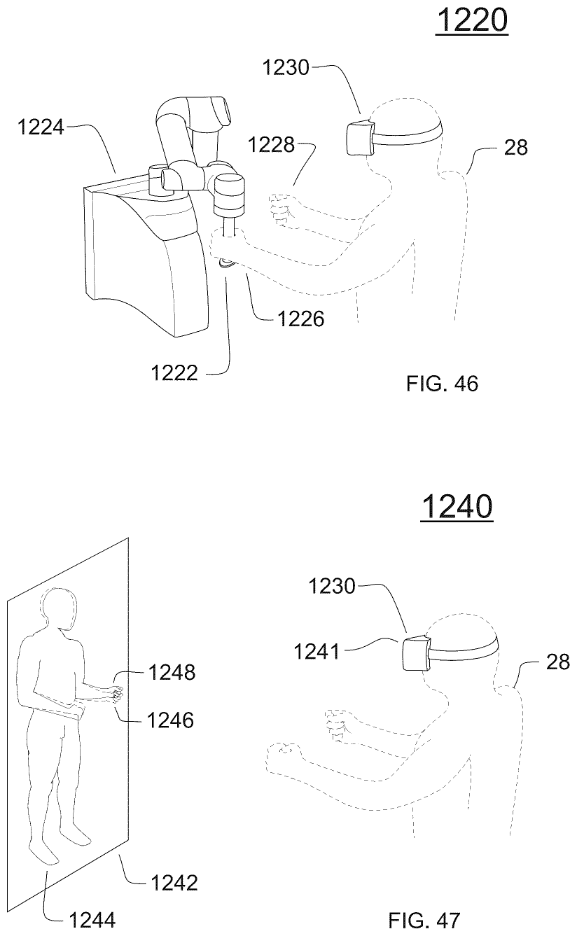

FIG. 46 is a schematic representation of a neuro-modulation apparatus combining the features of mirror therapy at 1220 wherein similar elements from previous figures are similarly numbered. User 28, who in this exemplary embodiment is suffering from stroke induced upper limb paresis, is gripping the end effector 1222 of cobot 1224 with a first hand 1226. The user's second hand 1228 is mimicking the motion of the first hand 1226 while the user observes the scene with a virtual or augmented reality headset 1230. In one embodiment the user's first hand 1226, which is gipping the end effector 1222 is the nominally healthy, non-stroke affected hand while the second hand 1228 is suffering from paresis. According to this embodiment, the user undergoes Neuro-Modulation Therapy according to combinations of the embodiments taught in the present disclosure. As previously discussed, unilateral neuro-modulation appears to have a positive effect bilaterally. Similarly, the headset 1230 displaying the second hand performing the mirror image of the motion of the first hand triggers mirror neuron activation to deliver a positive beneficial effect. The embodiment of FIG. 46 is therefore utilising, in the first instance, the bilateral neuroplastic benefits of unilateral neuro-modulation and in the second instance, the activation of mirror neuron neuroplasticity. In another embodiment the user's first hand 1226, which is attached to end effector 1222 is the hand suffering from paresis while the second arm/hand 1228 is nominally healthy and non-stroke affected. The user undergoes Neuro-Modulation Therapy unilaterally on the first hand, suffering paresis, which drives positive neuroplastic benefit. Concurrently the headset 1230 displays a virtual representation of the first hand performing the desired exercise optimally (rather than the actual paresis-impacted motion) which drives activation of mirror neurons which have been shown to enhance neuroplasticity. The embodiment of FIG. 46 is therefore utilising, in the first instance, the neuroplastic benefits of Neuro-Modulation Therapy on the first hand and in the second instance, the activation of mirror neuron neuroplasticity for the same first hand.

Neuro-modulation may also be applied to whole of body movements. Exercises such as yoga and tai chi consist of slow, controlled movements aimed at quality of motion and may be considered a meditative exercise, focusing mind and body. Neuro-modulation may take the form of similar exercises. FIG. 47 is a schematic representation of a neuro-modulation exercise at 1240 wherein similar elements from previous figures are similarly numbered. User 28 is wearing a virtual or augmented reality headset 1230. Headset 1230 may optionally contain a camera 1241 directed outward from the user to capture the field of view that the user would have seen had they not been wearing headset 1230. In one embodiment the user 28 stands in front of a standard mirror 1242 such that the camera 1241 captures the reflection of the user in the mirror 1242. Headset 1230 provides to the user 28 a representation of mirror 1242 and a mirrored image of the user's motion 1244. The user 28 is directed to perform a series of exercises such as yoga, tai chi, Pilates or other exercises via video demonstration using the headset 1230. The exercises may contain user feedback via an image of the user's actual motion (captured from the camera 1241) overlaid across an image of a directed exercise (a defined pattern within the context of the present disclosure). FIG. 47 depicts a snapshot of the continuous motion of the exercise at a point in time where the image of the user's motion 1246 captured by the camera 1241 is shown as an overlay on the optimum motion of the exercise, or defined path, 1248. This overlay provides real time feedback on the accuracy of the user's movement. The neuro-modulation apparatus may provide feedback on the accuracy of motion by, for example, colour coding body parts according to their degree of mismatch from the defined motion path. It would be readily apparent to one of ordinary skill in the art that numerous methods are available to inform the user of the quality of motion attained.

The exercises may also contain neuro-modulation feed-forward exercises via occlusion of a defined motion path according to the method taught by reference to FIGS. 35 and 36. According to this embodiment, the user is undergoing Neuro-Modulation Therapy and simultaneously activating mirror neurons delivering a potentially multiplicative neuroplastic benefit. The degree of error between the measured and desired movements may be provided as feedback to the user, for example using an overlay of the desired motion on the actual motion as described above, or using another representation of the error, for example by overlaying a colour indicative of the error over the respective body part. In another embodiment a separate camera unit is facing the user and operatively connected to the headset to portray the actual motion of the user.

It must also be noted that all of the neuro-modulation apparatuses taught in the present disclosure are intended to be adapted with mirror therapies in mind, where applicable. Of particular relevance is that unilateral Neuro-Modulation Therapy has shown bilateral benefit in knee rehabilitation studies. The addition of a standard mirror to each of the apparatuses, as applicable, may be beneficial in providing a multiplicative neuroplastic response by also recruiting mirror neuron activation.

Embodiments of the methods described herein have a wide range of applications. For example, the method of assessing neuromuscular control may be used to assess patient progress before, during, and after NMT treatment. This method also has application in surgery, as during the recovery phase of knee surgery, clinicians have no easy method to determine when the femoral nerve block has worn off and normal nerve function has been restored allowing the patient to ambulate safely. Embodiments of the methods described herein can be used to determine a pre-surgery baseline measure of neuromuscular control, and then again during recovery to determine when a femoral nerve block has worn off. This assists in providing safe patient recovery and mitigating medical profession negligence.

More generally embodiments of the method may be used for treating arthrogenic muscle inhibition (AMI), increasing recruitment of muscle motor units, reducing muscle fatigue (and the cognitive fatigue typically present with someone suffering the aforementioned issues), as well as optimising the performance of people requiring high levels of controls such as athletes, sports people, and dancers. Embodiments of the method may be used to monitor progression of neurodegenerative disorders such as such as Parkinson's disease, Duchenne Muscular Dystrophy (DMD), Multiple Sclerosis (MS), Amyotrophic Lateral Sclerosis (ALS), etc. In these embodiments, the method may comprise the steps of:
performing the method of assessing control of a target musculoskeletal joint, as described herein, at a plurality of different times;
comparing the assessment of neuromuscular control between at least two of the plurality of different times; and
determining a measure of the progression of neuromuscular control using the comparisons.

Embodiments thus provide a quantifiable measure for tracking neurodegenerative disease progression or response to treatment. In another form the method provides a quantifiable measure for tracking the progress of rehabilitation from neuromuscular injury or surgical intervention, as well as a method. This can then be extended to tracking of progress of multiple individuals to enable quantification and validation for specific conditions or for specific embodiments of the method, NMT apparatus or NMT systems. Embodiments also provide possible treatments for these conditions.

In addition to neurodegeneration, an area of concern for the ageing is loss of balance and the associated increased risk of a fall. Balance is the ability to distribute your weight in away that lets you stand or move without falling, or recover if you trip. Good balance requires the coordination of several parts of the body: the central nervous system, inner ear, eyes, muscles, bones and joints. As we age, we lose balance function through loss of sensory elements, the ability to integrate information, issue motor commands and because we lose musculoskeletal function. Diseases common in aging populations lead to further deterioration in balance function in some patients. Treatment of balance dysfunction in aging populations is based on the knowledge of normal aging processes and on an evaluation of the individual's balance loss and remaining balance elements. Prevention and rehabilitation play a major role in treatment. Embodiments as described herein provides both a method of quantifying balance and a therapeutic pathway. The majority of the present disclosure has been directed to single plane, single joint motion for the sake of simplicity while noting that the methods and system may be used for multi-plane and multi-joint control.

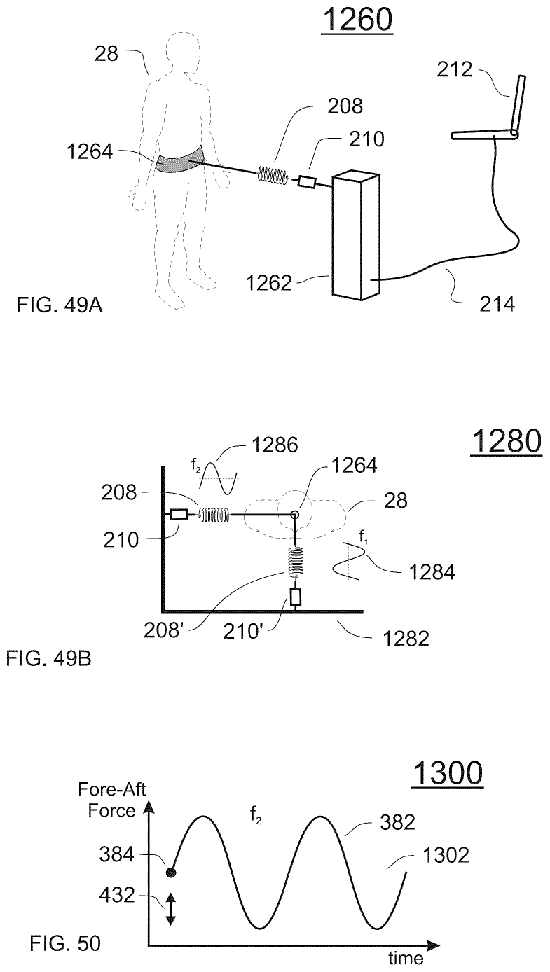

FIG. 49A is a schematic representation of an apparatus for characterisation of balance and Neuro-Modulation Therapy to improve balance at 1260, shown to characterise balance in only the sagittal plane for simplicity wherein similar elements from previous figures are similarly numbered. NMT balance apparatus 1262 comprises a body attachment arrangement 1264 such as a waist strap for attachment to the waist of the user 28. Other body attachment arrangements include chest straps, vests, belts, bands, hooks, Velcro™, carabiners, etc. Waist strap 1264 is attached to a resistive element 208 which is also rigidly attached to a sensing arrangement such as a force measurement apparatus 210 which is in turn rigidly attached to a fixed point such as a pillar. Furthermore, the NMT balance apparatus may comprise a computing apparatus 212 connected to the force measurement apparatus 210 via a data cable 214 or via other wired or wireless connections. FIG. 49B is a schematic representation of an apparatus for characterisation of balance and Neuro-Modulation Therapy to improve balance at 1280 which allows decomposition of the motion into orthogonal components, wherein similar elements from previous figures are similarly numbered. NMT balance apparatus 1282 comprises an attachment apparatus 1264 for attachment to the user 28. Attachment apparatus 1264 is attached to a resistive element 208 which is also rigidly attached to a sensing arrangement such as a force measurement apparatus 210 which is in turn rigidly attached to the apparatus 1282. Attachment apparatus 1264 is also orthogonally attached to a resistive element 208′ which is also rigidly attached to a sensing arrangement such as a force measurement apparatus 210′ which is in turn rigidly attached to the apparatus 1282. In this way NMT balance 1282 apparatus is able to characterise balance in two orthogonal directions to best quantify the degree of a user's balance. Similarly, such an arrangement provides a high degree of resolution in Neuro-Modulation Therapy. Furthermore, the NMT balance apparatus may comprise a computing apparatus 212 connected to the force measurement apparatus 210 via a data cable 214. As with other embodiments described herein force measurement apparatus 210 and 210′ may be replaced with position sensing apparatus as force and position are generally interchangeable within the present disclosure.

Attachment apparatus is optimally attached to the torso of the user by a waist strap, a chest strap or some other type of attachment that is rigidly attached to the user. NMT balance apparatus may be a stand-alone device as shown in FIG. 49A or may be attached to a wall or the corner of a room as depicted in FIG. 49B. It would be understood by one of ordinary skill in the art that the motion may be decomposed into non-orthogonal components and while this is anticipated in the present disclosure the orthogonal decomposition provides advantages. A user 28 of NMT balance apparatus 1282 may be asked to stand motionless in a first pose while attached to apparatus 1282. NMT balance apparatus 1282 is able to measure the temporal deviation in the user's position of the user from a neutral stance, resolved in orthogonal directions. Balance can be defined as the ability to distribute your weight in a way that lets you maintain a steady position while standing, sitting or in any other position. Signs of faltering balance may include a gentle sway while trying to stand motionless. A healthy adult will sense any slight deviation from the stationary position and a series of corrective actions by the muscles will return the body to its neutral position. The magnitude of said deviation and/or the user's reaction time are ways to quantify a degree of balance. This Romberg test is a typical diagnostic, non-technical, physical test that identifies a specific neurologic impairment associated with poor proprioception. This removes the visual and vestibular components that contribute to maintaining balance and can thus identify a proprioception-related neurologic disease. The Romberg test is said to be positive in a patient who is able to stand with their feet placed together and eyes open but paradoxically sways or falls while closing their eyes. Loss of balance can be defined as increased body swaying, foot movement to correct body sway, or falling.

Embodiments of the method described herein, along with the apparatus of FIG. 49B is therefore ideally suited to a detailed characterisation of balance. The exercises may include one or more balance exercises where the user is instructed to remain motionless in a first pose. The exercises may be performed while the user is attached to a monitoring apparatus 1282 or the monitoring apparatus may be a computer vision and/or sensor based system (e.g., accelerometers) to remotely measure and assess movement. The first pose may be a standing position on one leg, a standing position on both legs, a seated position, a kneeling position, or some other position. The target parameter represented (e.g., displayed) and measured is the deviation or displacement from an initial position (i.e. the body sway). The body sway may be measured over time under varying conditions such as the Romberg test or other challenges such as without limitation, a moving platform upon which the user stands or sits, and/or whilst watching a moving visual field (e.g. moving background) to visually disorient the user whilst they attempt to remain motionless, or any other challenge to balance or any combination of challenges. The moving platform may move in pitch, roll, yaw, translation or any combination thereof. In some embodiments the balance exercises may be a Romberg type test where the user is instructed to remain motionless in a first pose for a first period of time with their eyes open, and then to remain motionless in the first pose for a second period of time with their eyes closed. The pose may be standing with feet together, or another pose. In some embodiments one or more balance exercises may be swaying exercises where the user is instructed to follow a sway pattern in which the user sways in a predefined path at a predefined rate, and the target parameter is the deviation from the sway pattern. It is possible to conduct periodic assessments of balance (i.e. by repeating the same balance exercises) and build a balance trajectory over time. It would be obvious to one of ordinary skill in the art that application of various statistical techniques would be of benefit in this analysis and are therefore anticipated.

Neuro-Modulation Therapy using the apparatus of FIG. 49B provides a suitable therapeutic pathway to treat balance related issues and is particularly useful with age related balance issues and fall prevention. NMT as described by way of FIGS. 8A to 8F is a system that utilises an extension apparatus. In the case of FIG. 49, movement in any direction from the neutral standing position may be considered ‘extension’. In this way, the user may be asked to generate a specific back and forth sway pattern in one or more directions and to replicate a defined sway pattern versus time using the apparatus. In one embodiment the defined pattern is a simple, repeating pattern in one dimension although other more complex repeating or indeed non-repeating patterns may be used. Following from previous embodiments the defined sway pattern is presented to the user as a visual graph of force (or position) versus time by the computing apparatus 212. Referring to FIG. 49B a simple defined pattern for the user 28 swaying fore-aft may be a sinusoidal pattern 1284 of frequency f₁, while swaying left-right may be a sinusoidal pattern 1286 of frequency f₂. The simplest Neuro-Modulation Therapy would require the user to sway on only one dimension at a time, either right-left or fore-aft. The defined sway pattern would be represented on a screen and the user's and would be analogous to FIG. 17.

FIG. 50 is therefore a visual representation at 1300 of a sinusoidally varying defined sway pattern of the fore-aft target force 382 in accordance with the NMT apparatus of FIG. 49B wherein similar elements from previous figures are similarly numbered. The target force 382 varies around the neutral force 1302 exerted by the user standing in a neutral position. It will be readily apparent to those of ordinary skill in the art that force and position are interchangeable and therefore the neutral force 1302 would represent the neutral standing position. The dot 384 represents the instantaneous force applied to the NMT apparatus by the user in real time, which moves only in the vertical axis 432 (fore and aft) and the present time is shown near the left of the graph with a long future time window visible. Neuro-Modulation Therapy may therefore include only fore-aft defined sway patterns or only left-right defined sway patterns.

In another embodiment the Neuro-Modulation Therapy includes a simultaneous combination of fore-aft and left-right defined sway patterns. One embodiment includes repeated sinusoidal fore-aft and left-right defined sway patterns at respective sway frequencies f₁ and f₂. The relative frequencies and the phase relationship between the two sinusoids determine the type of motion.

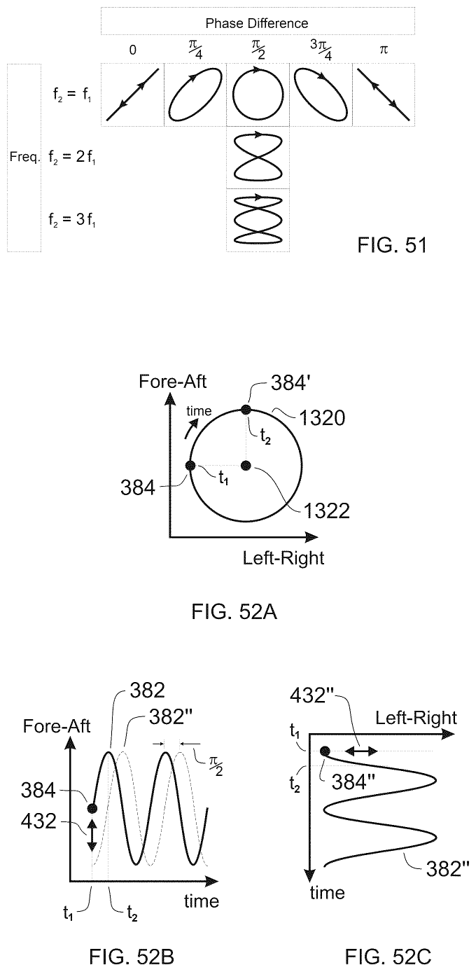

Motion becomes regularly symmetric in frequency—phase space at integer multiples of the frequency and at phase angles that are multiples of π/4 or 45 degrees. These stable patterns are referred to as Lissajous figures. FIG. 51 is a representation of a few Lissajous figures. Circular motion is represented as two orthogonal sinusoids of equal frequency (f₂=f₁) that are π/2 or 90 degrees out of phase.

FIG. 52A-C is a schematic representation of the decomposition of circular motion into its constituent orthogonal sinusoids. FIG. 52A is a schematic representation of a circular defined sway pattern 1320 challenging a user 28 to move in a circular motion 384 (or create a circular two-dimensional force profile) around a neutral standing position 1322, wherein similar elements from previous figures are similarly numbered. In this representation the user begins at a left leaning position 384 at time t₁ and traces a circular clockwise path in time, transitioning through a point leaning forward 384′ at time t₂, continuing to complete the circular path. FIG. 52A can therefore be considered to be both a method of characterising the balance of a user and the feedback motor learning component of Neuro-Modulation Therapy. FIG. 52B is a schematic representation of the fore-aft component 382 of the circular defined sway pattern 1320 force profile (or position), wherein similar elements from previous figures are similarly numbered. At time t₁, the fore-aft position is neutral 384 (neither leaning forward nor back) and the motion is only in the fore-aft direction 432. Curve 382 shows the user tracing out the fore-aft component of two full circular movements. Shown only for reference is the phase relationship of the left-right component 382″ of the motion—although this motion is clearly not in the fore-aft direction—demonstrating the π/2 or 90 degree phase relationship between the two sinusoids. FIG. 52C is a schematic representation of the left-right component 382″ of the circular defined sway pattern 1320 force profile (or position), wherein similar elements from previous figures are similarly numbered. At time t₁, the left-right position is leaning to the left 384″ and the motion is only in the left-right direction 432″. Curve 382″ shows the user tracing out the left-right component of two full circular movements.

In a balance characterisation or Neuro-Modulation therapeutic setting it is simpler to provide the user with a representation of the resulting circular motion 1320 rather than the components of that motion 382 and 382″. In addition to an analysis of sway in the neutral standing position for characterisation of balance under various challenges such as the Romberg test, the various embodiments described herein can be applied to treatment of balance deficiencies—Neuro-Modulation Therapy.

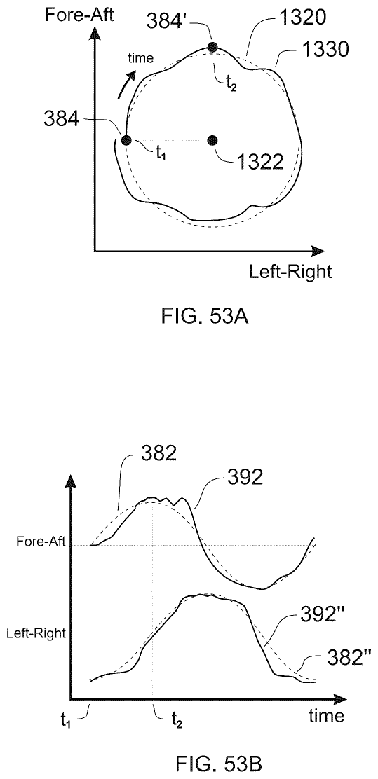

FIG. 53A is a schematic representation of a NMT assessment of balance using a circular defined sway pattern 1320 presented to the user wherein similar elements from previous figures are similarly numbered. A visual display apparatus presents a circular defined sway pattern 1320 with the user beginning from a left leaning stance 384 at time t₁. As the user traces out the circular target motion path 1320 in time the dot 384′ provides real time feedback to the user about the actual position at time t₂. The user can then compare the real time actual position to the target position which is the radial separation of the dot 384′ from the sweeping circular path 1320 and apply some correction in order to make the dot 384′ track the curve 1320 more smoothly and accurately, defined in this disclosure as real-time-feedback. This comprises the real time feedback component of Neuro-Modulation Therapy. In one embodiment of neuro-modulation balance assessment or therapy the user attempts to follow the target position curve 1320 for a defined period of time (one full clockwise circle in this example) while the neuro-modulation apparatus records the measured user position. FIG. 53A is therefore a schematic representation of the results of capture period representing said full clockwise circular path. FIG. 53B is a breakdown of the orthogonal components of the circular motion 1320, wherein similar elements from previous figures are similarly numbered. The fore-aft and left-right components are displayed together for simplicity. Fore-aft defined pattern 382 and actual measured fore-aft position 392 allow analysis of one orthogonal component of balance while left-right defined pattern 382″ and actual measured left-right position 392″ allow analysis of the other orthogonal component of balance.

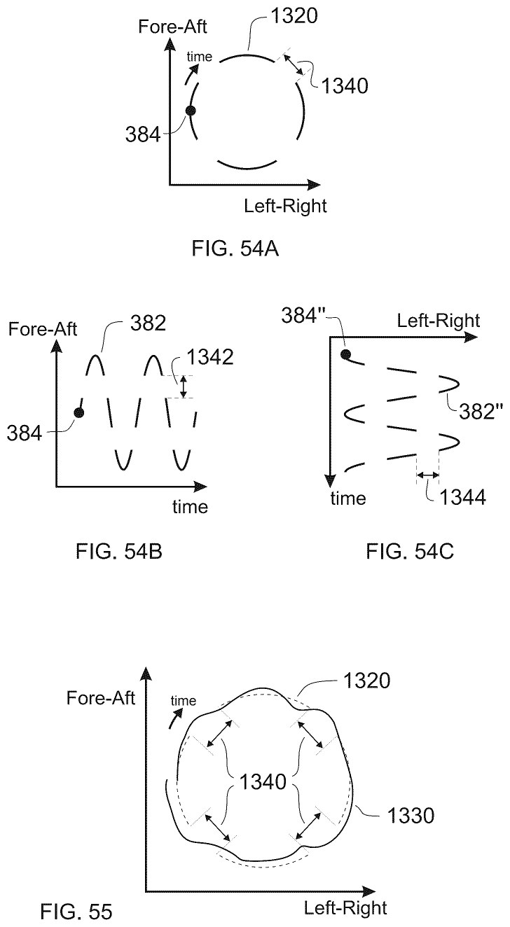

In a therapeutic sense, Neuro-Modulation Therapy is optimal when using a combination of both feed-forward and feed-back motor learning as previously disclosed. FIG. 54A is therefore a visual representation of a circular defined sway pattern 1320 shown on visual display apparatus in which different regions of the pattern are suppressed (occluded) according to an embodiment, wherein similar elements from previous figures are similarly numbered. The dot 384 represents the instantaneous position of the user in real time. A series of areas of the defined sway pattern 1320 are visually occluded 1340. The user needs to predict the path is the occluded areas and maintain the dot 384 as close as possible to the ideal path in position and time. This is the feed-forward component of motor learning. FIG. 54B is a schematic representation of the fore-aft component 382 of the defined sway pattern, wherein similar elements from previous figures are similarly numbered showing occluded areas 1342. FIG. 54C is similarly a schematic representation of the left-right component 382″ of the defined sway pattern, wherein similar elements from previous figures are similarly numbered showing occluded areas 1344. FIGS. 54B and 54C and are shown for completeness. FIG. 55 is a schematic representation of the occluded circular defined sway pattern 1320 showing the measured path 1330 and occluded regions 1340, wherein similar elements from previous figures are similarly numbered showing.

In various embodiments described herein the user may be standing, sitting or in any position while undergoing balance characterisation or Neuro-Modulation balance Therapy. Various combinations of the embodiments described herein are intended to be used together, including without limitation audible memory activation.

Another area of significant need is paralysis of stroke patients. Stroke can affect the entire body, resulting in paralysis or paresis (partial paralysis), cognitive and memory deficits, speech and visual issues, emotional difficulties, daily living challenges, and pain. Paralysis is a common outcome of stroke, often on one side of the body (hemiplegia). Paralysis may affect only the face, an arm or a leg, but most often, one entire side of the body and face is affected. Paralysis is sometimes temporary but can be permanent, depending on the region of the brain and the severity of the stroke. If paralysis persists for 6 months after a stroke it is considered chronic and there are currently no effective treatments. Given that stroke paralysis is generally related to damage within the central motor cortex, the neuroplasticity that appears to be induced under the Neuro-Modulation Therapy taught in this disclosure offers hope for stroke patients. The neuro-modulation therapies taught in the present disclosure are ideally suited to assist stroke patients in regaining motor functions. Recent advances in stroke research have shed light on a possible avenue for initiating this process. Sensory nerves from the arm and hand send signals to motor neurons in the spinal cord and motor cortex that control the muscles of the limb. Electrically stimulating the cervical root of sensory nerves amplifies the activity of muscles that have been weakened by stroke. This allows patients to regain rudimentary control of their movements. Such stimulation via electrodes placed in or near the spinal cord is assistive and strengthens muscle activation only when patients are trying to move. The effects of stimulation seem to be longer-lasting than scientists originally thought and persist for a period even after the electrodes are removed. Applying neuro-modulation after such stimulation may prolong the window of efficacy, allowing more Neuro-Modulation Therapy sessions to be conducted before paralysis returns. The cumulative effect of Neuro-Modulation Therapy over time allows the brain to adapt to the new normal of afferent information from the body, improving motor control. Neuro-modulation therefore offers significant opportunities for stroke patients.

In addition to therapeutic and rehabilitation applications, neuro-modulation methods described herein may be also used to further develop or refine a skill including for performance enhancement or performance optimisation by athletes and other persons such as dancers and surgeons requiring a high degree of precision and control of joints and muscles.

Embodiments of the methods for assessing neuromuscular control can also be modified to allow assessment of mild cognitive impairment by comparing the differences in neuromuscular control obtained when the user is under a varying cognitive loading. There has been a dramatic rise in the aging population in recent years. There are now 960 million people aged over 60 globally and the size of this population is predicted to rise to 1.4 billion by 2030. Frailty is one of the geriatric syndromes caused by a general decline in multiple vital systems, characterised by increased vulnerability of unfavourable clinical outcomes such as falls, disabilities, hospitalisation and death. Falls, underpinned by an insufficient neuromuscular response to a slip or trip are attributed to involving a reduction in muscle synergies and a larger latent coordinated muscle activity onset time. This renders the elderly incapable of recovering from the abrupt slip or trip accident regardless of their peak muscle strength. Personalised interventions are required to maintain (or possibly restore) a greater repertoire of neuromuscular control. The goal is to further improve optimal coordinated muscle levels in muscle force ranges that equate to roughly 30% of the patients' body weight. This will help older patients be less susceptible to falls and subsequent morbidity, leading to improved quality of life and a reduced burden on the health system.

The inter-relationship between physical frailty and cognitive impairment are further apparent, leading to further declining physical and cognitive function as well as poor quality of life. Cognitive frailty is the co-occurrence of physical frailty and cognitive decline in older people but without dementia. Improving neuromuscular control will reduce physical frailty and allow older patients increase their scope of activities of daily living and reduce associated cognitive impairment. Furthermore, it is well known that concentrating on fine motor skill execution places a cognitive load on the individual and fatigue increases the cognitive load to perform a given fine motor skill. Neuromuscular control therefore becomes a proxy measure of cognitive loading or capacity. Mild Cognitive Impairment (MCI) or mild neurocognitive disorder is an intermediate state between the expected cognitive decline of normal aging and the more serious decline of dementia. MCI patients suffer problems with memory, language, thinking and judgement. This state can progress further to dementia, mostly in the form of Alzheimer's disease. While strength training and aerobic exercise have displayed encouraging results including neurogenesis, neuroplasticity and reversing MCI, utilising personalised cognitive exercise in the form of neuromuscular control exercises that correspond to an individual's specific cognitive strength deficit has mutual benefit to create a greater functional footprint of strength and cognition to execute higher level activities of daily living. Cognitive deficits in the form of concussion or mild Traumatic Brain Injury (TBI) are the result of a direct impact to the head or an indirect ‘impact’ such as a whiplash type event. The results of concussion render an altered mental state for the patient. In high impact sports like rugby or boxing where elite athletes' decision making and precision movements determine success, concussion can render an athlete inept or unsteady and capable of further risk of injury to themselves and others. The need to swiftly assess or measure physical and cognitive states in the form of neuromuscular control is critically important to determine functional cognitive decision making and physical movement performance. Medical professionals use Sport Concussion Assessment Tools (SCAT3) protocols to examine injured athletes for concussion and provide cognitive, physical and clinical assessments. However, these tests are rudimentary and lack the precision required to meet the standards of athletes' elite level of performance. Greater accuracy, sport movement specific measures and the ability to repeatedly measure quickly when rapid changes occur over time are required. Athletes are known to under-perform on pre-season baseline cognitive tests that are used to measure concussion deficits as a way to cheat the system so they can remain playing if they suffer in-season head trauma. Also, measuring changes over time can chart improvement for deciding to return to play. Furthermore, neuromuscular control can be used to proxy measure of the progression of and possible treatment for Chronic Traumatic Encephalopathy (CTE) over time, a neurodegenerative disease caused by repeated head injuries first identified in American Football players.

Drugs and alcohol can have serious repercussions for the functioning of the entire nervous system, particularly the brain. Impaired neuropathological pathways impede cognitive decision making, physical reaction times and steadiness of people under the influence. Further, the effects of long-term substance abuse have displayed nerve cell atrophy and brain shrinkage. These effects can be measured over time to determine deterioration of the neuromuscular system.

Varying the cognitive load of an individual to measure the level of impairment challenges working memory resources, an example of which is linearly increasing digit string-length difficulty during number recall. Using these types of tests as baseline measures has limitations. Athletes are known to game-the-system in their favour during the off-season to score lower baselines and increase their chances of continuing to play in-season after suffering concussion. During neuromuscular control testing, cognitive load can be varied periodically. Increasing the level of difficulty in an aperiodic real-time manner can be executed by adding or reducing extrinsic load such as the number of distractions. An example of this could be asking the user to match or track the force required in some force-measuring sensor to an unpredictable real-time blue coloured curve on a screen but have a plurality of other distracting coloured curves appear spontaneously to compound cognitive load. Similarly, observing balance tests in athletes is highly subjective after a suspected concussion and doesn't differentiate from in-game variables like fatigue or tester reliability/bias. Combining cognitive load and physical tests to sports-specific-body-positions as well as the athlete's strength ranges poses improved reliability to identify athletes at risk to the effects of concussion. Neuromuscular control monitoring can be applied in an aperiodic manner and provide advantages over existing methods. The repeatability and precision it offers can identify whether athletes have a deficit or are trying to gain an in-season advantage.

There are benefits to measuring cognitive load and physical load concurrently. The advantages extend beyond the short time it takes to complete the test, to measuring efficaciously what is relevant for functionally-cognitive activities of daily living. These activities involve a level of cognition and body positioning within the range of force production, executed in a coordinated manner.

Operators of precision or particularly heavy equipment such as undertaken in mining or logistical environments are susceptible to neuromuscular impairment due to fatigue if they are required to concentrate for extended periods of time. These workers are often on shift-work where concentration is further impaired due to sleep deprivation. The same applies for pilots, taxi drivers, truck drivers and security workers. Simply using the length of the shift or logging hours of operation are insufficient and do not take into account the effect that the operation has on an individual's performance. Screening workers for competency and determining the efficacious nature of work enables recruiting the right workers to suitable duties. Neuromuscular tests can be specifically designed with the desired operational tasks required in employment. Testing workers prior to starting their shift as well as during their shift will help assess and monitor neuromuscular impairment with the objective of limiting the likelihood of avoidable incidents. This can also have significant economic benefit to employers such as lowering insurance premiums and creating a safer working environment. Embodiments of the methods described herein can be used to assessing whether the user is suffering from a mild cognitive impairment and the extent to which the user is suffering cognitive impairment is based on comparing the differences in neuromuscular control obtained when the user is under a varying cognitive loading.

This may comprise taking historical baseline measures from the user and then performing the same assessment when not suffering from a mild cognitive impairment or at an early stage of cognitive decline. In the case of athletes, the baseline could be determined during a preseason or offseason time, and then stored for use during the season (i.e., after a suspected concussion or head impact). In the case of patients with neurological disorder leading to a cognitive decline, a baseline may be taken at any stage decline as the decline process is typically monotonic. Similarly, embodiments could be used to determine a measure of the performance of cognitive function. In this embodiment at least one first set of exercises in a neuromuscular assessment has a higher degree of difficulty compared to another second set of exercises. That the first set of exercises impose a higher cognitive load on the user and so a measure of the performance of cognitive function is based on differences in neuromuscular performance between the first and second the sets of exercises. In another embodiment the method of performing a neuromuscular assessment uses a plurality of exercises of substantially the same degree of difficulty. Cognitive loading is then applied during only part of the assessment with the cognitive loading applied via alternate (non-neuromuscular) method. In one form the alternate method of applying cognitive loading may be achieved by asking the user to perform a mental cognitive task. This may comprise counting backwards from 100 by 7s (or some other combination of numbers so the user can't game the system by learning a sequence). In another form the alternate method may be achieved by asking the user to draw a given scene an example of which may be drawing a clock face with a given time shown. In another form the alternate method may be asking the user to perform certain pattern recognition and matching tasks. In yet another form, the alternate method may be by changing the colour of the defined patterns of the representations of target parameter on the visual display or adding multiple different patterns to the visual display. Any number of cognitive loading techniques may be applied as the alternate method and it is also possible to determine whether the user is truly under cognitive loading during these tasks by comparing their performance from session to session and cross correlating with the measure of neuromuscular control.

As discussed above embodiments of the method used to assess neuromuscular control can also be used as rehabilitation exercises, and thus their repeated use may form the basis of rehabilitation methodologies that are designed to retrain the brain to adapt to the new signalling environment in order to restore control and functionality. More generally embodiments of the method may be considered to methods for neuroplastic training or enhancing neuroplasticity, and may be used for treating or reducing arthrogenic muscle inhibition, increased cortical drive to the target muscle or muscles, improved excitability/inhibitory balance, improving motor unit recruitment, increasing muscle strength, improving reaction time, reducing neuromuscular fatigue, reducing co-activation of agonist/antagonist muscles, optimising both co-ordinated limb and whole body motion, reducing pain, reducing overactive protective mechanisms, reduced jamming sensation experienced within a joint, improved joint loading within the target joint and surrounding joints, reduced both biological joint and implant wear, improved biomechanical and kinematic motion, reduced limping, improved balance and proprioceptive control, improved posture, improved bipedal motion, improving through range joint stiffness, reduced joint swelling and inflammation, improving joint end of range of motion, improved adjacent body parts in similar fashion to the target joint, improved sleep quality and quantity, improving self-confidence, improving motivation and emotional status, improving functional activities of daily living, assessing and improving attention deficit hyperactivity disorder, assessing the effects of sleep deprivation, enhancing relaxation and mindfulness, enhanced breathing for various respiratory disease states, neuroplasticity, measuring the effects of drug and alcohol impact on the body, improving cognitive function, etc.

Embodiments of the method of assessing neuromuscular control such as illustrated in FIG. 22 and described herein, along with related rehabilitation methods and neuroplastic training methods as illustrated in FIG. 23 can equally be applied to healthy individuals to further improve musculoskeletal function or to improve the performance of athletes, including reducing the effects of arthrogenic muscle inhibition, assessing a plurality of joint neuromuscular control, faster and more complete recovery after injury or disease or surgery, assessing and enhancing performance for athletes and occupations requiring higher levels of performance, enhancing skill development, etc.

Athletes are always looking for ways of improving performance. Under normal conditions less than the full complement of motor units are actively engaged in what would be considered a normal full exertion attempt. In extreme circumstances these reserve motor units can be recruited into action. Embodiments of methods taught in the present disclosure appear to unlock these reserve motor units allowing more motor units to be engaged and furthermore allowing the motor units to fire synchronously, generating more power than would otherwise be possible. FIG. 3A offered a schematic representation of the reserve muscle fibres present in a normal, healthy motor neuron pool under nominal, maximal excitation. A non-negligible number of motor neurons remain inactive, even under full exertion. Each motor neuron takes its turn as the reserve motor neuron, allowing its downstream motor units to ‘rest’ periodically, even in a notionally full exertion state. The methods taught in the present disclosure appear to allow engagement of substantially all of these reserve motor neurons under full exertion.

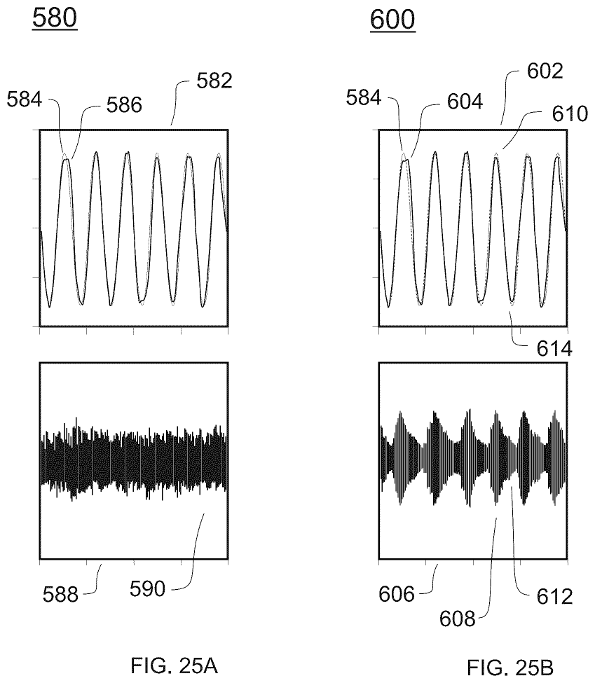

FIG. 25A is a schematic representation of an electromyography study of an exemplary subject suffering arthrogenic muscle inhibition at 580. Electromyography (EMG) measures muscle response or electrical activity in response to a nerve's stimulation of the muscle. The test is used to help detect neuromuscular abnormalities. During the test, one or more small needles (or electrodes) are inserted through the skin into the muscle. Some EMG apparatus use skin-attached electrodes. EMG measures the electrical activity of muscle during rest, slight contraction and forceful contraction. The chart 582 shows the defined pattern 584 and measured force 586 as taught previously that were used in this study. The chart 588 shows the output of the EMG sensors for a typical subject suffering AMI during the contraction and relaxation cycles of the defined pattern 584. The EMG signal 590 is very noisy and shows no substantial correlation to the periodic muscle contractions occurring. FIG. 25B by comparison is a schematic representation of an electromyography study on the same exemplary subject of FIG. 25A after a course of rehabilitation according to an embodiment at 600 where similar elements from previous figures are similarly numbered. Chart 602 shows the defined pattern 584, which was exactly the same as that used in the measurements shown in FIG. 25A and the measured force 604 post-rehabilitation. Chart 606 shows the output of the EMG sensor which was identically located as for the aforementioned study.

The EMG signal this time demonstrates a significant cyclical nature and is highly correlated with the measured force. Peaks in the EMG signal 608 correspond to periods of peak measured force 610 while troughs in the EMG signal 612 correspond to periods of minimal applied force 614.

This study demonstrates that a subject suffering arthrogenic muscle inhibition is able to recruit more muscle fibres after Neuro-Modulation Therapy. Similar results were obtained with normal, healthy subjects who underwent testing before and after a session of Neuro-Modulation Therapy. The EMG results demonstrated an increased ability to recruit muscle fibres post-treatment, even for the healthy subjects. This is consistent with increases in maximum strength and power measured post-treatment in both the subject suffering AMI and the healthy subject. That is, the recruitment of motor units has application beyond merely treating injury or rehabilitation from surgical intervention. Healthy populations may also benefit from increasing the recruitment of motor units. As would be appreciated by one of ordinary skill in the art, increasing the recruitment of motor units may provide benefits in overall strength, endurance and peak power generation by muscles for both athletes and non-athletes. That is, embodiments of the methods disclosed herein may be used to increase strength by increasing the overall number of motor units able to be recruited, as well as improving endurance by allowing the increased motor unit pool to deliver a previously maximal strength while ‘resting’ a larger proportion of the total available motor pool. Embodiments may also be used to improve the peak power generation of a muscle through providing greater synchronous motor unit firing as demonstrated by electromyography studies.

Thus, in addition to therapeutic and rehabilitation applications, neuro-modulation methods described herein, and associated apparatus and systems, may be also used to further develop or refine a skill by a healthy person, or at least a person not specifically suffering from a deficiency of the target muscles or joint. Such neuro-modulation based developmental methods include methods for performance enhancement or performance optimisation by athletes and other persons such as dancers and surgeons requiring a high degree of precision and control of joints and muscles. Intricate and fine motor control sports such as target shooting sports are a niche area such that small musculoskeletal control deficits in any area of the body can significantly inhibit the desired accuracy outcome. Target sports like darts, archery as well as bullseye pistol and rifle shooting use the highest levels of musculoskeletal control precision. Even deficits in breathing musculoskeletal control can impede the desired result in these sports as small loss of control at the release or firing point can lead to exponential errors the further away the bullseye target is. For these reasons, these types of target sports require many years of repetitive practice. It has been reported that athletes require 10 years of dedicated practise to attain elite status. However, these types of sports could be enhanced through training high-precision musculoskeletal control at the areas of the body they exist, to more consistently improve the release and firing points and subsequent higher target scores. This may speed up the accuracy training and reduce the years it takes to reach elite athlete status.

Other sports that could benefit from musculoskeletal control are both throwing and kicking sports. For example, a javelin thrower requires every segment involved from the ground-up placement of the feet to the point of release of the javelin to be optimally controlled to allow the right forces to be transferred from one body segment to the other and finally the javelin. If a joint lacks musculoskeletal control such as an unstable ankle, transferring appropriate force using the desired technique maybe lost due to undesirable movements, forces and directions. Similarly, kicking a soccer ball involves the force translation from the ground-up of foot placement of the opposite kicking leg via the pelvis and trunk to execute ball speed, spin, trajectory, distance and accuracy. Identifying and training with musculoskeletal control in mind and using identical body positions to the specific sports application allows a specific personalised approach to every body type and shape. Sports that utilise sliding and gliding such as ice skating use an intricate low friction/high balance interface. If an ice skater has even a small loss of musculoskeletal control in the trunk, particularly during a complex manoeuvre, the result could be a catastrophic loss of balance and resultant fall onto the hard ice. Sports that require athletes to fit within a physical framework like cycling can benefit in much the same way. Any loss or undesired movement during the pedal stroke can reduce the effectiveness of the mechanics from muscular force generated onto the pedals, to the cranks, to the chain, wheels and finally onto the ground via the tyres to create forward propulsion. Lack of lumbar and pelvic musculoskeletal control seen as an uncontrollable lateral wobbling of the pelvis in the seat can unduly dissipate and dampen the effectiveness of the force transmission required to resultantly move forwards efficaciously. All these sports undertake complementary strength resistance training in gymnasiums but could be further enhanced in sports-specific positions and movement that identify and train musculoskeletal control.

It will be readily apparent to one of ordinary skill in the art that embodiments of NMT apply to almost any sporting endeavour and coordinated body motion. These include, but are not limited to: tennis with shoulder/arm control for swing and hip/leg positioning in preparation for the swing; golf with back/torso control; soccer with leg control or throwing control in American football; dance; indeed any sporting or coordinated motion endeavour.

Another specific example of the coordinated body motion analysis and training is that of a rowing machine. The rowing machine exercises both upper body and lower body simultaneously and optimum rowing performance requires synchronised motion. When the upper body motion is synchronised with the lower body motion the rower can deliver peak power and optimise power delivery at minimum exertion, creating efficiency. Embodiments of NMT therefore utilises either a plurality of cobot apparatus or a plurality of apparatus similar to the apparatus of FIG. 8B wherein one apparatus is adapted to the upper body and another apparatus is adapted to the lower body of the rower. It would be readily apparent to one of ordinary skill in the art that other apparatuses may be adapted to perform a similar function. According to this embodiment the rower is presented with either a defined pattern, 382 of FIG. 16A, for the upper body or a defined pattern for the lower body or a defined pattern for both the upper and lower body. In this way the rower can focus on optimising their upper body stroke, their lower body stroke or their holistic stroke. Similarly, the training regime may occlude either the upper body's defined pattern, the lower body's defined pattern or both defined patterns simultaneously. Embodiments of NMT therefore teach the use of coordinated feed-back and feed-forward neuromuscular training programmes.

Embodiments of NMT are not limited to optimising the performance of sports people. Other persons such as dancers and surgeons may also benefit from the ability to lay down optimised ‘muscle memory’ maps using neuro-modulation. Dancers can perhaps be considered to rely more on controlled coordinated movement than most athletes. The highly structured dance moves involved with ballet are an example of intricate coordinated movement that would benefit from the use of cobots to measure and train coordinated movement according to embodiments as described herein, however this would equally apply to other forms of dance.

Embodiments as described herein disclose the effectiveness of Neuro-Modulation as a method of downregulating AMI, its associated muscle co-contraction and the sensation of pain that comes with joint immobility after injury or surgical insult. As discussed, AMI is the result of a mismatch between the body's internal model of motor function and the sensory feedback from an altered joint (in the case of total knee arthroplasty). The brain not only excels at pattern matching but it is equally good at creating patterns in response to repeated stimulus. It is an unfortunate fact that the brain can also create patterns in response to chronic pain and central sensitization (a prolonged neuro-immune response).

Patterns of chronic pain can be learnt and once these patterns have been ‘written’ in the brain can be very difficult to remove.

It is increasingly recognised that low-grade inflammation exists in the diseased osteoarthritic joint. This inflammation is the result of the innate immune system, which seeks to restore tissue homeostasis resulting from injury, surgery, disease or infection. In the example of osteoarthritis, inflammation progresses the disease, causing cartilage and meniscus damage, subchondral bone remodelling, osteophytes and synovitis. Further, pain is initiated by factors that activate sensory nociceptors innervating the skin and joint tissue. Nociceptors and immune cells both respond to tissue injury by promoting a combined neuro-inflammatory signalling that facilitates pain as well as tissue repair. Prolonged tissue damage may result in interactions between the innate immune system and the nervous system at different levels of the pain neuraxis. If damage does not resolve, such as in the disease of osteoarthritis, the persistent inflammatory environment causes peripheral and central sensitization of the nervous system. The result of this sensitization is long-term changes (patterning) in neurons, as well as infiltration of immune cells, further boosting the inflammatory state and promoting chronic pain. An innate immune response in the osteoarthritic joint generates a great deal of crosstalk between different joint tissues, mediated by inflammatory markers, causing hyperalgesia and allodynia. This promotes persistent chronic pain in a process of neuro-inflammation, which has been the focus of considerable research attention in the past decade.

There is a need to develop therapies that target the neuro-immune system, particularly for chronic pain, which costs $600 billion per year in the United States alone. Neuro-Modulation Therapy may be beneficial addressing peripheral and central sensitisation by modulating and retraining the nervous systems response to movement control and hence pain. As crosstalk between the tissues exist, modulating and re-calibrating sensory inputs and motor outputs in the form of neuromodulation may modulate not only a normalised neural response but a subsequent immune response—as they are both coupled. Neuro-Modulation Therapy of total knee arthroplasty patients that have been unresponsive to conventional rehab has demonstrated a reduction in chronic pain of at least 20% and in some cases chronic pain has been completely resolved, allowing some patients to undertake activities of daily living completely pain-free. In acute knee patients, the same 6-week course of neuromodulation therapy resulted in a 52% reduction in pain. If neuromodulation therapy was specifically targeted to modulate chronic pain, this could be further modulated, resulting in greater efficacious outcomes. In yet another broad aspect embodiments described herein provide a method of reducing chronic pain. The method is applicable to any chronic pain associated with a musculoskeletal condition but may be equally effective with a generally applied Neuro-Modulation Therapy targeting non musculoskeletal chronic pain. It would be readily apparent to one of ordinary skill in the art that the method may utilise any combination of the methods, apparatus or systems taught in the present disclosure.

Dissociative disorders include a wide variety of mental health conditions whose common core involve experiencing a loss of connection between thoughts, memories, feelings, surroundings, behaviour and identity. These conditions include and escape from reality in ways that are not wanted and not healthy. This causes problems in managing everyday life. Treatment for dissociative disorders have traditionally been psychotherapy and pharmacological but Neuro-Modulation Therapy may offer a new therapeutic tool. By way of example, schizophrenia, a dissociative disorder, can result in the perception of an out-of-body experience whereby the sufferer experiences a conscious separation or dissociation from their physical body. This may involve a disruption in the brain's maladaptive processing of sensory information and spatial awareness. Furthermore, this may account for a mismatch in the coupling of the sensorimotor mapping on the brain. Clinically, schizophrenia patients report oversensitivity to sensory stimulation that theoretically correlates with stimulus overload and leads to cognitive fragmentation. Studies have also shown that schizophrenic patients also have impaired central nervous system inhibition. These result in a disorganisation of body movements and spatial relationships. This involves the somatosensory cortex which processes sensory information from the body and the motor cortex which controls voluntary movement. Studies have shown that disruptions or alterations in these brain regions can lead to changes in body awareness and perception, including sensations of floating of disembodiment. The distortion and disconnect could result in the sensation of observing one's body from an external perspective, altering perceptions of body self-location.

Current treatments aimed at motor mapping interventions are speculative to date and require further investigative research. Embodiments described herein provide a clear mechanism for calibrating the sensory inputs associated with voluntary motor outputs and updating the internal model of motor control. Neuro-Modulation Therapy has the potential to rewire and normalise the coordinative mapping on the brain through neuroplasticity. Reorganisation of motor function signal processing may restore not just the bodies mapping representation from a maladaptive disorder, but create a new neuroplastic representation whereby patients feel connected to their own body, a return from disembodiment.

In yet another broad aspect embodiments described herein provide a method of treating dissociative disorders with Neuro-Modulation Therapy. It would be readily apparent to one of ordinary skill in the art that the method may utilise any combination of the methods, apparatus or systems taught in the present disclosure.

Arthrofibrosis is a fibrotic joint disorder that begins with an inflammatory reaction (from the immune cells) to insults in the form of injury, surgery or infection. Arthrofibrosis is therefore an excessive fibrotic scar or extracellular matrix which results in adhesions which contract the surrounding joint capsules, bursae and tendons. Patients experience pain, reduced range of joint movement and poor quality of life. The scar is primarily composed of a disproportionate ratio of collagen type 1 (high-tensile strength that resists normal stretch) compared to stretchy elastin. In addition to the altered tissue composition, the fibrotic extracellular matrix has excessive cross-linking that rendering it resistant to degradation and stretching. In arthrofibrosis of the knee joint patients report stiffness and reduced range of movement. This is commonly reported after anterior cruciate ligament reconstruction and total knee arthroplasty surgeries. The incidence of arthrofibrosis is largely under reported because formal diagnosis requires a subsequent surgery to visually inspect the joint. Nonetheless, it is reported in the literature up to 15% of total knee arthroplasty and 35% of anterior cruciate ligament reconstruction patients have arthrofibrosis.

At a cellular level, arthrofibrosis is characterised by upregulated myofibroblast proliferations with reduced apoptosis, adhesions and aggressive synthesis of extracellular matrix. Although extracellular matrix is necessary for healing and wound repair, dysregulation of production leads to pathologic arthrofibrosis, an over protective mechanism. It is interesting that this dysregulation leading to an overprotective mechanism is similar to the dysregulation of inhibition that occurs with the case of overprotective arthrogenic muscle inhibition. Most importantly, a lack of understanding of the role that inflammation plays in arthrofibrosis generally leads to overly aggressive physical therapy rehabilitation programme (Usher et al 2019). In an extensive review (Usher, K. M., Zhu, S., Mavropalias, G. et al. Pathological mechanisms and therapeutic outlooks for arthrofibrosis. Bone Res 7, 9 (2019). https://doi.org/10.1038/s41413-019-0047-x), Usher et al explains that aggressive conventional exercise can initiate or worsen arthrofibrosis because exercise triggers an inflammatory response resulting in yet more inflammatory cytokines and collagen production. Usher et al identified numerous papers frequently recommending “aggressive” physical therapy as soon as possible after surgery.

It is also recognised that a significant majority of post-surgical patients have muscle weakness related to the arthrogenic muscle inhibition. As we have already identified in the present disclosure, AMI results in reduced motor-unit recruitment and co-activation of opposing joint muscles acting to protect the joint. The present disclosure has already taught the many benefits of Neuro-Modulation Therapy for treating AMI and increasing motor-unit recruitment and reducing co-activation and NMT is an ideal substitute for aggressive rehabilitation exercise in arthrofibrosis. Neuro-Modulation Therapy uses light intensity exercises that are not aimed at pushing maximum strength, but rather based upon sufficiently activating the sensory system with the purpose of modulating a calibrated motor output response. When undertaken, Neuro-Modulation Therapy dampens the overactive protective mechanism of AMI and in doing so is likely to also dampen the overprotective mechanism of the neuro-immune signals responsible for arthrofibrosis. Thus NMT may be used for treatment of arthrofibrosis.

There have been recent advances in pharmacological agents and biologics to treat the pathogenesis of arthrofibrosis. However, despite the understanding of the pathogenesis of arthrofibrosis, there are no effective therapies to halt fibrosis let alone cure it (Usher et al). For those patients that have already experienced arthrofibrosis, a combination therapy of pharmacological agents or biologics with Neuro-Modulation Therapy could be considered. Pharmacological agents or biologics could be used to treat the pathogenesis of arthrofibrosis while Neuro-Modulation Therapy could be applied to both target uptake at the specific joint as well as calibrating the neuro-immune response.

As taught in the present disclosure Neuro-Modulation Therapy is particularly effective immediately following a total knee arthroplasty because it downregulates arthrogenic muscle inhibition and allows updating of the internal model with a non-inhibited, non-cocontracted gait. Delaying Neuro-Modulation Therapy is non optimal because the internal model is being rewritten with the inhibited, cocontracted gait (and thus if NMT delayed later it may require more extended treatment to overcome this). The neuromodulation research studies identified that acute total knee arthroplasty patients (starting neuromodulation therapy within 10 days after surgery) have outcomes 2.5 times greater than those chronic total knee arthroplasty patients (starting neuromodulation therapy 4 months after surgery) using patients reported outcome measures for the same 6 week programme. In the same way, it would be expected by a person skilled in the art that early intervention may have the greatest impact on reducing the onset of arthrofibrosis in the first place.

More generally NMT may be used as a treatment for neuro-immune conditions. There is a growing body of evidence linking the neurological and immune systems. While initially believed to act independently, there is now a greater understanding of the interconnectedness of the neurological and immune systems and their impact on cognition, behaviour, psychiatric illness and neurodegenerative disease. The role of immune system mediators such as cytokines and leukocytes in mental health disorders, neurological and musculoskeletal disease is an area of much investigation. Trials investigating the therapeutic effects of direct brain stimulation on autoimmune disease of the gastro-intestinal tract and other disorders have demonstrated positive outcomes in some cases. The neuro-immune system has been primarily studied with respect to conditions that affected cognitive function such as occurs in neurodegenerative disorders, brain injury and viral or bacterial infection, whereas classically the cerebellum was thought to be involved primarily in motor control. However, it has now become clear that the cerebellum has an important impact on the functioning of many brain regions through reciprocal circuit connections and is involved in a wide range of behaviours including cognitive function. Moreover, emerging research has revealed an involvement of the neuro-immune system in several conditions associated with cerebellar motor dysfunction such as ataxias, a group of neurological disorders exemplified by loss of balance and coordinated motor functions. For example, chemokines (which play a major role in the activation of host immune responses) are also effective in altering synaptic transmission in Purkinje neurons in the cerebellum. Application of the chemokine interleukin-8 to Purkinje neurons in mouse cerebellar slices increased the number of spontaneous inhibitory and excitatory synaptic events and increased the amplitude of excitatory synaptic events evoked by stimulation of the parallel fibres. The links between cerebellar function, motor control, cognitive disorders, chronic pain and the neuro-immune system run deep suggesting a pathway for Neuro-Modulation Therapy is applicable is all of these conditions. Neuro-Modulation Therapy is therefore at the crossroads of a wide range of conditions related to motor control and neuro-immune conditions.

The immune system and the nervous system have evolved to provide regulation of physiological homeostasis and protect against threats. The immune system defends against infection and injury through inflammation that neutralises invading pathogens and promotes tissue repair. If unresolved of dysregulated, inflammation can be problematic as in examples of inflammatory and autoimmune disorders. The nervous system integrates biological functions and provides a nearly instantaneous homeostatic control mechanism by release of neurotransmitters and other regulatory molecules. These two systems have perfected evolutionary features that allow for recognition of alterations and threats in the ever-changing microenvironment, mounting protective responses and developing memory to facilitate these protective responses upon encountering similar alterations in the future. Interaction between neurons and immune cells and their communication and functional cooperation is integral to homeostasis and species survival. Neuro-immune interaction with biological consequences have been identified in evolutionarily ancient animals. Neurons in the primitive nervous system of the soil nematode Caenorhabditis elegans regulate innate immune responses, including a non-canonical unfolded protein response pathway. Studies in rodents and humans have identified molecular components of neuro-immune interaction, reciprocally shared between the two systems. In addition to immune cells, the expression of pattern recognition receptors, including Toll-like receptors and cytokine receptors have been demonstrated on neurons, providing a molecular substrate for simultaneous modulation of immune and neuronal function.

The links between the immune and neurological system and the obvious application of Neuro-Modulation Therapy with arthrofibrosis thus offers a new and exciting opportunity for the use of Neuro-Modulation Therapy broadly in the area of neuro-immune therapies (i.e. for treatment of neuro-immune conditions).

An embodiment of the NMT apparatus shown in FIG. 8A was used in a trial of an embodiment of the rehabilitation method as described herein. In a first case study a group of patients were selected who had undergone Total Knee Arthroplasty surgery and were unresponsive to a conventional rehabilitation program. An embodiment of a rehabilitation method using an embodiment of the method of assessing neuromuscular control was performed. This comprises a series of feedback and feedforward exercises (actions) performed on the NMT apparatus in which the target parameter was force and the force varied according to a sinusoidal pattern, with part of the sinusoid occluded during the feedforward exercises. An initial assessment was performed to determine the baseline force/activation of the quadriceps muscles and range of motion of the knee joint for the patient. After 5 minutes (total) of exercises, the joint angle range of the knee joint was again assessed (determined) along with the relative increase in activation of the quadriceps muscles. Each session comprised often sets, or trials (1 set=1 trial), of exercises of 30 second duration in which the user was instructed to attempt to match the sinusoidal force pattern for the 30 second duration. The force pattern was a continuous sinusoidal pattern at a fixed frequency, in most cases around 0.3 Hz (=period of 3.3 seconds) displayed on a screen for the duration of the exercise (30 seconds). The force range (i.e., amplitude of sinusoid) was determined from maximum force range achieved by the patient obtained from the initial assessment. The session started with feedback exercises and proceeded to feedforward exercises with curve suppression of increasing complexity, for example 25%, 50% 75% occlusions in central region and then corners/turning points. A rest gap, typically of 30 seconds was provided between each set (or trial) which allowed for a brief recovery of the muscles as well as the opportunity for the user to review their summary and raw data on the previous set (or trial). Occasionally the rest gap was longer if the patient wished to study their previous results in more detail. The NMT apparatus was configured to repeatedly measure (or sample) the force during each trial, and the difference between the target force and measured force at each measurement point was obtained (i.e., a time series of force measurements). This was normalised (converted) to a percentage error: % error=100%×[target−measured]/target. Then the percentage of time during the 30 second trial that the percentage error was less than the good error threshold (% error<4.25%) was calculated as an accuracy measure. The within session change of knee range motion was 45% comprised of a 34% increase in the extension range and an 11% increase in the flexion range. Similarly, the quadriceps increased activation by 42%. Activation was measured as the change in the quadriceps muscle strength from baseline.

A second study was conducted on a cohort of patients who had undergone TKA and were unresponsive to conventional rehabilitation therapy (like the patients in the first study), professional athletes who had had ACL reconstruction surgery and at least 12 months of conventional rehabilitation and had returned to playing but exhibited AMI, and a group of patients who had patellofemoral subluxation/dislocation who were unresponsive to rehabilitation. Again, an initial assessment was performed and then each patient was provided with 5 minutes of exercises on the NMT apparatus as per the first study (i.e., 10 sets of 30 second duration exercises attempting to match a sinusoidal varying force progressing from feedback to feedforward exercises over the set, and the percentage change in activation of the quadriceps was measured (i.e., before vs after comparison). A control group was also tested. After only 5 minutes of NMT, the patient cohort as a whole reported a 33% increase in quadricep activation, with TKA patients reporting a 42% increase, the professional athletes (ACL reconstruction) showed an 18% increase, and the patellofemoral subluxation/dislocation group showed a 45% increase, whilst the control group showed an 8% decrease. These results show the response to NMT by patients was agnostic to type of injury and surgery. Notably the professional athletes who had undergone 12 months of conventional rehabilitation showed a significant increase after only 5 minutes of NMT therapy. This study thus shows that in addition to improving the outcome of post-surgical rehabilitation programs, embodiments as described herein can also be used to treat AMI.

As a further example, another study was performed on a patient with a TKA and who was unresponsive to 4 months of hydrotherapy, conventional rehabilitation and physical therapy, and walked with a limp. After a single 5 minute session of NMT as described above, the patient exhibited a 44% increase quadriceps activation of the knee which had undergone the TKA, whereas their control knee showed an 18% decrease in quadriceps activation. Further the joint angle range of the knee subject to the TKA increased by 13% following NMT. Further, after only 5 minutes of NMT the patient stopped walking with a limp.

A further study was performed on a 22 year old female (patient X) who presented with a 12 year chronic history of bilateral patella (kneecap) subluxation and dislocation at least 4-5 times a year. Patient X complained of constant swelling and pain in both knees, very weak muscles that surround the knee to the point she felt she would fall if going down stairs. Patient X had a successful bilateral patellofemoral ligament repair to tighten the lax supporting patella passive structures by an orthopaedic surgeon 18 months prior to the first visit to the laboratory for testing. Patient X spent 8 months using crutches to ambulate following the surgery and would still use them on occasion. Patient X completed the post-operative inpatient hospital program to the best of her ability but noted straight away that her thigh muscles wouldn't work properly after surgery, displaying a notable quadriceps lag upon lifting her leg. Patient X completed over 3 years of intensive rehabilitation at 2 separate clinics including physical therapy, daily rehabilitation, stretching and strengthening exercises at home, supervised weekly water-based exercises, acupuncture, heat and cold packs, knee braces and supervised gym exercises. It had cost her thousands of dollars. Unfortunately, it didn't resolve her bilateral knee problem. Patient X could walk a maximum of 400 metres until knee stiffness and quadriceps muscle fatigue became too great to continue.

Due to the unresponsive rehabilitation, patient X underwent an embodiment of NMT therapy. Tables 1 and 2 present a baseline assessment of control of patient X, and the results after a single session of NMT. The NMT session comprised 10×30 second duration exercises, with a 30 second rest gap between exercises (i.e., 5 minutes total of NMT). During each exercise the patient was instructed to attempt to match a sinusoidal force pattern at a frequency of 0.3 Hz for the 30 second duration. The force pattern was displayed on a screen for the duration of the exercise (30 seconds). The NMT apparatus was configured to repeatedly measure (or sample) the force during each trial, and the difference between the target force and measured force at each measurement point (time point) was obtained. This was normalised or converted to a percentage error: % error=100%×|target−measured|/target. Then the percentage of time during the 30 second trial that the percentage error was less than a good error threshold (0% error<4.250%) was then calculated as the accuracy measure. This was performed both left and right knees and the accuracy measures are reported in the first row of Table 1. Interestingly, when conducting the testing it was noted that patient X walked forwards slowly with very small steps using whole-body stiffness displaying no arm swing. Upon instructions to walk backwards, patient X had to concentrate very hard to place her feet compared to forwards walking, noting that her confidence had diminished 900%. She felt backwards walking was causing her quadriceps to buckle. Further when asked to lift a single straight leg up while lying down, her quadriceps muscle on the lifting leg would tremor and shake involuntarily, with Jerkiness and inability to execute the exercise properly.

TABLE 1
NMT therapy - baseline assessment of patient X, and results after a single session of NMT.

| | Left Knee | | Right Knee | |
|---|---|---|---|---|
| Assessment | | Immediately after | | Immediately after |
| (action) | Baseline | NMT session | Baseline | NMT session |
| 30 seconds | 44.8% accuracy | — | 48.6% accuracy | — |
| neuromuscular | | | | |
| accuracy test 3-7 | | | | |
| kg · f range | | | | |
| Knee flexion | 112° | 120° | 110° | 112° |
| range | | | | |
| Knee extension | 0° | −3° | 0° | −3° |
| range | | | | |
| Quadriceps | 9.1 kg · f | 15.0 kg · f | 10.8 kg · f | 15.3 kg · f |
| maximum | | (5.9 kg · f increase, | | (4.5 kg · f increase, |
| voluntary | | 65% increase | | 42% increase |
| isometric | | quadriceps | | quadriceps |
| contraction in 90° | | activation) | | activation) |
| knee flexion | | | | |
| Quadriceps left | 84% | 98% | 84% | 98% |
| and right strength | | | | |
| symmetry | | | | |

TABLE 2
NMT Therapy - Baseline assessment of control of patient X, and the
results after a single session of NMT - functional test results.

| Functional Tests | | Immediately |
|---|---|---|
| Results | Baseline | after NMT |
| Timed up and | 12.7 seconds | 12.4 seconds (0.3 second |
| go test (TUG) | | improvement) |
| Horizontal | 27 cm (uses a | 29 cm (uses a knee and hip bend |
| forward reach | locked out straight | strategy, improved 2 cm |
| test | knee strategy) | horizontal reach) |

A 14 week rehabilitation program (intervention) was performed. This comprised a 5 minute neuro-modulation session for each leg, conducted 3-5 times a day, 6 days a week. The strength range progressed from 3-7 kg·f to 8-13 kg·f over 14 weeks, with a knee joint range of 20-70°. After a 2-week washout period (no rehabilitation/NMT therapy) the patient's range of control was reassessed, and results are presented in Tables 3 and 4. Each 5-minute session comprised 30 second trials using a sinusoidally varying target force curve. The trials in a session typically comprised a range of feedback and feedforward exercises. Each week the program was reviewed and adjusted, with the complexity of feedforward exercises increasing over the course of the 14 week program. Over the 14 weeks, occlusion used in feedforward trials varied using central occlusions (25%, 50%, 75%) The final error scores attained above 90% error ranges in the 75% central occlusions, i.e., 90% of time during an exercise that the % error (100%×|measured−target|/target) was less below the 4.25% good error threshold with 75% occlusion.

TABLE 3
NMT Assessment of patient X after 14 weeks on NMT rehabilitation program.

| Test | Left Knee | Right knee |
|---|---|---|
| 30 seconds neuromuscular | 84.3% | 86.0% |
| accuracy test 8-13 kg · f range | | |
| Knee flexion range | 127° (improved 15°from | 124°(improved 14°from baseline |
| | baseline without stretching) | without stretching) |
| Knee extension range | −3°(improved 3° from baseline | −3° (improved 3° from baseline |
| | without stretching) | without stretching) |
| Quadriceps maximum voluntary | 20.5 kg · f | 21.0 kg · f |
| isometric contraction in 90° | (11.4 kg · f increase or 125% | (10.2 kg · f increase or 94% |
| knee flexion | increase from baseline) | increase from baseline) |
| Quadriceps left and right | 98% symmetry for strength | |
| strength symmetry | | |

TABLE 4
NMT Assessment of patient X after 14 weeks on NMT
rehabilitation program - functional test results.

| | Note - the previous baseline tests |
|---|---|
| | were not included as patient X was |
| Functional test | advanced beyond the functional range) |
| Double leg squat and return | ¾ squat depth to floor and return |
| Walking up stairs | 20 stairs alternate leg walking |
| Walking down stairs | 20 stairs alternate leg slow walking |

From comparing Tables 1 and 3, it can be observed that the accuracy improved from initial (Left) 44.85% to final 84.3% (improved 39.5%) and (Right) 48.6% to final 86.0% (improved 37.4%). Accuracy was defined as the percentage of time during the 30 second assessment trial that the percentage error was less than the good error threshold (% error<4.25%). After 10 months from the final testing, Patient X now reports they are able to undertake walking treks beyond 2 hours and have started jogging for fitness. We conclude that neuro-modulation training is trainable and is sustained 10 months after neuro-modulation training intervention. Neuro-modulation is safe and effective for patients who don't respond to traditional rehabilitation treatment.

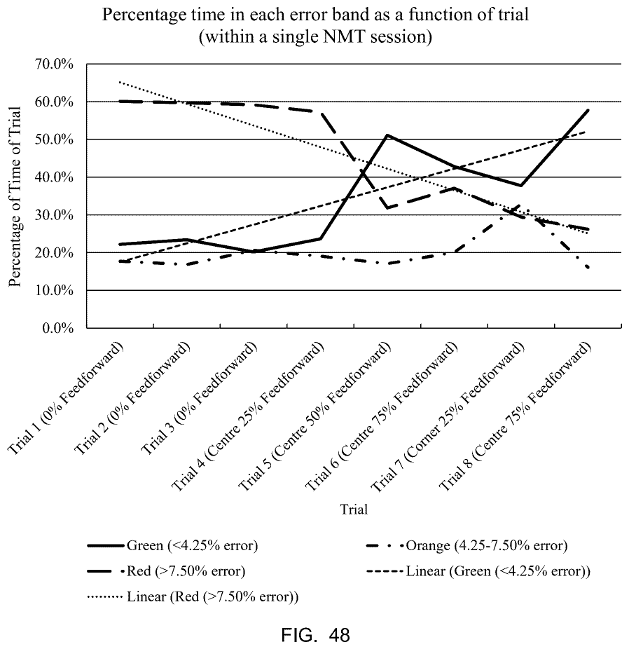

A study was also performed on a patient who underwent a medial patella ligament stabilisation operation to stop her patella from dislocating and who was unresponsive to traditional rehabilitation. The patient was tested pre and post a session of Neuro-Modulation Therapy. The initial NMT assessment determined the patient's strength deficit was 93% and the range of movement deficit was 35 degrees compared to the opposite side. The patient walked with a straight-leg gait and subsequent limp with significant asymmetry. The NMT session was then performed using an NMT apparatus similar to the extension apparatus shown in FIG. 8A. The session compared 8 sets (trials) of exercises, each of 30 second duration with an instruction to match a sinusoidally varying target force curve at a frequency of 0.3 Hz. Rest gaps were provided between each set and the entire session was completed within one hour. Table 5 displays the results of the NMT Therapy session and FIG. 48 is a plot of the percentage of time of atrial that the error was in each of three error bands for 8 trials forming a single Neuro-Modulation Therapy session for a patient using a neuro-modulation apparatus according to an embodiment. The three error bands comprise a green (i.e., good) error band (<4.25% error) represented as a solid black line, an orange error band (moderate 4.25-7.5%) represented as a dash-dot black line, and a red (bad) error band (error>7.5%) represented by a long dash black line. The first three sets, or trials, each comprised of a single 100% feedback exercise in which the force varied sinusoidally as illustrated in FIG. 16A. The minimum to maximum force range was increased slightly in each feedback test. These three trials (3 sets of 1 exercise) with 100% feedback (i.e. no suppression, 0% feedforward) act as a baseline. A further 5 sets of 30 second duration feedforward exercises, i.e., 5 trials, were performed using a mix of 25-75% suppression during each trial exercise as detailed below in Table 5. The progression of the suppression in trials 4-7 increased the complexity of the exercise in a graduated manner, e.g., 25%, 50%, 75% centre suppression, then 25% corner suppression). Trial 8 then reduced the complexity and was a repeat of trial 6 (centre 75% suppression/occlusion). The same min to max force range was used for all feedforward trials, and this was the same range as the last baseline feedback trial (trial 3).

During each trial the force was repeatedly measured and the difference between the target force and measured force was calculated and expressed as a percentage (e.g., % difference=100%×[Measured-Target]/Target). Then, the percentage of time during the 30 second trial that the difference error was within each of 3 predefined error bands was calculated. In this experiment the error bands were labelled as green=error≤4.25%; orange=4.25%<error≤7.5%; and red=error>7.5%. In the three baseline trials the difference error was in the red band for around 60% of the trial and in the green band for around 22% of the trial. A similar result was obtained for the first feedback exercise with 25% centre suppression. When the suppression was then increased to 50% central suppression in trial 5, there was a significant improvement with the percentage of time in the green band doubling (24% to 51%) and the percentage of time in the red band approximately halving from the previous trial (57% down to 32%). These results were then substantially maintained or improved for the remaining trials, such that by the last trial, the percentage of time in the green band had increased to 58%—i.e., an increase of around 36% over the baseline and the percentage of time in the red band had dropped to 26%—a decrease with respect to baseline of around 34%. Trial 8 was a replicate of trial 6, but showed a significant improvement over trial 6 (time in green zone up 15% and time in red zone down 10%). The percentage of time in the middle orange band was reasonably steady at around 20%, apart from trial 7, which was the more complex and challenging trial in the session, where the orange zone increased to 38%. Notably the percentage of time in the red band for trial 7 was still a decrease of around 10% (from 37% to 29%) even though the complexity had increased. The trendlines in FIG. 48 clear show the improvement with the green band trendline (short dash) increasing (positive slope) and red band trendline (dotted line) decreasing (negative slope). The green band trendline equation was y=0.0495x+0.1258; R²=0.69, and the red band trendline equation was y=−0.0572x+0.7085; R²=0.844. Table 5 also provides a breakdown of the errors in different phases of the target force curve (incline, decline, occluded and visible phases or portions).

Table 5 and FIG. 48 thus shown that the introduction of feedforward exercises (suppression) resulted in a significant improvement in the quality and smoothness of the movement after only a single session of NMT. Further after the NMT session the patient strength was remeasured, and was found to have increased by 165% and along with a 10° improvement in the range of movement. The patient walked unprompted without a notable limp and reported no pain whether walking forwards or backwards. The patient also reported they could feel a positive difference, but was unable to further quantify what the difference was. The outcome after one session of NMT also produced a significantly improved symmetrical gait.

TABLE 5
NMT Therapy-Results of a single session comprising 8 Trials

| Trial Number | 1 | 2 | 3 | 4 | 5 | 6 | 7 | 8 |
|---|---|---|---|---|---|---|---|---|
| Green | 22.17% | 23.41% | 20.19% | 23.64% | 51.11% | 42.8% | 37.73% | 57.71% |
| (<4.25% error) | | | | | | | | |
| Orange (4.25- | 17.71% | 16.87% | 20.63% | 19.08% | 17.07% | 20.1% | 32.84% | 16.09% |
| 7.50% error) | | | | | | | | |
| Red | 60.12% | 59.72% | 59.18% | 57.28% | 31.83% | 37.1% | 29.43% | 26.2% |
| (>7.50% error) | | | | | | | | |
| Min-Max | 4-10 kgf | 5-12 kgf | 6-14 kgf | 6-14 kgf | 6-14 kgf | 6-14 kgf | 6-14 kgf | 6-14 kgf |
| Force range | | | | | | | | |
| Curve direction | Up | Up | Up | Up | Up | Up | Up | Up |
| Suppression | None | None | None | Centre | Centre | Centre | Corner | Centre |
| (Occlusion) | | | | 25% | 50% | 75% | 25% | 75% |
| Trial description | Baseline | Baseline | Baseline | Simple | Simple | Simple | Complex | Simple |
| | | | | task | task | task | task | task |
| Feedback % | 100 | 100 | 100 | 75 | 50 | 25 | 75 | 25 |
| Feedforward % | 0 | 0 | 0 | 25 | 50 | 75 | 25 | 75 |
| Incline error | 11.65% | 10.36% | 12.5% | 9.71% | 5.23% | 6.08% | 6.61% | 5.58% |
| Decline error | 15.54% | 12.87% | 11.13% | 15.42% | 9.34% | 10.14% | 8.74% | 8.44% |
| Occluded error | | | | 13.88% | 5.55% | 6.77% | 10.16% | 4.81% |
| Visible error | | | | 12.04% | 8.43% | 9.79% | 6.19% | 9.83% |

In a further trial, a patient who, due to another medical condition, was unable to undergo traditional post-surgical TKA rehabilitation was provided with several sessions of Neuro-Modulation Therapy using a NMT extension apparatus as illustrated in FIG. 8A. An initial assessment determined the patient's strength was 18.9 kg and total range of movement was 63 degrees. A series of 1,242 sessions of NMT were performed over 6 weeks (average 3.45 sessions per day) where each session comprised 5 minutes total comprised often sets (trials) of 30 duration exercises (sinusoidal force curve; feedback or feedforward) spaced by 30 second rest periods. Over the 6 weeks, suppression/occlusion used in feedforward trials varied from central occlusions (25%, 50%, 75%), corner occlusions (25%, 50%, 75%), to more complex occlusions such as those illustrated in FIG. 17. A further assessment after the 1,242 sessions/6 weeks determined the patient's strength was now 28.0 kg and range of movement was 115 degrees—and improvement of 32% for strength and 45% for range of movement. These trials thus illustrate the benefits of NMT as NMT was the only rehabilitation performed and generated a significant improvement in quality of walking, significant reduction in knee stiffness and vastly improved quality of life, measured using a standardised research questionnaire.

The above studies thus demonstrate the benefits including improved range and smoothness (control) of joint movements, and thus emphasise the usefulness of embodiments of the methods described herein when used for post-surgery rehabilitation and treatment of AMI.

Total joint replacement is an invasive surgical intervention to replace the joint surfaces with implants often saved for end stage arthritis. The current and future growth rates worldwide are substantial, especially for knee and hip joint replacement. Weight-bearing joint replacements of the knee and hip make up 90% of the total joint replacement market. While every effort is made to implant and replicate the knee in its many forms, this is often not the case. Over thirty percent patient dissatisfaction rates are reported for total knee replacement. Conventional rehabilitation delivered by a therapist either within a hospital, clinic or home setting fails to return the patient to a satisfactory level of function.

Not all patients have the opportunity to undertake pre-operative rehabilitation that includes daily strengthening exercises of quadriceps, hamstrings, gluteals and calves. Some of these patients are unable to undertake general exercises in a weight-bearing position such as a double leg squat due to pain. Unfortunately, pain causes muscle inhibition and is counter-productive to strengthening the surrounding musculature. Post-operative total knee replacement rehabilitation starts at day zero within either a day-surgical or hospital setting. Early rehabilitation aims to reduce pain and swelling, restore quadriceps muscle activity and help in transferring in and out of the bed. Self-directed and mass-referred exercises form early intervention rehabilitation that involve basic knee movements and general strengthening of the surrounding knee muscles delivered using the generic formula of three sets of ten repetitions for beyond 12 weeks of recovery. The same exercises using the same generic set-repetition formula are expected to impact and improve recovery when the patient is discharged to go home. It is no wonder that patients plateau with rehabilitation and finally give up. However, it is also reported that despite high intensity strengthening exercises, the quadriceps fail to return to pre-operative levels. Mid flexion knee instability after knee replacement is a problem that patients experience such that the knee unexpectedly gives way, especially when traversing stairs. There are mechanisms beyond well-appreciated musculoskeletal consequences that point to systemic mal-adaptive changes in both the peripheral and central nervous system function that explain the quadriceps deficiency. Peripheral and central nervous system functional impairments can render traditional therapeutic approaches ineffective for patients with these impairments, perpetuating poor clinical outcomes. Neuromuscular control is aimed at restoring the musculoskeletal and nervous system function in the form of targeted cognitive exercise to retrain and recalibrate the whole system.

As noted above current rehabilitation methods focus on recovering maximum strength. However, we rarely use maximum strength in activities of daily living and so focusing on maximum strength may not provide a sufficient or complete representation of musculoskeletal function or the efficacy of any recovery therapy programme. More often than not, we use lower percentages of maximal joint force production for everyday activities such as walking or getting in and out of a car (typically 10-50% of our maximum strength). More importantly, we need to retain control of musculoskeletal function at the lower ranges of strength if we are to successfully navigate the multitude of activities of daily living. Further following injury, surgery or other joint insults, the afferent signalling environment radically change, and thus the neuromuscular system must learn how to operate the new joint morphology. Accordingly, embodiments described herein provide for new methods of therapy (Neuro-Modulation Therapy) and for assessing neuromuscular control that focus on the use of feedforward exercises to improve neuromuscular control. That is rather than just repeating exercises to regain strength, embodiments as described herein provide therapeutic methods that facilitate neuroplastic training by causing the brain to pay attention to the new sensory feedback, thus allowing the brain to relearn control of the target joint. Case studies using NMT have shown significant improvements in patients who have not responded to traditional rehabilitation.

More generally embodiments of the NMT method may be used to treat arthrogenic muscle inhibition (AMI), increase recruitment of muscle motor units, assist with improve balance and reduce the risk of falls, reduce muscle fatigue (and the cognitive fatigue typically present with someone suffering the aforementioned issues), shift workers and operators of precision or heavy equipment, as well as optimising the performance of people requiring high levels of physical control such as athletes and dancers. NMT may be used to assist or speed recovery from a range of treatments including Cartilage repair, Synvisc injections (synthetic viscous fluids injected into a joint), and other surgical treatments. Further NMT may be used to assess the progress of recovery from such treatments. Further, by repeatedly performing embodiments of the method to assess neuromuscular control at different times, the progression of neurodegenerative disorders such as Parkinson's disease, Duchenne Muscular Dystrophy (DMD), Multiple Sclerosis (MS), Amyotrophic Lateral Sclerosis (ALS) and many other debilitating conditions such as stroke, can be tracked enabling assessment of the effects of treatments (including rehabilitation programs). Such treatments can also be used to slow or ameliorate the effects of these diseases. Similarly, embodiments can also be used to assess whether the user is suffering from a mild cognitive impairment such as a concussion, as well as the extent to which the user is suffering cognitive impairment, for example by comparing the differences in neuromuscular control obtained when the user is under a varying cognitive loading. Furthermore, embodiments of the present method may be used in the treatment of chronic pain and dissociative psychological disorders.

In addition to therapeutic and rehabilitation applications as described herein, neuro-modulation methods, and associated apparatus and systems, may be also used to further develop or refine a skill including for performance enhancement or performance optimisation by athletes (amateur and professional) and other persons such as dancers and surgeons requiring a high degree of precision and control of joints and muscles . . . . The methods, and associated apparatus and systems, disclosed herein are adaptable to precision training and performance optimisation, including the improvement muscular coordination and symmetry.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof. Those of skill in the art would further appreciate that the various illustrative logical blocks, modules, circuits, and algorithm steps described in connection with the embodiments disclosed herein may be implemented as electronic hardware, computer software or instructions, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The steps of a method or algorithm described in connection with the embodiments disclosed herein may be embodied directly in hardware, in a software module executed by a processor, or in a combination of the two. The processing of signals may be performed directly in hardware, in a software module executed by a processor, or in a combination of the two. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described herein, or a combination thereof. Software modules, also known as computer programs, computer codes, or instructions, may contain a number a number of source code or object code segments or instructions, and may reside in any computer readable medium such as a RAM memory, flash memory, ROM memory, EPROM memory, registers, hard disk, a removable disk, a CD-ROM, a DVD-ROM, a Blu-ray disc, or any other form of computer readable medium. In some aspects the computer-readable media may comprise non-transitory computer-readable media (e.g., tangible media). In addition, for other aspects computer-readable media may comprise transitory computer-readable media (e.g., a signal). Combinations of the above should also be included within the scope of computer-readable media. In another aspect, the computer readable medium may be integral to the processor. The processor and the computer readable medium may reside in an ASIC or related device. The software codes may be stored in a memory unit and the processor may be configured to execute them. The memory unit may be implemented within the processor or external to the processor, in which case it can be communicatively coupled to the processor via various means as is known in the art.

Further, it should be appreciated that modules and/or other appropriate means for performing the methods and techniques described herein can be downloaded and/or otherwise obtained by a computing apparatus. For example, such an apparatus can be coupled to a server to facilitate the transfer of executable electronic files and the like for performing the methods described herein. Alternatively, various methods described herein can be provided via storage apparatus (e.g., RAM, ROM, a physical storage medium such as a compact disc (CD) or floppy disk, etc.), such that a computing apparatus can obtain the various methods upon coupling or providing the storage apparatus to the apparatus. Moreover, any other suitable technique for providing the methods and techniques described herein to an apparatus can be utilized. In one form embodiments may comprise a computer program product for performing the method or operations presented herein. For example, such a computer program product may comprise a computer (or processor) readable medium having instructions stored (and/or encoded) thereon, the instructions being executable by one or more processors to perform the operations described herein. For certain aspects, the computer program product may include packaging material. The computer program product may be an “App” available at an app store for download by a user onto a mobile computing apparatus.

In one embodiment a computing apparatus comprises at least one processor and at least one memory operatively connected to the at least one processor (or one of the processors) and may comprises additional devices or apparatus, and input and output devices/apparatus (the term apparatus and device will be used interchangeably). The memory may comprise instructions to cause the processor to execute a method described herein. The processor, memory and output apparatus may be included in a smartphone, tablet computer, laptop, a desktop computer or in a customised apparatus or system including embedded or integrated computing apparatus. The computing apparatus may be a unitary computing or programmable apparatus, or a distributed apparatus comprising several components operatively (or functionally) connected via wired or wireless connections. The computing apparatus may comprise a central processing unit (CPU), comprising an Input/Output Interface, an Arithmetic and Logic Unit (ALU) and a Control Unit and Program Counter element which is in communication with input and output apparatus through an Input/Output Interface. A graphical processing unit (GPU) may also be included. A display apparatus may be used as an output apparatus, and may be a flat screen display (e.g., LCD, LED, plasma, touch screen, etc.), a projector, CRT, AR/VR glasses, etc. The computing apparatus may comprise a single CPU (core) or multiple CPU's (multiple core), or multiple processors. The computing apparatus may use a parallel processor, a vector processor, or be a distributed computing apparatus including cloud-based servers. The memory may comprise RAM and ROM components, and may be provided within or external to the apparatus, and may be used to store the operating system and additional software modules or instructions. The processor(s) may be configured to load and executed the software modules or instructions stored in the memory. Apparatus and devices, including sensing and display apparatus, may comprise unitary apparatus or devices or may be distributed apparatus or devices in which components are separated (for example based on function) and communicate over wired or wireless links.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

As used herein, the terms “analysing” and “determining” encompasses a wide variety of actions. For example, “analysing” may include calculating, computing, processing, deriving, investigating, looking up (e.g., looking up in a table, a database or another data structure), ascertaining, receiving (e.g., receiving information), accessing (e.g., accessing data in a memory), resolving, selecting, choosing, establishing and the like. These actions may be performed by a suitably configured computer apparatus or processor acting on instructions in software code.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

In some cases, a single embodiment may, for succinctness and/or to assist in understanding the scope of the disclosure, combine multiple features. It is to be understood that in such a case, these multiple features may be provided separately (in separate embodiments), or in any other suitable combination. Alternatively, where separate features are described in separate embodiments, these separate features may be combined into a single embodiment unless otherwise stated or implied. This also applies to the claims which can be recombined in any combination. That is a claim may be amended to include a feature defined in any other claim. Further a phrase referring to “at least one of” a list of items refers to any combination of those items, including single members. As an example, “at least one of: a, b, or c” is intended to cover: a, b, c, a-b, a-c, b-c, and a-b-c.

It will be appreciated by those skilled in the art that the disclosure is not restricted in its use to the particular application or applications described. Neither is the present disclosure restricted in its preferred embodiment with regard to the particular elements and/or features described or depicted herein.

It will be appreciated that the disclosure is not limited to the embodiment or embodiments disclosed, but is capable of numerous rearrangements, modifications and substitutions without departing from the scope as set forth and defined by the following claims.

1. A method of Neuro-Modulation Therapy (NMT) comprising:
instructing a user, and/or using an apparatus to guide a user, to perform a plurality of exercises involving at least one target musculoskeletal joint, wherein the plurality of exercises comprises one or more feedforward exercises;
providing a representation of one or more target parameters for at least one of the plurality of exercises wherein the at least one of the plurality of exercises includes the one or more feedforward exercises, and the representation is provided for one or more representation time periods over a duration of the respective exercise, and during a feedforward exercise the representation is suppressed for at least a portion of the one or more representation time periods over the duration of the feedforward exercise;
measuring one or more target parameters for one or more capture time periods whilst the user is performing one or more of the plurality of exercises; and
providing a feedback representation to the user using the measured one or more target parameters.

2. The method claimed in claim 1, wherein the user is instructed to attempt to match the one or more target parameters in real time for the one or more representation time periods and each exercise comprises one or more actions, and each representation time period is the time to perform at least one complete action.

3. The method as claimed in claim 1, wherein the at least one of the plurality of exercises further comprises at least one feedback exercise and during a feedback exercise the representation is provided for all of the one or more representation time periods over the duration of the respective feedback exercise.

4. The method as claimed in claim 1, further comprising performing an assessment of a neuromuscular control of the at least one target musculoskeletal joint during one or more exercises of the plurality of exercises comprising:
determining one or more differences between the one or more target parameters and the respective measured parameter over the one or more capture time periods.

5. The method as claimed in claim 4 wherein the assessment is used to trigger a change in the portion of the one or more representation time periods that the representation is suppressed during a feedforward exercise to change a phase, a duration or a complexity of the suppression of the representation.

6. (canceled)

7. (canceled)

8. The method as claimed in claim 4, further comprising.
electronically reporting the assessment of the neuromuscular control of the at least one target musculoskeletal joint.

9. (canceled)

10. (canceled)

11. The method as claimed in claim 1, wherein the plurality of exercises are divided into a plurality of sessions each comprising one or more sets of one or more exercises.

12. The method as claimed in claim 11 wherein, over time, the one or more exercises in a session transition from at least 80% feedback exercises in a session to at least 80% feedforward exercises in a session, and an amount of suppression and a complexity of suppression in feedforward exercises is also increased over time.

13. (canceled)

14. (canceled)

15. (canceled)

16. (canceled)

17. (canceled)

18. (canceled)

19. (canceled)

20. The method as claimed in claim 1, wherein each exercise comprises one or more actions wherein each action is either a dynamic action comprising activating one or more muscles associated with the at least one target musculoskeletal joint to move the at least one target musculoskeletal joint over one or more joint angle ranges or a static action comprising activating one or more muscles associated with the at least one target musculoskeletal joint whilst holding the at least one target musculoskeletal joint in a static position at a fixed joint angle.

21. The method as claimed in claim 1, wherein the target parameter is varied over at least one of the one or more representation time periods.

22. (canceled)

23. (canceled)

24. (canceled)

25. The method as claimed in claim 1, wherein the target parameter varies predictably in one or more exercises, and the representation is suppressed for one or more suppression time periods of the one or more representation time periods.

26. The method as claimed in claim 25 wherein the one or more exercises comprises a plurality of exercises, and for each sequential exercise in the plurality of exercises the representation is suppressed according to a sequence of suppressions, wherein each suppression in the sequence of suppressions comprises a different time period, or a progressively longer suppression time period, or an increase in complexity.

27. (canceled)

28. (canceled)

29. The method as claimed in claim 1, wherein during at least one exercise of the plurality of exercises, the representation of the target parameter is a visual representation that varies unpredictably, and the representation of the target parameter is displayed for a short time window in advance of a current time.

30. The method as claimed in claim 29, wherein for a first group of one or more sets of one or more exercises, the representation of the one or more target parameters is a visual representation that varies unpredictably, and a target force level is displayed for a short time window in advance of a current time, and for a subsequent group of one or more sets of one or more exercises, the representation of the target parameter varies predictably and/or is displayed for a long time window in advance of the current time.

31. The method as claimed in claim 1, wherein the feedback representation comprises a representation of the measured target parameter in real-time.

32. (canceled)

33. (canceled)

34. (canceled)

35. The method as claimed in claim 1, wherein the one or more of the plurality of exercises are performed by the user by using an NMT apparatus comprising a resistive element, and a sensing apparatus configured to measure the one or more target parameters as a function of time for the one or more capture time periods whilst the user is performing the one or more exercises and a computing apparatus is configured to perform the steps of instructing the user, providing a representation of a target parameter, providing a feedback representation and determining one or more differences, wherein the computing apparatus is in communication with the sensing apparatus which provides the measurements of the one or more target parameters.

36. The method as claimed in claim 35, wherein the computing apparatus is a mobile computing apparatus comprising a display apparatus which is configured to display the one or more target parameters as a function of time and is configured to receive measurements from the sensing apparatus in real-time and is configured to display the feedback representation using the display apparatus.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. The method as claimed in claim 1, wherein the NMT is provided as a treatment for one or more conditions including Arthrogenic Muscle Inhibition (AMI), stroke, a neurological disorder, a mild cognitive impairment, a balance condition, chronic pain, dissociative disorders, neuro-immune disorders, or as a post-treatment rehabilitation program following treatment of at least one target musculoskeletal joint.

60. A non-transitory computer program product comprising instructions that, when executed by a computer apparatus comprising at least one processor, at least one memory, and a communications interface, cause the at least processor to:
instruct a user, and/or using an apparatus to guide a user, to perform a plurality of exercises involving at least one target musculoskeletal joint, wherein the plurality of exercises comprises one or more feedforward exercises;
provide a representation of one or more target parameters for at least one of the plurality of exercises wherein the at least one of the plurality of exercises includes the one or more feedforward exercises, and the representation is provided for one or more representation time periods over a duration of the respective exercise, and during a feedforward exercise the representation is suppressed for at least a portion of the one or more representation time periods over the duration of the feedforward exercise;
measure one or more target parameters for one or more capture time periods whilst the user is performing one or more of the plurality of exercises; and
provide a feedback representation to the user using the measured one or more target parameters.

61. A Neuro-Modulation Therapy (NMT) system comprising:
a neuromuscular therapy apparatus comprising at least one resistive element;
a sensing apparatus comprising at least one sensor configured to measure one or more target parameters when a user is using the neuromuscular therapy apparatus;
one or more output apparatus configured to output a representation of one or more of the one or more target parameters;
a computing apparatus comprising at least one processor, at least one memory, and a communications interface, wherein the communications interface is configured to receive a measurement of the one or more target parameters from the sensing apparatus and at least one processor is configured to control the one or more output apparatus and wherein the at least one memory comprises instructions for configuring the processor to:
instruct a user to perform a plurality of exercises involving at least one target musculoskeletal joint, wherein the plurality of exercises comprises one or more feedforward exercises;
providing, using the one or more output apparatus, the representation of the one or more target parameters for at least one of the plurality of exercises wherein the at least one of the plurality of exercises includes the one or more feedforward exercises, and the representation is provided for one or more representation time periods over a duration of the respective exercise, and during a feedforward exercise the representation is suppressed for at least a portion of the one or more representation time periods over the duration of the feedforward exercise;
measuring, using the sensing apparatus, one or more target parameters for one or more capture time periods whilst the user is performing one or more of the plurality of exercises; and
providing a feedback representation to the user using the measured one or more target parameters.

62. The system as claimed in claim 61, wherein the computing apparatus is a mobile computing apparatus comprising a display apparatus which is configured to display a representation of the one or more target parameters as a function of time and the mobile computing apparatus is configured to receive measurements from the sensing apparatus in real-time and is configured to display the feedback representation using the display apparatus.

63. The system as claimed in claim 61, wherein the neuromuscular therapy apparatus is an extension apparatus and the target parameter is force, and the force is measured by one or more load cells which are connected to the at least one resistive component and the extension apparatus is manufactured from substantially stiff or rigid components to maximise the force transmitted to the one or more load cells when an exercise is performed.

64. The system as claimed in claim 61, wherein the at least one sensor is at least one portable load cell and the communications interface is a wireless communications module, and the system further comprises an attachment arrangement configured to allow removable attachment of the sensing apparatus to the neuromuscular therapy apparatus and the neuromuscular therapy apparatus comprises one or more movable surfaces such that in use movement of one or more movable surfaces generates a load on at least one of the at least one portable load cell apparatus.

65. The system as claimed in claim 61, wherein the sensing apparatus comprises a force sensing apparatus comprising a force sensor, an inertial measurement unit (IMU), and a communications module which is configured to wirelessly transmit measured force data and position data from the IMU to a computing apparatus to estimate the applied force and joint angle as a function of time whilst performing the exercise.

66. The system as claimed in claim 61, wherein the target parameter is force, and the force is measured by one or more load cells which are connected to the at least one resistive component.

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. A Neuro-Modulation Therapy (NMT) apparatus comprising:
at least one resistive element;
a sensing apparatus comprising at least one sensor configured to measuring one or more target parameters when a user is using the NMT apparatus, wherein the sensing apparatus is configured to provide a measurement of the one or more target parameters to a computing apparatus comprising at least one processor, at least one memory, and a communications interface, wherein the computing apparatus is operatively connected to or integrates one or more output apparatus configured to output a representation of the one or more target parameters and the at least one memory comprises instructions for configuring the at least one processor to:
instruct a user to perform a plurality of exercises involving at least one target musculoskeletal joint, wherein the plurality of exercises comprises one or more feedforward exercises;
provide, using the one or more output apparatus, the representation of the one or more target parameters for at least one of the plurality of exercises wherein the at least one of the plurality of exercises includes the one or more feedforward exercises, and the representation is provided for one or more representation time periods over a duration of the respective exercise, and during a feedforward exercise the representation is suppressed for at least a portion of the one or more representation time periods over the duration of the feedforward exercise;
measure, using the sensing apparatus, one or more target parameters for one or more capture time periods whilst the user is performing one or more of the plurality of exercises; and
provide a feedback representation to the user using the measured one or more target parameters.

79. The apparatus as claimed in claim 78 wherein the NMT apparatus is an extension apparatus and the target parameter is force, and the force is measured by a load cell which is connected to the at least one resistive component and the extension apparatus is manufactured from substantially stiff or rigid components to maximise the force transmitted to the load cell when an exercise is performed.

80. The apparatus as claimed in claim 78, wherein the at least one sensor is a portable load cell and the communications interface is a wireless communications module, and further comprises an attachment arrangement configured to allow removable attachment of the sensing apparatus to the NMT apparatus and the NMT apparatus comprises one or more movable surfaces such that in use movement of one or more movable surfaces generates a load on the portable load cell apparatus.

81. The apparatus as claimed in claim 78, wherein the sensing apparatus comprises a force sensing apparatus comprising a force sensor, an inertial measurement unit (IMU), and a communications module which is configured to wirelessly transmit measured force data and position data from the IMU to a computing apparatus to estimate the applied force and joint angle as a function of time whilst performing the exercise.

82. The apparatus as claimed in claim 78, wherein the target parameter is force, and the force is measured by a load cell which is connected to the at least one resistive component.

83. (canceled)

84. (canceled)

85. (canceled)

86. The Neuro-Modulation Therapy (NMT) apparatus as claimed in claim 78, further comprising:
a computer program product comprising instructions for causing a processor to:
instruct a user to perform a plurality of exercises involving at least one target musculoskeletal joint, wherein the plurality of exercises comprises one or more feedforward exercises;
provide, using the one or more output apparatus, the representation of the one or more target parameters for at least one of the plurality of exercises wherein the at least one of the plurality of exercises includes the one or more feedforward exercises, and the representation is provided for one or more representation time periods over a duration of the respective exercise, and during a feedforward exercise the representation is suppressed for at least a portion of the one or more representation time periods over the duration of the feedforward exercise;
measure, using the sensing apparatus, one or more target parameters for one or more capture time periods whilst the user is performing one or more of the plurality of exercises; and
provide a feedback representation to the user using the measured one or more target parameters.
